(19) United States
(12) Patent Application Publication
(10) Pub. No.: US 2026/0265360 A1
(43) Pub. Date: Sep. 10, 2026

(54) USE OF ANTI-FAM19A5 ANTAGONISTS FOR TREATING HEARING DISORDERS

(71) Applicant: NEURACLE SCIENCE CO., LTD., Seoul (KR)

(72) Inventors: Jae Young SEONG, Seoul (KR); Yosub PARK, Seoul (KR); Jinwoong BOK, Bucheon-si (KR); Hyehyun MIN, Seoul (KR); Han Byul KIM, Seoul (KR)

(73) Assignee: NEURACLE SCIENCE CO., LTD., Seoul (KR)

(21) Appl. No.: 18/721,442

(22) PCT Filed: Feb. 21, 2024
(86) PCT No.: PCT/IB2024/051688

§ 371 Date: Jun. 18, 2024

Related U.S. Application Data
(60) Provisional application No. 63/486,187, filed on Feb. 21, 2023.
(60) Provisional application No. 63/595,942, filed on Nov. 3, 2023.

(51) Int. Cl. C07K 16/24; A61K 31/09; A61K 31/136; A61K 31/7105; A61K 31/713; A61K 38/00; A61P 27/16; C07K 7/08; C07K 14/705; C12N 15/113

(52) U.S. Cl. CPC C07K 16/24; A61K 31/09; A61K 31/136; A61K 31/7105; A61K 31/713; A61P 27/16; C07K 7/08; C07K 14/705; C12N 15/1136; A61K 38/00; C07K 2317/24; C07K 2317/565; C07K 2317/76; C07K 2317/92; C12N 2310/11; C12N 2310/141; C12N 2310/16

(57) ABSTRACT

The present disclosure relates to the use of FAM19A5 antagonists (e.g., anti-FAM19A5 antibody or LRRC4 family mimic molecules) to treat a wide range of hearing disorders. The present disclosure also provides methods of using such antagonists to enhance one or more properties of ribbon synapses.

### CROSS-REFERENCE TO RELATED APPLICATIONS

This PCT application claims the priority benefit of U.S. Provisional Application No. 63/486,187, filed on Feb. 21, 2023, and U.S. Provisional Application No. 63/595,942, filed on Nov. 3, 2023, each of which is herein incorporated by reference in its entirety.

### REFERENCE TO SEQUENCE LISTING SUBMITTED ELECTRONICALLY

The content of the sequence listing is submitted electronically (Name: 3763_022PC02_Seqlisting_ST26.xml; Size: 340,155 bytes; and Date of Creation: Feb. 20, 2024) with the application and herein incorporated by reference in its entirety.

### FIELD OF THE DISCLOSURE

The present disclosure provides methods for the treatment of hearing disorders in a subject (e.g., a human) using antagonists (e.g., antibodies or LRRC4 family mimic molecules) that specifically targets a family with sequence similarity 19, member A5 (FAM19A5) or a composition comprising such antagonists.

### BACKGROUND OF THE DISCLOSURE

Hearing impairment is a major global health issue with profound societal and economic impact affecting over 275 million people world-wide. The occurrence of hearing loss is rapidly rising, due to e.g. increasing noise exposure and aging populations. With no approved pharmaceutical therapies available today, the unmet medical need is very high. In particular there is a need for providing effective methods for prevention and subsequent treatment of hearing loss which allow for immediate as well as long term maintenance of preventive and/or therapeutic effects.

### BRIEF SUMMARY OF THE DISCLOSURE

Provided herein is method of treating and/or preventing a hearing disorder in a subject in need thereof, comprising administering to the subject an antagonist against a family with sequence similarity 19, member A5 (FAM19A5) protein (FAM19A5 antagonist). In some aspects the hearing disorder comprises a sensorineural hearing loss. In some aspects the sensorineural hearing loss is with tinnitus. In some aspects, the sensorineural hearing loss is without tinnitus. In some aspects, the sensorineural hearing loss comprises an ototoxic drug-induced hearing loss, noise-induced hearing loss, age-related hearing loss, sudden hearing loss, or combinations thereof.

Also provided herein is a method of reducing and/or preventing an impairment of ribbon synapses within an inner ear of a subject in need thereof, comprising administering to the subject an antagonist against a family with sequence similarity 19, member A5 (FAM19A5) protein (FAM19A5 antagonist). In some aspects, the impairment of ribbon synapses comprises a decrease in the number of ribbon synapses as compared to that of a corresponding subject who does not have an impairment of ribbon synapses within an inner ear (e.g., corresponding subject with normal hearing). In some aspects, the impairment of ribbon synapses comprises an abnormal function of the ribbon synapses.

Provided herein is a method of inducing a formation of ribbon synapses within an inner ear of a subject in need thereof, comprising administering to the subject an antagonist against a family with sequence similarity 19, member A5 (FAM19A5) protein (FAM19A5 antagonist).

In some aspects, after the administration, the number of ribbon synapses is increased within the inner ear of the subject, as compared to a reference subject (e.g., the subject prior to the administration and/or a corresponding subject who did not receive the administration). In some aspects, the number of ribbon synapses is increased by at least about 1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold fold, or at least about 50-fold, as compared to the reference subject.

Provided herein is a method of improving a function of ribbon synapses within an inner ear of a subject in need thereof, comprising administering to the subject an antagonist against a family with sequence similarity 19, member A5 (FAM19A5) protein (FAM19A5 antagonist).

In some aspects, the function comprises the ability to release one or more neurotransmitters in response to a signal from an inner hair cell. In some aspects, after the administration, the function of the ribbon synapses is improved by at least about 1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold fold, or at least about 50-fold.

Also provided herein is a method of reducing an expression and/or activity of a family with sequence similarity 19, member A5 (FAM19A5) protein within a spiral ganglion neuron of a subject in need thereof, comprising administering to the subject an antagonist against the FAM19A5 protein (FAM19A5 antagonist). In some aspects, after the administration, the expression and/or activity of the FAM19A5 protein within the spiral ganglion neuron of the subject is decreased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

For the methods provided above, in some aspects, the subject suffers from a hearing disorder. In some aspects, the hearing disorder comprises a sensorineural hearing loss. In some aspects, the sensorineural hearing loss is with tinnitus. In some aspects, the sensorineural hearing loss is without tinnitus. In some aspects, the sensorineural hearing loss comprises an ototoxic drug-induced hearing loss, noise-induced hearing loss, age-related hearing loss, sudden hearing loss, or combinations thereof.

For any of the methods provided herein, in some aspects, the FAM19A5 antagonist comprises: (i) an antisense-oligonucleotide, siRNA, shRNA, miRNA, dsRNA, aptamer, PNA (peptide nucleic acid), or a vector including the same; (ii) an antibody or an antigen-binding fragment thereof that specifically binds to the FAM19A5 protein (“anti-FAM19A5 antibody); (iii) a polynucleotide encoding the anti-FAM19A5 antibody; (iv) a leucine rich repeat containing 4 (LRRC4) family mimic molecule; (v) a LRRC4 family peptide; or (vi) any combination of (i) to (v).

In some aspects, the anti-FAM19A5 antibody exhibits a property selected from the group consisting of: (a) binds to soluble human FAM19A5 with a K_{D} of 10 nM or less as measured by enzyme-linked immunosorbent assay (ELISA); (b) binds to membrane bound human FAM19A5 with a K_{D} of 10 nM or less as measured by ELISA; and (c) both (a) and (b).

In some aspects, the anti-FAM19A5 antibody cross-competes for binding to a human FAM19A5 epitope with a reference antibody, wherein the reference antibody is selected from the group consisting of the antibodies in Tables 2-5. In some aspects, the anti-FAM19A5 antibody comprises a heavy chain CDR1, CDR2, and CDR3, and a light chain CDR1, CDR2, and CDR3, wherein (i) the heavy chain CDR1 comprises a CDR1 selected from the group consisting of CDR1s in Table 6; (ii) the heavy chain CDR2 comprises a CDR2 selected from the group consisting of CDR2s in Table 6; (iii) the heavy chain CDR3 comprises a CDR3 selected from the group consisting of CDR3s in Table 6; (iv) the light chain CDR1 comprises a CDR1 selected from the group consisting of CDR1s in Table 7; (v) the light chain CDR2 comprises a CDR2 selected from the group consisting of CDR2s in Table 7; and/or (vi) the light chain CDR3 comprises a CDR3 selected from the group consisting of CDR3s in Table 7. In some aspects, (i) the heavy chain CDR1 comprises the sequence set forth in SEQ ID NO: 220, (ii) the heavy chain CDR2 comprises the sequence set forth in SEQ ID NO: 221, (iii) the heavy chain CDR3 comprises the sequence set forth in SEQ ID NO: 16, (iv) the light chain CDR1 comprises the sequence set forth in SEQ ID NO: 225, (v) the light chain CDR2 comprises the sequence set forth in SEQ ID NO: 228, and (vi) the light chain CDR3 comprises the sequence set forth in SEQ ID NO: 227.

In some aspects, the anti-FAM19A5 antibody comprises a heavy chain variable region (VH) and a light chain variable region (VL), wherein (i) the VH comprises a VH selected from the group consisting of VHs in Table 8; and/or (ii) the VL comprises a VL selected from the group consisting of VLs in Table 9. In some aspects, the VH comprises the sequence set forth in SEQ ID NO: 235 and the VL comprises the sequence set forth in SEQ ID NO: 240.

In some aspects, the anti-FAM19A5 antibody comprises a Fab, Fab′, F(ab′) 2, Fv, or single chain Fv (scFv). In some aspects, the anti-FAM19A5 antibody is selected from the group consisting of an IgG1, an IgG2, an IgG3, an IgG4, and a variant thereof. In some aspects, the anti-FAM19A5 antibody is an IgG2, an IgG4, or a combination thereof. In some aspects, the anti-FAM19A5 antibody comprises an IgG2/lgG4 isotype antibody. In some aspects, the anti-FAM19A5 antibody is a chimeric antibody, a human antibody, or a humanized antibody.

For any of the methods provided herein, in some aspects, the FAM19A5 antagonist is a LRRC4 family mimic molecule, wherein the LRRC4 family mimic molecule is capable of inhibiting, reducing, and/or dissociating an interaction between the FAM19A5 protein and a member of a LRRC4 protein family. In some aspects, the LRRC4 family mimic molecule is not an antibody or an antigen-binding fragment thereof. In some aspects, the LRRC4 family mimic molecule comprises a polypeptide. In some aspects, the LRRC4 family mimic molecule comprises a small molecule.

In some aspects, the LRRC4 family mimic molecule is a small molecule of formula (I):

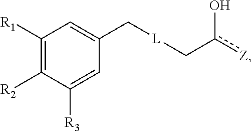

or a pharmaceutically acceptable salt thereof, wherein:
(i) R₁, R₂ and R₃ are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N,N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl;
(ii) [symbol] is a single or double bond;
(iii) Z is selected from a straight chain or branched (C₁-C₈)alkyl, a straight chain or branched (C₂-C₈)alkenyl, a straight chain or branched (C₂-C₈)alkynyl, (C₃-C₈)cycloalkyl, (C₅-C₈)cycloalkenyl, (3-8 membered)heterocycloalkyl, (C₇-C₁₄)bicycloalkyl, (C₇-C₁₄)bicycloalkenyl, (7-14 membered) heterobicycloalkyl, (C₆-C₁₀)aryl, (5-10-membered)heteroaryl, and —CH—C(O)—CH═CH-Q, wherein Q is (C₃-C₈)cycloalkyl, (C₅-C₈)cycloalkenyl, (3-8 membered)heterocycloalkyl, (C₆-C₁₀)aryl, and (5-6-membered)heteroaryl; wherein each cycloalkyl, cycloalkenyl, heterocyclylalkyl, aryl, and heteroaryl are optionally substituted with one, two, three, four, or five substituents independently selected from (C₁-C₆)alkoxy, (C₁-C₆)alkyl, halo, (C₁-C₆)haloalkoxy, nitro, amino, N-methylamino, N-ethylamino, N—N-propylamino, N,N-dimethylamino, formyl, and hydroxy, and
(iv) L is single, double or triple bond, and
wherein the LRRC4 family mimic molecule is not a small molecule selected from:

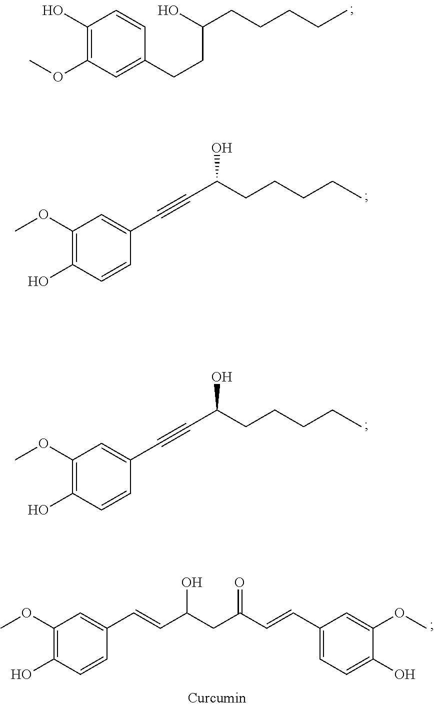

or a pharmaceutically acceptable salt thereof.

In some aspects, the LRRC4 family mimic molecule is a small molecule of formula (II):

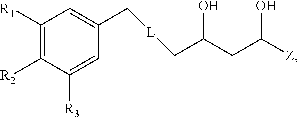

or a pharmaceutically acceptable salt thereof, wherein:
(i) R1, R2 and R3 are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N,N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl,
(ii) Z is selected from a straight chain or branched (C₁-C₈)alkyl, a straight chain or branched (C₂-C₈)alkenyl, a straight chain or branched (C₂-C₈)alkynyl, (C₃-C₈)cycloalkyl, (C₅-C₈)cycloalkenyl, (3-8 membered) heterocycloalkyl, (C₇-C₁₄)bicycloalkyl, (C₇-C₁₄)bicycloalkenyl, (7-14 membered)heterobicycloalkyl, (C₆-C₁₀)aryl, (5-10-membered)heteroaryl, and —CH═CH-Q, wherein Q is (C₃-C₈)cycloalkyl, (C₅-C₈)cycloalkenyl, (3-8 membered)heterocycloalkyl, (C₆-C₁₀)aryl, and (5-6-membered)heteroaryl; wherein each cycloalkyl, cycloalkenyl, heterocyclylalkyl, aryl, and heteroaryl are optionally substituted with one, two, three, four, or five substituents independently selected from (C₁-C₆)alkoxy, (C₁-C₆)alkyl, halo, (C₁-C₆)haloalkoxy, nitro, amino, N-methylamino, N-ethylamino, N—N-propylamino, N,N-dimethylamino, formyl, and hydroxy, and
(iii) L is single, double or triple bond.

In some aspects, the LRRC4 family mimic molecule is selected from:

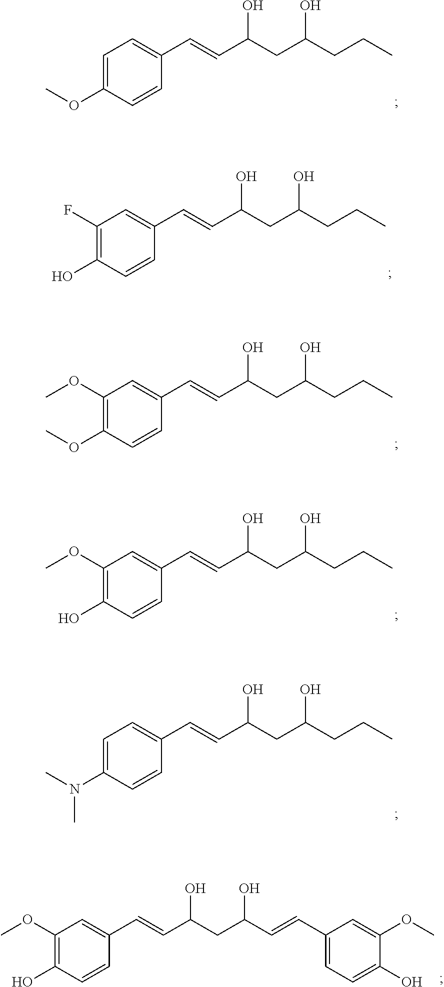

or a pharmaceutically acceptable salt thereof.

In some aspects, wherein the LRRC4 family mimic molecule is a small molecule of formula (III):

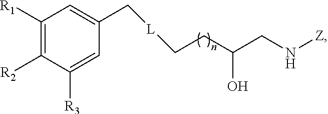

or a pharmaceutically acceptable salt thereof, wherein:
(i) R1, R2 and R3 are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N, N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl,
(ii) Z is selected from a straight chain or branched (C₁-C₈)alkyl, a straight chain or branched (C₂-C₈)alkenyl, a straight chain or branched (C₂-C₈)alkynyl, —Y—(C₃-C₈)cycloalkyl, —Y—(C₅-C₈)cycloalkenyl, —Y-(3-8 membered)heterocycloalkyl, —Y—(C₇-C₁₄)bicycloalkyl, —Y—(C₇-C₁₄)bicycloalkenyl, —Y-(7-14 membered) heterobicycloalkyl, —Y—(C₆-C₁₀)aryl, and —Y-(5-10-membered)heteroaryl, wherein Y is a bond or a C₁-C₃ straight or branched alkylene, and wherein the cycloalkyl, the cycloalkenyl, the heterocyclylalkyl, the aryl, and the heteroaryl are optionally substituted with one, two, three, four, or five substituents independently selected from C₁-C₆alkoxy, C₁-C₆alkyl, halo, C₁-C₆haloalkoxy, nitro, amino, N-methylamino, N-ethylamino, N—N-propylamino, N,N-dimethylamino, formyl, and hydroxy,
(iii) L is single, double or triple bond, and
(iv) n is 0 or 1.

In some aspects, the LRRC4 family mimic molecule is selected from:

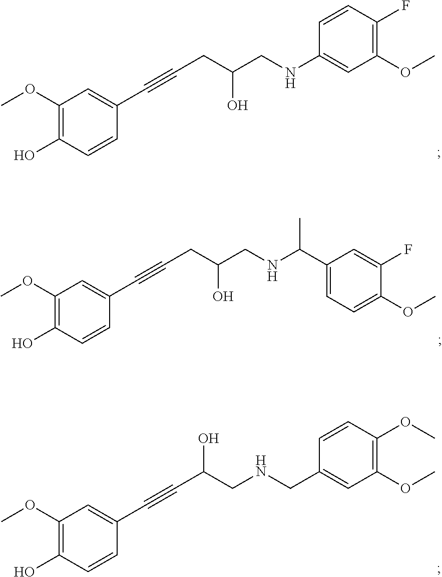

or a pharmaceutically acceptable salt thereof.

In some aspects, the LRRC4 family mimic molecule is a polypeptide comprising, consisting of, or consisting essentially of a domain of a LRRC4 protein family member, wherein the domain is capable of binding to the FAM19A5 protein, and wherein the polypeptide is shorter than the corresponding full-length LRRC4 protein family member (SEQ ID NO: 6; SEQ ID NO: 9; or SEQ ID NO: 84).

In some aspects, the FAM19A5 binding domain is about 10 to about 23 amino acids in length.

In some aspects, the LRRC4 family mimic molecule comprises an amino acid sequence having the following formula (from N-terminus to C-terminus):

$$A - ( T / S ) - B , \qquad ( Formula IV )$$

(i) A comprises X1-(T/S)-(Y/F)—F-X5; wherein:
X1 is tyrosine (Y), phenylalanine (F), valine (V), leucine (L), or isoleucine (I);
(T/S) is threonine (T) or serine(S);
(Y/F) is tyrosine (Y) or Phenylalanine (F); and
X5 is any amino acid; and
(ii) B comprises (V/I)-T-V-(E/V); wherein:
(V/I) is valine (V) or isoleucine (I); and
(E/V) is glutamic acid (E) or valine (V).

In some aspects, the LRRC4 family mimic molecule comprises an amino acid sequence having the following formula (from N-terminus to C-terminus):

$$A - ( T / S ) - B , \qquad ( Formula IV )$$

(i) A comprises (Y/W/M)-(T/Y)-(Y/W)-(F/Y/W)-(T/Y); wherein:
(Y/W/M) is tyrosine (Y), tryptophan (W), or methionine (M);
(T/Y) is threonine (T) or tyrosine (Y);
(Y/W) is tyrosine (Y) or tryptophan (W); and
(F/Y/W) is phenylalanine (F), tyrosine (Y), or tryptophan (W); and
(ii) B comprises X7-(T/S/Y)-X9-X10; wherein:
X7 is valine (V), tyrosine (Y), phenylalanine (F), leucine (L), tryptophan (W), or methionine (M);
(T/S/Y) is threonine (T), serine(S), or tyrosine (Y);
X9 is valine (V), isoleucine (I), tyrosine (Y), phenylalanine (F), leucine (L), tryptophan (W), or methionine (M); and
X10 is glutamic acid (E), aspartic acid (D), isoleucine (I), tyrosine (Y), phenylalanine (F), methionine (M), or tryptophan (W).

In some aspects, the LRRC4 family mimic molecule comprises an amino sequence having the following formula (from N-terminus to C-terminus):

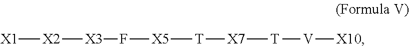

X1 is Y, F, V, L, or I;
X2 is T or S;
X3 is Y or F;
X5 is any amino acid;
X7 is V or I; and/or
X10 is E or V.

In some aspects, LRRC4 family mimic molecule comprises an amino acid sequence having the following formula: (from N-terminus to C-terminus):

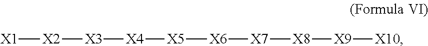

X1 is Y, F, V, L, I, W, or M;
X2 is T, S, or Y;
X3 is Y, F, or W;
X4 is F, Y, or W;
X5 is any amino acids, e.g., T, S, or Y;
X6 is T, S, or Y;
X7 is V, I, Y, F, L, W, or M;
X8 is T, S, or Y;
X9 is V, I, Y, F, L, W, or M; and/or
X10 is E, D, V, I, Y, F, M, or W.

In some aspects, X1 is Y, F, V, L, or I. In some aspects, X2 is T or S. In some aspects, X3 is Y or F. In some aspects, X4 is F. In some aspects, X5 is T or S. In some aspects, X6 is T. In some aspects, X7 is V or I. In some aspects, X8 is T. In some aspects, X9 is V. In some aspects, X10 is E or V. In some aspects, the amino acid at X2 is phosphorylated or O-glycosylated.

In some aspects, the LRRC4 family mimic molecule comprises, consists essentially of, or consists of the amino acid sequence set forth in SEQ ID NO: 48 (GYTYFTTVTVETLETQ). In some aspects, the LRRC4 family mimic molecule comprises, consists essentially of, or consists of the amino acid sequence set forth in SEQ ID NO: 47 (GYTYFTTVTVETLETQPGEE). In some aspects, the LRRC4 family mimic molecule comprises, consists essentially of, or comprises the amino acid sequence set forth in SEQ ID NO: 49 (GYTYFTTVTVETLETQPGEKEPPGPTTD). In some aspects, the LRRC4 family mimic molecule comprises, consists essentially of, or consists of the amino acid sequence set forth in SEQ ID NO: 20 (NYSFFTTVTVETTEISPEDTTRK). In some aspects, the LRRC4 family mimic molecule comprises, consists essentially of, or consists of the amino acid sequence set forth in SEQ ID NO: 78 (NFSYFSTVTVETMEPSQDERTTR). In some aspects, the LRRC4 family mimic molecule comprises, consists essentially of, or consists of the amino acid sequence set forth in SEQ ID NO: 44 (YTYFTTVTVE). In some aspects, the LRRC4 family mimic molecule comprises, consists essentially of, or consists of the amino acid sequence set forth in SEQ ID NO: 45 (YSFFTTVTVE). In some aspects, the LRRC4 family mimic molecule comprises, consists essentially of, or consists of the amino acid sequence set forth in SEQ ID NO: 46 (FSYFSTVTVE). In some aspects, the LRRC4 family mimic molecule comprises, consists essentially of, or consists of the amino acid sequence set forth in any one of SEQ ID NOs: 44-80. In some aspects, one or more of the amino acid residues are in the form of a D-amino acid.

For any of the methods provided herein, in some aspects, the FAM19A5 antagonist is administered to the subject via intracochlear injection, intravestibular injection, intravenous administration, or intratympanic administration. In some aspects, the subject is a human. For any of the methods provided herein, in some aspects, the method further comprises administering an additional therapeutic agent to the subject.

Some aspects of the present disclosure relates to means for treating a hearing disorder in a subject in need thereof comprising a pharmaceutical composition which comprises an antagonist against a family with sequence similarity 19, member A5 (FAM19A5) protein (FAM19A5 antagonist) and a pharmaceutically acceptable carrier. In some aspects, the hearing disorder comprises a sensorineural hearing loss. In some aspects, the sensorineural hearing loss is with tinnitus or without tinnitus. In some aspects, the sensorineural hearing loss comprises an ototoxic drug-induced hearing loss, noise-induced hearing loss, age-related hearing loss, sudden hearing loss, or combinations thereof. In some aspects, the FAM19A5 antagonist comprises: (i) an antisense-oligonucleotide, siRNA, shRNA, miRNA, dsRNA, aptamer, PNA (peptide nucleic acid), or a vector including the same; (ii) an antibody or an antigen-binding fragment thereof that specifically binds to the FAM19A5 protein (“anti-FAM19A5 antibody); (iii) a polynucleotide encoding the anti-FAM19A5 antibody; (iv) a leucine rich repeat containing 4 (LRRC4) family mimic molecule; (v) a LRRC4 family peptide; or (vi) any combination of (i) to (v).

Some aspects of the present disclosure relates to a method of treating a hearing disorder in a subject in need thereof, comprising administering to the subject means for antagonizing a family with sequence similarity 19, member A5 (FAM19A5) protein and a pharmaceutically acceptable carrier. In some aspects, the hearing disorder comprises a sensorineural hearing loss. In some aspects, the sensorineural hearing loss is with tinnitus or without tinnitus. In some aspects, the sensorineural hearing loss comprises an ototoxic drug-induced hearing loss, noise-induced hearing loss, age-related hearing loss, sudden hearing loss, or combinations thereof. In some aspects, the means comprise an antagonist against the FAM19A5 protein. In some aspects, the FAM19A5 antagonist comprises: (i) an antisense-oligonucleotide, siRNA, shRNA, miRNA, dsRNA, aptamer, PNA (peptide nucleic acid), or a vector including the same; (ii) an antibody or an antigen-binding fragment thereof that specifically binds to the FAM19A5 protein (“anti-FAM19A5 antibody); (iii) a polynucleotide encoding the anti-FAM19A5 antibody; (iv) a leucine rich repeat containing 4 (LRRC4) family mimic molecule; (v) a LRRC4 family peptide; or (vi) any combination of (i) to (v).

### BRIEF DESCRIPTION OF THE DRAWINGS

FIG. 1 shows a schematic of the cochlear sensory epithelium with the inner and outer hair cells and their afferent innervations as they appear in tissue immunostained for neurofilaments and synaptic ribbon proteins. Inner hair cells (IHCs) act as mechanoelectric transducers, releasing neurotransmitter to excite the sensory fibers of the cochlear nerve, whereas outer hair cells (OHCs) act as biological motors to amplify motion of the sensory epithelium.

FIG. 2 shows FAM19A5 gene expression in spiral ganglion neurons at embryonic day 15.5 (E15.5) in mice as measured using in situ hybridization (see bottom row, right image). For comparison, the expression of the following genes within the same spiral ganglion neurons are also provided: Atoh1 (hair cell marker; see top row, left image), Nefl (spiral ganglion neuron marker; see top row, right image), LRRC4B (see bottom row, left image), and LRRC4C (see bottom row, middle image).

FIG. 3 shows FAM19A5 gene expression in spiral ganglion neurons in postnatal day 7 (P7) mice (see top and bottom rows, right most images) as measured using in situ hybridization at 10× magnification (top row) and 20× magnification (bottom row). For comparison, the expression of Nefl (spiral ganglion neuron marker, LRRC4B and LRRC4C genes are also provided.

FIG. 4 shows the expression of FAM19A5 and LRRC4B genes (as measured using in situ hybridization) in spiral ganglion neurons at P7 mice. The expression of the genes are shown at 10× (top box) and 20× magnifications (bottom box).

FIGS. 5A, 5B, and 5C show the effect of FAM19A5 antagonist administration on hearing ability in mice. The effect on hearing was assessed by measuring auditory brainstem response (ABR) generated after FAM19A5 antagonist administration. FIG. 5A provides a schematic of the experimental design. As shown, anti-FAM19A5 antibody (1-30) or control human IgG antibody was administered to the mice intravenously once a week (30 mg/kg per dose). ABR (i.e., hearing test) was measured prior to the initial administration of the antibodies and then at day 1, week 2, and 8 week post antibody administration. FIG. 5B provides comparison of ABR threshold in the mice prior to the initial administration of the anti-FAM19A5 antibody (box) or the control IgG antibody (circle). FIG. 5C provides comparison of ABR threshold in the mice at two weeks post initial anti-FAM19A5 antibody (box) or control IgG antibody (circle) administration. In both FIGS. 5B and 5C, the ABR threshold was measured at a frequency ranging from 0 to 64 kHz.

FIGS. 6A, 6B, 6C, 6D, and 6E show the therapeutic effect of FAM19A5 antagonists on auditory brainstem response (ABR) threshold in a noise-induced hearing loss mouse model. FIG. 6A provides a schematic of the experimental design. As shown, anti-FAM19A5 antibody (1-30) or control human IgG antibody was administered to the mice intravenously once a week (30 mg/kg per dose) for a total of five administrations. The first dose of the antibodies was administered one day before noise exposure. For noise-induced hearing loss induction, the animals were subjected to temporary threshold shift (TTS) of 105 decibels for 30 minutes. ABR (i.e., hearing test) was measured both prior to the initial administration of the antibodies and then at various times after administration. FIGS. 6B, 6C, 6D, and 6E provide comparison of ABR threshold between mice administered with the control human IgG (circle), anti-FAM19A5 antibody (square), or before noise (triangle) at the following time points: (i) before TTS exposure, (ii) 1 day after TTS exposure, (iii) 2 weeks after TTS exposure, (iv) 4 weeks after TTS exposure, and (v) 8 weeks after TTS exposure, respectively. ABR threshold was measured at a frequency ranging from 4 kHz to 64 kHz.

FIGS. 7A, 7B, 7C, and 7D show the therapeutic effect of FAM19A5 antagonists on distortion product optoacoustic emission (DPOAE) threshold in a noise-induced hearing loss mouse model. The overall experimental design is the same as that described in FIG. 6A. DPOAE threshold data was measured after antibody administration and: (i) before TTS exposure (FIG. 7A), (ii) 1 day after TTS exposure (FIG. 7B), (iii) 2 weeks after TTS exposure (FIG. 7C), and (iv) 4 weeks after TTS exposure (FIG. 7D). DPOAE threshold was measured at a frequency ranging between about 4 kHz to about 32 kHz.

FIGS. 8A and 8B show the effect of FAM19A5 antagonists on distortion product optoacoustic emission (DPOAE) amplitude in a noise-induced hearing loss mouse model. The overall experimental design is the same as that described in FIG. 6A. DPOAE amplitude data was measured after antibody administration at 2 weeks after TTS exposure. FIG. 8A provides a comparison of the average DPOAE amplitude value as a function of different noise level for the following groups: (i) before noise (triangle), (ii) control antibody treated (circle), and (iii) anti-FAM19A5 treated (square). FIG. 8B provides a spectral level profile for each of the groups.

FIG. 9 shows the exemplary process of a stimulus originating in the inner ear cochlea, as it is transmitted to the auditory nerve, and then the signal is transmitted to the auditory cortex through the brain stem. Signal is transmitted through the auditory nerve (AN), cochlear nuclei (CN), superior olivary complex (SOC), lateral lemniscus (LL), and inferior colliculus (IC), before it reaches the auditory cortex.

FIGS. 10A and 10B provide illustrative example of how a sound wavelength is measured via signal amplitude using the auditory brainstem response (ABR). FIG. 10A shows how a sound wave length sequentially traverses from the cochlea to the auditory cortex as it is introduced to the auditory nerve (AN), Cochlear nuclei (CN), superior olivary complex (SOC), lateral lemniscus (LL), and inferior colliculus (IC), respectively. It is the amplitude and latency of the first wavelength in each of the AN, CN, SOC, LL and IC that is measured and used for ABR results. FIG. 10B provides an exemplary wave I amplitude profile as a function of noise level (i.e., tone-burst level).

FIGS. 11A and 11B show the effect of FAM19A5 antagonists on ABR I/O function in a noise-induced hearing loss mouse model. The overall experimental design is the same as that described in FIG. 6A. FIG. 11A provides wave I amplitude before noise (left graph) and at 2 weeks after noise (right graph).

FIG. 11B provides wave I amplitude at 4 weeks after noise. For each of FIGS. 11A and 11B, the following groups of animals are shown: (i) control antibody treated (circle) and (ii) anti-FAM19A5 antibody treated (square). For comparison purposes, wave I amplitude before noise exposure is also provided at the 2 weeks and 4 weeks timepoints (“BN”).

FIGS. 12A and 12B provide immunohistochemistry (IHC) analysis of the therapeutic effects of anti-FAM19A5 antibody (1-30) and control human IgG antibody administration in the middle cochlear region 8 weeks after temporary threshold shift (TTS) (i.e., noise induction). FIG. 12A shows the increased CtBP2 presence, a marker of cochlear ribbon synapses, as outlined with a dotted circle, in the IHC following the administration of anti-FAM19A5 antibody (right figure) in comparison to the control hIgG antibody (left figure). FIG. 12B shows the increased number of ribbon bodies per cell, in the IHC (1st chart) and OHC (2^{nd} chart) following the administration of the anti-FAM19A5 antibody (second bar in each of the graphs) and in comparison to hIgG (first bar in each of the graphs) and before noise group (last bar in each of the graphs).

FIGS. 13A, 13B, 13C, 13D, and 13E show the therapeutic effect of FAM19A5 antagonists (administered 2 hours after noise induction) on auditory brainstem response (ABR) threshold in a noise-induced hearing loss mouse model. The overall design was similar as that provided in FIG. 6A, except that the antibodies were administered to the animals at 2 hours post noise induction. FIG. 13A provides the results before noise exposure. FIG. 13B provides the results at one day post noise exposure. FIG. 13C provides the results at two weeks post noise exposure. FIG. 13D provides the results at four weeks post noise exposure. FIG. 13E provides the results at eight weeks post noise exposure. ABR threshold was measured at a frequency ranging from 4 kHz to 64 kHz.

FIGS. 14A and 14B show the effect of FAM19A5 antagonists on ABR I/O function in a noise-induced hearing loss mouse model. FIG. 14A provides comparisons of wave I amplitude (1st graph) and wave IV amplitude (2^{nd} graph) in animals treated with the anti-FAM19A5 antibody (square), before noise group (triangle) or control IgG antibody (circle) 2 weeks after noise exposure. FIG. 14B provides comparisons of wave I amplitude (1st graph) and wave IV amplitude (2^{nd} graph) in animals treated with the anti-FAM19A5 antibody (square), before noise group (triangle) or control IgG antibody (circle) 4 weeks after noise exposure. Tone bursts were measured across 0 and 90 dB.

FIGS. 15A, 15B, 15C, 15D, and 15E show the dose-dependent effects of FAM19A5 antagonist administration on ABR threshold in a noise-induced hearing loss mouse model. FIG. 15A provides a schematic of the experimental design. As shown, one day after noise exposure, the animals received varying concentrations of the anti-FAM19A5 antibody (1-30) (i.e., 0 mg/kg, 3 mg/kg, 7 mg/kg, 15 mg/kg, and 30 mg/kg). After noise exposure, control animals received a human anti-IgG antibody (30 mg/kg). ABR threshold was measured at one day after noise exposure and at two weeks after noise exposure. FIG. 15B provides a comparison of ABR threshold for the different treatment groups across a frequency of 0 kHz to 64 kHz at one day after noise exposure. FIG. 15C provides the same data shown in FIG. 15B except as bar graphs. FIG. 15D provides a comparison of ABR threshold for the different treatment groups across a frequency of 0 kHz to 64 kHz at two weeks after noise exposure. FIG. 15E provides the same data shown in FIG. 15C except as bar graphs. In each of FIGS. 15B-15E, ABR threshold prior to noise exposure is also shown (“BN”). In both FIGS. 15C and 15E, for each of the frequency groups shown, the first bar (from the left) corresponds to pre-noise exposure (“BN”), the second bar corresponds to animal streated with the control anti-IgG antibody (“hIgG”), the third bar corresponds to animals treated with 0 mg/kg of the anti-FAM19A5 antibody, the fourth bar corresponds to animals treated with 3 mg/kg of the anti-FAM19A5 antibody, the fifth bar corresponds to animals treated with 7 mg/kg of the anti-FAM19A5 antibody, the sixth bar corresponds to animals treated with 15 mg/kg of the anti-FAM19A5 antibody, and the last bar corresponds to animals treated with 30 mg/kg of the anti-FAM19A5 antibody.

FIGS. 16A and 16B provide a comparision of the therapeutic effect of FAM19A5 antagonist administration on different aged mice (i.e., 10-week old, 6-month old, and 12-month old). show the comparison of age related hearing loss between normal and aged mice using parameters such as In each of the figures, the effect of FAM19A5 antagonist on hearing loss is shown via ABR threshold (left graphs), wave I amplitude (middle graphs), and DPOAE (right graphs). FIG. 16A provides a comparison between 6-month old mice and 10-week old mice. FIG. 16B provides a comparison between 12-month old mice and 10-week old mice. ABR threshold was measured at a frequency ranging from 2 kHz to 64 kHz. Tone bursts were measured across 0 and 90 dB. DPOAE amplitude was measured between F2 frequency of 4 and 32.

FIGS. 17A, 17B, 17C, and 17D show the therapeutic effect of FAM19A5 antagonists (1-30) on auditory brainstem response (ABR) threshold in an aged mouse model. FIG. 17A provides a schematic of the experimental design. As shown, anti-FAM19A5 antibody (1-30) or control human IgG antibody was administered to the mice (1-year old) intravenously once a week (30 mg/kg per dose) for a total of eight administrations. ABR (i.e., hearing test) was measured both prior to the initial administration of the antibodies and then at one and two months after administration. FIG. 17B provides the results before injection. FIG. 17C provides the results at one month post administration. FIG. 17D provides the results at two months post administration. In each of FIGS. 16B-16D, ABR threshold was measured at a frequency ranging from 4 kHz to 64 kHz.

FIGS. 18A, 18B, and 18C show the therapeutic effect of FAM19A5 antagonists (1-30) as comparison of wave I amplitude profile in an aged mouse model. The mice were treated as shown and described in FIG. 17A. FIG. 18A provides the results before injection. FIG. 18B provides the results at one month post injection. FIG. 18C provides the results at two months post injection. Tone bursts were measured across 0 and 90 dB.

FIG. 19 shows Western blot results for the binding of monoclonal antibody clone 1-65 to epitope fragments F1 to F6 (lanes 3-8, respectively). FAM19A5-CK (lane 1), PSA-CK (lane 2), Peptide NDV-BSA (lane 9), and BSA (lane 10) were used as controls. The respective sizes of the different antigens used are shown to the right of the blot. The amount of antigen used per well is 300 ng. The primary antibody used for the Western blot is 1-65-scFv-rabbit-Fc-SSS (2 μg/ml), and the secondary antibody used for the experiment is anti-Rabbit IgG (Fc specific)-HRP (1:4000).

FIGS. 20A and 20B show ELISA results for the binding of several anti-FAM19A5 antibodies to epitope fragments F1 to F6. FIG. 20A shows results for the anti-FAM19A5 antibodies 1-65, 2-13, and 3-2. For the 3-2 antibody, two different isotypes are shown: human IgG1 (“h3-2”) and mouse IgG1 (“m3-2”).

FIG. 20B shows results for the P2-C12 antibody. For each of the antibodies, the 1^{st}, 2^{nd}, 3^{rd}, 4^{th}, 5^{th}, and 6^{th} bars (starting from the left) represent binding to epitope fragments F1, F2, F3, F4, F5, and F6, respectively. The bar farthest to the right (black) represents the positive control (i.e., His tagged FAM19A5 protein). The exact O.D. value are indicated at the top of each bar.

FIGS. 21A and 21B show the ELISA results for the binding of anti-FAM19A5 antibodies 1-65 (FIG. 21A) and P2-C₁₂ (FIG. 21B) to eight different FAM19A5 fragment 5 mutant peptides (F5-1 to F5-8). The exact O.D. value are indicated at the top of each bar.

FIGS. 22A-22C show the ELISA results for the binding of different anti-FAM19A5 antibodies to FAM19A5 mutants M1 to M8. FIG. 22A shows the results for anti-FAM19A5 antibodies 1-65, 1-28, 2-13, and 3-2. FIG. 22B shows the results for anti-FAM19A5 antibodies 13B4, 13F₇, and 15A9. FIG. 22C shows the results for anti-FAM19A5 antibodies P1-A08, P1-F02, P1-G09, P2-A01, P2-A03, P2-C12, P2-F07, and P2-F11. In each of FIGS. 22A-22C, the eight bars for each of the antibodies correspond to mutants M1 to M8 (moving from left to right).

FIG. 23A shows the schematic diagram of the two-site sandwich ELISA assay used to assess cross-competition among the different anti-FAM19A5 antibodies. FIG. 23B shows the results of the cross-competition analysis for six different anti-FAM19A5 antibodies: 1-65, P2-A03, P2-F11, 13B4, 2-13, and 3-2. The term “S/N” refers to the signal to noise ratio, which is measured as follows: [O.D. of 10 ng/ml antigen]/[O.D. of 0 ng/ml antigen]. The grey boxes shows cross-competition (i.e., S/N ratio lower than 2).

FIGS. 24A-24C show the ELISA results for the binding of anti-FAM19A5 antibodies 3-2 and 1-28 to thirteen different FAM19A5 epitope F2 fragment mutant peptides: (i) F2-01-BSA (#1), (ii) F2-02-BSA (#2), (iii) F2-03-BSA (#3), (iv) F2-04-BSA (#4), (v) F2-05-BSA (#5), (vi) F2-06-BSA (#6), (vii) F2-07-BSA (#7), (viii) F2-08-BSA (#8), (ix) F2-09-BSA (#9), (x) F2-10-BSA (#10), (xi) F2-11-BSA (#11), (xii) F2-12-BSA (#12), and (xiii) F2-13-BSA (#13). FIG. 24A shows the results for the 3-2 antibody with human IgG1 isotype. FIG. 24B shows the results for the 3-2 antibody with mouse IgG1 isotype. FIG. 24C shows the results for the 1-28 antibody. The exact O.D. values are indicated at the top of each bar.

FIGS. 25A-25J provide the results of the alanine scanning analysis, showing the specific amino acid residues in the epitope F2 fragment that are important for the binding of the various 3-2 antibody variants to FAM19A5 protein. FIGS. 25A, 25B, 25C, and 25D show the results for the following antibodies generated in HEK293F cells: (A) wild-type 3-2 antibody, (B) 1-30 antibody, (C) 1-32 antibody, and (D) 6-10 antibody, respectively. FIGS. 25E, 25F, 25G, 25H, 25I, and 25J show the results for the following antibodies generated in CHO cells: (E) 1-17 antibody, (F) 1-30 antibody, (G) 1-32 antibody, (H) 4-11 antibody, (I) 6-10 antibody, and (J) low-PI antibody, respectively. As discussed in Example 7, mutant peptides were generated, wherein each of the mutant peptide had an alanine substitution at a single amino acid residue within the epitope fragment F2. Binding of the antibodies to the different mutant peptides was measured using ELISA.

FIG. 26 provides a 3-dimensional structure of the FAM19A5 protein and the location of the key binding epitope for the 2-13 antibody.

FIGS. 27A and 27B show the ability of FAM19A5 antagonists described herein (e.g., anti-FAM19A5 antibody) in reversing FAM19A5-induced inhibition of neurite outgrowth in mouse primary spiral ganglion neurons. FIG. 27A provides representative confocal microscopy images of primary spiral ganglion neurons treated with one of the following: (1) control antibody (human IgG) alone, (2) 10 μM FAM19A5 protein alone, or (3) 10 μM FAM19A5 protein with 100 nM anti-FAM19A5 antibody (1-30). Scale bar=200 μm. FIG. 27B provides comparison of average total neurite length of primary spiral ganglion neurons from FIG. 27A and quantified via Image J. Number of spiral ganglion neurons counted per group; Control=18, FAM19A5=19, FAM19A5+1−30=19. Data represents the mean±S.E.M. Statistical significance was evaluated using one-way ANOVA followed by Tukey's multiple comparisons test; *, P<0.05, **, P<0.01 versus FAM19A5.

FIGS. 28A-28H show the ability of FAM19A5 antagonists described herein (e.g., anti-FAM19A5 antibody and FAM19A5-targeting peptides) in promoting neurite outgrowth of primary mouse spiral ganglion neurons. FIGS. 28A, 28C, 28E, and 28G provide representative confocal microscopy images of primary spiral ganglion neurons treated with either a control (human IgG) antibody or different FAM19A5 antagonists described herein. The different FAM19A5 antagonists were as follows: 1-30 antibody (FIG. 28A), 2-13 antibody (FIG. 28C), 1-65 SS01 antibody (FIG. 28E), and dFB-DY-JM31 peptide (FIG. 28F). In FIGS. 28A and 28G, scale bar represents 100 μm. In FIGS. 28C and 28G, scale bar represents 50 μm.

FIGS. 28B, 28D, 28F, and 28H provide comparison of average total neurite length of primary spiral ganglion neurons from FIGS. 28A, 28C, 28E, and 28G, respectively, and quantified via Image J. Number of spiral ganglion neurons counted per group; for FIGS. 28B, Control=22, 1−30=19, for FIGS. 28D, Control=26, 2−13=27, for FIGS. 28F, Control=29, 1-65 SS01=26, for FIGS. 28H, Control=30, dFB-DY-JM31=38. Data are presented as mean±SEM. Statistical analysis was performed using an unpaired t-test to evaluate significance, with * indicating P<0.05 and ** indicating P<0.01 compared to control groups.

FIGS. 29A and 29B show the ability of FAM19A5 antagonists described herein in reversing FAM19A5-induced decrease in the number of CtBP2 puncta in the inner hair cell of mouse cochlear explant. FIG. 29A presents representative confocal microscopy images of CtbP2 in cochlear explants treated with one of the following: (1) control (human IgG) antibody alone, (2) 1 μM FAM19A5 protein alone, or (3) 1 μM FAM19A5 protein with an anti-FAM19A5 antibody (1-30). Scale bar=20 μm. FIG. 29B provides comparison of the number of CtBP2 puncta in the inner hair cells of cochlear explants from FIG. 29A and quantified using Image J. Number of inner hair cells counted per group=15. Data represents the mean #S.E.M. Statistical significance was evaluated using one-way ANOVA followed by Tukey's multiple comparisons test; ***, P<0.001, ****, P<0.0001 versus FAM19A5.

FIGS. 30A and 30B show the ability of FAM19A5 antagonists described herein (e.g., anti-FAM19A5 antibody) in promoting synaptogenesis in the inner hair cell of mouse cochlear explants. FIG. 30A presents representative conforcal microscopy images showing ribbon synapse marker (CtBP2) expression in the inner hair cell of mouse cochlear explants treated with either a control (human IgG) antibody or 1 μM anti-FAM19A5 antibody (1-30). Scale bar=20 μm. FIG. 30B provides comparison of the number of CtBP2 puncta in the inner hair cells of mouse cochlear explants from FIG. 30A and quantified using Image J. Number of inner hair cells counted per group=15. Data represents the mean±S.E.M. Statistical significance was evaluated using unpaired t-test; **, P<0.01 versus control.

### DETAILED DESCRIPTION OF THE DISCLOSURE

The present disclosure is generally directed to methods of treating hearing disorders. More particularly, provided herein are methods of treating a hearing disorder in a subject in need thereof, comprising administering to the subject an antagonist that specifically targets FAM19A5 (FAM19A5 antagonist). The present disclosure describes for the first time that by regulating FAM19A5 activity (e.g., within the spiral ganglion neuron), it is possible to improve hearing. As further described herein, FAM19A5 antagonists provided herein can also be used to reduce impairment of a ribbon synapse within an inner ear of a subject (e.g., suffering from a hearing disorder) and/or induce the formation of ribbon synapses within the inner ear of a subject. Additional aspects are provided throughout the present disclosure.

To facilitate an understanding of the disclosure disclosed herein, a number of terms and phrases are defined. Additional definitions are set forth throughout the detailed description.

### I. Definitions

Throughout this disclosure, the term “a” or “an” entity refers to one or more of that entity; for example, “a molecule” is understood to represent one or more molecules. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “hearing disorder” refers to any disease, disorder, or condition related to an impairment of the sense of hearing. In some aspects, a hearing disorder is associated with a partial hearing loss. For example, compared to a reference subject (e.g., corresponding subject who does not suffer from impaired hearing), the hearing ability of a person suffering from a hearing disorder (e.g., described herein) is reduced by at least about 5%, at least about 10%, at least about 20%, at least about 30%, at least about 40%, at least about 50%, at least about 60%, at least about 70%, at least about 80%, at least about 90%, or about 100%. In some aspects, a hearing disorder is associated with complete hearing loss. The hearing ability of a subject can be assessed using any suitable methods known in the art. Non-limiting examples of methods that can be used to assess whether a subject suffers from a hearing disorder include Pure Tone Audiometry (PTA), Extended Tone Audiometry (ETA), Speech Discrimination Score (SDS), Word-In-Noise (WIN), Tinnitus Functional Index (TFI), and Tinnitus Intensity (TL). In some aspects, a hearing disorder useful for the present disclosure comprises a sensorineural hearing loss. As used herein, the term “sensorineural hearing loss” refers to hearing loss caused by a disorder in a sound sensing function of the cochlear canal, or a dysfunction in the auditory nerve or central nerve system which transmits auditory stimuli to the brain. Additional non-limiting examples of hearing disorders that are relevant for the present disclosure are provided elsewhere herein.

As used herein, the term “tinnitus” refers to a phantom auditory perception without an external auditory stimulus. Two main types of tinnitus include (1) “objective tinnitus,” which is caused by sounds generated somewhere in the body (e.g., turbulent flow of blood or muscle contractions in the head); (2) “subjective tinnitus,” which is the perception of meaningless sounds without any physical sound being present. Objective tinnitus is rare and can be heard by an observer, in contrast to subjective tinnitus, which can only be heard by the individual who has the tinnitus. Tinnitus sounds can take a variety of forms such as buzzing, ringing, or a range of other sounds. Tinnitus can occur in one ear or both ears either constantly or sporadically. As is apparent from the present disclosure, in some aspects, a hearing disorder that can be treated with the present disclosure (e.g., sensorineural hearing loss) is associated with tinnitus. In some aspects, a hearing disorder that can be treated with the present disclosure (e.g., sensorineural hearing loss) is not associated tinnitus.

As used herein, the term “ribbon synapse” refers to a type of neuronal synapse characterized by the presence of an electron-dense structure (i.e., “synaptic ribbon”) that holds synaptic vesicles (which hold various neurotransmitters) close to the active zone (i.e., site of neurotransmitter release). Ribbon synapses are characterized by a tight vesicle-calcium channel coupling that promote rapid neurotransmitter release and sustained signal transmission. Ribbon synapses undergo a cycle of exocytosis and endocytosis in response to graded changes of membrane potential. Unless indicated otherwise, ribbon synapses described herein refer to those that play a role in hearing. Within the auditory system, ribbon synapses are commonly present between inner hair cells (IHCs) and spiral ganglion neurons. Wei M., et al., Neural Plast 2020:8815990 (November 2020).

As used herein, the term “auditory system” refers to the sensory system for the sense of hearing in a subject (e.g., a human subject).

The term “inner hair cells” (IHCs) refer to a type of “cochlear hair cell” (i.e., sensory cells of the auditory system). At least in humans, cochlear hair cells consist of one row of inner hair cells and three rows of outer hair cells. The inner hair cells are the actual sensory receptors (transforms the sound vibration in the fluids of the cochlea into electrical signals), and 95% of the fibers of the auditory nerve that project to the brain arise from this subpopulation.

The term “neuron” includes electrically excitable cells that process and transmit information through electrical and chemical signals. A typical neuron is composed of a cell body (soma), dendrites, and an axon (also referred to herein as “nerve fiber”). The soma (the cell body) of a neuron contains the nucleus. The dendrites of a neuron are cellular extensions with many branches, where the majority of input to the neuron occurs. The axon is a finer, cable-like projection extending from the soma and carries nerve signals away from the soma and certain types of information back to the soma.

As used herein, the term “nerve fiber” refers to a portion of the neuron, namely the axon, which carries action potentials from one end of the neuron to the other. In some aspects, a nerve fiber comprises a “cochlear nerve fiber.” The cochlear nerve fibers originate from neurons of the spiral ganglion and project peripherally to cochlear hair cells and centrally to the cochlear nuclei (cochlear nucleus) of the brain stem. They mediate the sense of hearing.

As used herein, the term “spiral ganglion” refers to the sensory ganglion of the cochlear nerve. The cells of the spiral ganglion send fibers peripherally to the cochlear hair cells and centrally to the cochlear nuclei (cochlear nucleus) of the brain stem.

The term “cochlea,” as used herein, refers to a spiral-shaped cavity of the inner ear that resembles a snail shell and contains nerve endings essential for hearing. The cochlea includes three fluid-filled chambers: scala tympani and scala vestibuli (both of which contain perilymph), and scala media (which contains endolymph). The scala tympani and the scala vestibuli are contiguous with each other, merging at the tip of the snail shell, the helicotrema. The stapes transmits vibrations to the fenestra ovalis (oval window) on the outside of the cochlea, which vibrates the perilymph in the scala vestibuli. This in turn vibrates the endolymph in the scala media, thus causing movements of the hair bundles of the hair cells, which are acoustic sensor cells that convert vibration into electrical potentials. The hair cells are arranged in four rows in the organ of Corti along the entire length of the cochlear coil. Three rows consist of outer hair cells (OHCs) and one row consists of inner hair cells (IHCs). The IHCs provide the main neural output of the cochlea. The outer hair cells, instead, mainly receive neural input from the brain, which influences their motility as part of the cochlea's mechanical pre-amplifier.

The term “family with sequence similarity 19, member A5” or “FAM19A5” refers to a protein that belongs to the TAFA family (also known as FAM19 family) of five highly homologous proteins and is predominantly expressed in brain and the spinal cord. FAM19A5 is also known as “TAFA5” or “Chemokine-like protein TAFA-5.”

In humans, the gene encoding FAM19A5 is located on chromosome 22. There are multiple human FAM19A5 (UniProt: Q7Z5A7) isoforms, which are believed to be produced by alternative splicing: isoform 1 (UniProt: Q7Z5A7-1), which consists of 132 amino acids, isoform 2 (UniProt: Q7Z5A7-2), which consists of 125 amino acids, and isoform 3 (UniProt: Q7Z5A7-3), which consists of 53 amino acids. Human FAM19A5 protein is believed to exist as both membrane bound and soluble (secreted) forms. Isoform 1 is believed to be a membrane protein with one transmembrane region. Isoform 2, which was reported in Tang T. Y. et al., Genomics 83(4):727-34 (2004) as a secreted protein (soluble), contains a signal peptide at amino acid positions 1-25. Isoform 1 is believed to be a membrane protein and predicted based on EST data. Table 1 (below) provides the amino acid sequences of the three known human FAM19A5 isoforms. Unless indicated otherwise, the term “FAM19A5” includes any variants or isoforms of the FAM19A5 protein which are naturally expressed by cells. Accordingly, in some aspects, a polypeptide described herein (e.g., comprising a FAM19A5 binding domain of a member of the LRRC4 protein family) can inhibit the binding of FAM19A5 isoform 1, isoform 2, and/or isoform 3 to the LRRC4 protein family members.

TABLE 1
FAM19A5 Protein Sequences

| | |
|---|---|
| Human FAM19A5 Protein | MAPSPRTGSRQDATALPSMSSTFWAFMILASLLIAYCSQLAAGTCEIV |
| (Isoform 1) | TLDRDSSQPRRTIARQTARCACRKGQIAGTTRARPACVDARIIKTKQW |
| (UniProt: Q7Z5A7-1, | CDMLPCLEGEGCDLLINRSGWTCTQPGGRIKTTTVS |
| transmembrane protein): | (SEQ ID NO: 1) |
| this isoform has been | |
| chosen as the canonical | |
| sequence. | |
| Human FAM19A5 Protein | MQLLKALWALAGAALCCFLVLVIHAQFLKEGQLAAGTCEIVTLDRDSS |
| (Isoform 2) | QPRRTIARQTARCACRKGQIAGTTRARPACVDARIIKTKQWCDMLPCL |
| (UniProt: Q7Z5A7-2, | EGEGCDLLINRSGWTCTQPGGRIKTTTVS (SEQ ID NO: 2) |
| soluble protein) | |
| Human FAM19A5 Protein | MYHHREWPARIIKTKQWCDMLPCLEGEGCDLLINRSGWTCTQPGGRIK |
| (Isoform 3) | TTTVS (SEQ ID NO: 3) |
| (UniProt: Q7Z5A7-3) | |

The term “antagonist against a FAM19A5” or “FAM19A5 antagonist” refers to all antagonists that suppress the expression and/or activity of FAM19A5. Such antagonist can be a peptide, a nucleic acid, or a compound. More specifically, the antagonist can be an antisense-oligonucleotide, siRNA, shRNA, miRNA, dsRNA, aptamer, PNA (peptide nucleic acid) targeting FAM19A5, or a vector including the same. In some aspects, the antagonist can be an antibody, or an antigen-binding portion thereof, that specifically binds to the FAM19A5 protein (“anti-FAM19A5 antibody”). As further described herein, in some aspects, a FAM19A5 antagonist useful for the present disclosure comprises polynucleotides encoding an anti-FAM19A5 antibody.

As also further described herein, in some aspects, a FAM19A5 antagonist comprises a mimic molecule that is capable of specifically targeting FAM19A5 and thereby, inhibit, reduce, and/or dissociate the binding between FAM19A5 and a LRRC4 protein family member (i.e., “LRRC4 family mimic molecule”). A “mimic molecule” refers to a molecule that resembles another molecule (“reference molecule”) in structure and/or function. For instance, in some aspects, a mimic molecule can share a partial structure or sequence with the reference molecule, such that mimic molecule can exhibit one or more properties of the reference molecule. However, as is apparent from the present disclosure, a structural or sequence similarity is not always required. In some aspects, a mimic molecule can differ in structure but behave similarly to the reference molecule. As described herein, in some aspects, a mimic molecule comprises a small molecule. In some aspects, a mimic molecule comprises a peptide. In some aspects, a mimic molecule is not an antibody or an antigen-binding portion thereof.

As is apparent from the present disclosure, while the LRRC4 family mimic molecules described herein share certain properties with the members of the LRRC4 protein family, the LRRC4 family mimic molecules differ (structurally and/or functionally) from naturally existing members of the LRRC4 protein family. For instance, in some aspects, a LRRC4 family mimic molecule useful for the present disclosure comprises a small molecule. As further described elsewhere in the present disclosure, in some aspects, a LRRC4 family mimic molecule comprises a polypeptide, wherein the polypeptide comprises, consists of, or consists essentially of a domain of a member of the LRRC4 protein family, wherein the domain is capable of binding to the FAM19A5 protein (also referred to herein as the “FAM19A5 binding domain”). For such aspects, the polypeptides can comprise one or more amino acid substitutions within the FAM19A5 binding domain. As described elsewhere in the present disclosure, in some aspects, such amino acid substitutions can improve one or more properties of the polypeptides, e.g., increase the stability and/or binding affinity of the polypeptides to the FAM19A5 protein. In some aspects, the polypeptides can comprise the FAM19A5 binding domain but lack one or more other domains of the members of the LRRC4 protein family. For instance, in some aspects, a polypeptide comprises the FAM19A5 binding domain, but does not comprise the transmembrane domain. In some aspects, a polypeptide comprises the FAM19A5 binding domain, but does not comprise the intracellular domain of members of the LRRC4 protein family (e.g., postsynaptic density-binding (PB) domain). In some aspects, a polypeptide comprises the FAM19A5 binding domain, but does not comprise both the transmembrane domain and the intracellular domain. Accordingly, in some aspects, polypeptides described herein are shorter than the naturally existing LRRC4 protein family members. Additionally, in carrying out their biological activity (e.g., neural circuit formation), members of the LRRC4 protein family (LRRC4, LRRC4B, and LRRC4C) interact with their ligand (netrin-G2, receptor tyrosine phosphatase LAR, and netrin-G1, respectively). See, e.g., Li et al., Mol Cancer 13:266 (December 2014). Because the polypeptides of the present disclosure do not comprise all the domains of LRRC4 protein family members, in some aspects, the polypeptides do not bind to the LRRC4 protein family ligands and instead, specifically target the FAM19A5 protein. Accordingly, in some aspects, the polypeptides described herein do not replace the endogenous LRRC4 protein family members. Instead, in some aspects, by inhibiting, reducing, and/or dissociating the interaction between FAM19A5 and members of the LRRC4 protein family, the polypeptides of the present disclosure can free up the endogenous LRRC4 family proteins and allow them to carry out their natural biological activity.

Unless indicated otherwise, the term “FAM19A5 antagonist” comprises both an anti-FAM19A5 antibody and a LRRC4 family mimic molecule.

The term “leucine-rich repeat-containing 4 protein family” or “LRRC4 protein family” (including derivatives thereof) refers to a family of proteins that are key synaptic organizers and have been described to play a role in various steps of neural circuit formation, including neuronal migration, neurite outgrowth, and both the formation and functional assembly of synaptic contacts. See, e.g., Woo et al., Mol Cell Neurosci 42(1): 1-10 (September 2009). The LRRC4 protein family includes three members: (1) LRRC4, (2) LRRC4B, and (3) LRRC4C (collectively referred to herein as “LRRC4 protein family member” or “member of the LRRC4 protein family” (or derivatives thereof)) . . . . The members of the LRRC4 protein family generally contains nine leucine-rich repeat (LRR) domains flanking LRR N-terminus and C-terminus. These LRR domains are known to interact with fibronectin type III domains of presynaptic receptor protein tyrosine phosphatase (RPTP) proteins. See, e.g., Won et al., Mol Cells 41(7): 622-630 (July 2018). The LRR domains are followed by immunoglobulin-like C2-type (IG) and threonine (Thr)-rich domains, which together form the extracellular portion of members of the LRRC4 protein family. Unlike the other members, the LRRC4B protein has an extra glycine (Gly)-rich domain between the IG and Thr-rich domains. In addition to the extracellular portion, members of the LRRC4 protein family additionally include a transmembrane (TM) domain and a postsynaptic density-binding (PB) domain at the C-terminus of the protein.

In humans, gene encoding the LRRC4 protein is located on chromosome 7 (nucleotides 128,027,071-128,032,107 of GenBank Accession Number NC_000007.14; minus strand orientation). LRRC4 protein synonyms are known and examples include: “Nasopharyngeal Carcinoma-Associated Gene 14 Protein,” “Brain Tumor-Associated Protein BAG,” “Netrin-G2 Ligand,” “NAG14,” “NGL-2,” and “BAG.” The amino acid sequence for the LRRC4 protein is 653 amino acids in length and provided in Table 2 (below). The full-length ectodomain of the LRRC4 protein corresponds to amino acid residues 39-527 of SEQ ID NO: 4 (i.e., SEQ ID NO: 6). Unless indicated otherwise, the term “LRRC4 protein” (including its synonyms) includes any variants or isoforms of the LRRC4 protein which are naturally expressed by cells.

TABLE 2
LRRC4 Protein Sequence

| | |
|---|---|
| Human LRRC4 protein | MKLLWQVTVHHHTWNAILLPFVYLTAQVWILCAAIAAAASAGPQ |
| (UniProt: Q9HBW1) | NCPSVCSCSNQFSKVVCTRRGLSEVPQGIPSNTRYLNLMENNIQ |
| (signal peptide is bolded) | MIQADTFRHLHHLEVLQLGRNSIRQIEVGAFNGLASLNTLELFD |
| | NWLTVIPSGAFEYLSKLRELWLRNNPIESIPSYAFNRVPSLMRL |
| | DLGELKKLEYISEGAFEGLFNLKYLNLGMCNIKDMPNLTPLVGL |
| | EELEMSGNHFPEIRPGSFHGLSSLKKLWVMNSQVSLIERNAFDG |
| | LASLVELNLAHNNLSSLPHDLFTPLRYLVELHLHHNPWNCDCDI |
| | LWLAWWLREYIPTNSTCCGRCHAPMHMRGRYLVEVDQASFQCSA |
| | PFIMDAPRDLNISEGRMAELKCRTPPMSSVKWLLPNGTVLSHAS |
| | RHPRISVLNDGTLNFSHVLLSDTGVYTCMVTNVAGNSNASAYLN |
| | VSTAELNTSNYSFFTTVTVETTEISPEDTTRKYKPVPTTSTGYQ |
| | PAYTTSTTVLIQTTRVPKQVAVPATDTTDKMQTSLDEVMKTTKI |
| | IIGCFVAVTLLAAAMLIVFYKLRKRHQQRSTVTAARTVEIIQVD |
| | EDIPAATSAAATAAPSGVSGEGAVVLPTIHDHINYNTYKPAHGA |
| | HWTENSLGNSLHPTVTTISEPYIIQTHTKDKVQETQI |
| | (SEQ ID NO: 4) |
| Human LRRC4 | ASAGPQNCPSVCSCSNQFSKVVCTRRGLSEVPQGIPSNTRYLNL |
| ectodomain protein (i.e., | MENNIQMIQADTFRHLHHLEVLQLGRNSIRQIEVGAFNGLASLN |
| amino acids 39-527 of | TLELFDNWLTVIPSGAFEYLSKLRELWLRNNPIESIPSYAFNRV |
| SEQ ID NO: 4) | PSLMRLDLGELKKLEYISEGAFEGLFNLKYLNLGMCNIKDMPNL |
| | TPLVGLEELEMSGNHFPEIRPGSFHGLSSLKKLWVMNSQVSLIE |
| | RNAFDGLASLVELNLAHNNLSSLPHDLFTPLRYLVELHLHHNPW |
| | NCDCDILWLAWWLREYIPTNSTCCGRCHAPMHMRGRYLVEVDQA |
| | SFQCSAPFIMDAPRDLNISEGRMAELKCRTPPMSSVKWLLPNGT |
| | VLSHASRHPRISVLNDGTLNFSHVLLSDTGVYTCMVTNVAGNSN |
| | ASAYLNVSTAELNTSNYSFFTTVTVETTEISPEDTTRKYKPVPT |
| | TSTGYQPAYTTSTTVLIQTTRVPKQVAVPATDTTDKMQTSLDEV |
| | MKTTK (SEQ ID NO: 6) |

In humans, the gene encoding the LRRC4B protein is located on chromosome 19 (nucleotides 50,516,892-50,568,435 of GenBank Accession Number NC_000019.10; minus strand orientation). Synonyms of the LRRC4B protein are known and non-limiting examples include: “Netrin-G3 Ligand,” “LRIG4,” “NGL-3,” “HSM,” and “DKFZp761A179.” The amino acid sequence for the LRRC4B protein is 713 amino acids in length and provided in Table 3 (below). The full-length ectodomain of the LRRC4B protein corresponds to amino acid residues 36-576 of SEQ ID NO: 7 (i.e., SEQ ID NO: 9). Unless indicated otherwise, the term “LRRC4B protein” (including its synonyms) includes any variants or isoforms of the LRRC4B protein which are naturally expressed by cells.

TABLE 3
LRRC4B Protein Sequence

| | |
|---|---|
| Human LRRC4B protein | MARARGSPCPPLPPGRMSWPHGALLFLWLFSPPLGAGGGGVAVTSAAG |
| (UniProt: Q9NT99) | GGSPPATSCPVACSCSNQASRVICTRRDLAEVPASIPVNTRYLNLQEN |
| (signal peptide is bolded) | GIQVIRTDTFKHLRHLEILQLSKNLVRKIEVGAFNGLPSLNTLELFDN |
| | RLTTVPTQAFEYLSKLRELWLRNNPIESIPSYAFNRVPSLRRLDLGEL |
| | KRLEYISEAAFEGLVNLRYLNLGMCNLKDIPNLTALVRLEELELSGNR |
| | LDLIRPGSFQGLTSLRKLWLMHAQVATIERNAFDDLKSLEELNLSHNN |
| | LMSLPHDLFTPLHRLERVHLNHNPWHCNCDVLWLSWWLKETVPSNTTC |
| | CARCHAPAGLKGRYIGELDQSHFTCYAPVIVEPPTDLNVTEGMAAELK |
| | CRTGTSMTSVNWLTPNGTLMTHGSYRVRISVLHDGTLNFTNVTVQDTG |
| | QYTCMVTNSAGNTTASATLNVSAVDPVAAGGTGSGGGGPGGSGGVGGG |
| | SGGYTYFTTVTVETLETQPGEEALQPRGTEKEPPGPTTDGVWGGGRPG |
| | DAAGPASSSTTAPAPRSSRPTEKAFTVPITDVTENALKDLDDVMKTTK |
| | IIIGCFVAITFMAAVMLVAFYKLRKQHQLHKHHGPTRTVEIINVEDEL |
| | PAASAVSVAAAAAVASGGGVGGDSHLALPALERDHLNHHHYVAAAFKA |
| | HYSSNPSGGGCGGKGPPGLNSIHEPLLFKSGSKENVQETQI |
| | (SEQ ID NO: 7) |
| Human LRRC4B | AGGGGVAVTSAAGGGSPPATSCPVACSCSNQASRVICTRRDLAEVPAS |
| ectodomain protein (i.e., | IPVNTRYLNLQENGIQVIRTDTFKHLRHLEILQLSKNLVRKIEVGAFN |
| amino acids 36-576 of | GLPSLNTLELFDNRLTTVPTQAFEYLSKLRELWLRNNPIESIPSYAFN |
| SEQ ID NO: 7) | RVPSLRRLDLGELKRLEYISEAAFEGLVNLRYLNLGMCNLKDIPNLTA |
| | LVRLEELELSGNRLDLIRPGSFQGLTSLRKLWLMHAQVATIERNAFDD |
| | LKSLEELNLSHNNLMSLPHDLFTPLHRLERVHLNHNPWHCNCDVLWLS |
| | WWLKETVPSNTTCCARCHAPAGLKGRYIGELDQSHFTCYAPVIVEPPT |
| | DLNVTEGMAAELKCRTGTSMTSVNWLTPNGTLMTHGSYRVRISVLHDG |
| | TLNFTNVTVQDTGQYTCMVTNSAGNTTASATLNVSAVDPVAAGGTGSG |
| | GGGPGGSGGVGGGSGGYTYFTTVTVETLETQPGEEALQPRGTEKEPPG |
| | PTTDGVWGGGRPGDAAGPASSSTTAPAPRSSRPTEKAFTVPITDVTEN |
| | ALKDLDDVMKTTK (SEQ ID NO: 9) |

In humans, the gene encoding the LRRC4C protein is located on chromosome 11 (nucleotides 40,107,066-41,460,419 of GenBank Accession Number NC_000011.10; minus strand orientation). Synonyms of the LRRC4C protein are known and examples include: “NGL-1,” “Netrin-G1 Ligand,” and “KIAA1580.” The amino acid sequence for the LRRC4C protein is 640 amino acids in length and provided in Table 4 (below). The full-length ectodomain of the LRRC4C protein corresponds to amino acid residues 45-527 of SEQ ID NO: 82 (i.e., SEQ ID NO: 84). Unless indicated otherwise, the term “LRRC4C protein” (including its synonyms) includes any variants or isoforms of the LRRC4C protein which are naturally expressed by cells.

TABLE 4
LRRC4C Protein Sequence

| | |
|---|---|
| Human LRRC4C protein | MLNKMTLHPQQIMIGPRFNRALFDPLLVVLLALQLLVVAGLVRAQTCP |
| (UniProt: Q9HCJ2) | SVCSCSNQFSKVICVRKNLREVPDGISTNTRLLNLHENQIQIIKVNSF |
| (signal peptide is bolded) | KHLRHLEILQLSRNHIRTIEIGAFNGLANLNTLELFDNRLTTIPNGAF |
| | VYLSKLKELWLRNNPIESIPSYAFNRIPSLRRLDLGELKRLSYISEGA |
| | FEGLSNLRYLNLAMCNLREIPNLTPLIKLDELDLSGNHLSAIRPGSFQ |
| | GLMHLQKLWMIQSQIQVIERNAFDNLQSLVEINLAHNNLTLLPHDLFT |
| | PLHHLERIHLHHNPWNCNCDILWLSWWIKDMAPSNTACCARCNTPPNL |
| | KGRYIGELDQNYFTCYAPVIVEPPADLNVTEGMAAELKCRASTSLTSV |
| | SWITPNGTVMTHGAYKVRIAVLSDGTLNFTNVTVQDTGMYTCMVSNSV |
| | GNTTASATLNVTAATTTPFSYFSTVTVETMEPSQDEARTTDNNVGPTP |
| | VVDWETTNVTTSLTPQSTRSTEKTFTIPVTDINSGIPGIDEVMKTTKI |
| | IIGCFVAITLMAAVMLVIFYKMRKQHHRQNHHAPTRTVEIINVDDEIT |
| | GDTPMESHLPMPAIEHEHLNHYNSYKSPFNHTTTVNTINSIHSSVHEP |
| | LLIRMNSKDNVQETQI (SEQ ID NO: 82) |
| Human LRRC4C | QTCPSVCSCSNQFSKVICVRKNLREVPDGISTNTRLLNLHENQIQIIK |
| ectodomain protein (i.e., | VNSFKHLRHLEILQLSRNHIRTIEIGAFNGLANLNTLELFDNRLTTIP |
| amino acids 45-527 of | NGAFVYLSKLKELWLRNNPIESIPSYAFNRIPSLRRLDLGELKRLSYI |
| SEQ ID NO: 82) | SEGAFEGLSNLRYLNLAMCNLREIPNLTPLIKLDELDLSGNHLSAIRP |
| | GSFQGLMHLQKLWMIQSQIQVIERNAFDNLQSLVEINLAHNNLTLLPH |
| | DLFTPLHHLERIHLHHNPWNCNCDILWLSWWIKDMAPSNTACCARCNT |
| | PPNLKGRYIGELDQNYFTCYAPVIVEPPADLNVTEGMAAELKCRASTS |
| | LTSVSWITPNGTVMTHGAYKVRIAVLSDGTLNFTNVTVQDTGMYTCMV |
| | SNSVGNTTASATLNVTAATTTPFSYFSTVTVETMEPSQDEARTTDNNV |
| | GPTPVVDWETTNVTTSLTPQSTRSTEKTFTIPVTDINSGIPGIDEVMK |
| | TTK (SEQ ID NO: 84) |

As used herein, the term “FAM19A5 binding domain” refers to a segment/fragment of a member of the LRRC4 protein family that is capable of binding to a FAM19A5 protein.

As used herein, the term “alkenyl” refers to a group containing hydrogen and carbon and containing at least one carbon-carbon double bond.

As used herein, the term “alkoxy” refers to an alkyl group attached to the parent molecular moiety through an oxygen atom.

As used herein, the term “alkyl” refers to a group containing hydrogen and carbon and containing no double bonds.

As used herein, the term “alkynyl” refers to a group containing hydrogen and carbon and containing at least one carbon-carbon triple bond.

As used herein, the term “amino” refers to —NH₂.

As used herein, the term “bicycloalkenyl” refers to a fused, spirocyclic, or bridged bicyclic hydrocarbon ring system containing at least one double bond.

As used herein, the term “bicycloalkyl” refers to a fused, spirocyclic, or bridged bicyclic cycloalkyl ring.

As used herein, the term “cycloalkenyl” refers to an unsaturated non-aromatic monocyclic hydrocarbon ring system having zero heteroatoms. Representative examples of cycloalkenyl groups include, but are not limited to, cyclopentenyl, cyclohexenyl, cycloheptenyl, and cyclooctenyl.

As used herein, the term “cycloalkyl” refers to a saturated monocyclic hydrocarbon ring system having zero heteroatoms. Representative examples of cycloalkyl groups include, but are not limited to, cyclopropyl, cyclobutyl, cyclopentyl, and cyclohexyl.

As used herein, the term “formyl” refers to —CHO.

As used herein, the terms “halo” and “halogen” refer to Cl, Br, I, or F.

As used herein, the term “haloalkoxy” refers to a haloalkyl group attached to the parent molecular moiety through an oxygen atom.

As used herein, the term “haloalkyl” refers to an alkyl group substituted with one, two, three, or four halogen atoms.

As used herein, the term “heteroaryl” refers to an aromatic ring containing one, two, or three heteroatoms independently selected from nitrogen, oxygen, and sulfur. Representative examples of heteroaryl groups include, but are not limited to, furyl, imidazolyl, pyrazolyl, pyridinyl, pyrrolyl, thiazolyl, and thienyl.

As used herein, the term “heterobicycloalkyl” refers to a non-aromatic bicyclic ring system containing one, two, three, or four heteroatoms independently selected from nitrogen, oxygen, and sulfur and optionally containing one or more double bonds. The heterobicycloalkyl groups of the present disclosure can be attached to the parent molecular moiety through any carbon atom or nitrogen atom in the group.

As used herein, the term “heterocycloalkyl” refers to a non-aromatic ring containing one, two, three, or four heteroatoms independently selected from nitrogen, oxygen, and sulfur and optionally containing one or more double bonds. The heterocycloalkyl groups of the present disclosure can be attached to the parent molecular moiety through any carbon atom or nitrogen atom in the group. Representative examples of heterocycloalkyl groups include, but are not limited to, morpholinyl, piperazinyl, piperidinyl, tetrahydrofuranyl, tetrahydropyranyl, and thiomorpholinyl.

The terms “antibody” and “antibodies” are terms of art and can be used interchangeably herein and refer to a molecule with an antigen binding site that specifically binds an antigen. The terms as used herein include whole antibodies and any antigen binding fragments (i.e., “antigen-binding portions”) or single chains thereof. An “antibody” refers, in some aspects, to a glycoprotein comprising at least two heavy (H) chains and two light (L) chains inter-connected by disulfide bonds, or an antigen binding portion thereof. In some aspects, an “antibody” refers to a single chain antibody comprising a single variable domain, e.g., VHH domain. Each heavy chain is comprised of a heavy chain variable region (abbreviated herein as VH) and a heavy chain constant region. In certain naturally occurring antibodies, the heavy chain constant region is comprised of three domains, CH1, CH2 and CH3. In certain naturally occurring antibodies, each light chain is comprised of a light chain variable region (abbreviated herein as VL) and a light chain constant region. The light chain constant region is comprised of one domain, CL.

The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

The term “Kabat numbering” and like terms are recognized in the art and refer to a system of numbering amino acid residues in the heavy and light chain variable regions of an antibody, or an antigen-binding portion thereof. In some aspects, the CDRs of an antibody can be determined according to the Kabat numbering system (see, e.g., Kabat E A & Wu T T (1971) Ann NY Acad Sci 190:382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In some aspects, the CDRs of the antibodies described herein have been determined according to the Kabat numbering scheme.

The phrases “amino acid position numbering as in Kabat,” “Kabat position,” and grammatical variants thereof refer to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence can contain fewer or additional amino acids corresponding to a shortening of, or insertion into, a FW or CDR of the variable domain. For example, a heavy chain variable domain can include a single amino acid insert (residue 52a according to Kabat) after residue 52 of H2 and inserted residues (e.g., residues 82a, 82b, and 82c, etc. according to Kabat) after heavy chain FW residue 82. See TABLE 5.

TABLE 5
Amino acid numbering system for antibodies

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

IMGT (ImMunoGeneTics) also provides a numbering system for the immunoglobulin variable regions, including the CDRs. See, e.g., Lefranc, M. P. et al., Dev. Comp. Immunol. 27:55-77 (2003), which is herein incorporated by reference. The IMGT numbering system was based on an alignment of more than 5,000 sequences, structural data, and characterization of hypervariable loops and allows for easy comparison of the variable and CDR regions for all species. According to the IMGT numbering schema VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97.

Antibodies can be of any type (e.g., IgG, IgE, IgM, IgD, IgA or IgY), any class (e.g., IgD, IgG2, IgG3, IgG4, IgA1, or IgA2), or any subclass (e.g., IgG1, IgG2, IgG3, and IgG4 in humans, and IgG1, IgG2a, IgG2b, and IgG3 in mice) of immunoglobulin molecule. Immunoglobulins, e.g., IgG1, exist in several allotypes, which differ from each other in at most a few amino acids. An antibody disclosed herein can be from any of the commonly known isotypes, classes, subclasses, or allotypes. In some aspects, the antibodies described herein are of the IgG1, IgG2, IgG3 or IgG4 subclass or any hybrid thereof. In some aspects, the antibodies are of the human IgG1 subclass, human IgG2 subclass, or human IgG4 subclass.

“Antibody” includes, by way of example, both naturally occurring and non-naturally occurring antibodies; monoclonal and polyclonal antibodies; chimeric and humanized antibodies; human and non-human antibodies; wholly synthetic antibodies; single chain antibodies; monospecific antibodies; multispecific antibodies (including bispecific antibodies); tetrameric antibodies comprising two heavy chain and two light chain molecules; an antibody light chain monomer; an antibody heavy chain monomer; an antibody light chain dimer, an antibody heavy chain dimer; an antibody light chain-antibody heavy chain pair; intrabodies; heteroconjugate antibodies; monovalent antibodies; single chain antibodies; camelized antibodies; affybodies; anti-idiotypic (anti-Id) antibodies (including, e.g., anti-anti-Id antibodies), and single-domain antibodies (sdAbs), which include binding molecules consisting of a single monomeric variable antibody domain that are fully capable of antigen binding (e.g., a VH domain or a VL domain). Harmen M. M. and Haard H. J. Appl Microbiol Biotechnol. 77(1): 13-22 (2007)).

The terms “antigen-binding portion” and “antigen-binding fragment” of an antibody, as used herein, are interchangeable and refer to one or more fragments of an antibody that retain the ability to specifically bind to an antigen (e.g., human FAM19A5). Such “fragments” are, for example, between about 8 and about 1500 amino acids in length, suitably between about 8 and about 745 amino acids in length, suitably about 8 to about 300, for example about 8 to about 200 amino acids, or about 10 to about 50 or 100 amino acids in length. It has been shown that the antigen-binding function of an antibody can be performed by fragments of a full-length antibody. Examples of binding fragments encompassed within the term “antigen-binding portion” of an antibody, e.g., an anti-FAM19A5 antibody described herein, include (i) a Fab fragment, a monovalent fragment consisting of the VL, VH, CL, and CH1 domains; (ii) a F(ab′) 2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, and disulfide-linked Fvs (sdFv) (v) a dAb fragment (Ward et al., (1989) Nature 341:544-546), which consists of a VH domain; and (vi) an isolated complementarity determining region (CDR) or (vii) a combination of two or more isolated CDRs which can optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al., (1988) Science 242:423-426; and Huston et al., (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

As used herein, the terms “variable region” and “variable domain” are used interchangeably and are common in the art. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR).

Without wishing to be bound by any particular mechanism or theory, it is believed that the CDRs of the light and heavy chains are primarily responsible for the interaction and specificity of the antibody with antigen. In some aspects, the variable region is a human variable region. In some aspects, the variable region comprises rodent or murine CDRs and human framework regions (FRs). In some aspects, the variable region is a primate (e.g., non-human primate) variable region. In some aspects, the variable region comprises rodent or murine CDRs and primate (e.g., non-human primate) framework regions (FRs).

As used herein, the term “heavy chain” (HC) when used in reference to an antibody can refer to any distinct type, e.g., alpha (α), delta (δ), epsilon (ε), gamma (γ) and mu (μ), based on the amino acid sequence of the constant domain, which give rise to IgA, IgD, IgE, IgG, and IgM classes of antibodies, respectively, including subclasses of IgG, e.g., IgG1, IgG2, IgG3, and IgG4.

As used herein, the term “light chain” (LC) when used in reference to an antibody can refer to any distinct type, e.g., kappa (κ) or lambda (λ) based on the amino acid sequence of the constant domains. Light chain amino acid sequences are well known in the art. In some aspects, the light chain is a human light chain.

The terms “VL” and “VL domain” are used interchangeably to refer to the light chain variable region of an antibody.

The terms “VH” and “VH domain” are used interchangeably to refer to the heavy chain variable region of an antibody.

An “isolated antibody,” as used herein, is intended to refer to an antibody which is substantially free of other antibodies having different antigenic specificities (e.g., an isolated antibody that specifically binds to FAM19A5 is substantially free of antibodies that specifically bind antigens other than FAM19A5). An isolated antibody that specifically binds to an epitope of FAM19A5 can, however, have cross-reactivity to other FAM19A5 proteins from different species.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ and is expressed as a molar concentration (M), whereas K_{A} is calculated from the quotient of kₒₙ/k_{off}. kₒₙ refers to the association rate constant of, e.g., an antibody to an antigen, and k_{off} refers to the dissociation of, e.g., an antibody to an antigen. The kₒₙ and k_{off} can be determined by techniques known to one of ordinary skill in the art, such as immunoassays (e.g., enzyme-linked immunosorbent assay (ELISA)), BIACORE® or kinetic exclusion assay (KinExA).

As used herein, the terms “specifically binds,” “specifically recognizes,” “specific binding,” “selective binding,” and “selectively binds,” are analogous terms and refer to molecules (e.g., FAM19A5 antagonists described herein) that bind to an antigen (e.g., FAM19A5 protein) as such binding is understood by one skilled in the art. For example, a molecule that specifically binds to an antigen can bind to other peptides or polypeptides, generally with lower affinity as determined by, e.g., immunoassays, BIACORE®, KinExA 3000 instrument (Sapidyne Instruments, Boise, ID), or other assays known in the art. In some aspects, molecules that specifically bind to an antigen bind to the antigen with a K_{A} that is at least about 2 logs, at least about 2.5 logs, at least about 3 logs, at least about 4 logs or greater than the K_{A} when the molecules bind to another antigen.

As used herein, the term “antigen” refers to any natural or synthetic immunogenic substance, such as a protein, peptide, or hapten. An antigen can be FAM19A5 or a fragment thereof.

As used herein, an “epitope” is a term in the art and refers to a localized region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14, 15, or 20 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from (e.g., from FAM19A5) are tested for reactivity with a given antibody (e.g., anti-FAM19A5 antibody). Methods of determining spatial conformation of epitopes include techniques in the art and those described herein, for example, x-ray crystallography, 2-dimensional nuclear magnetic resonance and HDX-MS (see, e.g., Epitope Mapping Protocols in Methods in Molecular Biology, Vol. 66, G. E. Morris, Ed. (1996)).

The term “binds to the same epitope” with reference to two or more antibodies means that the antibodies bind to the same segment of amino acid residues, as determined by a given method. Techniques for determining whether antibodies bind to the “same epitope on FAM19A5” with the antibodies described herein include, for example, epitope mapping methods, such as, x-ray analyses of crystals of antigen: antibody complexes which provides atomic resolution of the epitope and hydrogen/deuterium exchange mass spectrometry (HDX-MS). Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, 2 and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, can be determined using known competition experiments. In some aspects, an antibody competes with, and inhibits binding of another antibody to a target by at least 10%, 20%, 30%, 40%, 50%, 60%, 70%, 80%, 90% or 100%. The level of inhibition or competition can be different depending on which antibody is the “blocking antibody” (i.e., the cold antibody that is incubated first with the target). Competition assays can be conducted as described, for example, in Ed Harlow and David Lane, Cold Spring Harb Protoc; 2006; doi: 10.1101/pdb.prot4277 or in Chapter 11 of “Using Antibodies” by Ed Harlow and David Lane, Cold Spring Harbor Laboratory Press, Cold Spring Harbor, NY, USA 1999. Competing antibodies bind to the same epitope, an overlapping epitope or to adjacent epitopes (e.g., as evidenced by steric hindrance).

The term “endogenous,” when used to describe members of the LRRC4 protein family, refers to LRRC4 family proteins that naturally exist in a subject. As described herein, the mimic molecules of the present disclosure differ (structurally and/or functionally) from endogenous LRRC4 protein family members.

Molecules (e.g., LRRC4 family mimic molecules) that “compete with another protein for binding to a target” refer to molecules that inhibit (partially or completely) the binding of the other protein (e.g., naturally existing members of the LRRC4 protein family) to the target. Whether two compounds compete with each other for binding to a target, i.e., whether and to what extent a LRRC4 family mimic molecule described herein inhibits the binding of the naturally existing members of the LRRC4 protein family to a FAM19A5 protein, can be determined using known competition experiments. In some aspects, a LRRC4 family mimic molecule described herein competes with, and inhibits the binding of the naturally existing members of the LRRC4 protein family to the FAM19A5 protein by at least about 10%, at least about 20%, at least about 30%, at least about 40%, at least about 50%, at least about 60%, at least about 70%, at least about 80%, at least about 90% or at least about 100%. Competition assays can be conducted as described herein or, for example, in Ed Harlow and David Lane, Cold Spring Harb Protoc; 2006; doi: 10.1101/pdb.prot4277 or in Chapter 11 of “Using Antibodies” by Ed Harlow and David Lane, Cold Spring Harbor Laboratory Press, Cold Spring Harbor, NY, USA 1999.

Other competitive binding assays that can be used with the present disclosure include: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)).

The term “naturally-occurring” or “naturally-existing,” as used herein, refers to the fact that an object (e.g., protein) can be found in nature. For example, a polypeptide or polynucleotide sequence that is present in an organism (including viruses) that can be isolated from a source in nature and which has not been intentionally modified by man in the laboratory is naturally-occurring. As further described elsewhere in the present disclosure, LRRC4 family mimic molecules useful for the present disclosure are not naturally-occurring.

A “polypeptide” refers to a chain comprising at least two consecutively linked amino acid residues, with no upper limit on the length of the chain. One or more amino acid residues in the protein can contain a modification such as, but not limited to, glycosylation, phosphorylation, or disulfide bond formation. A “protein” can comprise one or more polypeptides.

The term “nucleic acids” or “nucleic acid molecule,” as used herein, is intended to include DNA molecules and RNA molecules. A nucleic acid molecule can be single-stranded or double-stranded, and can be cDNA.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome.

Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”) In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell that comprises a nucleic acid that is not naturally present in the cell, and can be a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny cannot, in fact, be identical to the parent cell, but are still included within the scope of the term “host cell” as used herein.

As used herein, “administering” refers to the physical introduction of a molecule (e.g., FAM19A5 antagonist) or a composition comprising the molecule to a subject, using any of the various methods and delivery systems known to those skilled in the art. Non-limiting examples of routes of administration that can be used include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, a molecule described herein (e.g., FAM19A5 antagonist) can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

As used herein, the term “subject” includes any human or non-human animal. The term “non-human animal” includes all vertebrates, e.g., mammals and non-mammals, such as non-human primates, sheep, dog, cow, chickens, amphibians, reptiles, etc.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease. Treatment can be of a subject having a disease or a subject who does not have a disease (e.g., for prophylaxis).

### II. Methods of Treating

Disclosed herein are methods of treating a hearing disorder in a subject in need thereof, comprising administering to the subject an antagonist (e.g., isolated monoclonal antibody, or an antigen-binding fragment thereof) that specifically binds to FAM19A5 (also referred to herein as “FAM19A5 antagonist”). Also disclosed herein are means for treating a hearing disorder in a subject in need thereof comprising a pharmaceutical composition which comprises an antagonist against a family with sequence similarity 19, member A5 (FAM19A5) protein (FAM19A5 antagonist) and a pharmaceutically acceptable carrier. The present disclosure also provides methods of treating a hearing disorder in a subject in need thereof, comprising administering to the subject such means for treating a hearing disorder. As further described herein, in some aspects, a FAM19A5 antagonist comprises an anti-FAM19A5 antibody, such as that provided in Tables 6-9. In some aspects, a FAM19A5 antagonist comprises a LRRC4 family mimic molecule, such as those described herein.

As described and demonstrated herein, FAM19A5 antagonists described herein (e.g., anti-FAM19A5 antibody, LRRC4 family mimic molecule, or both) can be used to treat a wide-range of hearing disorders. In some aspects, a hearing disorder that can be treated with the present disclosure comprises a sensorineural hearing loss. Unless indicated otherwise, any sensorineural hearing loss known in the art can be treated with the present disclosure. In some aspects, the sensorineural hearing loss is associated with tinnitus. In some aspects, the sensorineural hearing loss is not associated with tinnitus. Non-limiting examples of sensorineural hearing loss that can be treated with the present disclosure comprises an ototoxic drug-induced hearing loss, noise-induced hearing loss, age-related hearing loss (also known as presbycusis), sudden hearing loss, hearing loss associated with Meniere's disease, autoimmune hearing loss, ischaemic hearing loss, head injury-associated hearing loss, genetic hearing loss, hearing loss associated with a viral or bacterial infection, hearing loss resulting from damage or disorder of the organ of corti, or combinations thereof. In some aspects, a sensorineural hearing loss that can be treated comprises an ototoxic drug-induced hearing loss. In some aspects, a sensorineural hearing loss that can be treated comprises a noise-induced hearing loss. In some aspects, a noise-induced hearing loss can be caused by a long-duration exposure to noise, for example, loud music, heavy equipment or mechanical device, airplanes, bombardment, or noise caused by other persons. In some aspects, a sensorineural hearing loss that can be treated comprises an age-related hearing loss. In some aspects, a sensorineural hearing loss that can be treated comprises a sudden hearing loss. In some aspects, a sensorineural hearing loss that can be treated comprises a hearing loss associated with a disease (e.g., Meniere's disease). In some aspects, a sensorineural hearing loss that can be treated comprises an autoimmune hearing loss. In some aspects, a sensorineural hearing loss that can be treated comprises an ischaemic hearing loss. In some aspects, a sensorineural hearing loss that can be treated comprises a head injury-associated hearing loss. In some aspects, a sensorineural hearing loss that can be treated comprises a genetic hearing loss. In some aspects, a sensorineural hearing loss that can be treated comprises a hearing loss associated with a viral or bacterial infection (e.g., labyrinthitis or encephalomeningitis). In some aspects, a sensorineural hearing loss that can be treated comprises a hearing loss resulting from damage or disorder of the organ of corti.

Optimal hearing is dependent on effective synaptic transmission at the ribbon synapse of auditory hair cells. Nouvian R., et al., J Membr Biol 209(2-3): 153-65 (February-March 2006); and LoGiudice L. and Matthews, G., Neuroscientist 15(4): 380-391 (August 2009). And, abnormal ribbon synapse function has been associated with many hearing impairments. Yuan X., et al., Drug Des Devel Ther 14:268502693 (July 2020); and Moser T., et al., Otol Neurotol 34(6): 995-1004 (August 2013). Not to be bound by any one theory, in some aspects, FAM19A5 antagonists provided herein (e.g., anti-FAM19A5 antibody, LRRC4 family mimic molecule, or both) can help reduce and/or improve ribbon synapse function. Accordingly, some aspects of the present disclosure relates to methods of reducing and/or preventing an impairment of a ribbon synapse within an inner ear of a subject in need thereof, comprising administering to the subject a FAM19A5 antagonist (e.g., anti-FAM19A5 antibody, LRRC4 family mimic molecule, or both). In some aspects, after the administration of a FAM19A5 antagonist, the impairment of a ribbon synapse is reduced by at least about 5%, at least about 10%, at least about 20%, at least about 30%, at least about 40%, at least about 50%, at least about 60%, at least about 70%, at least about 80%, at least about 90%, or about 100%, as compared to a reference subject (e.g., the subject prior to the FAM19A5 antagonist administration and/or a corresponding subject who did not receive an administration of the FAM19A5 antagonist).

Unless indicated otherwise, an “impairment of a ribbon synapse” refers to any abnormality of a ribbon synapse within the inner ear of the subject. In some aspects, the impairment is structural. For example, in some aspects, an impairment of a ribbon synapse comprises a reduced number of ribbon synapses within the inner ear. As is apparent from the present disclosure, in some aspects, the reduced number of ribbon synapses within the inner ear is associated with impaired hearing. In some aspects, a subject suffers from an impairment of a ribbon synapse where the number of ribbon synapses within the inner ear is reduced by at least about 5%, at least about 10%, at least about 20%, at least about 30%, at least about 40%, at least about 50%, at least about 60%, at least about 70%, at least about 80%, at least about 90%, or about 100%, as compared to a reference subject (e.g., corresponding subject who does not suffer from impaired hearing). Accordingly, some aspects of the present disclosure is related to methods of reducing and/or preventing the loss of ribbon synapses within the inner ear of a subject in need thereof, comprising administering to the subject a FAM19A5 antagonist described herein (e.g., anti-FAM19A5 antibody, LRRC4 family mimic molecule, or both).

In some aspects, after the administration of the FAM19A5 antagonist (e.g., anti-FAM19A5 antibody, LRRC4 family mimic molecule, or both), the number of ribbon synapses within the inner ear of the subject is increased, as compared to a reference subject (e.g., the subject prior to the FAM19A5 antagonist administration and/or a corresponding subject who did not receive an administration of the FAM19A5 antagonist). Accordingly, some aspects of the present disclosure relates to the method of increasing the number of ribbon synapses (e.g., by inducing the formation of the ribbon synapses) within the inner ear of the subject, comprising administering to the subject a FAM19A5 antagonist described herein (e.g., anti-FAM19A5 antibody, LRRC4 family mimic molecule, or both). In some aspects, as compared to the reference subject, the number of ribbon synapses is increased by at least about 5%, at least about 10%, at least about 20%, at least about 30%, at least about 40%, at least about 50%, at least about 60%, at least about 70%, at least about 80%, at least about 90%, or at least about 100% or more after the administration. In some aspects, as compared to the reference subject, the number of ribbon synapses is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 12.5-fold, at least about 15-fold, at least about 20-fold, at least about 25-fold, at least about 30-fold, at least about 35-fold, at least about 40-fold, at least about 45-fold, or at least about 50-fold after the administration. Not to be bound by any one theory, in some aspects, the increased number of ribbon synapses within the inner ear is associated with improved hearing.

In some aspects, an impairment of a ribbon synapse is functional. For example, in some aspects, an impairment of a ribbon synapse comprises reduced signal transmission after an auditory stimulation (e.g., sound vibration). In some aspects, the signal transmission is reduced by at least 5%, at least about 10%, at least about 20%, at least about 30%, at least about 40%, at least about 50%, at least about 60%, at least about 70%, at least about 80%, at least about 90%, or about 100%, as compared to that of a reference subject (e.g., corresponding subject who does not suffer from impaired hearing). Accordingly, some aspects of the present disclosure is related to methods of improving one or more functions (e.g., signal transmission) of a ribbon synapse within the inner ear of a subject, comprising administering to the subject a FAM19A5 antagonist provided herein (e.g., anti-FAM19A5 antibody, LRRC4 family mimic molecule, or both). In some aspects, after the administration, the one or more functions of a ribbon synapse is improved (e.g., enhanced or increased) by at least about 5%, at least about 10%, at least about 20%, at least about 30%, at least about 40%, at least about 50%, at least about 60%, at least about 70%, at least about 80%, at least about 90%, or at least about 100% or more, as compared to a reference subject (e.g., the subject prior to the FAM19A5 antagonist administration and/or a corresponding subject who did not receive an administration of the FAM19A5 antagonist). In some aspects, as compared to the reference subject, the number of ribbon synapses is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 12.5-fold, at least about 15-fold, at least about 20-fold, at least about 25-fold, at least about 30-fold, at least about 35-fold, at least about 40-fold, at least about 45-fold, or at least about 50-fold after the administration. Not to be bound by any one theory, in some aspects, an improvement in one or more functions of a ribbon synapse within the inner ear of a subject is associated with improved hearing.

In some aspects, an impairment of a ribbon synapse is both structural (e.g., reduced number of ribbon synapses within the inner ear) and functional (e.g., reduced signal transmission after an auditory simulation). For example, in some aspects, after administering a FAM19A5 antagonist (e.g., anti-FAM19A5 antibody, LRRC4 family mimic molecule, or both) to a subject in need thereof, both the number and one or more functions of ribbon synapses within the inner ear of the subject is improved (e.g., increased or enhanced).

As demonstrated herein, in some aspects, hearing disorders that can be treated with the present disclosure is associated with an increased FAM19A5 activity. For example, in some aspects, a spiral ganglion neuron of a subject suffering from a hearing disorder exhibits an increased expression of FAM19A5. Unless indicated otherwise, the increase in FAM19A5 expression can be at the gene level (e.g., FAM19A5 mRNA) and/or at the protein level. In some aspects, the FAM19A5 expression in a spiral ganglion neuron of a subject suffering from a hearing disorder is increased by at least about 5%, at least about 10%, at least about 20%, at least about 30%, at least about 40%, at least about 50%, at least about 60%, at least about 70%, at least about 80%, at least about 90%, or at least about 100% or more, as compared to a reference subject (e.g., a corresponding subject who does not suffer from a hearing disorder). In some aspects, compared to the spiral ganglion neuron of the reference subject, FAM19A5 expression is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 12.5-fold, at least about 15-fold, at least about 20-fold, at least about 25-fold, at least about 30-fold, at least about 35-fold, at least about 40-fold, at least about 45-fold, or at least about 50-fold. As will be apparent to those skilled in the arts, an increase in FAM19A5 expression can be associated with FAM19A5 activity. Accordingly, in some aspects, the FAM19A5 activity in a spiral ganglion neuron of a subject suffering from a hearing disorder is increased by at least about 5%, at least about 10%, at least about 20%, at least about 30%, at least about 40%, at least about 50%, at least about 60%, at least about 70%, at least about 80%, at least about 90%, or at least about 100% or more, as compared to the reference subject (e.g., a corresponding subject who does not suffer from a hearing disorder). In some aspects, compared to the reference subject, FAM19A5 activity in a spiral ganglion neuron of a subject suffering from a hearing disorder is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 12.5-fold, at least about 15-fold, at least about 20-fold, at least about 25-fold, at least about 30-fold, at least about 35-fold, at least about 40-fold, at least about 45-fold, or at least about 50-fold.

Not to be bound by any one theory, in some aspects, administering a FAM19A5 antagonist described herein (e.g., anti-FAM19A5 antibody, LRRC4 family mimic molecule, or both) results in decreased FAM19A5 expression and/or activity within a spiral ganglion neuron. As is apparent from the present disclosure, in some aspects, the decrease in FAM19A5 expression and/or activity within a spiral ganglion neuron can help improve hearing. Accordingly, some aspects of the present disclosure relates to methods of reducing an expression and/or activity of FAM19A5 within a spiral ganglion neuron of a subject in need thereof, comprising administering to the subject a FAM19A5 antagonist described herein (e.g., anti-FAM19A5 antibody, LRRC4 family mimic molecule, or both). In some aspects, after the administration of the FAM19A5 antagonist (e.g., anti-FAM19A5 antibody, LRRC4 family mimic molecule, or both), the expression and/or activity of FAM19A5 within the spiral ganglion neuron of the subject is decreased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

As demonstrated herein, in some aspects, a FAM19A5 antagonist described herein (e.g., anti-FAM19A5 antibody, LRRC4 family mimic molecule, or both) is capable of increasing a neurite outgrowth of a neuron (e.g., spiral ganglion neuron). As used herein, the term “neurite outgrowth” refers to the process in which neurons produce new projections (axon, dendrite, or both) as they grow. Accordingly, some aspects of the present disclosure is related to methods of increasing neurite outgrowth of a spiral ganglion neuron of a subject in need thereof, comprising administering to the subject a FAM19A5 antagonist described herein (e.g., anti-FAM19A5 antibody, LRRC4 family mimic molecule, or both). In some aspects, after the administration of the FAM19A5 antagonist, neurite outgrowth is increased by at least about 10%, at least about 20%, at least about 30%, at least about 40%, at least about 50%, at least about 60%, at least about 70%, at least about 80%, at least about 90%, or at least about 100%. In some aspects, after the administration of the FAM19A5 antagonist, neurite outgrowth is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 12.5-fold, at least about 15-fold, at least about 20-fold, at least about 25-fold, at least about 30-fold, at least about 35-fold, at least about 40-fold, at least about 45-fold, or at least about 50-fold. In some aspects, a FAM19A5 antagonist described herein (e.g., anti-FAM19A5 antibody, LRRC4 family mimic molecule, or both) is capable of reversing FAM19A5-induced inhibition of neurite outgrowth.

In some aspects, a FAM19A5 antagonist described herein (e.g., anti-FAM19A5 antibody, LRRC4 family mimic molecule, or both) is capable of promoting synaptogenesis. As used herein, the term “synaptogenesis” refers to the process by which synapses are formed between neurons (e.g., spiral ganglion neurons). Accordingly, in some aspects, the present disclosure provides a method of increasing synaptogenesis in a subject in need thereof, comprising administering to the subject a FAM19A5 antagonist described herein (e.g., anti-FAM19A5 antibody, LRRC4 family mimic molecule, or both). In some aspects, after the administration of the FAM19A5 antagonist, synaptogenesis is increased by at least about 10%, at least about 20%, at least about 30%, at least about 40%, at least about 50%, at least about 60%, at least about 70%, at least about 80%, at least about 90%, or at least about 100%. In some aspects, after the administration of the FAM19A5 antagonist, synaptogenesis is increased by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 12.5-fold, at least about 15-fold, at least about 20-fold, at least about 25-fold, at least about 30-fold, at least about 35-fold, at least about 40-fold, at least about 45-fold, or at least about 50-fold. In some aspects, a FAM19A5 antagonist described herein is capable of reversing FAM19A5-induced inhibition of synaptogenesis.

As described elsewhere in the present disclosure, for any of the methods provided herein, a FAM19A5 antagonist (e.g., anti-FAM19A5 antibody, LRRC4 family mimic molecule, or both) can be administered to the subject using any suitable administration routes known in the art. In some aspects, the FAM19A5 antagonist (e.g., anti-FAM19A5 antibody, LRRC4 family mimic molecule, or both) is administered to the subject via intracochlear injection, intravestibular injection, intravenous administration, intratympanic administration, or combinations thereof. In some aspects, the FAM19A5 antagonist (e.g., anti-FAM19A5 antibody, LRRC4 family mimic molecule, or both) is administered to the subject via intracochlear injection. In some aspects, the FAM19A5 antagonist (e.g., anti-FAM19A5 antibody, LRRC4 family mimic molecule, or both) is administered to the subject via intravestibular injection. In some aspects, the FAM19A5 antagonist (e.g., anti-FAM19A5 antibody, LRRC4 family mimic molecule, or both) is administered to the subject via intravenous administration. In some aspects, the FAM19A5 antagonist (e.g., anti-FAM19A5 antibody, LRRC4 family mimic molecule, or both) is administered to the subject via intratympanic administration.

In some aspects, any of the methods provided herein (e.g., provided above) can further comprise administering an additional therapeutic agent to the subject. For example, some aspects of the present disclosure comprises treating a hearing disorder in a subject in need thereof, comprising administering to the subject a FAM19A5 antagonist (e.g., anti-FAM19A5 antibody, LRRC4 family mimic molecule, or both) and an additional therapeutic agent. Non-limiting examples of such additional therapeutic agents are known in the art (e.g., standard of care, e.g., steroid for sudden deafness). Dose and administration of the one or more additional therapeutic agents are known in the art, e.g., as instructed by the product label of the respective drug.

Where a FAM19A5 antagonist (e.g., anti-FAM19A5 antibody, LRRC4 family mimic molecule, or both) is administered to a subject in combination with an additional therapeutic agent, in some aspects, the FAM19A5 antagonist and the additional therapeutic agent can be administered to the subject concurrently. For example, in some aspects, the FAM19A5 antagonist (e.g., anti-FAM19A5 antibody, LRRC4 family mimic molecule, or both) and the additional therapeutic agent can be administered to the subject as a single composition. In some aspects, the FAM19A5 antagonist (e.g., anti-FAM19A5 antibody, LRRC4 family mimic molecule, or both) and the additional therapeutic agent can be administered concurrently but as separate compositions. In some aspects, the FAM19A5 antagonist (e.g., anti-FAM19A5 antibody, LRRC4 family mimic molecule, or both) and the additional therapeutic agent can be administered to the subject sequentially. For example, in some aspects, the FAM19A5 antagonist (e.g., anti-FAM19A5 antibody, LRRC4 family mimic molecule, or both) is administered to the subject prior to the administration of the additional therapeutic agent. In some aspects, the FAM19A5 antagonist (e.g., anti-FAM19A5 antibody, LRRC4 family mimic molecule, or both) is administered to the subject after the administration of the additional therapeutic agent.

### III. FAM19A5 Antagonists

One or more FAM19A5 antagonists can be used with the present methods. In some aspects, a FAM19A5 antagonist comprises an antisense oligonucleotide, siRNA, shRNA, miRNA, dsRNA, aptamer, PNA (peptide nucleic acid) that specifically targets FAM19A5, or a vector including the same. In some aspects, a FAM19A5 antagonist comprises an anti-FAM19A5 antibody, a polynucleotide encoding the anti-FAM19A5 antibody, or a vector comprising the polynucleotide thereof. Accordingly, in some aspects, a FAM19A5 agonist useful for the methods provided herein comprises an antisense oligonucleotide that specifically targets FAM19A5. In some aspects, a FAM19A5 antagonist comprises a siRNA that specifically targets FAM19A5. In some aspects, a FAM19A5 antagonist comprises a shRNA that specifically targets FAM19A5. In some aspects, a FAM19A5 antagonist comprises a miRNA that specifically targets FAM19A5. In some aspects, a FAM19A5 antagonist comprises a dsRNA that specifically targets FAM19A5. In some aspects, a FAM19A5 antagonist comprises an aptamer that specifically targets FAM19A5. In some aspects, a FAM19A5 antagonist comprises a PNA that specifically targets FAM19A5. In some aspects, a FAM19A5 antagonist comprise an anti-FAM19A5 antibody. In some aspects, a FAM19A5 antagonist comprises a polynucleotide encoding an anti-FAM19A5 antibody. In some aspects, a FAM19A5 antagonist useful for the present disclosure comprises a LRRC4 family mimic molecule.

As is apparent from the present disclosure, FAM19A5 antagonists useful for the present disclosure can be characterized by particular structural and/or functional features. For example, in some aspects, a FAM19A5 antagonist described herein specifically binds human FAM19A5, including soluble FAM19A5 and membrane bound FAM19A5. In addition to binding specifically to soluble and/or membrane bound human FAM19A5, the FAM19A5 antagonist described herein can also (a) bind to soluble human FAM19A5 with a K_{D} of 10 nM or less; (b) bind to membrane bound human FAM19A5 with a K_{D} of 10 nM or less; or both (a) and (b).

In some aspects, a FAM19A5 antagonist suitable for the methods disclosed herewith cross-competes for binding to (or inhibits binding of) a human FAM19A5 with an anti-FAM19A5 antibody comprising CDRs or variable regions disclosed herein. Non-limiting examples of such anti-FAM19A5 antibodies are provided elsewhere in the present disclosure.

For instance, in some aspects, a FAM19A5 antagonist described herein cross-competes for binding to a human FAM19A5 with a reference antibody, wherein the reference antibody comprises the heavy and light chain CDRs of one or more of the following antibodies described herein: 1-65, 3-2, 2-13, 1-28, P2-C12, 13B4, 13F7, 15A9, P1-A03, P1-A08, P1-F02, P2-A01, P2-A03, P2-F07, P2-F11, SS01-13, SS01-13-s5, S5-2.GKNG, 1-7A-IT, Low-PI, 1-30, 1-17, 1-32, 4-11, 6-10, 2-13D, 2-13D-37, 2-13D-37-1.5W-41, 2-13D-37-3W-16. In some aspects, a FAM19A5 antagonist useful for the present disclosure cross-competes for binding to a human FAM19A5 with a reference antibody, wherein the reference antibody comprises the heavy and light chain variable regions of one or more of the following antibodies described herein: 1-65, 3-2, 2-13, 1-28, P2-C12, 13B4, 13F7, 15A9, P1-A03, P1-A08, P1-F02, P2-A01, P2-A03, P2-F07, P2-F11, SS01-13, SS01-13-s5, S5-2.GKNG, 1-7A-IT, Low-PI, 1-30, 1-17, 1-32, 4-11, 6-10, 2-13D, 2-13D-37, 2-13D-37-1.5W-41, 2-13D-37-3W-16.

In some aspects, a FAM19A5 antagonist useful for the present disclosure cross-competes for binding to a human FAM19A5 with a reference antibody, wherein the reference antibody comprises a heavy chain variable region (VH) CDR1, CDR2, and CDR3, and a light chain variable region (VL) CDR1, CDR2, and CDR3, wherein: (i) the VH CDR1 of the reference antibody comprises the amino acid sequence set forth in any one of SEQ ID NOs: 11, 14, 17, 20, 89, 95, 101, 107, 113, 119, 125, 131, 137, 143, 149, 218, 220, 222, and 223; (ii) the VH CDR2 of the reference antibody comprises the amino acid sequence set forth in any one of SEQ ID NOs: 12, 15, 18, 21, 90, 96, 102, 108, 114, 120, 126, 132, 138, 144, 150, 252, 253, 219, and 221; (iii) the VH CDR3 of the reference antibody comprises the amino acid sequence set forth in any one of SEQ ID NOs: 13, 16, 19, 22, 91, 97, 103, 109, 115, 121, 127, 133, 139, 145, 151, 246, and 247; (iv) the VL CDR1 of the reference antibody comprises the amino acid sequence set forth in any one of SEQ ID NOs: 23, 26, 29, 32, 92, 98, 104, 110, 116, 122, 128, 134, 140, 146, 152, 208, 225, and 248; (v) the VL CDR2 of the reference antibody comprises the amino acid sequence set forth in any one of SEQ ID NOs: 24, 27, 30, 33, 93, 99, 105, 111, 117, 123, 129, 135, 141, 147, 153, 209, 213, 224, 226, 228, 229, 230, and 232; and/or (vi) the VL CDR3 of the reference antibody comprises the amino acid sequence set forth in any one of SEQ ID NOs: 25, 28, 31, 34, 94, 100, 106, 112, 118, 124, 130, 136, 142, 148, 154, 227, 231, and 233.

In some aspects, a FAM19A5 antagonist described herein binds to the same FAM19A5 epitope as a reference antibody. In some aspects, both the FAM19A5 antagonist and the reference antibody bind to one or more of the FAM19A5 epitopes described herein (e.g., comprising the amino acid sequence set forth in any one of SEQ ID NOs: 89-94). In some aspects, a FAM19A5 antagonist described herein binds to the same FAM19A5 epitope as a reference antibody, wherein the reference antibody comprises the heavy and light chain CDRs of one or more of the following antibodies described herein: 1-65, 3-2, 2-13, 1-28, P2-C12, 13B4, 13F7, 15A9, P1-A03, P1-A08, P1-F02, P2-A01, P2-A03, P2-F07, P2-F11, SS01-13, SS01-13-s5, S5-2.GKNG, 1-7A-IT, Low-PI, 1-30, 1-17, 1-32, 4-11, 6-10, 2-13D, 2-13D-37, 2-13D-37-1.5W-41, 2-13D-37-3W-16. In some aspects, a FAM19A5 antagonist binds to the same FAM19A5 epitope as a reference antibody, wherein the reference antibody comprises the heavy and light chain variable regions of one or more of the following antibodies described herein: 1-65, 3-2, 2-13, 1-28, P2-C12, 13B4, 13F7, 15A9, P1-A03, P1-A08, P1-F02, P2-A01, P2-A03, P2-F07, P2-F11, SS01-13, SS01-13-s5, S5-2.GKNG, 1-7A-IT, Low-PI, 1-30, 1-17, 1-32, 4-11, 6-10, 2-13D, 2-13D-37, 2-13D-37-1.5W-41, 2-13D-37-3W-16.

In some aspects, a FAM19A5 antagonist described herein binds to the same FAM19A5 epitope as a reference antibody, wherein the reference antibody comprises a heavy chain variable region (VH) CDR1, CDR2, and CDR3, and a light chain variable region (VL) CDR1, CDR2, and CDR3, wherein: (i) the VH CDR1 of the reference antibody comprises the amino acid sequence set forth in any one of SEQ ID NOs: 11, 14, 17, 20, 89, 95, 101, 107, 113, 119, 125, 131, 137, 143, 149, 218, 220, 222, and 223; (ii) the VH CDR2 of the reference antibody comprises the amino acid sequence set forth in any one of SEQ ID NOs: 12, 15, 18, 21, 90, 96, 102, 108, 114, 120, 126, 132, 138, 144, 150, 252, 253, 219, and 221; (iii) the VH CDR3 of the reference antibody comprises the amino acid sequence set forth in any one of SEQ ID NOs: 13, 16, 19, 22, 91, 97, 103, 109, 115, 121, 127, 133, 139, 145, 151, 246, and 247; (iv) the VL CDR1 of the reference antibody comprises the amino acid sequence set forth in any one of SEQ ID NOs: 23, 26, 29, 32, 92, 98, 104, 110, 116, 122, 128, 134, 140, 146, 152, 208, 225, and 248; (v) the VL CDR2 of the reference antibody comprises the amino acid sequence set forth in any one of SEQ ID NOs: 24, 27, 30, 33, 93, 99, 105, 111, 117, 123, 129, 135, 141, 147, 153, 209, 213, 224, 226, 228, 229, 230, and 232; and/or (vi) the VL CDR3 of the reference antibody comprises the amino acid sequence set forth in any one of SEQ ID NOs: 25, 28, 31, 34, 94, 100, 106, 112, 118, 124, 130, 136, 142, 148, 154, 227, 231, and 233.

In some aspects, FAM19A5 antagonists provided herein binds to FAM19A5 (e.g., human FAM19A5) with a 20%, 25%, 30%, 35%, 40%, 45%, 50%, 55%, 60%, 65%, 70%, 75%, 80%, 85%, 90%, 95% or higher affinity than to another protein in the FAM19A5 family as measured by, e.g., a immunoassay (e.g., ELISA), surface plasmon resonance, or kinetic exclusion assay. In some aspects, a FAM19A5 antagonist provided herein binds to FAM19A5 (e.g., human FAM19A5) with no cross reactivity with another protein in the FAM19A5 family as measured by, e.g., an immunoassay.

### III. A. Exemplary Anti-FAM19A5 Antibodies

In some aspects, a FAM19A5 antagonist that can be used with the methods provided herein comprises an antibody. In some aspects, particular antibodies that can be used in the methods disclosed herein are antibodies, e.g., monoclonal antibodies, having the CDR and/or variable region sequences disclosed herein, as well as antibodies having at least 80% identity (e.g., at least 85%, at least 90%, at least 95%, or at least 99% identity) to their variable region or CDR sequences. The amino acid sequences for the VH and VL CDRs for the different exemplary anti-FAM19A5 antibodies are provided in Tables 6 and 7, respectively. The CDRs for the following antibodies were identified using the Kabat numbering scheme (see supra): 1-65, 3-2, 2-13, 1-28, P2-C12, 13B4, 13F7, 15A9, P1-A03, P1-A08, P1-F02, P2-A01, P2-A03, P2-F07, P2-F11, SS01-13, SS01-13-s5, and S5-2.GKNG. The CDRs for the following antibodies were identified using the IMGT numbering system (see supra): 1-7A-IT, Low-PI, 1-30, 1-17, 1-32, 4-11, 6-10, 2-13D, 2-13D-37, 2-13D-37-1.5W-41, and 2-13D-37-3W-16. The VH and VL amino acid sequences of different anti-FAM19A5 antibodies of the present disclosure are provided in Tables 8 and 9, respectively.

TABLE 6
Variable heavy chain CDR amino acid sequences

| | | | |
|---|---|---|---|
| Antibody | VH-CDR1 | VH-CDR2 | VH-CDR3 |

According to Kabat System

| | | | |
|---|---|---|---|
| Anti-FAM19A5 | SHGMF | EITNDGSGTNYGSAVKG | STYECPGGFSCWGDTGQIDA |
| (“2-13”) | (SEQ ID NO: 11) | (SEQ ID NO: 12) | (SEQ ID NO: 13) |
| Anti-FAM19A5 | SFNMF | QISSSGSSTNYAPAVRG | SSYDCPYGHCSSGVDSAGEIDA |
| (“3-2”) | (SEQ ID NO: 14) | (SEQ ID NO: 15) | (SEQ ID NO: 16) |
| Anti-FAM19A5 | SYQMG | VINKSGSDTS | GSASYITAATIDA |
| (“1-65”) | (SEQ ID NO: 17) | (SEQ ID NO: 18) | (SEQ ID NO: 19) |
| Anti-FAM19A5 | GFDFSDYG | IRSDGSNP | AKDGNGYCALDAYRSGGYSCGVY |
| (“1-28”) | (SEQ ID NO: 20) | (SEQ ID NO: 21) | PGSIDA |
| | | | (SEQ ID NO: 22) |
| Anti-FAM19A5 | TYAVT | YINWRGGTSYANWAKG | DASSGAAFGSYGMDP |
| (“P2-C12”) | (SEQ ID NO: 89) | (SEQ ID NO: 90) | (SEQ ID NO: 91) |
| Anti-FAM19A5 | SSNWWS | EIYHGGTTNYNPSLKG | WQLVGGLDV |
| (“13B4”) | (SEQ ID NO: 95) | (SEQ ID NO: 96) | (SEQ ID NO: 97) |
| Anti-FAM19A5 | GYSWT | EISHFGSANYNPSLKS | ALRGTYSRFYYGMDV |
| (“13F7”) | (SEQ ID NO: 101) | (SEQ ID NO: 102) | (SEQ ID NO: 103) |
| Anti-FAM19A5 | SYYWS | YIYPSGSTNYNPSLKS | VNPFGYYYAMDV |
| (“15A9”) | (SEQ ID NO: 107) | (SEQ ID NO: 108) | (SEQ ID NO: 109) |
| Anti-FAM19A5 | SDYMS | IIYPSTTTYYASWAKG | GSNWSSGMNL |
| (“P1-A03”) | (SEQ ID NO: 113) | (SEQ ID NO: 114) | (SEQ ID NO: 115) |
| Anti-FAM19A5 | TYYMS | IVYPSGTTYYANWAKG | GDSFGYGL |
| (“P1-A08”) | (SEQ ID NO: 119) | (SEQ ID NO: 120) | (SEQ ID NO: 121) |
| Anti-FAM19A5 | NYYMG | IIYASGSTYYASWAKG | IDIGVGDYGWAYDRLDL |
| (“P1-F02”) | (SEQ ID NO: 125) | (SEQ ID NO: 126) | (SEQ ID NO: 127) |
| Anti-FAM19A5 | GYYMS | IIYPSGSTDYASWAKG | VAGYVGYGYETFFDI |
| (“P2-A01”) | (SEQ ID NO: 131) | (SEQ ID NO: 132) | (SEQ ID NO: 133) |
| Anti-FAM19A5 | NYDMS | FMDTDGSAYYATWAKG | RGSSYYGGIDI |
| (“P2-A03”) | (SEQ ID NO: 137) | (SEQ ID NO: 138) | (SEQ ID NO: 139) |
| Anti-FAM19A5 | SYYMN | IIYPSGTTYYAGWAKG | TVSGYFDI |
| (“P2-F07”) | (SEQ ID NO: 143) | (SEQ ID NO: 144) | (SEQ ID NO: 145) |
| Anti-FAM19A5 | SYGVS | YIANNYNPHYASWAKG | DNYGMDP |
| (“P2-F11”) | (SEQ ID NO: 149) | (SEQ ID NO: 150) | (SEQ ID NO: 151) |
| Anti-FAM19A5 | SYQMG | VINKSGSDTS | GSASYITAATIDA |
| (“SS01-13”) | (SEQ ID NO: 17) | (SEQ ID NO: 18) | (SEQ ID NO: 19) |
| Anti-FAM19A5 | SYQMG | AINKSGSDTS | GSASYITAATIDA |
| (“SS01-13-s5”) | (SEQ ID NO: 17) | (SEQ ID NO: 252) | (SEQ ID NO: 19) |
| Anti-FAM19A5 | SYQMG | AINKGGSDTS | GSASYITAATIDA |
| (“S5-2.GKNG”) | (SEQ ID NO: 17) | (SEQ ID NO: 253) | (SEQ ID NO: 19) |

According to IMGT system

| | | | |
|---|---|---|---|
| Anti-FAM19A5 | GFTFSSFNMF (SEQ | QISSSGSSTNYAPAVKG | SSYDCPYGHCSSGVDSAGEIDA |
| (“1-7A-IT”) | ID NO: 218) | (SEQ ID NO: 219) | (SEQ ID NO: 16) |
| Anti-FAM19A5 | GFDFESFNMF (SEQ | QISSSEEDENYAPAVKG | SSYDCPYGHCSSGVDSAGEIDA |
| (“Low-PI”) | ID NO: 220) | (SEQ ID NO: 221) | (SEQ ID NO: 16) |
| Anti-FAM19A5 | GFDFESFNMF (SEQ | QISSSEEDENYAPAVKG | SSYDCPYGHCSSGVDSAGEIDA |
| (“1-30”) | ID NO: 220) | (SEQ ID NO: 221) | (SEQ ID NO: 16) |
| Anti-FAM19A5 | GFDFESFNMF (SEQ | QISSSEEDENYAPAVKG | SSYDCPYGHCSSGVDSAGEIDA |
| (“1-17”) | ID NO: 220) | (SEQ ID NO: 221) | (SEQ ID NO: 16) |
| Anti-FAM19A5 | GFDFESFNMF (SEQ | QISSSEEDENYAPAVKG | SSYDCPYGHCSSGVDSAGEIDA |
| (“1-32”) | ID NO: 220) | (SEQ ID NO: 221) | (SEQ ID NO: 16) |
| Anti-FAM19A5 | GFDFESFNMF (SEQ | QISSSEEDENYAPAVKG | SSYDCPYGHCSSGVDSAGEIDA |
| (“4-11”) | ID NO: 220) | (SEQ ID NO: 221) | (SEQ ID NO: 16) |
| Anti-FAM19A5 | GFDFESFNMF (SEQ | QISSSEEDENYAPAVKG | SSYDCPYGHCSSGVDSAGEIDA |
| (“6-10”) | ID NO: 220) | (SEQ ID NO: 221) | (SEQ ID NO: 16) |
| Anti-FAM19A5 | GFTFSSHGMF (SEQ | EITNDGSGTNYGSAVKG | STYECPGGFSCWGDTGQIDA |
| (“2-13D”) | ID NO: 222) | (SEQ ID NO: 12) | (SEQ ID NO: 13) |
| Anti-FAM19A5 | GFDFSSHGMF (SEQ | EITNDGSGTNYGSAVKG | STYECPGGFSCWGDTGQIDA |
| (“2-13D-37”) | ID NO: 223) | (SEQ ID NO: 12) | (SEQ ID NO: 13) |
| Anti-FAM19A5 | GFDFSSHGMF (SEQ | EITNDGSGTNYGSAVKG | SSYVCPGGFSCWGDTGQIDA |
| (“2-13D-37- | ID NO: 223) | (SEQ ID NO: 12) | (SEQ ID NO: 246) |
| 1.5W-41”) | | | |
| Anti-FAM19A5 | GFDFSSHGMF (SEQ | EITNDGSGTNYGSAVKG | SNYACPGGFSCWGDTGQIDA |
| (“2-13D-37-3W- | ID NO: 223) | (SEQ ID NO: 12) | (SEQ ID NO: 247) |
| 16”) | | | |

TABLE 7
Variable light chain CDR amino acid sequences (according to Kabat system)

| | | | |
|---|---|---|---|
| Antibody | VL-CDR1 | VL-CDR2 | VL-CDR3 |

According to Kabat System

| | | | |
|---|---|---|---|
| Anti-FAM19A5 | SGGSYSYG | WDDERPS | GTEDISGTAGV |
| (“2-13”) | (SEQ ID NO: 23) | (SEQ ID NO: 24) | (SEQ ID NO: 25) |
| Anti-FAM19A5 | SGGGSYAGSYYYG | ESNKRPS (SEQ ID NO: 27) | GSWDSSNGGI (SEQ ID |
| (“3-2”) | (SEQ ID NO: 26) | | NO: 28) |
| Anti-FAM19A5 | SGGGSSGYGYG | WNDKRPS (SEQ ID NO: 30) | GNDDYSSDSGYVGV (SEQ |
| (“1-65”) | (SEQ ID NO: 29) | | ID NO: 31) |
| Anti-FAM19A5 | GYGYG | QND | GSEDSSTLAGI |
| (“1-28”) | (SEQ ID NO: 32) | (SEQ ID NO: 33) | (SEQ ID NO: 34) |
| Anti-FAM19A5 | QASQSISSYLS | EASKLAS | QQGYSSTNVWNA |
| (“P2-C12”) | (SEQ ID NO: 92) | (SEQ ID NO: 93) | (SEQ ID NO: 94) |
| Anti-FAM19A5 | SGDKLGNVYAS | QDNKRPS | QAWDSSTAV |
| (“13B4”) | (SEQ ID NO: 98) | (SEQ ID NO: 99) | (SEQ ID NO: 100) |
| Anti-FAM19A5 | RSSQSLLHSNGYNYLD | LGSNRAS | MQARQTPLT |
| (“13F7”) | (SEQ ID NO: 104) | (SEQ ID NO: 105) | (SEQ ID NO: 106) |
| Anti-FAM19A5 | RASQSISTSLN | GASTLQS | QESASIPRT |
| (“15A9”) | (SEQ ID NO: 110) | (SEQ ID NO: 111) | (SEQ ID NO: 112) |
| Anti-FAM19A5 | LASEDIYSGIS | GASNLES | LGGYSYSSTGLT |
| (“P1-A03”) | (SEQ ID NO: 116) | (SEQ ID NO: 117) | (SEQ ID NO: 118) |
| Anti-FAM19A5 | TADTLSRSYAS | RDTSRPS | ATSDGSGSNYQYV |
| (“P1-A08”) | (SEQ ID NO: 122) | (SEQ ID NO: 123) | (SEQ ID NO: 124) |
| Anti-FAM19A5 | LASEDIYSGIS | GASNLES | LGGYSYSSIT |
| (“P1-F02”) | (SEQ ID NO: 128) | (SEQ ID NO: 129) | (SEQ ID NO: 130) |
| Anti-FAM19A5 | LASEDIYSGIS | GASNLES | LGGVTYSSTGTHLT |
| (“P2-A01”) | (SEQ ID NO: 134) | (SEQ ID NO: 135) | (SEQ ID NO: 136) |
| Anti-FAM19A5 | QASQSIGGNLA | RASTLAS | QSPAYDPAAYVGNA |
| (“P2-A03”) | (SEQ ID NO: 140) | (SEQ ID NO: 141) | (SEQ ID NO: 142) |
| Anti-FAM19A5 | LASEDIYSALA | GTSNLES | QGYSSYPLT |
| (“P2-F07”) | (SEQ ID NO: 146) | (SEQ ID NO: 147) | (SEQ ID NO: 148) |
| Anti-FAM19A5 | QASQSVYNNKNLA | AASTLAS | QGEFSCSSADCNA |
| (“P2-F11”) | (SEQ ID NO: 152) | (SEQ ID NO: 153) | (SEQ ID NO: 154) |
| Anti-FAM19A5 | SGGASSGYGYG (SEQ | KDDERPS (SEQ ID NO: | GNDDYSSDSGYVGV (SEQ |
| (“SS01-13”) | ID NO: 208) | 209) | ID NO: 31) |
| Anti-FAM19A5 | SGGASSGYGYG (SEQ | KDSERPS (SEQ ID NO: | GNDDYSSDSGYVGV (SEQ |
| (“SS01-13-S5”) | ID NO: 208) | 213) | ID NO: 31) |
| Anti-FAM19A5 | SGGASSGYGYG (SEQ | KDSERPS (SEQ ID NO: | GNDDYSSDSGYVGV (SEQ |
| (“S5-2.GKNG”) | ID NO: 208) | 213) | ID NO: 31) |

According to IMGT system

| | | | |
|---|---|---|---|
| Anti-FAM19A5 | SGGGSYAGSYYYG | ENNKRPS (SEQ ID NO: | GSWDSSNGGI (SEQ ID |
| (“1-7A-IT”) | (SEQ ID NO: 26) | 224) | NO: 28) |
| Anti-FAM19A5 | SGGGSEEEQYYYG | EDEERPS (SEQ ID NO: | GSWDSEDEDH (SEQ ID |
| (“Low-PI”) | (SEQ ID NO: 225) | 226) | NO: 227) |
| Anti-FAM19A5 | SGGGSEEEQYYYG | QDEERPS (SEQ ID NO: | GSWDSEDEDH (SEQ ID |
| (“1-30”) | (SEQ ID NO: 225) | 228) | NO: 227) |
| Anti-FAM19A5 | SGGGSYAGSYYYG | EDEQRPS (SEQ ID NO: | GSWDSEDEDH (SEQ ID |
| (“1-17”) | (SEQ ID NO: 26) | 229) | NO: 227) |
| Anti-FAM19A5 | SGGGSYAGSYYYG | QDEERPS (SEQ ID NO: | GSWDSEDEDH (SEQ ID |
| (“1-32”) | (SEQ ID NO: 26) | 228) | NO: 227) |
| Anti-FAM19A5 | SGGGSYAGSYYYG | EDHERPS (SEQ ID NO: | GSWDSSDEDH (SEQ ID |
| (“4-11”) | (SEQ ID NO: 26) | 230) | NO: 231) |
| Anti-FAM19A5 | SGGGSYAGSYYYG | QDLLRPS (SEQ ID NO: | GSWDSLSSSH (SEQ ID |
| (“6-10”) | (SEQ ID NO: 26) | 232) | NO: 233) |
| Anti-FAM19A5 | SGGVYSYG (SEQ ID | WDDERPS (SEQ ID NO: 24) | GTEDISGTAGV (SEQ ID |
| (“2-13D”) | NO: 248) | | NO: 25) |
| Anti-FAM19A5 | SGGVYSYG (SEQ ID | WDDERPS (SEQ ID NO: 24) | GTEDISGTAGV (SEQ ID |
| (“2-13D-37”) | NO: 248) | | NO: 25) |
| Anti-FAM19A5 | SGGVYSYG (SEQ ID | WDDERPS (SEQ ID NO: 24) | GTEDISGTAGV (SEQ ID |
| (“2-13D-37- | NO: 248) | | NO: 25) |
| 1.5W-41”) | | | |
| Anti-FAM19A5 | SGGVYSYG (SEQ ID | WDDERPS (SEQ ID NO: 24) | GTEDISGTAGV (SEQ ID |
| (“2-13D-37-3W- | NO: 248) | | NO: 25) |
| 16”) | | | |

TABLE 8
Variable heavy chain amino acid sequence

| Antibody | VH Amino Acid Sequence (SEQ ID NO) |
|---|---|
| Anti-FAM19A5 | AVTLDESGGGLQTPGGALSLVCKASGFTFSSHGMFWVRQTPGKGLEYVAEITNDGSGTNY |
| (“2-13”) | GSAVKGRATISRDNGQSTVRLQLNNLRAEDTGTYFCARSTYECPGGFSCWGDTGQIDAWG |
| | HGTEVIVSS (SEQ ID NO: 35) |
| Anti-FAM19A5 | AVTLDESGGGLQTPGGALSLVCKASGFTFSSFNMFWVRQAPGKGLEYVAQISSSGSSTNY |
| (“3-2”) | APAVRGRATISRDNGQSTVRLQLNNPGAEDTGTYYCAKSSYDCPYGHCSSGVDSAGEIDA |
| | WGHGTEVIVSS (SEQ ID NO: 36) |
| Anti-FAM19A5 | AVTLDESGGGLQTPGGALSLVCKASGFTFSSYQMGWVRQAPGKGLEWVGVINKSGSDTSY |
| (“1-65”) | GSAVKGRATISRDNGQSTVRLQLNNLRAEDTGTYFCAKGSASYITAATIDAWGHGTEVIV |
| | SS (SEQ ID NO: 37) |
| Anti-FAM19A5 | AVTLDESGGGLQTPGGALSLVCKASGFDFSDYGMGWVRQAPGKGLEWVAAIRSDGSNPSY |
| (“1-28”) | GSAVKGRATISKDNGRSTVRLQLNNLRAEDTATYYCAKDGNGYCALDAYRSGGYSCGVYP |
| | GSIDAWGHGTEVIVSS (SEQ ID NO: 38) |
| Anti-FAM19A5 | QSLEESGGRLVTPGTPLTLTCTVSGFSLSTYAVTWVRQAPGKGLEWIGYINWRGGTSYAN |
| (“P2-C12”) | WAKGRFTISKTSSTTVDLKMTSPTTEDTATYFCARDASSGAAFGSYGMDPWGPGTLVTVS |
| | S (SEQ ID NO: 155) |
| Anti-FAM19A5 | QVQLQESGPGLVKPSGTLSLNCAVSGGSISSSNWWSWVRQPPGKGLEWIGEIYHGGTTNY |
| (“13B4”) | NPSLKGRVTMSVDKTKNQFSLRLSSVTAVDTAVYYCARWQLVGGLDVWGQGTTVTVSS |
| | (SEQ ID NO: 156) |
| Anti-FAM19A5 | QVQLQEWGAGLLKPSETLSLTCAINAESFNGYSWTWIRQTPGKGLEWIGEISHFGSANYN |
| (“13F7”) | PSLKSRATISADKSKNQFSLKLTSVTAVDTAVYYCARALRGTYSRFYYGMDVWGQGTTVT |
| | VSS (SEQ ID NO: 157) |
| Anti-FAM19A5 | QVQLQESGPGLVKPSETLSLTCTVSGGSISSYYWSWIRQPPGKGLEWIGYIYPSGSTNYN |
| (“15A9”) | PSLKSRVTISVDTSKNQFSLNLKSVTAVDTAVYYCARVNPFGYYYAMDVWGQGTTVTVSS |
| | (SEQ ID NO: 158) |
| Anti-FAM19A5 | QSVEESGGRLVTPGTPLTLTCTVSGFSLSSDYMSWVRQAPGEGLEWIGIIYPSTTTYYAS |
| (“P1-A03”) | WAKGRFTISKTSSTTVELKMTSLTTEDTATYFCARGSNWSSGMNLWGPGTLVTVSS |
| | (SEQ ID NO: 159) |
| Anti-FAM19A5 | QSLEESGGRLVTPGTPLTLTCTASGFSLSTYYMSWVRQAPGKGLEWIGIVYPSGTTYYAN |
| (“P1-A08”) | WAKGRFTISTASTTVDLMITSPTTEDTATYFCARGDSFGYGLWGPGTLVTVSS (SEQ ID |
| | NO: 160) |
| Anti-FAM19A5 | QSLEESGGRLVTPGTPLTLTCTASGFSLSNYYMGWVRQAPGEGLEWIGIIYASGSTYYAS |
| (“P1-F02”) | WAKGRFTISKTSTTVDLKMTSLTTEDTATYFCARIDIGVGDYGWAYDRLDLWGQGTLVTV |
| | SS (SEQ ID NO: 161) |
| Anti-FAM19A5 | QEQLVESGGRLVTPGTPLTLSCTASGFFLSGYYMSWVRQAPGKGLEWIGIIYPSGSTDYA |
| (“P2-A01”) | SWAKGRFTISKTSTTVDLKITTPTTEDTATYFCARVAGYVGYGYETFFDIWGPGTLVTVS |
| | L (SEQ ID NO: 162) |
| Anti-FAM19A5 | QSVEESGGRLVTPGTPLTLTCTVSGFSLNNYDMSWVRQAPGKGLEYIGFMDTDGSAYYAT |
| (“P2-A03”) | WAKGRFTISRTSTTVDLKMTSPTTEDTATYFCARRGSSYYGGIDIWGPGTPVTVSL |
| | (SEQ ID NO: 163) |
| Anti-FAM19A5 | QSLEESGGRLVTPGTPLTLTCTASGFSLSSYYMNWVRQAPGKGLEWIGIIYPSGTTYYAG |
| (“P2-F07”) | WAKGRFTISKTSTTVDLKITSPTSEDTATYFCARTVSGYFDIWGPGTLVTVSL (SEQ ID |
| | NO: 164) |
| Anti-FAM19A5 | QEQLVESGGRLVTPGTTLTLTCTVSGFSLSSYGVSWVRQAPGKGLEWIGYIANNYNPHYA |
| (“P2-F11”) | SWAKGRFTISKTSSTTVDLKMTSLTTEDTATYFCARDNYGMDPWGPGTLVTVSS (SEQ |
| | ID NO: 165) |
| Anti-FAM19A5 | AVTLDESGGGLQTPGGALSLSCKASGFTFSSYQMGWVRQAPGKGLEWVGVINKSGSDTSY |
| (“SS01-13”) | GSAVKGRATISRDNGQSTLYLQMNNLRAEDTAVYFCAKGSASYITAATIDAWGHGTEVIV |
| | SS (SEQ ID NO: 211) |
| Anti-FAM19A5 | AVTLDESGGGLQTPGGALRLSCKASGFTFSSYQMGWVRQAPGKGLEWVSAINKSGSDTSY |
| (“SS01-13-s5”) | GSAVKGRATISRDNGQSTLYLQMNSLRAEDTAVYFCAKGSASYITAATIDAWGHGTEVIV |
| | SS (SEQ ID NO: 214) |
| Anti-FAM19A5 | AVTLDESGGGLQTPGGALRLSCKASGFTFSSYQMGWVRQAPGKGLEWVSAINKGGSDTSY |
| (“S5-2.GKNG”) | GSAVKGRATISRDNGQSTLYLQMNSLRAEDTAVYFCAKGSASYITAATIDAWGHGTEVIV |
| | SS (SEQ ID NO: 249) |
| Anti-FAM19A5 | AVTLDESGGGLQTPGGALRLSCKASGFTFSSFNMFWVRQAPGKGLEYVSQISSSGSSTNY |
| (“1-7A-IT”) | APAVKGRATISRDNGQSTLYLQMNSLRAEDTGTYYCAKSSYDCPYGHCSSGVDSAGEIDA |
| | WGHGTEVIVSS (SEQ ID NO: 234) |
| Anti-FAM19A5 | AVTLDESGGGLQTPGGALRLSCKASGFDFESFNMFWVRQAPGKGLEYVSQISSSEEDENY |
| (“Low-PI”) | APAVKGRATISRDNGQSTLYLQMNSLRAEDTGTYYCAKSSYDCPYGHCSSGVDSAGEIDA |
| | WGHGTEVIVSS (SEQ ID NO: 235) |
| Anti-FAM19A5 | AVTLDESGGGLQTPGGALRLSCKASGFDFESFNMFWVRQAPGKGLEYVSQISSSEEDENY |
| (“1-30”) | APAVKGRATISRDNGQSTLYLQMNSLRAEDTGTYYCAKSSYDCPYGHCSSGVDSAGEIDA |
| | WGHGTEVIVSS (SEQ ID NO: 235) |
| Anti-FAM19A5 | AVTLDESGGGLQTPGGALRLSCKASGFDFESFNMFWVRQAPGKGLEYVSQISSSEEDENY |
| (“1-17”) | APAVKGRATISRDNGQSTLYLQMNSLRAEDTGTYYCAKSSYDCPYGHCSSGVDSAGEIDA |
| | WGHGTEVIVSS (SEQ ID NO: 235) |
| Anti-FAM19A5 | AVTLDESGGGLQTPGGALRLSCKASGFDFESFNMFWVRQAPGKGLEYVSQISSSEEDENY |
| (“1-32”) | APAVKGRATISRDNGQSTLYLQMNSLRAEDTGTYYCAKSSYDCPYGHCSSGVDSAGEIDA |
| | WGHGTEVIVSS (SEQ ID NO: 235) |
| Anti-FAM19A5 | AVTLDESGGGLQTPGGALRLSCKASGFDFESFNMFWVRQAPGKGLEYVSQISSSEEDENY |
| (“4-11”) | APAVKGRATISRDNGQSTLYLQMNSLRAEDTGTYYCAKSSYDCPYGHCSSGVDSAGEIDA |
| | WGHGTEVIVSS (SEQ ID NO: 235) |
| Anti-FAM19A5 | AVTLDESGGGLQTPGGALRLSCKASGFDFESFNMFWVRQAPGKGLEYVSQISSSEEDENY |
| (“6-10”) | APAVKGRATISRDNGQSTLYLOMNSLRAEDTGTYYCAKSSYDCPYGHCSSGVDSAGEIDA |
| | WGHGTEVIVSS (SEQ ID NO: 235) |
| Anti-FAM19A5 | AVTLDESGGGLQTPGGALRLSCSASGFTFSSHGMFWVRQAPGKGLEYVSEITNDGSGTNY |
| (“2-13D”) | GSAVKGRATISRDNGQSTLYLQMNSLRAEDTGTYFCARSTYECPGGFSCWGDTGQIDAWG |
| | HGTEVIVSS (SEQ ID NO: 236) |
| Anti-FAM19A5 | AVTLDESGGGLQTPGGALRLSCSASGFDFSSHGMFWVRQAPGKGLEYVSEITNDGSGTNY |
| (“2-13D-37”) | GSAVKGRATISRDNGQSTLYLQMNSLRAEDTGTYFCARSTYECPGGFSCWGDTGQIDAWG |
| | HGTEVIVSS (SEQ ID NO: 250) |
| Anti-FAM19A5 | AVTLDESGGGLQTPGGALRLSCSASGFDFSSHGMFWVRQAPGKGLEYVSEITNDGSGTNY |
| (“2-13D-37- | GSAVKGRATISRDNGQSTLYLQMNSLRAEDTGTYFCARSSYVCPGGFSCWGDTGQIDAWG |
| 1.5W-41”) | HGTEVIVSS (SEQ ID NO: 237) |
| Anti-FAM19A5 | AVTLDESGGGLQTPGGALRLSCSASGFDFSSHGMFWVRQAPGKGLEYVSEITNDGSGTNY |
| (“2-13D-37-3W- | GSAVKGRATISRDNGQSTLYLQMNSLRAEDTGTYFCARSNYACPGGFSCWGDTGQIDAWG |
| 16”) | HGTEVIVSS (SEQ ID NO: 251) |

TABLE 9
Variable light chain amino acid sequence

| Antibody | VL Amino Acid Sequence (SEQ ID NO) |
|---|---|
| Anti-FAM19A5 | ALTQPSSVSANPGETVKITCSGGSYSYGWFQQKSPGSALVTVIYWDDERPSDIPSRFSGA |
| (“2-13”) | LSGSTNTLTITGVQADDEAVYFCGTEDISGTAGVFGAGTTLTVL (SEQ ID NO: 39) |
| Anti-FAM19A5 | ALTQPSSVSANPGETVKITCSGGGSYAGSYYYGWYQQKAPGSAPVTLIYESNKRPSDIPS |
| (“3-2”) | RFSGSTSGSTATLTITGVQADDEAIYYCGSWDSSNGGIFGAGTTLTVL (SEQ ID NO: 40) |
| Anti-FAM19A5 | ALTQPSSVSANPGETVKITCSGGGSSGYGYGWYQQKSPSSAPLTVIYWNDKRPSDIPSRF |
| (“1-65”) | SGSKSGSTHTLTITGVQAEDEAVYFCGNDDYSSDSGYVGVFGAGTTLTVL (SEQ ID NO: 41) |
| Anti-FAM19A5 | ALTQPSSVSANLEGTVEITCSGSGYGYGWYQQKSPGSAPVTVIYQNDKRPSDIPSRFSGS |
| (“1-28”) | KSGSTGTLTITGVQVEDEAVYYCGSEDSSTLAGIFGAGTTLTVL (SEQ ID NO: 42) |
| Anti-FAM19A5 | ELDMTQTPSSVSAAVGGTVTIKCQASQSISSYLSWYQQKPGQPPKLLIYEASKLASGVPS |
| (“P2-C12”) | RFSGSGYGTEFTLTISDLECADAATYYCQQGYSSTNVWNAFGGGTNVEIK |
| | (SEQ ID NO: 166) |
| Anti-FAM19A5 | SYELTQPLSVSVSPGQTASITCSGDKLGNVYASWYQQKPGOSPTLVIYQDNKRPSGIPER |
| (“13B4”) | FSGSNSGKTATLTISGTQALDEADYYCQAWDSSTAVFGGGTKLTVL (SEQ ID NO: 167) |
| Anti-FAM19A5 | DIVMTQTPLSLPVAPGEPASISCRSSQSLLHSNGYNYLDWYVQKPGQPPQLLIYLGSNRA |
| (“13F7”) | SGVPDRFSGSGSGTDFTLKISRVEAEDVGVYYCMQARQTPLTFGGGTKVEIK (SEQ ID |
| | NO: 168) |
| Anti-FAM19A5 | DIQMTQSPSSLSASVGDRITISCRASQSISTSLNWYQQTPGKAPRLLIYGASTLQSGVPS |
| (“15A9”) | RFSGGGSGTDFSLTITSLQPEDFATYYCQESASIPRTFGQGTKLDIK (SEQ ID NO: 169) |
| Anti-FAM19A5 | ELVMTQTPPSLSASVGETVRIRCLASEDIYSGISWYQQKPEKPPTLLISGASNLESGVPP |
| (“P1-A03”) | RFSGSGSGTDYTLTIGGVQAEDAATYYCLGGYSYSSTGLTFGAGTNVEIK (SEQ ID NO: |
| | 170) |
| Anti-FAM19A5 | ELVLTQSPSVQVNLGQTVSLTCTADTLSRSYASWYQQKPGQAPVLLIYRDTSRPSGVPDR |
| (“P1-A08”) | FSGSSSGNTATLTISGAQAGDEADYYCATSDGSGSNYQYVFGGGTQLTVT (SEQ ID NO: |
| | 171) |
| Anti-FAM19A5 | ELDMTQTPPSLSASVGETVRIRCLASEDIYSGISWYQQKPGKPPTLLIYGASNLESGVPP |
| (“P1-F02”) | RFSGSGSGTDYTLTIGGVQAEDAATYYCLGGYSYSSITFGAGTNVEIK (SEQ ID NO: |
| | 172) |
| Anti-FAM19A5 | ELVMTQTPPSLSASVGETVRIRCLASEDIYSGISWYQQKPGKPPTLLIYGASNLESGVPP |
| (“P2-A01”) | RFSGSGSGSDYTLTIGGVQAEDAATYYCLGGVTYSSTGTHLTFGAGTNVEIK (SEQ ID |
| | NO: 173) |
| Anti-FAM19A5 | ELDLTQTPASVSEPVGGTVTIKCQASQSIGGNLAWYQQKPGQPPKLLIYRASTLASGVPS |
| (“P2-A03”) | RFKGSGSGTDFTLTISDLECADAATYYCQSPAYDPAAYVGNAFGGGTELEIL (SEQ ID |
| | NO: 174) |
| Anti-FAM19A5 | ELDLTQTPPSLSASVGGTVTINCLASEDIYSALAWYQQKPGKPPTLLISGTSNLESGVPP |
| (“P2-F07”) | RFSGSGSGTDYTLTIGGVQAEDAATYFCQGYSSYPLTFGAGTNVEIK (SEQ ID NO: 175) |
| Anti-FAM19A5 | ELDLTQTPSSVSAAVGGTVTINCQASQSVYNNKNLAWYQQKPGQPPKLLIYAASTLASGV |
| (“P2-F11”) | SSRFKGSGSGTQFTLTISDVQCDDAATYYCQGEFSCSSADCNAFGGGTELEIL (SEQ ID |
| | NO: 176) |
| Anti-FAM19A5 | ALTQPSSVSANPGETVRITCSGGASSGYGYGWYQQKPSSAPLTVIYKDDERPSDIPSRFS |
| (“SS01-13”) | GSSSGSTHTLTITGVQAEDEAVYFCGNDDYSSDSGYVGVFGAGTTLTVL (SEQ ID NO: |
| | 212) |
| Anti-FAM19A5 | ALTQPSSVSANPGETARITCSGGASSGYGYGWYQQKPSSAPLTVIYKDSERPSDIPSRFS |
| (“SS01-13-s5”) | GSSSGSTHTLTISGVQAEDEAVYFCGNDDYSSDSGYVGVFGAGTTLTVL (SEQ ID NO: |
| | 215) |
| Anti-FAM19A5 | ALTQPSSVSANPGETARITCSGGASSGYGYGWYQQKPSSAPLTVIYKDSERPSDIPSRFS |
| (“S5-2.GKNG”) | GSSSGSTHTLTISGVQAEDEAVYFCGNDDYSSDSGYVGVFGAGTTLTVL (SEQ ID NO: |
| | 215) |
| Anti-FAM19A5 | ALTQPSSVSANPGETVKITCSGGGSYAGSYYYGWYQQKPGSAPVTLIYENNKRPSDIPSR |
| (“1-7A-IT”) | FSGSTSGSTATLTITGVQAGDEADYYCGSWDSSNGGIFGAGTTLTVL (SEQ ID NO: 238) |
| Anti-FAM19A5 | ALTQPSSVSANPGETVKITCSGGGSEEEQYYYGWYQQKPGSAPVTLIYEDEERPSDIPSR |
| (“Low-PI”) | FSGSTSGSTATLTITGVQAGDEADYYCGSWDSEDEDHFGAGTTLTVL (SEQ ID NO: 239) |
| Anti-FAM19A5 | ALTQPSSVSANPGETVKITCSGGGSEEEQYYYGWYQQKPGSAPVTLIYQDEERPSDIPSR |
| (“1-30”) | FSGSTSGSTATLTITGVQAGDEADYYCGSWDSEDEDHFGAGTTLTVL (SEQ ID NO: 240) |
| Anti-FAM19A5 | ALTQPSSVSANPGETVKITCSGGGSYAGSYYYGWYQQKPGSAPVTLIYEDEORPSDIPSR |
| (“1-17”) | FSGSTSGSTATLTITGVQAGDEADYYCGSWDSEDEDHFGAGTTLTVL (SEQ ID NO: 241) |
| Anti-FAM19A5 | ALTQPSSVSANPGETVKITCSGGGSYAGSYYYGWYQQKPGSAPVTLIYQDEERPSDIPSR |
| (“1-32”) | FSGSTSGSTATLTITGVQAGDEADYYCGSWDSEDEDHFGAGTTLTVL (SEQ ID NO: 242) |
| Anti-FAM19A5 | ALTQPSSVSANPGETVKITCSGGGSYAGSYYYGWYQQKPGSAPVTLIYEDHERPSDIPSR |
| (“4-11”) | FSGSTSGSTATLTITGVQAGDEADYYCGSWDSSDEDHFGAGTTLTVL (SEQ ID NO: 243) |
| Anti-FAM19A5 | ALTQPSSVSANPGETVKITCSGGGSYAGSYYYGWYQQKPGSAPVTLIYODLLRPSDIPSR |
| (“6-10”) | FSGSTSGSTATLTITGVQAGDEADYYCGSWDSLSSSHFGAGTTLTVL (SEQ ID NO: 244) |
| Anti-FAM19A5 | ALTQPSSVSANPGETAKITCSGGVYSYGWFQQKPGSALVTVIYWDDERPSDIPSRFSGAL |
| (“2-13D”) | SGSTNTLTITGVQAEDEADYYCGTEDISGTAGVFGAGTTLTVL (SEQ ID NO: 245) |
| Anti-FAM19A5 | ALTQPSSVSANPGETAKITCSGGVYSYGWFQQKPGSALVTVIYWDDERPSDIPSRFSGAL |
| (“2-13D-37”) | SGSTNTLTITGVQAEDEADYYCGTEDISGTAGVFGAGTTLTVL (SEQ ID NO: 245) |
| Anti-FAM19A5 | ALTQPSSVSANPGETAKITCSGGVYSYGWFQQKPGSALVTVIYWDDERPSDIPSRFSGAL |
| (“2-13D-37- | SGSTNTLTITGVQAEDEADYYCGTEDISGTAGVFGAGTTLTVL (SEQ ID NO: 245) |
| 1.5W-41”) | |
| Anti-FAM19A5 | ALTQPSSVSANPGETAKITCSGGVYSYGWFQQKPGSALVTVIYWDDERPSDIPSRFSGAL |
| (“2-13D-37-3W- | SGSTNTLTITGVQAEDEADYYCGTEDISGTAGVFGAGTTLTVL (SEQ ID NO: 245) |
| 16”) | |

Accordingly, in some aspects, an anti-FAM19A5 antibody useful for the present disclosure comprises heavy and light chain variable regions, wherein the heavy chain variable region comprises the amino acid sequence set forth in any one of SEQ ID NOs: 35-38, 155-165, 211, 214, 234-237, or 249-251 (see Table 8). In some aspects, an anti-FAM19A5 antibody useful for the present disclosure comprises the CDRs of the heavy chain variable region selected from the group consisting of SEQ ID NOs: 35-38, 155-165, 211, 214, 234-237, or 249-251 (see Table 8).

In some aspects, an anti-FAM19A5 antibody useful for the present disclosure comprises heavy and light chain variable regions, wherein the light chain variable region comprises the amino acid sequence set forth in any one of SEQ ID NOs: 39-42, 166-176, 212, 215, or 238-245 (see Table 9). In some aspects, an anti-FAM19A5 antibody useful for the present disclosure comprises the CDRs of the light chain variable region selected from the group consisting of SEQ ID NOs: 39-42, 166-176, 212, 215, or 238-245 (see Table 9).

In some aspects, an anti-FAM19A5 antibody that can be used with the methods provided herein comprises the CDRs of the heavy chain variable region selected from the group consisting of SEQ ID NOs: 35-38, 155-165, 211, 214, 234-237, or 249-251, and the CDRs of the light chain variable region selected from the group consisting of SEQ ID NOs: 39-42, 166-176, 212, 215, or 238-245.

In some aspects, an anti-FAM19A5 antibody that can be used with the methods provided herein comprises a heavy chain variable region and a light chain variable region, wherein the heavy chain variable region comprises an amino acid sequence which is at least about 80%, at least about 85%, at least about 90%, at least about 95%, at least about 96%, at least about 97%, at least about 98%, at least about 99%, or about 100% identical to the amino acid sequence set forth as SEQ ID NOs: 35-38, 155-165, 211, 214, 234-237, or 249-251 (see Table 8). In some aspects, an anti-FAM19A5 antibody comprises a heavy chain variable region and a light chain variable region, wherein the light chain variable region comprises an amino acid sequence which is at least about 80%, at least about 85%, at least about 90%, at least about 95%, at least about 96%, at least about 97%, at least about 98%, at least about 99%, or about 100% identical to the amino acid sequence set forth as SEQ ID NOs: 39-42, 166-176, 212, 215, or 238-245 (see Table 9). In some aspects, an anti-FAM19A5 antibody useful for the present disclosure comprise a heavy chain variable region and a light chain variable region, wherein the heavy chain variable region comprises an amino acid sequence which is at least about 80%, at least about 85%, at least about 90%, at least about 95%, at least about 96%, at least about 97%, at least about 98%, at least about 99%, or about 100% identical to the amino acid sequence set forth as SEQ ID NOs: 35-38, 155-165, 211, 214, 234-237, or 249-251, and wherein the light chain variable region comprises an amino acid sequence which is at least about 80%, at least about 85%, at least about 90%, at least about 95%, at least about 96%, at least about 97%, at least about 98%, at least about 99%, or about 100% identical to the amino acid sequence set forth as SEQ ID NOs: 39-42, 166-176, 212, 215, or 238-245.

In some aspects, an anti-FAM19A5 antibody comprises a heavy chain variable region (VH) and a light chain variable region (VL), wherein:
(1) the VH and VL comprises the sequences set forth in SEQ ID NOs: 35 and 39, respectively;
(2) the VH and VL comprises the sequences set forth in SEQ ID NOs: 36 and 40, respectively;
(3) the VH and VL comprises the sequences set forth in SEQ ID NOs: 37 and 41, respectively;
(4) the VH and VL comprises the sequences set forth in SEQ ID NOs: 38 and 42, respectively;
(5) the VH and VL comprises the sequences set forth in SEQ ID NOs: 155 and 166, respectively;
(6) the VH and VL comprises the sequences set forth in SEQ ID NOs: 156 and 167, respectively;
(7) the VH and VL comprises the sequences set forth in SEQ ID NOs: 157 and 168, respectively;
(8) the VH and VL comprises the sequences set forth in SEQ ID NOs: 158 and 169, respectively;
(9) the VH and VL comprises the sequences set forth in SEQ ID NOs: 159 and 170, respectively;
(10) the VH and VL comprises the sequences set forth in SEQ ID NOs: 160 and 171, respectively;
(11) the VH and VL comprises the sequences set forth in SEQ ID NOs: 161 and 172, respectively;
(12) the VH and VL comprises the sequences set forth in SEQ ID NOs: 162 and 173, respectively;
(13) the VH and VL comprises the sequences set forth in SEQ ID NOs: 163 and 174, respectively;
14) the VH and VL comprises the sequences set forth in SEQ ID NOs: 164 and 175, respectively;
(15) the VH and VL comprises the sequences set forth in SEQ ID NOs: 165 and 176, respectively;
(16) the VH and VL comprises the sequences set forth in SEQ ID NOs: 211 and 212, respectively;
(17) the VH and VL comprises the sequences set forth in SEQ ID NOs: 214 and 215, respectively;
(18) the VH and VL comprises the sequences set forth in SEQ ID NOs: 249 and 215, respectively;
(19) the VH and VL comprises the sequences set forth in SEQ ID NOs: 234 and 238, respectively;
(20) the VH and VL comprises the sequences set forth in SEQ ID NOs: 235 and 239, respectively;
(21) the VH and VL comprises the sequences set forth in SEQ ID NOs: 235 and 240, respectively; (22) the VH and VL comprises the sequences set forth in SEQ ID NOs: 235 and 241, respectively;
(23) the VH and VL comprises the sequences set forth in SEQ ID NOs: 235 and 242, respectively;
(24) the VH and VL comprises the sequences set forth in SEQ ID NOs: 235 and 243, respectively;
(25) the VH and VL comprises the sequences set forth in SEQ ID NOs: 235 and 244, respectively
(26) the VH and VL comprises the sequences set forth in SEQ ID NOs: 236 and 245, respectively
(27) the VH and VL comprises the sequences set forth in SEQ ID NOs: 250 and 245, respectively;
(28) the VH and VL comprises the sequences set forth in SEQ ID NOs: 237 and 245, respectively; or
(29) the VH and VL comprises the sequences set forth in SEQ ID NOs: 251 and 245, respectively.

In some aspects, an anti-FAM19A5 antibody comprises a VH and a VL, wherein the VH comprises the amino acid sequence set forth in SEQ ID NO: 35 and the VL comprises the amino acid sequence set forth in SEQ ID NO: 39. In some aspects, an anti-FAM19A5 antibody comprises a VH and a VL, wherein the VH comprises the amino acid sequence set forth in SEQ ID NO: 36 and the VL comprises the amino acid sequence set forth in SEQ ID NO: 40. In some aspects, an anti-FAM19A5 antibody comprises a VH and a VL, wherein the VH comprises the amino acid sequence set forth in SEQ ID NO: 37 and the VL comprises the amino acid sequence set forth in SEQ ID NO: 41. In some aspects, an anti-FAM19A5 antibody comprises a VH and a VL, wherein the VH comprises the amino acid sequence set forth in SEQ ID NO: 38 and the VL comprises the amino acid sequence set forth in SEQ ID NO: 42. In some aspects, an anti-FAM19A5 antibody comprises a VH and a VL, wherein the VH comprises the amino acid sequence set forth in SEQ ID NO: 155 and the VL comprises the amino acid sequence set forth in SEQ ID NO: 166. In some aspects, an anti-FAM19A5 antibody comprises a VH and a VL, wherein the VH comprises the amino acid sequence set forth in SEQ ID NO: 156 and the VL comprises the amino acid sequence set forth in SEQ ID NO: 167. In some aspects, an anti-FAM19A5 antibody comprises a VH and a VL, wherein the VH comprises the amino acid sequence set forth in SEQ ID NO: 157 and the VL comprises the amino acid sequence set forth in SEQ ID NO: 168. In some aspects, an anti-FAM19A5 antibody comprises a VH and a VL, wherein the VH comprises the amino acid sequence set forth in SEQ ID NO: 158 and the VL comprises the amino acid sequence set forth in SEQ ID NO: 169. In some aspects, an anti-FAM19A5 antibody comprises a VH and a VL, wherein the VH comprises the amino acid sequence set forth in SEQ ID NO: 159 and the VL comprises the amino acid sequence set forth in SEQ ID NO: 170. In some aspects, an anti-FAM19A5 antibody comprises a VH and a VL, wherein the VH comprises the amino acid sequence set forth in SEQ ID NO: 160 and the VL comprises the amino acid sequence set forth in SEQ ID NO: 171. In some aspects, an anti-FAM19A5 antibody comprises a VH and a VL, wherein the VH comprises the amino acid sequence set forth in SEQ ID NO: 161 and the VL comprises the amino acid sequence set forth in SEQ ID NO: 172. In some aspects, an anti-FAM19A5 antibody comprises a VH and a VL, wherein the VH comprises the amino acid sequence set forth in SEQ ID NO: 162 and the VL comprises the amino acid sequence set forth in SEQ ID NO: 173. In some aspects, an anti-FAM19A5 antibody comprises a VH and a VL, wherein the VH comprises the amino acid sequence set forth in SEQ ID NO: 163 and the VL comprises the amino acid sequence set forth in SEQ ID NO: 174. In some aspects, an anti-FAM19A5 antibody comprises a VH and a VL, wherein the VH comprises the amino acid sequence set forth in SEQ ID NO: 164 and the VL comprises the amino acid sequence set forth in SEQ ID NO: 175. In some aspects, an anti-FAM19A5 antibody comprises a VH and a VL, wherein the VH comprises the amino acid sequence set forth in SEQ ID NO: 165 and the VL comprises the amino acid sequence set forth in SEQ ID NO: 176. In some aspects, an anti-FAM19A5 antibody comprises a VH and a VL, wherein the VH comprises the amino acid sequence set forth in SEQ ID NO: 211 and the VL comprises the amino acid sequence set forth in SEQ ID NO: 212. In some aspects, an anti-FAM19A5 antibody comprises a VH and a VL, wherein the VH comprises the amino acid sequence set forth in SEQ ID NO: 214 and the VL comprises the amino acid sequence set forth in SEQ ID NO: 215. In some aspects, an anti-FAM19A5 antibody comprises a VH and a VL, wherein the VH comprises the amino acid sequence set forth in SEQ ID NO: 249 and the VL comprises the amino acid sequence set forth in SEQ ID NO: 215. In some aspects, an anti-FAM19A5 antibody comprises a VH and a VL, wherein the VH comprises the amino acid sequence set forth in SEQ ID NO: 234 and the VL comprises the amino acid sequence set forth in SEQ ID NO: 238. In some aspects, an anti-FAM19A5 antibody comprises a VH and a VL, wherein the VH comprises the amino acid sequence set forth in SEQ ID NO: 235 and the VL comprises the amino acid sequence set forth in SEQ ID NO: 239. In some aspects, an anti-FAM19A5 antibody comprises a VH and a VL, wherein the VH comprises the amino acid sequence set forth in SEQ ID NO: 235 and the VL comprises the amino acid sequence set forth in SEQ ID NO: 240. In some aspects, an anti-FAM19A5 antibody comprises a VH and a VL, wherein the VH comprises the amino acid sequence set forth in SEQ ID NO: 235 and the VL comprises the amino acid sequence set forth in SEQ ID NO: 241. In some aspects, an anti-FAM19A5 antibody comprises a VH and a VL, wherein the VH comprises the amino acid sequence set forth in SEQ ID NO: 235 and the VL comprises the amino acid sequence set forth in SEQ ID NO: 242. In some aspects, an anti-FAM19A5 antibody comprises a VH and a VL, wherein the VH comprises the amino acid sequence set forth in SEQ ID NO: 235 and the VL comprises the amino acid sequence set forth in SEQ ID NO: 243. In some aspects, an anti-FAM19A5 antibody comprises a VH and a VL, wherein the VH comprises the amino acid sequence set forth in SEQ ID NO: 235 and the VL comprises the amino acid sequence set forth in SEQ ID NO: 244. In some aspects, an anti-FAM19A5 antibody comprises a VH and a VL, wherein the VH comprises the amino acid sequence set forth in SEQ ID NO: 236 and the VL comprises the amino acid sequence set forth in SEQ ID NO: 245. In some aspects, an anti-FAM19A5 antibody comprises a VH and a VL, wherein the VH comprises the amino acid sequence set forth in SEQ ID NO: 250 and the VL comprises the amino acid sequence set forth in SEQ ID NO: 245. In some aspects, an anti-FAM19A5 antibody comprises a VH and a VL, wherein the VH comprises the amino acid sequence set forth in SEQ ID NO: 237 and the VL comprises the amino acid sequence set forth in SEQ ID NO: 245. In some aspects, an anti-FAM19A5 antibody comprises a VH and a VL, wherein the VH comprises the amino acid sequence set forth in SEQ ID NO: 251 and the VL comprises the amino acid sequence set forth in SEQ ID NO: 245.

In some aspects, the anti-FAM19A5 antibody comprises (1) the heavy chain CDR1, CDR2 and CDR3 of 2-13, or combinations thereof, and/or the light chain CDR1, CDR2, and CDR3 of 2-13, or any combinations thereof; (2) the heavy chain CDR1, CDR2 and CDR3 of 3-2, or combinations thereof, and/or the light chain CDR1, CDR2, and CDR3 of 3-2, or any combinations thereof; (3) the heavy chain CDR1, CDR2 and CDR3 of 1-65, or combinations thereof, and/or the light chain CDR1, CDR2, and CDR3 of 1-65, or any combinations thereof; (4) the heavy chain CDR1, CDR2 and CDR3 of 1-28, or combinations thereof, and/or the light chain CDR1, CDR2, and CDR3 of 1-28, or any combinations thereof; (5) the heavy chain CDR1, CDR2, and CDR3 of P2-C12, or combinations thereof, and/or the light chain CDR1, CDR2, and CDR3 of P2-C12, or any combinations thereof; (6) the heavy chain CDR1, CDR2, and CDR3 of 13B4, or combinations thereof, and/or the light chain CDR1, CDR2, and CDR3 of 13B4, or any combinations thereof; (7) the heavy chain CDR1, CDR2, and CDR3 of 13F₇, or combinations thereof, and/or the light chain CDR1, CDR2, and CDR3 of 13F₇, or any combinations thereof; (8) the heavy chain CDR1, CDR2, and CDR3 of 15A9, or combinations thereof, and/or the light chain CDR1, CDR2, and CDR3 of 15A9, or any combinations thereof; (9) the heavy chain CDR1, CDR2, and CDR3 of P1-A03, or combinations thereof, and/or the light chain CDR1, CDR2, and CDR3 of P1-A03, or any combinations thereof; (10) the heavy chain CDR1, CDR2, and CDR3 of P1-A08, or combinations thereof, and/or the light chain CDR1, CDR2, and CDR3 of P1-A08, or any combinations thereof; (11) the heavy chain CDR1, CDR2, and CDR3 of P1-F02, or combinations thereof, and/or the light chain CDR1, CDR2, and CDR3 of P1-F02, or any combinations thereof; (12) the heavy chain CDR1, CDR2, and CDR3 of P2-A01, or combinations thereof, and/or the light chain CDR1, CDR2, and CDR3 of P2-A01, or any combinations thereof; (13) the heavy chain CDR1, CDR2, and CDR3 of P2-A03, or combinations thereof, and/or the light chain CDR1, CDR2, and CDR3 of P2-A03, or any combinations thereof; (14) the heavy chain CDR1, CDR2, and CDR3 of P2-F07, or combinations thereof, and/or the light chain CDR1, CDR2, and CDR3 of P2-F07, or any combinations thereof; (15) the heavy chain CDR1, CDR2, and CDR3 of P2-F11, or combinations thereof, and/or the light chain CDR1, CDR2, and CDR3 of F2-F11, or any combinations thereof; (16) the heavy chain CDR1, CDR2, and CDR3 of SS01-13, or combinations thereof, and/or the light chain CDR1, CDR2, and CDR3 of SS01-13, or any combinations thereof; (17) the heavy chain CDR1, CDR2, and CDR3 of SS01-13-s5, or combinations thereof, and/or the light chain CDR1, CDR2, and CDR3 of SS01-13-s5, or any combinations thereof; (18) the heavy chain CDR1, CDR2, and CDR3 of S5-2.GKNG, or combinations thereof, and/or the light chain CDR1, CDR2, and CDR3 of S5-2.GKNG, or any combinations thereof; (19) the heavy chain CDR1, CDR2, and CDR3 of 1-7A-IT, or combinations thereof, and/or the light chain CDR1, CDR2, and CDR3 of 1-7A-IT, or any combinations thereof; (20) the heavy chain CDR1, CDR2, and CDR3 of Low-PI, or combinations thereof, and/or the light chain CDR1, CDR2, and CDR3 of Low-PI, or any combinations thereof; (21) the heavy chain CDR1, CDR2, and CDR3 of 1-30, or combinations thereof, and/or the light chain CDR1, CDR2, and CDR3 of 1-30, or any combinations thereof; (22) the heavy chain CDR1, CDR2, and CDR3 of 1-17, or combinations thereof, and/or the light chain CDR1, CDR2, and CDR3 of 1-17, or any combinations thereof; (23) the heavy chain CDR1, CDR2, and CDR3 of 1-32, or combinations thereof, and/or the light chain CDR1, CDR2, and CDR3 of 1-32, or any combinations thereof; (24) the heavy chain CDR1, CDR2, and CDR3 of 4-11, or combinations thereof, and/or the light chain CDR1, CDR2, and CDR3 of 4-11, or any combinations thereof; (25) the heavy chain CDR1, CDR2, and CDR3 of 6-10, or combinations thereof, and/or the light chain CDR1, CDR2, and CDR3 of 6-10, or any combinations thereof; (26) the heavy chain CDR1, CDR2, and CDR3 of 2-13D, or combinations thereof, and/or the light chain CDR1, CDR2, and CDR3 of 2-13D, or any combinations thereof; (27) the heavy chain CDR1, CDR2, and CDR3 of 2-13D-37, or combinations thereof, and/or the light chain CDR1, CDR2, and CDR3 of 2-13D-37, or any combinations thereof; (28) the heavy chain CDR1, CDR2, and CDR3 of 2-13D-37-1.5W-41, or combinations thereof, and/or the light chain CDR1, CDR2, and CDR3 of 2-13D-37-1.5W-41, or any combinations thereof; or (29) the heavy chain CDR1, CDR2, and CDR3 of 2-13D-37-3W-16, or combinations thereof, and/or the light chain CDR1, CDR2, and CDR3 of 2-13D-37-3W-16, or any combinations thereof. The amino acid sequences of the VH CDR1, CDR2, and CDR3 for the different anti-FAM19A5 antibodies disclosed herein are provided in Table 6. The amino acid sequences of the VL CDR1, CDR2, and CDR3 for the different anti-FAM19A5 antibodies disclosed herein are provided in Table 7.

In some aspects, an anti-FAM19A5 antibody useful for the present disclosure comprises: (a) a VH CDR1 comprising the amino acid sequence set forth in SEQ ID NO: 11, (b) a VH CDR2 comprising the amino acid sequence set forth in SEQ ID NO: 12, (c) a VH CDR3 comprising the amino acid sequence set forth in SEQ ID NO: 13, (d) a VL CDR1 comprising the amino acid sequence set forth in SEQ ID NO: 23, (e) a VL CDR2 comprising the amino acid sequence set forth in SEQ ID NO: 24, and (f) a VL CDR3 comprising the amino acid sequence set forth in SEQ ID NO: 25. In some aspects, an anti-FAM19A5 antibody useful for the present disclosure comprises: (a) a VH CDR1 comprising the amino acid sequence set forth in SEQ ID NO: 14, (b) a VH CDR2 comprising the amino acid sequence set forth in SEQ ID NO: 15, (c) a VH CDR3 comprising the amino acid sequence set forth in SEQ ID NO: 16, (d) a VL CDR1 comprising the amino acid sequence set forth in SEQ ID NO: 26, (e) a VL CDR2 comprising the amino acid sequence set forth in SEQ ID NO: 27, and (f) a VL CDR3 comprising the amino acid sequence set forth in SEQ ID NO: 28. In some aspects, an anti-FAM19A5 antibody useful for the present disclosure comprises: (a) a VH CDR1 comprising the amino acid sequence set forth in SEQ ID NO: 17, (b) a VH CDR2 comprising the amino acid sequence set forth in SEQ ID NO: 18, (c) a VH CDR3 comprising the amino acid sequence set forth in SEQ ID NO: 19, (d) a VL CDR1 comprising the amino acid sequence set forth in SEQ ID NO: 29, (e) a VL CDR2 comprising the amino acid sequence set forth in SEQ ID NO: 30, and (f) a VL CDR3 comprising the amino acid sequence set forth in SEQ ID NO: 31. In some aspects, an anti-FAM19A5 antibody useful for the present disclosure comprises: (a) a VH CDR1 comprising the amino acid sequence set forth in SEQ ID NO: 20, (b) a VH CDR2 comprising the amino acid sequence set forth in SEQ ID NO: 21, (c) a VH CDR3 comprising the amino acid sequence set forth in SEQ ID NO: 22, (d) a VL CDR1 comprising the amino acid sequence set forth in SEQ ID NO: 32, (e) a VL CDR2 comprising the amino acid sequence set forth in SEQ ID NO: 33, and (f) a VL CDR3 comprising the amino acid sequence set forth in SEQ ID NO: 34. In some aspects, an anti-FAM19A5 antibody useful for the present disclosure comprises: (a) a VH CDR1 comprising the amino acid sequence set forth in SEQ ID NO: 89, (b) a VH CDR2 comprising the amino acid sequence set forth in SEQ ID NO: 90, (c) a VH CDR3 comprising the amino acid sequence set forth in SEQ ID NO: 91, (d) a VL CDR1 comprising the amino acid sequence set forth in SEQ ID NO: 92, (e) a VL CDR2 comprising the amino acid sequence set forth in SEQ ID NO: 93, and (f) a VL CDR3 comprising the amino acid sequence set forth in SEQ ID NO: 94. In some aspects, an anti-FAM19A5 antibody useful for the present disclosure comprises: (a) a VH CDR1 comprising the amino acid sequence set forth in SEQ ID NO: 95, (b) a VH CDR2 comprising the amino acid sequence set forth in SEQ ID NO: 96, (c) a VH CDR3 comprising the amino acid sequence set forth in SEQ ID NO: 97, (d) a VL CDR1 comprising the amino acid sequence set forth in SEQ ID NO: 98, (e) a VL CDR2 comprising the amino acid sequence set forth in SEQ ID NO: 99, and (f) a VL CDR3 comprising the amino acid sequence set forth in SEQ ID NO: 100. In some aspects, an anti-FAM19A5 antibody useful for the present disclosure comprises: (a) a VH CDR1 comprising the amino acid sequence set forth in SEQ ID NO: 101, (b) a VH CDR2 comprising the amino acid sequence set forth in SEQ ID NO: 102, (c) a VH CDR3 comprising the amino acid sequence set forth in SEQ ID NO: 103, (d) a VL CDR1 comprising the amino acid sequence set forth in SEQ ID NO: 104, (e) a VL CDR2 comprising the amino acid sequence set forth in SEQ ID NO: 105, and (f) a VL CDR3 comprising the amino acid sequence set forth in SEQ ID NO: 106. In some aspects, an anti-FAM19A5 antibody useful for the present disclosure comprises: (a) a VH CDR1 comprising the amino acid sequence set forth in SEQ ID NO: 107, (b) a VH CDR2 comprising the amino acid sequence set forth in SEQ ID NO: 108, (c) a VH CDR3 comprising the amino acid sequence set forth in SEQ ID NO: 109, (d) a VL CDR1 comprising the amino acid sequence set forth in SEQ ID NO: 110, (e) a VL CDR2 comprising the amino acid sequence set forth in SEQ ID NO: 111, and (f) a VL CDR3 comprising the amino acid sequence set forth in SEQ ID NO: 112. In some aspects, an anti-FAM19A5 antibody useful for the present disclosure comprises: (a) a VH CDR1 comprising the amino acid sequence set forth in SEQ ID NO: 113, (b) a VH CDR2 comprising the amino acid sequence set forth in SEQ ID NO: 114, (c) a VH CDR3 comprising the amino acid sequence set forth in SEQ ID NO: 115, (d) a VL CDR1 comprising the amino acid sequence set forth in SEQ ID NO: 116, (e) a VL CDR2 comprising the amino acid sequence set forth in SEQ ID NO: 117, and (f) a VL CDR3 comprising the amino acid sequence set forth in SEQ ID NO: 118. In some aspects, an anti-FAM19A5 antibody useful for the present disclosure comprises: (a) a VH CDR1 comprising the amino acid sequence set forth in SEQ ID NO: 119, (b) a VH CDR2 comprising the amino acid sequence set forth in SEQ ID NO: 120, (c) a VH CDR3 comprising the amino acid sequence set forth in SEQ ID NO: 121, (d) a VL CDR1 comprising the amino acid sequence set forth in SEQ ID NO: 122, (e) a VL CDR2 comprising the amino acid sequence set forth in SEQ ID NO: 123, and (f) a VL CDR3 comprising the amino acid sequence set forth in SEQ ID NO: 124. In some aspects, an anti-FAM19A5 antibody useful for the present disclosure comprises: (a) a VH CDR1 comprising the amino acid sequence set forth in SEQ ID NO: 125, (b) a VH CDR2 comprising the amino acid sequence set forth in SEQ ID NO: 126, (c) a VH CDR3 comprising the amino acid sequence set forth in SEQ ID NO: 127, (d) a VL CDR1 comprising the amino acid sequence set forth in SEQ ID NO: 128, (e) a VL CDR2 comprising the amino acid sequence set forth in SEQ ID NO: 129, and (f) a VL CDR3 comprising the amino acid sequence set forth in SEQ ID NO: 130. In some aspects, an anti-FAM19A5 antibody useful for the present disclosure comprises: (a) a VH CDR1 comprising the amino acid sequence set forth in SEQ ID NO: 131, (b) a VH CDR2 comprising the amino acid sequence set forth in SEQ ID NO: 132, (c) a VH CDR3 comprising the amino acid sequence set forth in SEQ ID NO: 133, (d) a VL CDR1 comprising the amino acid sequence set forth in SEQ ID NO: 134, (e) a VL CDR2 comprising the amino acid sequence set forth in SEQ ID NO: 135, and (f) a VL CDR3 comprising the amino acid sequence set forth in SEQ ID NO: 136. In some aspects, an anti-FAM19A5 antibody useful for the present disclosure comprises: (a) a VH CDR1 comprising the amino acid sequence set forth in SEQ ID NO: 137, (b) a VH CDR2 comprising the amino acid sequence set forth in SEQ ID NO: 138, (c) a VH CDR3 comprising the amino acid sequence set forth in SEQ ID NO: 139, (d) a VL CDR1 comprising the amino acid sequence set forth in SEQ ID NO: 140, (e) a VL CDR2 comprising the amino acid sequence set forth in SEQ ID NO: 141, and (f) a VL CDR3 comprising the amino acid sequence set forth in SEQ ID NO: 142. In some aspects, an anti-FAM19A5 antibody useful for the present disclosure comprises: (a) a VH CDR1 comprising the amino acid sequence set forth in SEQ ID NO: 143, (b) a VH CDR2 comprising the amino acid sequence set forth in SEQ ID NO: 144, (c) a VH CDR3 comprising the amino acid sequence set forth in SEQ ID NO: 145, (d) a VL CDR1 comprising the amino acid sequence set forth in SEQ ID NO: 146, (e) a VL CDR2 comprising the amino acid sequence set forth in SEQ ID NO: 147, and (f) a VL CDR3 comprising the amino acid sequence set forth in SEQ ID NO: 148. In some aspects, an anti-FAM19A5 antibody useful for the present disclosure comprises: (a) a VH CDR1 comprising the amino acid sequence set forth in SEQ ID NO: 149, (b) a VH CDR2 comprising the amino acid sequence set forth in SEQ ID NO: 150, (c) a VH CDR3 comprising the amino acid sequence set forth in SEQ ID NO: 151, (d) a VL CDR1 comprising the amino acid sequence set forth in SEQ ID NO: 152, (e) a VL CDR2 comprising the amino acid sequence set forth in SEQ ID NO: 153, and (f) a VL CDR3 comprising the amino acid sequence set forth in SEQ ID NO: 154. In some aspects, an anti-FAM19A5 antibody useful for the present disclosure comprises: (a) a VH CDR1 comprising the amino acid sequence set forth in SEQ ID NO: 17, (b) a VH CDR2 comprising the amino acid sequence set forth in SEQ ID NO: 18, (c) a VH CDR3 comprising the amino acid sequence set forth in SEQ ID NO: 19, (d) a VL CDR1 comprising the amino acid sequence set forth in SEQ ID NO: 208, (e) a VL CDR2 comprising the amino acid sequence set forth in SEQ ID NO: 209, and (f) a VL CDR3 comprising the amino acid sequence set forth in SEQ ID NO: 31. In some aspects, an anti-FAM19A5 antibody useful for the present disclosure comprises: (a) a VH CDR1 comprising the amino acid sequence set forth in SEQ ID NO: 17, (b) a VH CDR2 comprising the amino acid sequence set forth in SEQ ID NO: 252, (c) a VH CDR3 comprising the amino acid sequence set forth in SEQ ID NO: 19, (d) a VL CDR1 comprising the amino acid sequence set forth in SEQ ID NO: 208, (e) a VL CDR2 comprising the amino acid sequence set forth in SEQ ID NO: 213, and (f) a VL CDR3 comprising the amino acid sequence set forth in SEQ ID NO: 31. In some aspects, an anti-FAM19A5 antibody useful for the present disclosure comprises: (a) a VH CDR1 comprising the amino acid sequence set forth in SEQ ID NO: 17, (b) a VH CDR2 comprising the amino acid sequence set forth in SEQ ID NO: 253, (c) a VH CDR3 comprising the amino acid sequence set forth in SEQ ID NO: 19, (d) a VL CDR1 comprising the amino acid sequence set forth in SEQ ID NO: 208, (e) a VL CDR2 comprising the amino acid sequence set forth in SEQ ID NO: 213, and (f) a VL CDR3 comprising the amino acid sequence set forth in SEQ ID NO: 31. In some aspects, an anti-FAM19A5 antibody useful for the present disclosure comprises: (a) a VH CDR1 comprising the amino acid sequence set forth in SEQ ID NO: 218, (b) a VH CDR2 comprising the amino acid sequence set forth in SEQ ID NO: 219, (c) a VH CDR3 comprising the amino acid sequence set forth in SEQ ID NO: 16, (d) a VL CDR1 comprising the amino acid sequence set forth in SEQ ID NO: 26, (e) a VL CDR2 comprising the amino acid sequence set forth in SEQ ID NO: 224, and (f) a VL CDR3 comprising the amino acid sequence set forth in SEQ ID NO: 28. In some aspects, an anti-FAM19A5 antibody useful for the present disclosure comprises: (a) a VH CDR1 comprising the amino acid sequence set forth in SEQ ID NO: 220, (b) a VH CDR2 comprising the amino acid sequence set forth in SEQ ID NO: 221, (c) a VH CDR3 comprising the amino acid sequence set forth in SEQ ID NO: 16, (d) a VL CDR1 comprising the amino acid sequence set forth in SEQ ID NO: 225, (e) a VL CDR2 comprising the amino acid sequence set forth in SEQ ID NO: 226, and (f) a VL CDR3 comprising the amino acid sequence set forth in SEQ ID NO: 227. In some aspects, an anti-FAM19A5 antibody useful for the present disclosure comprises: (a) a VH CDR1 comprising the amino acid sequence set forth in SEQ ID NO: 220, (b) a VH CDR2 comprising the amino acid sequence set forth in SEQ ID NO: 221, (c) a VH CDR3 comprising the amino acid sequence set forth in SEQ ID NO: 16, (d) a VL CDR1 comprising the amino acid sequence set forth in SEQ ID NO: 225, (e) a VL CDR2 comprising the amino acid sequence set forth in SEQ ID NO: 228, and (f) a VL CDR3 comprising the amino acid sequence set forth in SEQ ID NO: 227. In some aspects, an anti-FAM19A5 antibody useful for the present disclosure comprises: (a) a VH CDR1 comprising the amino acid sequence set forth in SEQ ID NO: 220, (b) a VH CDR2 comprising the amino acid sequence set forth in SEQ ID NO: 221, (c) a VH CDR3 comprising the amino acid sequence set forth in SEQ ID NO: 16, (d) a VL CDR1 comprising the amino acid sequence set forth in SEQ ID NO: 26, (e) a VL CDR2 comprising the amino acid sequence set forth in SEQ ID NO: 229, and (f) a VL CDR3 comprising the amino acid sequence set forth in SEQ ID NO: 227. In some aspects, an anti-FAM19A5 antibody useful for the present disclosure comprises: (a) a VH CDR1 comprising the amino acid sequence set forth in SEQ ID NO: 220, (b) a VH CDR2 comprising the amino acid sequence set forth in SEQ ID NO: 221, (c) a VH CDR3 comprising the amino acid sequence set forth in SEQ ID NO: 16, (d) a VL CDR1 comprising the amino acid sequence set forth in SEQ ID NO: 26, (e) a VL CDR2 comprising the amino acid sequence set forth in SEQ ID NO: 228, and (f) a VL CDR3 comprising the amino acid sequence set forth in SEQ ID NO: 227. In some aspects, an anti-FAM19A5 antibody useful for the present disclosure comprises: (a) a VH CDR1 comprising the amino acid sequence set forth in SEQ ID NO: 220, (b) a VH CDR2 comprising the amino acid sequence set forth in SEQ ID NO: 221, (c) a VH CDR3 comprising the amino acid sequence set forth in SEQ ID NO: 16, (d) a VL CDR1 comprising the amino acid sequence set forth in SEQ ID NO: 26, (e) a VL CDR2 comprising the amino acid sequence set forth in SEQ ID NO: 230, and (f) a VL CDR3 comprising the amino acid sequence set forth in SEQ ID NO: 231. In some aspects, an anti-FAM19A5 antibody useful for the present disclosure comprises: (a) a VH CDR1 comprising the amino acid sequence set forth in SEQ ID NO: 220, (b) a VH CDR2 comprising the amino acid sequence set forth in SEQ ID NO: 221, (c) a VH CDR3 comprising the amino acid sequence set forth in SEQ ID NO: 16, (d) a VL CDR1 comprising the amino acid sequence set forth in SEQ ID NO: 26, (e) a VL CDR2 comprising the amino acid sequence set forth in SEQ ID NO: 232, and (f) a VL CDR3 comprising the amino acid sequence set forth in SEQ ID NO: 233. In some aspects, an anti-FAM19A5 antibody useful for the present disclosure comprises: (a) a VH CDR1 comprising the amino acid sequence set forth in SEQ ID NO: 222, (b) a VH CDR2 comprising the amino acid sequence set forth in SEQ ID NO: 12, (c) a VH CDR3 comprising the amino acid sequence set forth in SEQ ID NO: 13, (d) a VL CDR1 comprising the amino acid sequence set forth in SEQ ID NO: 248, (e) a VL CDR2 comprising the amino acid sequence set forth in SEQ ID NO: 24, and (f) a VL CDR3 comprising the amino acid sequence set forth in SEQ ID NO: 25. In some aspects, an anti-FAM19A5 antibody useful for the present disclosure comprises: (a) a VH CDR1 comprising the amino acid sequence set forth in SEQ ID NO: 223, (b) a VH CDR2 comprising the amino acid sequence set forth in SEQ ID NO: 12, (c) a VH CDR3 comprising the amino acid sequence set forth in SEQ ID NO: 13, (d) a VL CDR1 comprising the amino acid sequence set forth in SEQ ID NO: 248, (e) a VL CDR2 comprising the amino acid sequence set forth in SEQ ID NO: 24, and (f) a VL CDR3 comprising the amino acid sequence set forth in SEQ ID NO: 25. In some aspects, an anti-FAM19A5 antibody useful for the present disclosure comprises: (a) a VH CDR1 comprising the amino acid sequence set forth in SEQ ID NO: 223, (b) a VH CDR2 comprising the amino acid sequence set forth in SEQ ID NO: 12, (c) a VH CDR3 comprising the amino acid sequence set forth in SEQ ID NO: 246, (d) a VL CDR1 comprising the amino acid sequence set forth in SEQ ID NO: 248, (e) a VL CDR2 comprising the amino acid sequence set forth in SEQ ID NO: 24, and (f) a VL CDR3 comprising the amino acid sequence set forth in SEQ ID NO: 25. In some aspects, an anti-FAM19A5 antibody useful for the present disclosure comprises: (a) a VH CDR1 comprising the amino acid sequence set forth in SEQ ID NO: 223, (b) a VH CDR2 comprising the amino acid sequence set forth in SEQ ID NO: 12, (c) a VH CDR3 comprising the amino acid sequence set forth in SEQ ID NO: 247, (d) a VL CDR1 comprising the amino acid sequence set forth in SEQ ID NO: 248, (e) a VL CDR2 comprising the amino acid sequence set forth in SEQ ID NO: 24, and (f) a VL CDR3 comprising the amino acid sequence set forth in SEQ ID NO: 25.

Non-limiting examples of anti-FAM19A5 antibodies that can be used with the present disclosure are provided in US20220372122A1, US20200299373A1, US20210054062A1, and US20220144932A1, each of which is incorporated herein by reference in its entirety.

In some aspects, an anti-FAM19A5 antibody useful for the present disclosure binds to at least one epitope of mature human FAM19A5 (also referred to herein as “FAM19A5 epitope”), as determined, e.g., by binding of the antibody to fragments of human FAM19A5. In some aspects, a FAM19A5 epitope useful for the present disclosure comprises an amino acid sequence that is at least 90%, at least about 95%, at least about 96%, at least about 97%, at least about 98%, at least about 99%, or about 100% identical in sequence to the sequence set forth in SEQ ID NO: 178, 179, 180, 181, 182, or 183, or a fragment of thereof.

Accordingly, in some aspects, an anti-FAM19A5 antibody useful for the present disclosure binds to a FAM19A5 epitope, which comprises an amino acid sequence that is at least 90%, at least about 95%, at least about 96%, at least about 97%, at least about 98%, at least about 99%, or about 100% identical in sequence to the sequence set forth in SEQ ID NO: 178 or a fragment thereof. In some aspects, the FAM19A5 epitope comprises the sequence set forth in SEQ ID NO: 178 or a fragment thereof. In some aspects, the FAM19A5 epitope consists of the sequence set forth in SEQ ID NO: 178 or a fragment thereof. In some aspects, an anti-FAM19A5 antibody binds to a FAM19A5 epitope, which comprises an amino acid sequence that is at least 90%, at least about 95%, at least about 96%, at least about 97%, at least about 98%, at least about 99%, or about 100% identical in sequence to the sequence set forth in SEQ ID NO: 179 or a fragment thereof. In some aspects, the FAM19A5 epitope comprises the sequence set forth in SEQ ID NO: 179 or a fragment thereof. In some aspects, the FAM19A5 epitope consists of the sequence set forth in SEQ ID NO: 179 or a fragment thereof. In some aspects, an anti-FAM19A5 antibody binds to a FAM19A5 epitope, which comprises an amino acid sequence that is at least 90%, at least about 95%, at least about 96%, at least about 97%, at least about 98%, at least about 99%, or about 100% identical in sequence to the sequence set forth in SEQ ID NO: 180 or a fragment thereof. In some aspects, the FAM19A5 epitope comprises the sequence set forth in SEQ ID NO: 180 or a fragment thereof. In some aspects, the FAM19A5 epitope consists of the sequence set forth in SEQ ID NO: 180 or a fragment thereof. In some aspects, an anti-FAM19A5 antibody binds to a FAM19A5 epitope, which comprises an amino acid sequence that is at least 90%, at least about 95%, at least about 96%, at least about 97%, at least about 98%, at least about 99%, or about 100% identical in sequence to the sequence set forth in SEQ ID NO: 181 or a fragment thereof. In some aspects, the FAM19A5 epitope comprises the sequence set forth in SEQ ID NO: 181 or a fragment thereof. In some aspects, the FAM19A5 epitope consists of the sequence set forth in SEQ ID NO: 181 or a fragment thereof. In some aspects, an anti-FAM19A5 antibody binds to a FAM19A5 epitope, which comprises an amino acid sequence that is at least 90%, at least about 95%, at least about 96%, at least about 97%, at least about 98%, at least about 99%, or about 100% identical in sequence to the sequence set forth in SEQ ID NO: 182 or a fragment thereof. In some aspects, the FAM19A5 epitope comprises the sequence set forth in SEQ ID NO: 182 or a fragment thereof. In some aspects, the FAM19A5 epitope consists of the sequence set forth in SEQ ID NO: 182 or a fragment thereof. In some aspects, an anti-FAM19A5 antibody binds to a FAM19A5 epitope, which comprises an amino acid sequence that is at least 90%, at least about 95%, at least about 96%, at least about 97%, at least about 98%, at least about 99%, or about 100% identical in sequence to the sequence set forth in SEQ ID NO: 183 or a fragment thereof. In some aspects, the FAM19A5 epitope comprises the sequence set forth in SEQ ID NO: 183 or a fragment thereof. In some aspects, the FAM19A5 epitope consists of the sequence set forth in SEQ ID NO: 183 or a fragment thereof.

In some aspects, the anti-FAM19A5 antibody binds to at least one FAM19A5 epitope identified as EP6, EP7, and/or EP8, wherein EP6 comprises, consists essentially of, or consists of the amino acids KTKQWCDML (SEQ ID NO: 189), wherein EP7 comprises, consists essentially of, or consists of the amino acids GCDLLINR (SEQ ID NO: 190), and wherein EP8 comprises, consists essentially of, or consists of the amino acids TCTQPGGR (SEQ ID NO: 191). In some aspects, the anti-FAM19A5 antibody only binds to EP6, EP7, and/or EP8. In some aspects, the anti-FAM19A5 antibody further binds to an additional FAM19A5 epitope selected from the group consisting of SEQ ID NO: 184 (also referred to herein as “EP1”), SEQ ID NO: 185 (also referred to herein as “EP2”), SEQ ID NO: 186 (also referred to herein as “EP3”), SEQ ID NO: 187 (also referred to herein as “EP4”), SEQ ID NO: 188 (also referred to herein as “EP5”), and any combinations thereof. In some aspects, anti-FAM19A5 antibodies or antigen binding portions thereof bind to at least one FAM19A5 epitope identified as EP2, EP4, and/or EP8, wherein EP2 comprises, consists essentially of, or consists of the amino acids DSSQP (SEQ ID NO: 185), wherein EP4 comprises, consists essentially of, or consists of the amino acids ARCACRK (SEQ ID NO: 187), and wherein EP8 comprises, consists essentially of, or consists of the amino acids TCTQPGGR (SEQ ID NO: 191). In some aspects, the at least one epitope has the amino acid sequence that is at least 90%, at least about 95%, at least about 96%, at least about 97%, at least about 98%, at least about 99%, or about 100% identical to EP2, EP4, or EP8. In some aspects, anti-FAM19A5 antibodies or antigen binding portion thereof only bind to EP2. In some aspects, anti-FAM19A5 antibodies or antigen binding portion thereof bind to EP4 and EP8. In some aspects, anti-FAM19A5 antibodies or antigen binding portion thereof bind to one or more FAM19A5 epitopes selected from the group consisting of SEQ ID NO: 184 (i.e., EP1), SEQ ID NO: 186 (i.e., EP3), SEQ ID NO: 188 (i.e., EP5), SEQ ID NO: 189 (i.e., EP6), and SEQ ID NO: 190 (i.e., EP7), and any combinations thereof.

In some aspects, an anti-FAM19A5 antibody binds to at least one epitope, which has the amino acid sequence of QLAAGTCEIVTLDR (SEQ ID NO: 178 or amino acid residues 32 to 45 of SEQ ID NO: 2), or binds to a fragment located within the at least one epitope (e.g., a fragment within the amino acid sequence of SEQ ID NO: 178, e.g., an epitope having at least 1, 2, 3, 4, 5, 6, 7, 8, 9, 10, 11, 12, 13, or 14 amino acids of SEQ ID NO: 178). In some aspects, an anti-FAM19A5 antibody binds to at least one epitope, which has the amino acid sequence of CDMLPCLEGEGCDLLINRSG (SEQ ID NO: 182 or amino acid residues 90 to 109 of SEQ ID NO: 2), or binds to a fragment located within the at least one epitope (e.g., a fragment within the amino acid sequence of SEQ ID NO: 182, e.g., an epitope having at least 1, 2, 3, 4, 5, 6, 7, 8, 9, 10, 11, 12, 13, 14, 15, 16, 17, 18, 19, or 20 amino acids of SEQ ID NO: 182). In some aspects, an anti-FAM19A5 antibody binds to at least one epitope, which has the amino acid sequence of NRSGWTCTQPGGRIKTTTVS (SEQ ID NO: 183 or amino acid residues 106 to 125 of SEQ ID NO: 2), or binds to a fragment located within the at least one epitope (e.g., a fragment within the amino acid sequence of SEQ ID NO: 183, e.g., an epitope having at least 1, 2, 3, 4, 5, 6, 7, 8, 9, 10, 11, 12, 13, 14, 15, 16, 17, 18, 19, or 20 amino acids of SEQ ID NO: 183).

In some aspects, an anti-FAM19A5 antibody binds to at least one epitope, which has the amino acid sequence of TLDRDSSQPRRTIARQTARC (SEQ ID NO: 179 or amino acid residues 42 to 61 of SEQ ID NO: 2), or binds to a fragment located within the at least one epitope (e.g., a fragment within the amino acid sequence of SEQ ID NO: 179, e.g., an epitope having at least 1, 2, 3, 4, 5, 6, 7, 8, 9, 10, 11, 12, 13, 14, 15, 16, 17, 18, 19, or 20 amino acids of SEQ ID NO: 179). In some aspects, an anti-FAM19A5 antibody useful for the present disclosure binds to one or more amino acids corresponding to amino acid residues 44 to52 (i.e., DRDSSQPRR; SEQ ID NO: 192) of SEQ ID NO: 2, e.g., amino acid residues 45, 46, 50, 51, and 52 (RD---PRR), e.g., amino acid residues 45, 50, 51, and 52 (i.e., R----PRR), e.g., amino acid residues 43, 50, and 51 (i.e., R----PR). In some aspects, an anti-FAM19A5 antibody binds to at one or more amino acids corresponding to amino acid residues 46 to 51 (i.e., DSSQPR; SEQ ID NO: 196), e.g., amino acid residues 46, 50, and 52 (i.e., D---P-R), e.g., amino acid residues 46, 47, 48, and 50 (i.e., DSS-P) of SEQ ID NO: 2.

In some aspects, an anti-FAM19A5 antibody binds to at least one epitope, which has the amino acid sequence of TARCACRKGQIAGTTRARPA (SEQ ID NO: 180 or amino acid residues 58 to 77 of SEQ ID NO: 2), or binds to a fragment located within the at least one epitope (e.g., a fragment within the amino acid sequence of SEQ ID NO: 180, e.g., an epitope having at least 1, 2, 3, 4, 5, 6, 7, 8, 9, 10, 11, 12, 13, 14, 15, 16, 17, 18, 19, or 20 amino acids of SEQ ID NO: 180). In some aspects, an anti-FAM19A5 antibody binds to at one or more amino acids corresponding to amino acid residue 63 to 75 (i.e., CRKGQIAGTTRAR; SEQ ID NO: 199) of SEQ ID NO: 2.

In some aspects, an anti-FAM19A5 antibody binds to at least one epitope, which has the amino acid sequence of ARPACVDARIIKTKQWCDML (SEQ ID NO: 181 or amino acid residues 74 to 93 of SEQ ID NO: 2), or binds to a fragment located within the at least one epitope (e.g., a fragment within amino acid sequence of SEQ ID NO: 181, e.g., an epitope having at least 1, 2, 3, 4, 5, 6, 7, 8, 9, 10, 11, 12, 13, 14, 15, 16, 17, 18, 19, or 20 amino acids of SEQ ID NO: 181). In some aspects, an anti-FAM19A5 antibody that can be used with the present disclosure binds to at one or more amino acids corresponding to amino acid residue 76 to 89 (i.e., PACVDARIIKTKQW; SEQ ID NO: 200) of SEQ ID NO: 2.

In some aspects, the one or more additional FAM19A5 epitopes are selected from QLAAGTCEIVTLDR (SEQ ID NO: 178, epitope F1), TLDRDSSQPRRTIARQTARC (SEQ ID NO: 179, epitope F2), TARCACRKGQIAGTTRARPA (SEQ ID NO: 180, epitope F3), ARPACVDARIIKTKQWCDML (SEQ ID NO: 181, epitope F4), CDMLPCLEGEGCDLLINRSG (SEQ ID NO: 182, epitope F5), or NRSGWTCTQPGGRIKTTTVS (SEQ ID NO: 183, epitope F6), or a fragment located within the amino acid sequence of SEQ ID NO: 178, SEQ ID NO: 179, SEQ ID NO: 180, SEQ ID NO: 181, SEQ ID NO: 182, or SEQ ID NO: 183, or any combination thereof. A fragment located within the amino acid sequence of SEQ ID NO: 178, SEQ ID NO: 179, SEQ ID NO: 180, SEQ ID NO: 181, SEQ ID NO: 182, or SEQ ID NO: 183, includes a fragment having 1, 2, 3, 4, 5, 6, 7, 8, 9, 10, 11, 12, 13, 14, 15, 16, 17, 18, 19, or 20 amino acids of any of SEQ ID NO: 178, SEQ ID NO: 179, SEQ ID NO: 180, SEQ ID NO: 181, SEQ ID NO: 182, or SEQ ID NO: 183. In some aspects, the anti-FAM19A5 antibody or antigen-binding portion thereof of the disclosure binds to any of the one or more additional epitopes in their native conformation (i.e., un-denatured). In some aspects, the anti-FAM19A5 antibody or antigen-binding portion thereof binds to both glycosylated and unglycosylated of the one or more additional FAM19A5 epitopes.

In some aspects, an anti-FAM19A5 antibody useful for the present disclosure is capable of binding to multiple FAM19A5 epitopes (such as those described herein). In some aspects, an anti-FAM19A5 antibody binds to at least two different FAM19A5 epitopes described herein (e.g., comprising an amino acid sequence set forth in any one of SEQ ID NOs: 178-183 or a fragment thereof). In some aspects, an anti-FAM19A5 antibody binds to at least three different FAM19A5 epitopes described herein. In some aspects, an anti-FAM19A5 antibody binds to at least four different FAM19A5 epitopes described herein. In some aspects, an anti-FAM19A5 antibody binds to at least five different FAM19A5 epitopes described herein.

In some aspects, an anti-FAM19A5 antibody of the present disclosure binds to SEQ ID NO: 179 or a fragment thereof in its native conformation (i.e., un-denatured). In some aspects, the anti-FAM19A5 antibody or antigen-binding portion thereof binds to both glycosylated and unglycosylated human FAM19A5

### III. B Exemplary LRRC4 Family Mimic Molecules

As further described herein, in some aspects, FAM19A5 antagonists useful for the present disclosure comprises a LRRC4 family mimic molecule.

Where the LRRC4 family mimic molecules comprise small molecule compounds, in some aspects, the mimic molecule is:

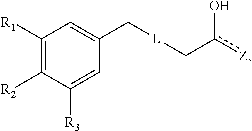

or a pharmaceutically acceptable salt thereof, wherein:
(i) R₁, R₂ and R₃ are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N,N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl;
(ii) [symbol] is a single or double bond;
(iii) Z is selected from a straight chain or branched (C₁-C₈)alkyl, a straight chain or branched (C₂-C₈)alkenyl, a straight chain or branched (C₂-C₈)alkynyl, (C₃-C₈)cycloalkyl, (C₅-C₈)cycloalkenyl, (3-8 membered)heterocycloalkyl, (C₇-C₁₄)bicycloalkyl, (C₇-C₁₄)bicycloalkenyl, (7-14 membered) heterobicycloalkyl, (C₆-C₁₀)aryl, (5-10-membered)heteroaryl, and —CH—C(O)—CH═CH-Q, wherein Q is (C₃-C₈)cycloalkyl, (C₅-C₈)cycloalkenyl, (3-8 membered)heterocycloalkyl, (C₆-C₁₀)aryl, and (5-6-membered)heteroaryl; wherein each cycloalkyl, cycloalkenyl, heterocyclylalkyl, aryl, and heteroaryl are optionally substituted with one, two, three, four, or five substituents independently selected from (C₁-C₆)alkoxy, (C₁-C₆)alkyl, halo, (C₁-C₆)haloalkoxy, nitro, amino, N-methylamino, N-ethylamino, N—N-propylamino, N,N-dimethylamino, formyl, and hydroxy, and
(iv) L is single, double or triple bond.

In some aspects, R₁, R₂ and R₃ are selected from hydrogen, hydroxy, methoxy, ethoxy, n-propyloxy, iso-propyloxy, n-butoxy, trifluoromethoxy, difluoromethoxy, and fluoromethoxy. In some aspects, R₁, R₂ and R₃ are selected from hydrogen, hydroxy, methoxy, ethoxy, n-propyloxy, iso-propyloxy, n-butoxy. In some aspects, R₁, R₂ and R₃ are selected from hydrogen, hydroxy, and methoxy. In some aspects, R₁ and R₂ are selected from hydroxy and methoxy and R₃ is hydrogen.

In some aspects, Z is selected from a straight chain or branched (C₁-C₅)alkyl, a straight chain or branched (C₂-C₈)alkenyl, a straight chain or branched (C₂-C₈)alkynyl, and —CH—C(O)—CH═CH-Q, wherein Q is selected from (C₆-C₁₀)aryl, and (5-6-membered)heteroaryl; wherein the aryl, and the heteroaryl are optionally substituted with one, two, or three substituents independently selected from (C₁-C₆)alkoxy, (C₁-C₆)alkyl, halo, (C₁-C₆)haloalkoxy, and hydroxy. In some aspects, Z is selected from a straight chain or branched (C₁-C₈)alkyl, a straight chain or branched (C₂-C₈)alkenyl, and —CH—C(O)—CH═CH-Q, wherein Q is (C₆-C₁₀)aryl optionally substituted with one, two, or three substituents independently selected from (C₁-C₆)alkoxy, (C₁-C₆)alkyl, halo, (C₁-C₆)haloalkoxy, and hydroxyl.

In some aspects, the LRRC4 family mimic molecule is a small molecule of formula (I) wherein:
(i) R₁, R₂ and R₃ are selected from hydrogen, hydroxy, methoxy, ethoxy, n-propyloxy, iso-propyloxy, n-butoxy, trifluoromethoxy, difluoromethoxy, and fluoromethoxy;
(ii) [symbol] is a single or double bond;
(iii) Z is selected from a straight chain or branched (C₁-C₅)alkyl, a straight chain or branched (C₂-C₈)alkenyl, a straight chain or branched (C₂-C₈)alkynyl, and —CH—C(O)—CH═CH-Q, wherein Q is selected from (C₆-C₁₀)aryl, and (5-6-membered)heteroaryl; wherein the aryl, and the heteroaryl are optionally substituted with one, two, or three substituents independently selected from (C₁-C₆)alkoxy, (C₁-C₆)alkyl, halo, (C₁-C₆)haloalkoxy, and hydroxy; and
(iv) L is a double or triple bond.

In some aspects, the LRRC4 family mimic molecule is a small molecule of formula (I) wherein:
(i) R₁, R₂ and R₃ are selected from hydrogen, hydroxy, methoxy, ethoxy, n-propyloxy, iso-propyloxy, and n-butoxy;
(ii) [symbol] is a single or double bond;
(iii) Z is selected from a straight chain or branched (C₁-C₈)alkyl, a straight chain or branched (C₂-C₈)alkenyl, and —CH—C(O)—CH═CH-Q, wherein Q is (C₆-C₁₀)aryl optionally substituted with one, two, or three substituents independently selected from (C₁-C₆)alkoxy, (C₁-C₆)alkyl, halo, (C₁-C₆)haloalkoxy, and hydroxy; and
(iv) L is a double or triple bond.

In some aspects, the LRRC4 family mimic molecule is selected from:

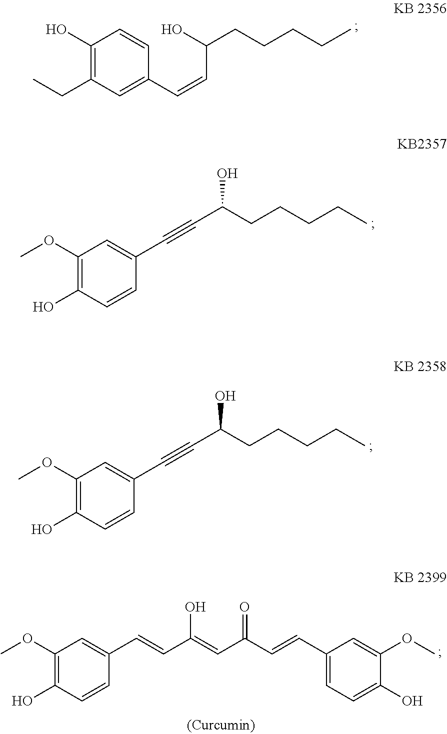

or a pharmaceutically acceptable salt thereof.

In some aspects, the LRRC4 family mimic molecule is:

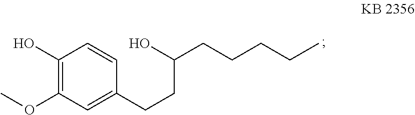 [SMILES: CCCCCC(O)CCc1ccc(O)c(OC)c1]

or a pharmaceutically acceptable salt thereof.

In some aspects, the LRRC4 family mimic molecule is:

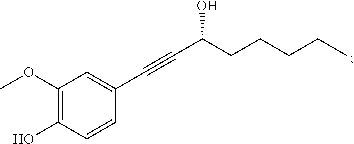 [SMILES: CCCCC[C@@H](O)C#Cc1ccc(O)c(OC)c1]

or a pharmaceutically acceptable salt thereof.

In some aspects, the LRRC4 family mimic molecule is:

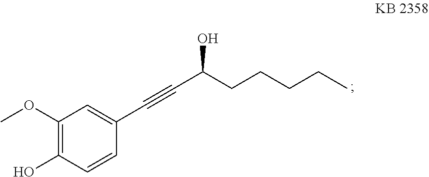 [SMILES: CCCCC[C@H](O)C#Cc1ccc(O)c(OC)c1]

or a pharmaceutically acceptable salt thereof.

In some aspects, the LRRC4 family mimic molecule is:

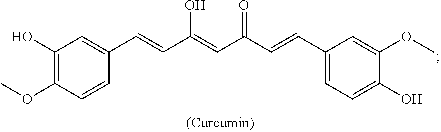

or a pharmaceutically acceptable salt thereof.

In some aspects, the LRRC4 family mimic molecule is:

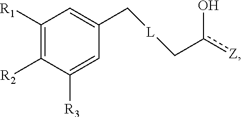

or a pharmaceutically acceptable salt thereof, wherein:
(i) R₁, R₂ and R₃ are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N,N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl;
(ii) [symbol] is a single or double bond;
(iii) Z is selected from a straight chain or branched (C₁-C₈)alkyl, a straight chain or branched (C₂-C₅)alkenyl, a straight chain or branched (C₂-C₈)alkynyl, (C₃-C₈)cycloalkyl, (C₅-C₈)cycloalkenyl, (3-8 membered)heterocycloalkyl, (C₇-C₁₄)bicycloalkyl, (C₇-C₁₄)bicycloalkenyl, (7-14 membered) heterobicycloalkyl, (C₆-C₁₀)aryl, (5-10-membered)heteroaryl, and —CH—C(O)—CH—CH-Q, wherein Q is (C₃-C₈)cycloalkyl, (C₅-C₈)cycloalkenyl, (3-8 membered)heterocycloalkyl, (C₆-C₁₀)aryl, and (5-6-membered)heteroaryl; wherein each cycloalkyl, cycloalkenyl, heterocyclylalkyl, aryl, and heteroaryl are optionally substituted with one, two, three, four, or five substituents independently selected from (C₁-C₆)alkoxy, (C₁-C₆)alkyl, halo, (C₁-C₆)haloalkoxy, nitro, amino, N-methylamino, N-ethylamino, N—N-propylamino, N,N-dimethylamino, formyl, and hydroxy, and
(iv) L is single, double or triple bond, and wherein the LRRC4 family mimic molecule is not selected from:

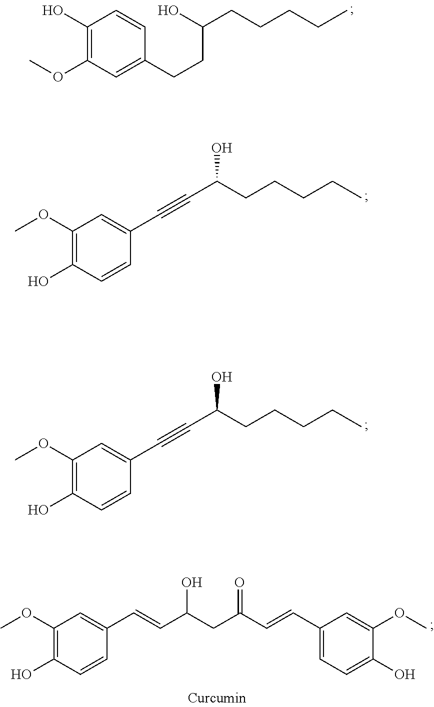

or a pharmaceutically acceptable salt thereof.

In some aspects, the LRRC4 family mimic molecule is:

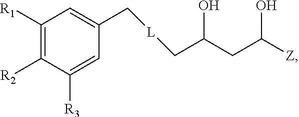

or a pharmaceutically acceptable salt thereof, wherein:
(i) R1, R2 and R3 are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N,N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl,
(ii) Z is selected from a straight chain or branched (C₁-C₈)alkyl, a straight chain or branched (C₂-C₈)alkenyl, a straight chain or branched (C₂-C₈)alkynyl, (C₃-C₈)cycloalkyl, (C₅-C₈)cycloalkenyl, (3-8 membered) heterocycloalkyl, (C₇-C₁₄)bicycloalkyl, (C₇-C₁₄)bicycloalkenyl, (7-14 membered)heterobicycloalkyl, (C₆-C₁₀)aryl, (5-10-membered)heteroaryl, and —CH═CH-Q, wherein Q is (C₃-C₈)cycloalkyl, (C₅-C₈)cycloalkenyl, (3-8 membered)heterocycloalkyl, (C₆-C₁₀)aryl, and (5-6-membered)heteroaryl; wherein each cycloalkyl, cycloalkenyl, heterocyclylalkyl, aryl, and heteroaryl are optionally substituted with one, two, three, four, or five substituents independently selected from (C₁-C₆)alkoxy, (C₁-C₆)alkyl, halo, (C₁-C₆)haloalkoxy, nitro, amino, N-methylamino, N-ethylamino, N—N-propylamino, N,N-dimethylamino, formyl, and hydroxy, and
(iii) L is single, double or triple bond.

In some aspects, R₁, R₂ and R₃ are selected from hydrogen, fluoro, chloro, bromo, hydroxy, methoxy, ethoxy, n-propyloxy, iso-propyloxy, n-butoxy, trifluoromethoxy, difluoromethoxy, fluoromethoxy, amino, N-methylamino, N-ethylamino, N—N-propylamino, and N,N-dimethylamino. In some aspects, R₁, R₂ and R₃ are selected from hydrogen, fluoro, chloro, bromo, hydroxy, methoxy, ethoxy, n-propyloxy, amino, N-methylamino, N-ethylamino, N—N-propylamino, and N,N-dimethylamino. In some aspects, R₁, R₂ and R₃ are selected from hydrogen, fluoro, hydroxy, methoxy, and N,N-dimethylamino. In some aspects, R₁ and R₂ are selected from fluoro, hydroxy, methoxy, and N,N-dimethylamino, and R₃ is hydrogen.

In some aspects, Z is selected from a straight chain or branched (C₁-C₈)alkyl, a straight chain or branched (C₂-C₈)alkenyl, a straight chain or branched (C₂-C₈)alkynyl, and —CH═CH-Q, wherein Q is selected from (C₆-C₁₀)aryl, and (5-6-membered)heteroaryl; wherein the aryl, and the heteroaryl are optionally substituted with one, two, or three substituents independently selected from (C₁-C₆)alkoxy, (C₁-C₆)alkyl, halo, (C₁-C₆)haloalkoxy, and hydroxy. In some aspects, Z is selected from a straight chain or branched (C₁-C₅)alkyl, a straight chain or branched (C₂-C₈)alkenyl, and —CH═CH-Q, wherein Q is (C₆-C₁₀)aryl optionally substituted with one, two, or three substituents independently selected from (C₁-C₆)alkoxy, (C₁-C₆)alkyl, halo, (C₁-C₆)haloalkoxy, and hydroxyl.

In some aspects, the LRRC4 family mimic molecule is a small molecule of formula (II) wherein:
(i) R₁, R₂ and R₃ are selected from hydrogen, fluoro, chloro, bromo, hydroxy, methoxy, ethoxy, n-propyloxy, iso-propyloxy, n-butoxy, trifluoromethoxy, difluoromethoxy, fluoromethoxy, amino, N-methylamino, N-ethylamino, N—N-propylamino, and N,N-dimethylamino. In some aspects, R₁, R₂ and R₃ are selected from hydrogen, fluoro, chloro, bromo, hydroxy, methoxy, ethoxy, n-propyloxy, amino, N-methylamino, N-ethylamino, N—N-propylamino, and N,N-dimethylamino;
(ii) Z is selected from a straight chain or branched (C₁-C₈)alkyl, a straight chain or branched (C₂-C₈)alkenyl, a straight chain or branched (C₂-C₈)alkynyl, and —CH═CH-Q, wherein Q is selected from (C₆-C₁₀)aryl, and (5-6-membered)heteroaryl; wherein the aryl, and the heteroaryl are optionally substituted with one, two, or three substituents independently selected from (C₁-C₆)alkoxy, (C₁-C₆)alkyl, halo, (C₁-C₆)haloalkoxy, and hydroxy; and
(iii) L is a double bond.

In some aspects, the LRRC4 family mimic molecule is a small molecule of formula (ii) wherein:
(i) R₁, R₂ and R₃ are selected from hydrogen, fluoro, chloro, bromo, hydroxy, methoxy, ethoxy, n-propyloxy, amino, N-methylamino, N-ethylamino, N—N-propylamino, and N,N-dimethylamino;
(ii) Z is selected from a straight chain or branched (C₁-C₈)alkyl, a straight chain or branched (C₂-C₈)alkenyl, and —CH═CH-Q, wherein Q is (C₆-C₁₀)aryl optionally substituted with one, two, or three substituents independently selected from (C₁-C₆)alkoxy, (C₁-C₆)alkyl, halo, (C₁-C₆)haloalkoxy, and hydroxy; and
(iii) L is a double bond.

In some aspects, the LRRC4 family molecule is selected from:

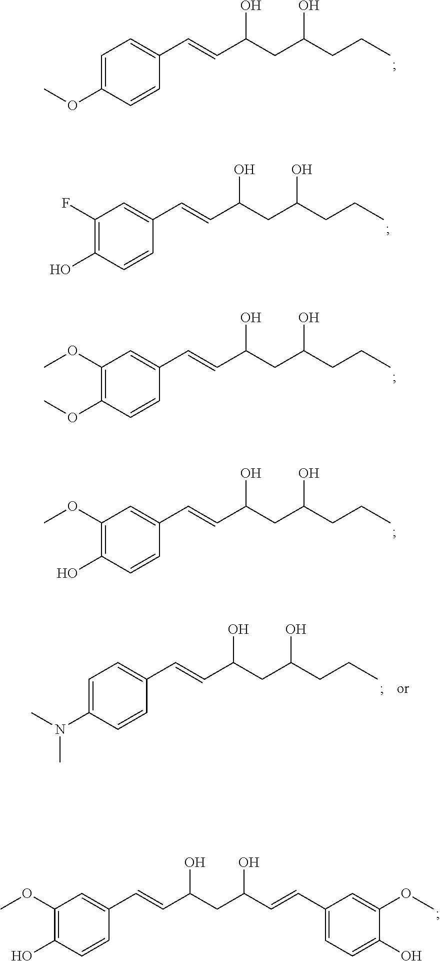

or a pharmaceutically acceptable salt thereof.

In some aspects, the LRRC4 family mimic molecule is:

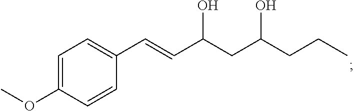 [SMILES: CCCC(O)CC(O)/C=C/c1ccc(OC)cc1]

or a pharmaceutically acceptable salt thereof.

In some aspects, the LRRC4 family mimic molecule is:

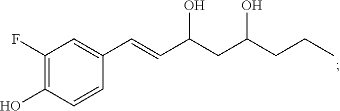 [SMILES: CCCC(O)CC(O)/C=C/c1ccc(O)c(F)c1]

or a pharmaceutically acceptable salt thereof.

In some aspects, the LRRC4 family mimic molecule is:

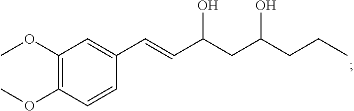 [SMILES: CCCC(O)CC(O)/C=C/c1ccc(OC)c(OC)c1]

or a pharmaceutically acceptable salt thereof.

In some aspects, the LRRC4 family mimic molecule is:

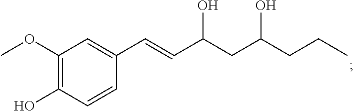 [SMILES: CCCC(O)CC(O)/C=C/c1ccc(O)c(OC)c1]

or a pharmaceutically acceptable salt thereof.

In some aspects, the LRRC4 family mimic molecule is:

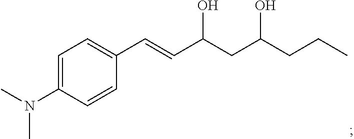 [SMILES: CCCC(O)CC(O)/C=C/c1ccc(N(C)C)cc1]

or a pharmaceutically acceptable salt thereof.

In some aspects, the LRRC4 family mimic molecule is:

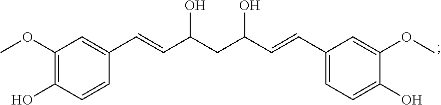

or a pharmaceutically acceptable salt thereof.

In some aspects, the LRRC4 family mimic molecule is:

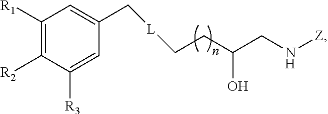

or a pharmaceutically acceptable salt thereof, wherein:

(i) R1, R2 and R3 are independently selected from hydrogen, fluoro, chloro, bromo, methyl, ethyl, n-propyl, iso-propyl, n-butyl, iso-butyl, t-butyl, n-pentyl, iso-pentyl, fluoromethyl, difluoromethyl, trifluoromethyl, 2-fluoroethyl, 1-fluoroethyl, 2,2-difluoroethyl, 1,2-difluoroethyl, 1,1-difluoroethyl, 2,2,2-trifluoroethyl, methoxy, ethoxy, n-propyloxy, iso-propyloxy, n-butoxy, trifluoromethoxy, difluoromethoxy, fluoromethoxy, acetyl, propionyl, n-butanoyl, iso-butanoyl, n-pentanoyl, nitro, amino, N-methylamino, N-ethylamino, N-n-propylamino, N,N-dimethylamino, N-acetylamino, N-propionylamino, N-(trifluoroacetyl)amino, formyl, hydroxy, methylthio, ethylthio, n-propylthio, methylsulfonyl, ethylsulfonyl, n-propylsulfonyl, phenyl, hydroxymethyl, 1-hydroxyethyl and 2-hydroxyethyl,
(ii) Z is selected from a straight chain or branched (C₁-C₅)alkyl, a straight chain or branched (C₂-C₈)alkenyl, a straight chain or branched (C₂-C₈)alkynyl, —Y—(C₃-C₈)cycloalkyl, —Y—(C₅-C₈)cycloalkenyl, —Y-(3-8 membered)heterocycloalkyl, —Y—(C₇-C₁₄)bicycloalkyl, —Y—(C₇-C₁₄)bicycloalkenyl, —Y-(7-14 membered)heterobicycloalkyl, —Y—(C₆-C₁₀)aryl, and —Y-(5-10-membered)heteroaryl, wherein Y is a bond or a C₁-C₃ straight or branched alkylene, and wherein the cycloalkyl, the cycloalkenyl, the heterocyclylalkyl, the aryl, and the heteroaryl are optionally substituted with one, two, three, four, or five substituents independently selected from C₁-C₆alkoxy, C₁-C₆alkyl, halo, C₁-C₆haloalkoxy, nitro, amino, N-methylamino, N-ethylamino, N—N-propylamino, N,N-dimethylamino, formyl, and hydroxy,
(iii) L is single, double or triple bond, and
(iv) n is 0 or 1.

In some aspects, R₁, R₂ and R₃ are selected from hydrogen, hydroxy, methoxy, ethoxy, n-propyloxy, iso-propyloxy, n-butoxy, trifluoromethoxy, difluoromethoxy, and fluoromethoxy. In some aspects, R₁, R₂ and R₃ are selected from hydrogen, hydroxy, methoxy, ethoxy, n-propyloxy, iso-propyloxy, n-butoxy. In some aspects, R₁, R₂ and R₃ are selected from hydrogen, hydroxy, and methoxy. In some aspects, R₁ and R₂ are selected from hydroxy and methoxy and R₃ is hydrogen.

In some aspects, Z is selected from —Y—(C₃-C₈)cycloalkyl, —Y—(C₅-C₈)cycloalkenyl, —Y-(3-8 membered)heterocycloalkyl, —Y—(C₇-C₁₄)bicycloalkyl, —Y—(C₇-C₁₄)bicycloalkenyl, —Y-(7-14 membered) heterobicycloalkyl, —Y—(C₆-C₁₀)aryl, and —Y-(5-10-membered)heteroaryl, wherein Y is a bond or a C₁-C₃ straight or branched alkylene, and wherein the cycloalkyl, the cycloalkenyl, the heterocyclylalkyl, the aryl, and the heteroaryl are optionally substituted with one, two, three, four, or five substituents independently selected from C₁-C₆alkoxy, C₁-C₆alkyl, halo, C₁-C₆haloalkoxy, nitro, amino, N-methylamino, N-ethylamino, N—N-propylamino, N,N-dimethylamino, formyl, and hydroxy. In some aspects, Z is selected from —Y—(C₆-C₁₀)aryl, and —Y-(5-10-membered)heteroaryl wherein Y is a bond or a C₁-C₃ straight or branched alkylene, and wherein the aryl and the heteroaryl are optionally substituted with one, two, or three substituents independently selected from C₁-C₆alkoxy, and halo.

In some aspects, the LRRC4 family mimic molecule is a small molecule of formula (III) wherein:
(i) R₁, R₂ and R₃ are selected from hydrogen, hydroxy, methoxy, ethoxy, n-propyloxy, iso-propyloxy, n-butoxy, trifluoromethoxy, difluoromethoxy, and fluoromethoxy;
(ii) Z is selected from —Y—(C₃-C₈)cycloalkyl, —Y—(C₅-C₈)cycloalkenyl, —Y-(3-8 membered)heterocycloalkyl, —Y—(C₇-C₁₄)bicycloalkyl, —Y—(C₇-C₁₄)bicycloalkenyl, —Y-(7-14 membered)heterobicycloalkyl, —Y—(C₆-C₁₀)aryl, and —Y-(5-10-membered)heteroaryl, wherein Y is a bond or a C₁-C₃ straight or branched alkylene, and wherein the cycloalkyl, the cycloalkenyl, the heterocyclylalkyl, the aryl, and the heteroaryl are optionally substituted with one, two, three, four, or five substituents independently selected from C₁-C₆alkoxy, C₁-C₆alkyl, halo, C₁-C₆haloalkoxy, nitro, amino, N-methylamino, N-ethylamino, N—N-propylamino, N,N-dimethylamino, formyl, and hydroxy; and
(iii) L is a triple bond.

In some aspects, the LRRC4 family mimic molecule is a small molecule of formula (III) wherein:
(i) R₁, R₂ and R₃ are selected from hydrogen, hydroxy, methoxy, ethoxy, n-propyloxy, iso-propyloxy, and n-butoxy;
(ii) Z is selected from —Y—(C₆-C₁₀)aryl, and —Y-(5-10-membered)heteroaryl wherein Y is a bond or a C₁-C₃ straight or branched alkylene, and wherein the aryl and the heteroaryl are optionally substituted with one, two, or three substituents independently selected from C₁-C₆alkoxy, and halo; and
(iii) L is a triple bond.

In some aspects, the LRRC4 family mimic molecule is selected from:

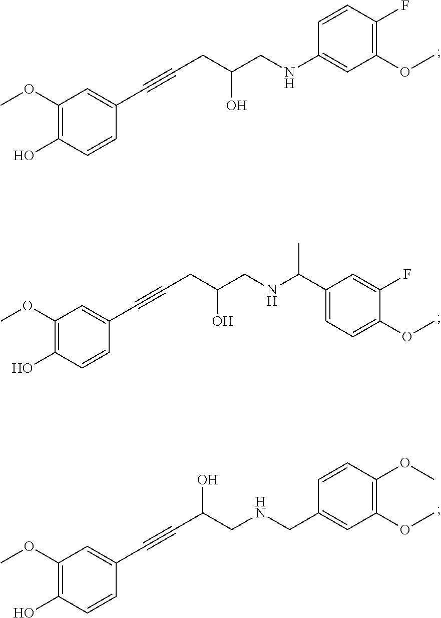

or a pharmaceutically acceptable salt thereof.

In some aspects, the LRRC4 family mimic molecule is:

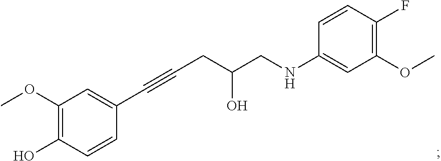

or a pharmaceutically acceptable salt thereof.

In some aspects, the LRRC4 family mimic molecule is:

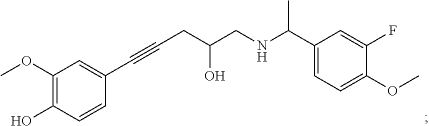

or a pharmaceutically acceptable salt thereof.

In some aspects, the LRRC4 family mimic molecule is:

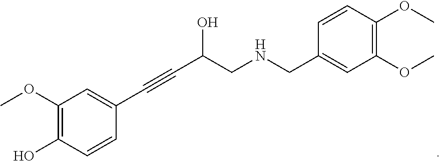

As described herein, where the LRRC4 family mimic molecule useful for the present disclosure comprises a polypeptide, in some aspects, the polypeptide comprises at least the FAM19A5 binding domain of members of the LRRC4 protein family. Unless indicated otherwise, the overall length of the FAM19A5 binding domain is not particularly limited, as long as the domain is capable of binding to the FAM19A5 protein. In some aspects, the FAM19A5 binding domain is at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, or at least about 30 amino acids in length. In some aspects, the FAM19A5 binding domain is about 10 to about 23 amino acids in length. In some aspects, the FAM19A5 binding domain is about 10, about 11, about 12, about 13, about 14, about 15, about 16, about 17, about 18, about 19, about 20, about 21, about 22, or about 23 amino acids in length. In some aspects, the FAM19A5 binding domain of a LRRC4 family mimic molecule is about 10 amino acids in length.

In some aspects, the polypeptide of a LRRC4 family mimic molecule comprises an amino acid sequence having the following formula (from N-terminus to C-terminus):

$$A - ( T / S ) - B , \qquad ( Formula IV ) ( SEQ ID NO : 317 )$$

(i) “A” comprises X1-(T/S)-(Y/F)—F-X5, and (ii) “B” comprises (V/I)-T-V-(E/V), and wherein:
X1 is tyrosine (Y), phenylalanine (F), valine (V), leucine (L), or isoleucine (I);
(T/S) is threonine (T) or serine(S);
(Y/F) is tyrosine (Y) or Phenylalanine (F);
X5 is any amino acids;
(V/I) is valine (V) or isoleucine (I); and
(E/V) is glutamic acid (E) or valine (V).

In some aspects, the polypeptide of a LRRC4 family mimic molecule described herein comprises an amino acid sequence having the formula (from N-terminus to C-terminus):

$$A - ( T / S ) - B , \qquad ( Formula IV ) ( SEQ ID NO : 318 )$$

(i) “A” comprises (Y/W/M)-(T/Y)-(Y/W)-(F/Y/W)-(T/Y), and (ii) “B” comprises X7-(T/S/Y)-X9-X10, and wherein
(Y/W/M) is tyrosine (Y), tryptophan (W), or methionine (M);
(T/Y) is threonine (T) or tyrosine (Y);
(Y/W) is tyrosine (Y) or tryptophan (W);
(F/Y/W) is phenylalanine (F), tyrosine (Y), or tryptophan (W);
X7 is valine (V), tyrosine (Y), phenylalanine (F), leucine (L), tryptophan (W), or methionine (M);
(T/S/Y) is threonine (T), serine(S), or tyrosine (Y);
X9 is valine (V), isoleucine (I), tyrosine (Y), phenylalanine (F), leucine (L), tryptophan (W), or methionine (M); and
X10 is glutamic acid (E), aspartic acid (D), isoleucine (I), tyrosine (Y), phenylalanine (F), methionine (M), or tryptophan (W).

In some aspects, a LRRC4 family mimic molecule useful for the present disclosure comprises a polypeptide that comprises an amino acid sequence having the following formula (from N-terminus to C-terminus):

(Formula V)
(SEQ ID NO: 319)
X1-X2-X3-F-X5-T-X7-T-V-X10,

X1 is Y, F, V, L, or I;
X2 is T or S;
X3 is Y or F;
X5 is any amino acid;
X7 is V or I; and/or
X10 is E or V,
and wherein the polypeptide is capable of binding to a FAM19A5 protein, and thereby inhibiting, reducing, and/or dissociating the interaction between the FAM19A5 protein and members of the LRRC4 protein family.

In some aspects, a LRRC4 family mimic molecule described herein comprises a polypeptide that comprises an amino acid sequence having the following formula (from N-terminus to C-terminus):

(Formula VI)
(SEQ ID NO: 320)
X3-X4-X5-X6-X7-X8-X9-X10,

X1 is Y, F, V, L, I, W, or M;
X2 is T, S, or Y;
X3 is Y, F, or W;
X4 is F, Y, or W;
X5 is any amino acids, e.g., T, S, or Y;
X6 is T, S, or Y;
X7 is V, I, Y, F, L, W, or M;
X8 is T, S, or Y;
X9 is V, I, Y, F, L, W, or M; and/or
X10 is E, D, V, I, Y, F, M, or W,
and wherein the polypeptide is capable of binding to a FAM19A5 protein, and thereby inhibiting, reducing, and/or dissociating the interaction between the FAM19A5 protein and members of the LRRC4 protein family.

For any of the above LRRC4 family mimic molecules, in some aspects, (i) X1 is Y, F, V, L, or I; (ii) X2 is T or S; (iii) X3 is Y or F; (iv) X4 is F; (v) X5 is T or S; (vi) X6 is T; (vii) X7 is V or I; (viii) X8 is T; (ix) X9 is V; (x) X10 is E or V; and (xi) any combinations of (i)-(x). In some aspects, X1 is Y, F, V, L, or I. In some aspects, X2 is T or S. In some aspects, X3 is Y or F. In some aspects, X4 is F. In some aspects, X5 is T or S. In some aspects, X6 is T. In some aspects, X7 is V or I. In some aspects, X8 is T. In some aspects, X9 is V. In some aspects, X10 is E or V. In some aspects, the amino acid at position X2 is phosphorylated. In some aspects, the amino acid at position X2 is O-glycosylated.

In some aspects, a polypeptide of a LRRC4 family mimic molecule described herein comprises the amino acid sequence set forth in SEQ ID NO: 44 (YTYFTTVTVE), with one, two, three, four, five, or six amino acids different from the amino acid sequence (e.g., substitutions). In some aspects, a polypeptide of a LRRC4 family mimic molecule disclosed herein consists of the amino acid sequence set forth in SEQ ID NO: 44 (YTYFTTVTVE), with one, two, three, four, five, or six amino acids different from the amino acid sequence (e.g., substitutions). In some aspects, a polypeptide of a LRRC4 family mimic molecule disclosed herein consists essentially of the amino acid sequence set forth in SEQ ID NO: 44 (YTYFTTVTVE), with one, two, three, four, five, or six amino acids different from the amino acid sequence (e.g., substitutions).

In some aspects, a polypeptide of a LRRC4 family mimic molecule comprises the amino acid sequence set forth in SEQ ID NO: 44 (YTYFTTVTVE). In some aspects, the polypeptide of a LRRC4 family mimic molecule consists of the amino acid sequence set forth in SEQ ID NO: 44 (YTYFTTVTVE). In some aspects, the polypeptide of a LRRC4 family mimic molecule consists essentially of the amino acid sequence set forth in SEQ ID NO: 44 (YTYFTTVTVE). As demonstrated herein, the amino acid sequence set forth in SEQ ID NO: 44 (YTYFTTVTVE) corresponds to the FAM19A5 binding domain of the LRRC4B protein.

In some aspects, a polypeptide of a LRRC4 family mimic molecule comprises the amino acid sequence set forth in SEQ ID NO: 45 (YSFFTTVTVE). In some aspects, the polypeptide of a LRRC4 family mimic molecule consists of the amino acid sequence set forth in SEQ ID NO: 45 (YSFFTTVTVE). In some aspects, the polypeptide of a LRRC4 family mimic molecule consists essentially of the amino acid sequence set forth in SEQ ID NO: 45 (YSFFTTVTVE). As demonstrated herein, the amino acid sequence set forth in SEQ ID NO: 45 (YSFFTTVTVE) corresponds to the FAM19A5 binding domain of the LRRC4 protein.

In some aspects, a polypeptide of a LRRC4 family mimic molecule comprises the amino acid sequence set forth in SEQ ID NO: 46 (FSYFSTVTVE). In some aspects, the polypeptide of a LRRC4 family mimic molecule consists of the amino acid sequence set forth in SEQ ID NO: 46 (FSYFSTVTVE). In some aspects, the polypeptide of a LRRC4 family mimic molecule consists essentially of the amino acid sequence set forth in SEQ ID NO: 46 (FSYFSTVTVE). As demonstrated herein, the amino acid sequence set forth in SEQ ID NO: 46 (FSYFSTVTVE) corresponds to the FAM19A5 binding domain of the LRRC4C protein.

As described herein, the FAM19A5 binding domains of members of the LRRC4 protein family are largely conserved among vertebrates. Accordingly, not to be bound by any one theory, one or more amino acid residues of the amino acid sequence set forth in any one of SEQ ID NOs: 44 (YTYFTTVTVE), 45 (YSFFTTVTVE), and 46 (FSYFSTVTVE) can be substituted with an amino acid present in the corresponding residue in other vertebrates. Examples of such substitutions are provided herein.

In some aspects, one or more amino acid residues of the amino acid sequence set forth in any one of SEQ ID NOs: 44 (YTYFTTVTVE), 45 (YSFFTTVTVE), and 46 (FSYFSTVTVE) can be substituted with an amino acid sharing similar biochemical properties. For instance, in the amino acid sequence set forth in SEQ ID NO: 44 (YTYFTTVTVE), in some aspects, the Y at position 1 can be substituted with other hydrophobic amino acids (e.g., F, V, L, I, W, or M). In some aspects, the T at position 2 can be substituted with other amino acids having a similar hydroxyl (OH) group in its side chain (e.g., S or Y). In some aspects, the Y at position 3 can be substituted with other amino acids having common aromatic ring in its side chain that can participate in Van der Waals interaction (e.g., F or W). In some aspects, the F at position 4 can be substituted with amino acids, such as Y or W. In some aspects, the T at position 5 can be substituted with amino acids, such as S or Y. In some aspects, the T at position 6 can be substituted with amino acids, such as Sor Y. In some aspects, the V at position 7 can be substituted with other amino acids having hydrophobic bulky side chains (e.g., I, Y, F, L, W, or M). In some aspects, the T at position 8 can be substituted with other amino acids, such as S or Y. In some aspects, the V at position 9 can be substituted with other amino acids, such as I, Y, F, L, W, or M. In some aspects, the E at position 10 can be substituted with other amino acids that have an acidic side chain (e.g., I, Y, F, M, or W).

In some aspects, a polypeptide of a LRRC4 family mimic molecule described herein comprises an amino acid sequence that is at least about 40%, at least about 50%, at least about 60%, at least about 70%, at least about 80%, or at least about 90% identical to the amino acid sequence set forth in SEQ ID NO: 44 (YTYFTTVTVE), wherein the polypeptide is capable of binding to a FAM19A5 protein, and thereby inhibiting, reducing, and/or dissociating the interaction between the FAM19A5 protein and members of the LRRC4 protein family. In some aspects, a polypeptide of a LRRC4 family mimic molecule comprises an amino acid sequence that is at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to the amino acid sequence set forth in SEQ ID NO: 9, wherein the polypeptide is capable of binding to a FAM19A5 protein, and thereby inhibiting, reducing, and/or dissociating the interaction between the FAM19A5 protein and members of the LRRC4 protein family. In some aspects, a polypeptide of a LRRC4 family mimic molecule comprises an amino acid sequence that is at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to the amino acid sequence set forth in SEQ ID NO: 6, wherein the polypeptide is capable of binding to a FAM19A5 protein, and thereby inhibiting, reducing, and/or dissociating the interaction between the FAM19A5 protein and members of the LRRC4 protein family. In some aspects, a polypeptide of a LRRC4 family mimic molecule comprises an amino acid sequence that is at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to the amino acid sequence set forth in SEQ ID NO: 84, wherein the polypeptide is capable of binding to a FAM19A5 protein, and thereby inhibiting, reducing, and/or dissociating the interaction between the FAM19A5 protein and members of the LRRC4 protein family.

As is apparent from the present disclosure, in some aspects, polypeptides of the LRRC4 family mimic molecules described herein (e.g., comprising a FAM19A5 binding domain of members of the LRRC4 protein family) comprise one or more amino acid modifications. In some aspects, the one or more amino acid modifications can increase the binding affinity of the LRRC4 family mimic molecule to the FAM19A5 protein. Accordingly, in some aspects, the binding affinity of a LRRC4 family mimic molecule described herein to a FAM19A5 protein is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 35-fold, at least about 40-fold, at least about 45-fold, or at least about 50-fold, compared to a reference (e.g., corresponding LRRC4 family mimic molecule without the amino acid modification(s) or naturally-existing members of the LRRC4 protein family). In some aspects, the one or more amino acid modifications can improve the stability of the LRRC4 family mimic molecule. Accordingly, in some aspects, the stability of a LRRC4 family mimic molecule described herein is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 35-fold, at least about 40-fold, at least about 45-fold, or at least about 50-fold, compared to a reference (e.g., corresponding LRRC4 family mimic molecule without the amino acid modification(s) or naturally-existing members of the LRRC4 protein family).

In some aspects, the one or more amino acid modifications can improve the ability of the LRRC4 family mimic molecules described herein to inhibit the interaction between a FAM19A5 protein and members of the LRRC4 protein family (e.g., by increasing the binding affinity and/or stability). Accordingly, in some aspects, the ability of the LRRC4 family mimic molecule to inhibit the interaction between a FAM19A5 protein and members of the LRRC4 protein family is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 35-fold, at least about 40-fold, at least about 45-fold, or at least about 50-fold, compared to a reference (e.g., corresponding LRRC4 family mimic molecule without the amino acid modification(s) or naturally-existing members of the LRRC4 protein family).

Non-limiting examples of amino acid modifications that are useful for the present disclosure are provided throughout the present disclosure. For instance, in some aspects, a LRRC4 family mimic molecule described herein comprises one of the FAM19A5 binding domains of a members of the LRRC4 protein family—i.e., YTYFTTVTVE (SEQ ID NO: 44), YSFFTTVTVE (SEQ ID NO: 45), or FSYFSTVTVE (SEQ ID NO: 46)—and one or more amino acids at the N-terminus, C-terminus, or both at the N-terminus and C-terminus of the polypeptide. In some aspects, a polypeptide useful for the present disclosure comprises at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 additional amino acids at the N-terminus of the polypeptide. In some aspects, the polypeptide comprises at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about additional 20 amino acids at the C-terminus of the polypeptide. In some aspects, the polypeptide comprises: (i) at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 additional amino acids at the N-terminus of the polypeptide; and (ii) at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 additional amino acids at the C-terminus of the polypeptide. In some aspects, the one or more amino acids differ from the amino acids present at the particular residues in a naturally existing LRRC4 protein family member.

For instance, in some aspects, a polypeptide described herein comprises the amino acid sequence set forth in SEQ ID NO: 47 (GYTYFTTVTVETLETQPGEE) with one or more amino acid modifications (e.g., substitutions). In some aspects, a polypeptide described herein comprises the amino acid sequence set forth in SEQ ID NO: 47 (GYTYFTTVTVETLETQPGEE) with two amino acid modifications (e.g., substitutions). In some aspects, the amino acid modifications are at residues T12 and L13 of SEQ ID NO: 47. In some aspects, a polypeptide described herein consists of the amino acid sequence set forth in SEQ ID NO: 47 (GYTYFTTVTVETLETQPGEE) with one or more amino acid modifications (e.g., substitutions). In some aspects, a polypeptide described herein consists of the amino acid sequence set forth in SEQ ID NO: 47 (GYTYFTTVTVETLETQPGEE) with two amino acid modifications (e.g., substitutions). In some aspects, the amino acid modifications are at residues T12 and L13 of SEQ ID NO: 47. In some aspects, a polypeptide described herein consists essentially of the amino acid sequence set forth in SEQ ID NO: 47 (GYTYFTTVTVETLETQPGEE) with one or more amino acid modifications (e.g., substitutions). In some aspects, a polypeptide described herein consists essentially of the amino acid sequence set forth in SEQ ID NO: 47 (GYTYFTTVTVETLETQPGEE) with two amino acid modifications (e.g., substitutions). In some aspects, the amino acid modifications are at residues T12 and L13 of SEQ ID NO: 47.

In some aspects, a polypeptide described herein comprises the amino acid sequence set forth in SEQ ID NO: 48 (GYTYFTTVTVETLETQ) with one or more amino acid modifications (e.g., substitutions). In some aspects, a polypeptide described herein comprises the amino acid sequence set forth in SEQ ID NO: 48 (GYTYFTTVTVETLETQ) with two amino acid modifications (e.g., substitutions). In some aspects, the amino acid modifications are at residues T12 and L13 of SEQ ID NO: 48. In some aspects, a polypeptide described herein consists of the amino acid sequence set forth in SEQ ID NO: 48 (GYTYFTTVTVETLETQ) with one or more amino acid modifications (e.g., substitutions). In some aspects, a polypeptide described herein consists of the amino acid sequence set forth in SEQ ID NO: 48 (GYTYFTTVTVETLETQ) with two amino acid modifications (e.g., substitutions). In some aspects, the amino acid modifications are at residues T12 and L13 of SEQ ID NO: 48. In some aspects, a polypeptide described herein consists essentially of the amino acid sequence set forth in SEQ ID NO: 48 (GYTYFTTVTVETLETQ) with one or more amino acid modifications (e.g., substitutions). In some aspects, a polypeptide described herein consists essentially of the amino acid sequence set forth in SEQ ID NO: 48 (GYTYFTTVTVETLETQ) with two amino acid modifications (e.g., substitutions). In some aspects, the amino acid modifications are at residues T12 and L13 of SEQ ID NO: 48.

In some aspects, a polypeptide described herein comprises the amino acid sequence set forth in SEQ ID NO: 49 (GYTYFTTVTVETLETQPGEKEPPGPTTD) with one or more amino acid modifications (e.g., substitutions). In some aspects, a polypeptide described herein comprises the amino acid sequence set forth in SEQ ID NO: 49 (GYTYFTTVTVETLETQPGEKEPPGPTTD) with two amino acid modifications (e.g., substitutions). In some aspects, the amino acid modifications are at residues T12 and L13 of SEQ ID NO: 49. In some aspects, a polypeptide described herein consists of the amino acid sequence set forth in SEQ ID NO: 49 (GYTYFTTVTVETLETQPGEKEPPGPTTD) with one or more amino acid modifications (e.g., substitutions). In some aspects, a polypeptide described herein consists of the amino acid sequence set forth in SEQ ID NO: 49 (GYTYFTTVTVETLETQPGEKEPPGPTTD) with two amino acid modifications (e.g., substitutions). In some aspects, the amino acid modifications are at residues T12 and L13 of SEQ ID NO: 49. In some aspects, a polypeptide described herein consists essentially of the amino acid sequence set forth in SEQ ID NO: 49 (GYTYFTTVTVETLETQPGEKEPPGPTTD) with one or more amino acid modifications (e.g., substitutions). In some aspects, a polypeptide described herein consists essentially of the amino acid sequence set forth in SEQ ID NO: 49 (GYTYFTTVTVETLETQPGEKEPPGPTTD) with two amino acid modifications (e.g., substitutions). In some aspects, the amino acid modifications are at residues T12 and L13 of SEQ ID NO: 49.

In some aspects, a polypeptide described herein comprises the amino acid sequence set forth in SEQ ID NO: 50 (GYTYFTTVTVETLETQPGEEA) with one or more amino acid modifications (e.g., substitutions). In some aspects, a polypeptide described herein comprises the amino acid sequence set forth in SEQ ID NO: 50 (GYTYFTTVTVETLETQPGEEA) with two amino acid modifications (e.g., substitutions). In some aspects, the amino acid modifications are at residues T12 and L13 of SEQ ID NO: 50. In some aspects, a polypeptide described herein consists of the amino acid sequence set forth in SEQ ID NO: 50 (GYTYFTTVTVETLETQPGEEA) with one or more amino acid modifications (e.g., substitutions). In some aspects, a polypeptide described herein consists of the amino acid sequence set forth in SEQ ID NO: 50 (GYTYFTTVTVETLETQPGEEA) with two amino acid modifications (e.g., substitutions). In some aspects, the amino acid modifications are at residues T12 and L13 of SEQ ID NO: 50. In some aspects, a polypeptide described herein consists essentially of the amino acid sequence set forth in SEQ ID NO: 50 (GYTYFTTVTVETLETQPGEEA) with one or more amino acid modifications (e.g., substitutions). In some aspects, a polypeptide described herein consists essentially of the amino acid sequence set forth in SEQ ID NO: 50 (GYTYFTTVTVETLETQPGEEA) with two amino acid modifications (e.g., substitutions). In some aspects, the amino acid modifications are at residues T12 and L13 of SEQ ID NO: 50.

In some aspects, a polypeptide described herein (e.g., comprising the FAM19A5 binding domain of LRRC4B) comprises the amino acid sequence GYTYFTTVTVEPYETQPGEE (SEQ ID NO: 51). In some aspects, a polypeptide described herein (e.g., comprising the FAM19A5 binding domain of LRRC4B) consists of the amino acid sequence GYTYFTTVTVEPYETQPGEE (SEQ ID NO: 51). In some aspects, a polypeptide described herein (e.g., comprising the FAM19A5 binding domain of LRRC4B) consists essentially of the amino acid sequence GYTYFTTVTVEPYETQPGEE (SEQ ID NO: 51).

In some aspects, a polypeptide described herein (e.g., comprising the FAM19A5 binding domain of LRRC4B) comprises the amino acid sequence GYTYFTTVTVEMRETQPGEE (SEQ ID NO: 52). In some aspects, a polypeptide described herein (e.g., comprising the FAM19A5 binding domain of LRRC4B) consists of the amino acid sequence GYTYFTTVTVEMRETQPGEE (SEQ ID NO: 52). In some aspects, a polypeptide described herein (e.g., comprising the FAM19A5 binding domain of LRRC4B) consists essentially of the amino acid sequence GYTYFTTVTVEMRETQPGEE (SEQ ID NO: 52).

In some aspects, a polypeptide described herein (e.g., comprising the FAM19A5 binding domain of LRRC4B) comprises the amino acid sequence GYTYFTTVTVEIFETQPGEE (SEQ ID NO: 53). In some aspects, a polypeptide described herein (e.g., comprising the FAM19A5 binding domain of LRRC4B) consists of the amino acid sequence GYTYFTTVTVEIFETQPGEE (SEQ ID NO: 53). In some aspects, a polypeptide described herein (e.g., comprising the FAM19A5 binding domain of LRRC4B) consists essentially of the amino acid sequence GYTYFTTVTVEIFETQPGEE (SEQ ID NO: 53).

In some aspects, a polypeptide described herein (e.g., comprising the FAM19A5 binding domain of LRRC4B) comprises the amino acid sequence GYTYFTTVTVEHFETQPGEE (SEQ ID NO: 54). In some aspects, a polypeptide described herein (e.g., comprising the FAM19A5 binding domain of LRRC4B) consists of the amino acid sequence GYTYFTTVTVEHFETQPGEE (SEQ ID NO: 54). In some aspects, a polypeptide described herein (e.g., comprising the FAM19A5 binding domain of LRRC4B) consists essentially of the amino acid sequence GYTYFTTVTVEHFETQPGEE (SEQ ID NO: 54).

In some aspects, a polypeptide described herein (e.g., comprising the FAM19A5 binding domain of LRRC4B) comprises the amino acid sequence GYTYFTTVTVEWYETQPGEE (SEQ ID NO: 55). In some aspects, a polypeptide described herein (e.g., comprising the FAM19A5 binding domain of LRRC4B) consists of the amino acid sequence GYTYFTTVTVEWYETQPGEE (SEQ ID NO: 55). In some aspects, a polypeptide described herein (e.g., comprising the FAM19A5 binding domain of LRRC4B) consists essentially of the amino acid sequence GYTYFTTVTVEWYETQPGEE (SEQ ID NO: 55).

In some aspects, a polypeptide described herein (e.g., comprising the FAM19A5 binding domain of LRRC4B) comprises the amino acid sequence GYTYFTTVTVEQRETQPGEE (SEQ ID NO: 56). In some aspects, a polypeptide described herein (e.g., comprising the FAM19A5 binding domain of LRRC4B) consists of the amino acid sequence GYTYFTTVTVEQRETQPGEE (SEQ ID NO: 56). In some aspects, a polypeptide described herein (e.g., comprising the FAM19A5 binding domain of LRRC4B) consists essentially of the amino acid sequence GYTYFTTVTVEQRETQPGEE (SEQ ID NO: 56).

In some aspects, a polypeptide described herein (e.g., comprising the FAM19A5 binding domain of LRRC4B) comprises the amino acid sequence GYTYFTTVTVEWFETQPGEE (SEQ ID NO: 57). In some aspects, a polypeptide described herein (e.g., comprising the FAM19A5 binding domain of LRRC4B) consists of the amino acid sequence GYTYFTTVTVEWFETQPGEE (SEQ ID NO: 57). In some aspects, a polypeptide described herein (e.g., comprising the FAM19A5 binding domain of LRRC4B) consists essentially of the amino acid sequence GYTYFTTVTVEWFETQPGEE (SEQ ID NO: 57).

In some aspects, a polypeptide described herein (e.g., comprising the FAM19A5 binding domain of LRRC4B) comprises the amino acid sequence GYTYFTTVTVEERETQPGEE (SEQ ID NO: 58). In some aspects, a polypeptide described herein (e.g., comprising the FAM19A5 binding domain of LRRC4B) consists of the amino acid sequence GYTYFTTVTVEERETQPGEE (SEQ ID NO: 58). In some aspects, a polypeptide described herein (e.g., comprising the FAM19A5 binding domain of LRRC4B) consists essentially of the amino acid sequence GYTYFTTVTVEERETQPGEE (SEQ ID NO: 58).

In some aspects, a polypeptide described herein (e.g., comprising the FAM19A5 binding domain of LRRC4B) comprises the amino acid sequence GYTYFTTVTVEDYETQPGEE (SEQ ID NO: 59). In some aspects, a polypeptide described herein (e.g., comprising the FAM19A5 binding domain of LRRC4B) consists of the amino acid sequence GYTYFTTVTVEDYETQPGEE (SEQ ID NO: 59). In some aspects, a polypeptide described herein (e.g., comprising the FAM19A5 binding domain of LRRC4B) consists essentially of the amino acid sequence GYTYFTTVTVEDYETQPGEE (SEQ ID NO: 59).

In some aspects, a polypeptide described herein (e.g., comprising the FAM19A5 binding domain of LRRC4B) comprises the amino acid sequence GYTYFTTVTVEFFETQPGEE (SEQ ID NO: 60). In some aspects, a polypeptide described herein (e.g., comprising the FAM19A5 binding domain of LRRC4B) consists of the amino acid sequence GYTYFTTVTVEFFETQPGEE (SEQ ID NO: 60). In some aspects, a polypeptide described herein (e.g., comprising the FAM19A5 binding domain of LRRC4B) consists essentially of the amino acid sequence GYTYFTTVTVEFFETQPGEE (SEQ ID NO: 60).

In some aspects, a polypeptide described herein (e.g., comprising the FAM19A5 binding domain of LRRC4B) comprises the amino acid sequence GYTYFTTVTVEHYETQPGEE (SEQ ID NO: 61). In some aspects, a polypeptide described herein (e.g., comprising the FAM19A5 binding domain of LRRC4B) consists of the amino acid sequence GYTYFTTVTVEHYETQPGEE (SEQ ID NO: 61). In some aspects, a polypeptide described herein (e.g., comprising the FAM19A5 binding domain of LRRC4B) consists essentially of the amino acid sequence GYTYFTTVTVEHYETQPGEE (SEQ ID NO: 61).

In some aspects, a polypeptide described herein (e.g., comprising the FAM19A5 binding domain of LRRC4B) comprises the amino acid sequence GYTYFTTVTVEMMETQPGEE (SEQ ID NO: 62). In some aspects, a polypeptide described herein (e.g., comprising the FAM19A5 binding domain of LRRC4B) consists of the amino acid sequence GYTYFTTVTVEMMETQPGEE (SEQ ID NO: 62). In some aspects, a polypeptide described herein (e.g., comprising the FAM19A5 binding domain of LRRC4B) consists essentially of the amino acid sequence GYTYFTTVTVEMMETQPGEE (SEQ ID NO: 62).

In some aspects, a polypeptide described herein (e.g., comprising the FAM19A5 binding domain of LRRC4B) comprises the amino acid sequence GYTYFTTVTVEDFETQPGEE (SEQ ID NO: 63). In some aspects, a polypeptide described herein (e.g., comprising the FAM19A5 binding domain of LRRC4B) consists of the amino acid sequence GYTYFTTVTVEDFETQPGEE (SEQ ID NO: 63). In some aspects, a polypeptide described herein (e.g., comprising the FAM19A5 binding domain of LRRC4B) consists essentially of the amino acid sequence GYTYFTTVTVEDFETQPGEE (SEQ ID NO: 63).

In some aspects, a polypeptide described herein (e.g., comprising the FAM19A5 binding domain of LRRC4B) comprises the amino acid sequence GYTYFTTVTVEDIETQPGEE (SEQ ID NO: 64). In some aspects, a polypeptide described herein (e.g., comprising the FAM19A5 binding domain of LRRC4B) consists of the amino acid sequence GYTYFTTVTVEDIETQPGEE (SEQ ID NO: 64). In some aspects, a polypeptide described herein (e.g., comprising the FAM19A5 binding domain of LRRC4B) consists essentially of the amino acid sequence GYTYFTTVTVEDIETQPGEE (SEQ ID NO: 64).

In some aspects, a polypeptide described herein (e.g., comprising the FAM19A5 binding domain of LRRC4B) comprises the amino acid sequence GYTYFTTVTVELIETQPGEE (SEQ ID NO: 65). In some aspects, a polypeptide described herein (e.g., comprising the FAM19A5 binding domain of LRRC4B) consists of the amino acid sequence GYTYFTTVTVELIETQPGEE (SEQ ID NO: 65). In some aspects, a polypeptide described herein (e.g., comprising the FAM19A5 binding domain of LRRC4B) consists essentially of the amino acid sequence GYTYFTTVTVELIETQPGEE (SEQ ID NO: 65).

In some aspects, a polypeptide described herein (e.g., comprising the FAM19A5 binding domain of LRRC4B) comprises the amino acid sequence GYTYFTTVTVEEIETQPGEE (SEQ ID NO: 66). In some aspects, a polypeptide described herein (e.g., comprising the FAM19A5 binding domain of LRRC4B) consists of the amino acid sequence GYTYFTTVTVEEIETQPGEE (SEQ ID NO: 66). In some aspects, a polypeptide described herein (e.g., comprising the FAM19A5 binding domain of LRRC4B) consists essentially of the amino acid sequence GYTYFTTVTVEEIETQPGEE (SEQ ID NO: 66).

In some aspects, a polypeptide described herein (e.g., comprising the FAM19A5 binding domain of LRRC4B) comprises the amino acid sequence GYTYFTTVTVEAFETQPGEE (SEQ ID NO: 67). In some aspects, a polypeptide described herein (e.g., comprising the FAM19A5 binding domain of LRRC4B) consists of the amino acid sequence GYTYFTTVTVEAFETQPGEE (SEQ ID NO: 67). In some aspects, a polypeptide described herein (e.g., comprising the FAM19A5 binding domain of LRRC4B) consists essentially of the amino acid sequence GYTYFTTVTVEAFETQPGEE (SEQ ID NO: 67).

In some aspects, a polypeptide described herein (e.g., comprising the FAM19A5 binding domain of LRRC4B) comprises the amino acid sequence GYTYFTTVTVEHHETQPGEE (SEQ ID NO: 68). In some aspects, a polypeptide described herein (e.g., comprising the FAM19A5 binding domain of LRRC4B) consists of the amino acid sequence GYTYFTTVTVEHHETQPGEE (SEQ ID NO: 68). In some aspects, a polypeptide described herein (e.g., comprising the FAM19A5 binding domain of LRRC4B) consists essentially of the amino acid sequence GYTYFTTVTVEHHETQPGEE (SEQ ID NO: 68).

In some aspects, a polypeptide described herein (e.g., comprising the FAM19A5 binding domain of LRRC4B) comprises the amino acid sequence GYTYFTTVTVEPFETQPGEE (SEQ ID NO: 69). In some aspects, a polypeptide described herein (e.g., comprising the FAM19A5 binding domain of LRRC4B) consists of the amino acid sequence GYTYFTTVTVEPFETQPGEE (SEQ ID NO: 69). In some aspects, a polypeptide described herein (e.g., comprising the FAM19A5 binding domain of LRRC4B) consists essentially of the amino acid sequence GYTYFTTVTVEPFETQPGEE (SEQ ID NO: 69).

In some aspects, a polypeptide described herein (e.g., comprising the FAM19A5 binding domain of LRRC4B) comprises the amino acid sequence GYTYFTTVTVEDWETQPGEE (SEQ ID NO: 70). In some aspects, a polypeptide described herein (e.g., comprising the FAM19A5 binding domain of LRRC4B) consists of the amino acid sequence GYTYFTTVTVEDWETQPGEE (SEQ ID NO: 70). In some aspects, a polypeptide described herein (e.g., comprising the FAM19A5 binding domain of LRRC4B) consists essentially of the amino acid sequence GYTYFTTVTVEDWETQPGEE (SEQ ID NO: 70).

In some aspects, a polypeptide described herein (e.g., comprising the FAM19A5 binding domain of LRRC4B) comprises the amino acid sequence GYTYFTTVTVEPYETQPGEEA (SEQ ID NO: 71). In some aspects, a polypeptide described herein (e.g., comprising the FAM19A5 binding domain of LRRC4B) consists of the amino acid sequence GYTYFTTVTVEPYETQPGEEA (SEQ ID NO: 71). In some aspects, a polypeptide described herein (e.g., comprising the FAM19A5 binding domain of LRRC4B) consists essentially of the amino acid sequence GYTYFTTVTVEPYETQPGEEA (SEQ ID NO: 71).

In some aspects, a polypeptide described herein (e.g., comprising the FAM19A5 binding domain of LRRC4B) comprises the amino acid sequence GYTYFTTVTVEHFETQPGEEA (SEQ ID NO: 72). In some aspects, a polypeptide described herein (e.g., comprising the FAM19A5 binding domain of LRRC4B) consists of the amino acid sequence GYTYFTTVTVEHFETQPGEEA (SEQ ID NO: 72). In some aspects, a polypeptide described herein (e.g., comprising the FAM19A5 binding domain of LRRC4B) consists essentially of the amino acid sequence GYTYFTTVTVEHFETQPGEEA (SEQ ID NO: 72).

In some aspects, a polypeptide described herein (e.g., comprising the FAM19A5 binding domain of LRRC4B) comprises the amino acid sequence GYTYFTTVTVEQRETQPGEEA (SEQ ID NO: 73). In some aspects, a polypeptide described herein (e.g., comprising the FAM19A5 binding domain of LRRC4B) consists of the amino acid sequence GYTYFTTVTVEQRETQPGEEA (SEQ ID NO: 73). In some aspects, a polypeptide described herein (e.g., comprising the FAM19A5 binding domain of LRRC4B) consists essentially of the amino acid sequence GYTYFTTVTVEQRETQPGEEA (SEQ ID NO: 73).

In some aspects, a polypeptide described herein (e.g., comprising the FAM19A5 binding domain of LRRC4B) comprises the amino acid sequence GYTYFTTVTVEWYETQPGEEA (SEQ ID NO: 74). In some aspects, a polypeptide described herein (e.g., comprising the FAM19A5 binding domain of LRRC4B) consists of the amino acid sequence GYTYFTTVTVEWYETQPGEEA (SEQ ID NO: 74). In some aspects, a polypeptide described herein (e.g., comprising the FAM19A5 binding domain of LRRC4B) consists essentially of the amino acid sequence GYTYFTTVTVEWYETQPGEEA (SEQ ID NO: 74).

In some aspects, a polypeptide described herein (e.g., comprising the FAM19A5 binding domain of LRRC4B) comprises the amino acid sequence GYTYFTTVTVEMRETQPGEEA (SEQ ID NO: 75). In some aspects, a polypeptide described herein (e.g., comprising the FAM19A5 binding domain of LRRC4B) consists of the amino acid sequence GYTYFTTVTVEMRETQPGEEA (SEQ ID NO: 75). In some aspects, a polypeptide described herein (e.g., comprising the FAM19A5 binding domain of LRRC4B) consists essentially of the amino acid sequence GYTYFTTVTVEMRETQPGEEA (SEQ ID NO: 75).

In some aspects, a polypeptide described herein (e.g., comprising the FAM19A5 binding domain of LRRC4B) comprises the amino acid sequence GYTYFTTVTVEIFETQPGEEA (SEQ ID NO: 76). In some aspects, a polypeptide described herein (e.g., comprising the FAM19A5 binding domain of LRRC4B) consists of the amino acid sequence GYTYFTTVTVEIFETQPGEEA (SEQ ID NO: 76). In some aspects, a polypeptide described herein (e.g., comprising the FAM19A5 binding domain of LRRC4B) consists essentially of the amino acid sequence GYTYFTTVTVEIFETQPGEEA (SEQ ID NO: 76).

In some aspects, a LRRC4 family mimic molecule described herein comprises one or more components that can improve the ability of the polypeptide to inhibit the interaction between a FAM19A5 protein and members of the LRRC4 protein family. For instance, in some aspects, a molecule comprises (i) any of the polypeptides described herein and (ii) one or more additional amino acids at the N-terminus of the polypeptide, the C-terminus of the polypeptide, or both the N-terminus and the C-terminus of the polypeptide. In some aspects, a molecule useful for the present disclosure comprises at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 additional amino acids at the N-terminus of the polypeptide. In some aspects, a molecule comprises at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about additional 20 amino acids at the C-terminus of the polypeptide. In some aspects, a molecule comprises: (i) at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 additional amino acids at the N-terminus of the polypeptide; and (ii) at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20 additional amino acids at the C-terminus of the polypeptide.

In some aspects, a LRRC4 family mimic molecule comprises: (i) a polypeptide having the amino acid sequence set forth in SEQ ID NO: 44 (YTYFTTVTVE) and (ii) at least 1 additional amino acid at the N-terminus of the polypeptide. In some aspects, a LRRC4 family mimic molecule comprises: (i) a polypeptide having the amino acid sequence set forth in SEQ ID NO: 44 (YTYFTTVTVE) and (ii) at least 1 additional amino acid at the C-terminus of the polypeptide. In some aspects, a LRRC4 family mimic molecule comprises: (i) a polypeptide having the amino acid sequence set forth in SEQ ID NO: 44 (YTYFTTVTVE) and (ii) at least one additional amino acid at both the N-terminus and the C-terminus. In some aspects, a LRRC4 family mimic molecule useful for the present disclosure comprises the amino acid sequence set forth in SEQ ID NO: 47 (GYTYFTTVTVETLETQPGEE). In some aspects, a LRRC4 family mimic molecule consists of the amino acid sequence set forth in SEQ ID NO: 47 (GYTYFTTVTVETLETQPGEE). In some aspects, a LRRC4 family mimic molecule consists essentially of the amino acid sequence set forth in SEQ ID NO: 47 (GYTYFTTVTVETLETQPGEE). In some aspects, a LRRC4 family mimic molecule useful for the present disclosure comprises the amino acid sequence set forth in SEQ ID NO: 48 (GYTYFTTVTVETLETQ). In some aspects, a LRRC4 family mimic molecule consists of the amino acid sequence set forth in SEQ ID NO: 48 (GYTYFTTVTVETLETQ). In some aspects, a LRRC4 family mimic molecule consists essentially of the amino acid sequence set forth in SEQ ID NO: 48 (GYTYFTTVTVETLETQ). In some aspects, a LRRC4 family mimic molecule useful for the present disclosure comprises the amino acid sequence set forth in SEQ ID NO: 49 (GYTYFTTVTVETLETQPGEKEPPGPTTD). In some aspects, a LRRC4 family mimic molecule consists of the amino acid sequence set forth in SEQ ID NO: 49 (GYTYFTTVTVETLETQPGEKEPPGPTTD). In some aspects, a LRRC4 family mimic molecule consists essentially of the amino acid sequence set forth in SEQ ID NO: 49 (GYTYFTTVTVETLETQPGEKEPPGPTTD).

In some aspects, a LRRC4 family mimic molecule useful for the present disclosure comprises: (i) a polypeptide having the amino acid sequence set forth in SEQ ID NO: 45 (YSFFTTVTVE) and (ii) at least 1 additional amino acid at the N-terminus of the polypeptide. In some aspects, a LRRC4 family mimic molecule comprises: (i) a polypeptide having the amino acid sequence set forth in SEQ ID NO: 45 (YSFFTTVTVE) and (ii) at least 1 additional amino acid at the C-terminus of the polypeptide. In some aspects, a LRRC4 family mimic molecule comprises: (i) a polypeptide having the amino acid sequence set forth in SEQ ID NO: 45 (YSFFTTVTVE) and (ii) at least one additional amino acid at both the N-terminus and C-terminus. In some aspects, a LRRC4 family mimic molecule useful for the present disclosure comprises the amino acid sequence set forth in SEQ ID NO: 77 (NYSFFTTVTVETTEISPEDTTRK). In some aspects, a LRRC4 family mimic molecule consists of the amino acid sequence set forth in SEQ ID NO: 77 (NYSFFTTVTVETTEISPEDTTRK). In some aspects, a LRRC4 family mimic molecule consists essentially of the amino acid sequence set forth in SEQ ID NO: 77 (NYSFFTTVTVETTEISPEDTTRK).

In some aspects, a LRRC4 family mimic molecule useful for the present disclosure comprises: (i) a polypeptide having the amino acid sequence set forth in SEQ ID NO: 46 (FSYFSTVTVE) and (ii) at least 1 additional amino acid at the N-terminus of the polypeptide. In some aspects, a LRRC4 family mimic molecule comprises: (i) a polypeptide having the amino acid sequence set forth in SEQ ID NO: 46 (FSYFSTVTVE) and (ii) at least 1 additional amino acid at the C-terminus of the polypeptide. In some aspects, a LRRC4 family mimic molecule comprises: (i) a polypeptide having the amino acid sequence set forth in SEQ ID NO: 46 (FSYFSTVTVE) and (ii) at least one additional amino acid at both the N-terminus and the C-terminus. In some aspects, a LRRC4 family mimic molecule useful for the present disclosure comprises the amino acid sequence set forth in SEQ ID NO: 78 (NFSYFSTVTVETMEPSQDERTTR). In some aspects, a LRRC4 family mimic molecule consists of the amino acid sequence set forth in SEQ ID NO: 78 (NFSYFSTVTVETMEPSQDERTTR). In some aspects, a LRRC4 family mimic molecule consists essentially of the amino acid sequence set forth in SEQ ID NO: 78 (NFSYFSTVTVETMEPSQDERTTR).

In some aspects, a polypeptide of a LRRC4 family mimic molecule comprises an amino acid sequence that is at least about 40%, at least about 50%, at least about 60%, at least about 70%, at least about 80%, or at least about 90% identical to the amino acid sequence set forth in SEQ ID NO: 44 (YTYFTTVTVE), wherein the polypeptide is capable of binding to a FAM19A5 protein and wherein the amino acid sequence further comprises one or more hydrophobic amino acids at the N-terminus. In some aspects, the hydrophobic amino acids comprise at least two amino acids, at least three amino acids, at least four amino acids, at least five amino acids, at least six amino acids, at least seven amino acids, at least eight amino acids, at least nine amino acids, at least 10 amino acids, at least 15 amino acids, at least 20 amino acids, at least 25 amino acids, at least 30 amino acids, at least 35 amino acids, at least 40 amino acids, at least 45 amino acids, or at least 50 amino acids at the N terminus.

In some aspects, a polypeptide of a LRRC4 family mimic molecule comprises an amino acid sequence that is at least about 40%, at least about 50%, at least about 60%, at least about 70%, at least about 80%, or at least about 90% identical to the amino acid sequence set forth in SEQ ID NO: 44 (YTYFTTVTVE), wherein the polypeptide is capable of binding to a FAM19A5 protein and wherein the amino acid sequence further comprises at the N-terminus and/or at the -C terminus one or more amino acids. In some aspects, the one or more amino acids linked to the N-terminus and/or C-terminus comprise one or more amino acid sequences derived from a LRRC4B protein. In some aspects, the one or more amino acids linked to the N-terminus comprises at least two amino acids, at least three amino acids, at least four amino acids, at least five amino acids, at least six amino acids, at least seven amino acids, at least eight amino acids, at least 10 amino acids, at least 15 amino acids, at least 20 amino acids, at least 25 amino acids, at least 30 amino acids, at least 35 amino acids, at least 40 amino acids, at least 45 amino acids, or at least 50 amino acids at the N-terminus. In some aspects, the one or more amino acids linked to the C-terminus comprise at least two amino acids, at least three amino acids, at least four amino acids, at least five amino acids, at least six amino acids, at least seven amino acids, at least eight amino acids, at least 10 amino acids, at least 15 amino acids, at least 20 amino acids, at least 25 amino acids, at least 30 amino acids, at least 35 amino acids, at least 40 amino acids, at least 45 amino acids, or at least 50 amino acids at the C-terminus. In some aspects, the one or more amino acids linked to the N-terminus and/or C-terminus are linked via a linker. In some aspects, the linker is a peptide linker.

In some aspects, the one or more additional amino acids that are added at the N-terminus and/or the C-terminus can comprise any suitable amino acids known in the art. In some aspects, the one or more additional amino acids are hydrophilic amino acids. In some aspects, the one or more additional amino acids can comprise D-amino acids. Not to be bound by any one theory, in some aspects, the addition of one or more D-amino acids at the N-terminus and/or the C-terminus of the polypeptide can enhance the persistence of the LRRC4 family mimic molecule, e.g., when administered to a subject. For instance, the inclusion of the D-amino acids can protect the polypeptide from protease and peptidase degradation within the blood of the subject. Accordingly, in some aspects, a polypeptide useful for the present disclosure can comprise both D-amino acids and L-amino acids. For instance, in some aspects, a polypeptide described herein comprises a D-amino acid at the N-terminus and L-amino acid at all other amino acid residues. In some aspects, a polypeptide described herein comprises a D-amino acid at the C-terminus and L-amino acid at all other amino acid residues. In some aspects, a polypeptide described herein comprises a D-amino acid at both the N-terminus and the C-terminus, and L-amino acid at all other amino acid residues.

As described herein, in some aspects, a LRRC4 family mimic molecule described above comprises a polypeptide having an amino acid sequence set forth in any one of SEQ ID NOs: 44 (YTYFTTVTVE), 45 (YSFFTTVTVE), and 46 (FSYFSTVTVE), with one, two, three, four, five, or six amino acids different from the amino acid sequence (e.g., substitutions).

In some aspects, a LRRC4 family mimic molecule useful for the present disclosure comprises additional modifications at the N-terminus, C-terminus, or both the N-terminus and the C-terminus of the polypeptide, wherein the additional modifications can increase the stability of the polypeptide. For instance, in some aspects, the N-terminal end of the polypeptide has been methylated. Non-limiting examples of additional modifications that can be performed at the N-terminus and/or C-terminus include: Fmoc, PEGylation, acetylation, or combinations thereof. In some aspects, to increase stability, the polypeptide can be cyclized. Any suitable methods known in the art can be used to make such modifications.

As further described elsewhere in the present disclosure, in some aspects, a molecule useful for the present disclosure comprises a FAM19A5 binding domain of members of the LRRC4 family protein and an additional moiety, which is capable of improving one or more properties of the molecules (e.g., binding affinity of the molecules to the FAM19A5 protein). As demonstrated herein (see, e.g., Example 10), Applicant has identified that the addition of the juxta-membrane sequence of members of the LRRC4 protein family can greatly improve the binding affinity of the molecules to FAM19A5 protein. The juxta-membrane sequence is highly conserved among members of the LRRC4 family and set forth in SEQ ID NO: 79 (LDEVMKTTK) (LRRC4 and LRRC4B) and SEQ ID NO: 80 (IDEVMKTTK) (LRRC4C).

Accordingly, in some aspects, a molecule described herein comprises the FAM19A5 binding domain of the LRRC4 protein (i.e., YSFFTTVTVE; SEQ ID NO: 45) and the juxta-membrane sequence set forth in SEQ ID NO: 79 (LDEVMKTTK). In some aspects, a molecule described herein comprises the FAM19A5 binding domain of the LRRC4 protein (i.e., YSFFTTVTVE; SEQ ID NO: 45) and the juxta-membrane sequence set forth in SEQ ID NO: 80 (IDEVMKTTK). In some aspects, a molecule described herein comprises the FAM19A5 binding domain of the LRRC4B protein (i.e., YTYFTTVTVE; SEQ ID NO: 44) and the juxta-membrane sequence set forth in SEQ ID NO: 79 (LDEVMKTTK). In some aspects, a molecule described herein comprises the FAM19A5 binding domain of the LRRC4 protein (i.e., YTYFTTVTVE; SEQ ID NO: 44) and the juxta-membrane sequence set forth in SEQ ID NO: 80 (IDEVMKTTK). In some aspects, a molecule described herein comprises the FAM19A5 binding domain of the LRRC4B protein (i.e., FSYFSTVTVE; SEQ ID NO: 46) and the juxta-membrane sequence set forth in SEQ ID NO: 79 (LDEVMKTTK). In some aspects, a molecule described herein comprises the FAM19A5 binding domain of the LRRC4 protein (i.e., FSYFSTVTVE; SEQ ID NO: 46) and the juxta-membrane sequence set forth in SEQ ID NO: 80 (IDEVMKTTK). In some aspects, the juxta-membrane is added to the C-terminus of the molecule.

As is apparent from the present disclosure, any of the modifications described herein to improve one or more properties of a molecule (e.g., amino acid substitutions, addition of a juxta-membrane sequence, D-amino acids) can be used in combination. For instance, in some aspects, a molecule useful for the present disclosure (e.g., polypeptide) comprises: (i) the amino acid sequence set forth in SEQ ID NO: 47 (GYTYFTTVTVETLETQPGEE) with amino acid modifications at residues T12 and L13; and (ii) a juxta-membrane sequence (e.g., SEQ ID NO: 79 or SEQ ID NO: 80) at the C-terminus of the molecule. In some aspects, a molecule useful for the present disclosure (e.g., polypeptide) comprises: (i) the amino acid sequence set forth in SEQ ID NO: 47 (GYTYFTTVTVETLETQPGEE) with amino acid modifications at residues T12 and L13; (ii) D-amino acids at the N-terminus and/or C-terminus; and (iii) a juxta-membrane sequence (e.g., SEQ ID NO: 79 or SEQ ID NO: 80) at the C-terminus of the molecule. In some aspects, a molecule useful for the present disclosure (e.g., polypeptide) comprises: (i) the amino acid sequence set forth in SEQ ID NO: 48 (GYTYFTTVTVETLETQ) with amino acid modifications at residues T12 and L13; and (ii) a juxta-membrane sequence (e.g., SEQ ID NO: 79 or SEQ ID NO: 80) at the C-terminus of the molecule. In some aspects, a molecule useful for the present disclosure (e.g., polypeptide) comprises: (i) the amino acid sequence set forth in SEQ ID NO: 48 (GYTYFTTVTVETLETQ) with amino acid modifications at residues T12 and L13; (ii) D-amino acids at the N-terminus and/or C-terminus; and (iii) a juxta-membrane sequence (e.g., SEQ ID NO: 79 or SEQ ID NO: 80) at the C-terminus of the molecule. In some aspects, a molecule useful for the present disclosure (e.g., polypeptide) comprises: (i) the amino acid sequence set forth in SEQ ID NO: 49 (GYTYFTTVTVETLETQPGEKEPPGPTTD) with amino acid modifications at residues T12 and L13; and (ii) a juxta-membrane sequence (e.g., SEQ ID NO: 79 or SEQ ID NO: 80) at the C-terminus of the molecule. In some aspects, a molecule useful for the present disclosure (e.g., polypeptide) comprises: (i) the amino acid sequence set forth in SEQ ID NO: 49 (GYTYFTTVTVETLETQPGEKEPPGPTTD) with amino acid modifications at residues T12 and L13; (ii) D-amino acids at the N-terminus and/or C-terminus; and (iii) a juxta-membrane sequence (e.g., SEQ ID NO: 79 or SEQ ID NO: 80) at the C-terminus of the molecule. In some aspects, a molecule useful for the present disclosure (e.g., polypeptide) comprises: (i) the amino acid sequence set forth in SEQ ID NO: 50 (GYTYFTTVTVETLETQPGEEA) with amino acid modifications at residues T12 and L13; and (ii) a juxta-membrane sequence (e.g., SEQ ID NO: 79 or SEQ ID NO: 80) at the C-terminus of the molecule. In some aspects, a molecule useful for the present disclosure (e.g., polypeptide) comprises: (i) the amino acid sequence set forth in SEQ ID NO: 50 (GYTYFTTVTVETLETQPGEEA) with amino acid modifications at residues T12 and L13; (ii) D-amino acids at the N-terminus and/or C-terminus; and (iii) a juxta-membrane sequence (e.g., SEQ ID NO: 79 or SEQ ID NO: 80) at the C-terminus of the molecule.

In some aspects, a LRRC4 family mimic molecule described herein can comprise one or more additional peptides that allow the molecule to be specifically targeted to different tissues, e.g., when administered to a subject. For instance, in some aspects, a LRRC4 family mimic molecule comprises a peptide that allows the molecule to penetrate across the blood-brain barrier (also referred to herein as “BBB shuttles”). Examples of such BBB shuttles are known in the art. Non-limiting examples are provided in Table 10 (below). See, e.g., Oller-Salvia et al., Chem Soc Rev 45:4690 (2016).

TABLE 10
BBB Shuttles

| SEQ ID | | |
|---|---|---|
| NO | Peptide | Sequence |
| 255 | Angiopep-2 | TFFYGGSRGKRNNFKTEEY-OH |
| 256 | ApoB (3371-3409) | SSVIDALQYKLEGTTRLTRKRGLKLATALSLSNKFVEGS |
| 257 | ApoE (159-167)₂ | (LRKLRKRLL)₂ |
| 258 | Peptide-22 | Ac-C(&)MPRLRGC(&)-NH₂ |
| 259 | THR | THRPPMWSPVWP-NH₂ |
| 260 | THR retro-enantio | PWVPSWMPPRHT-NH₂ |
| 261 | CRT | C(&)RTIGPSVC(&) |
| 262 | Leptin30 | YQQILTSMPSRNVIQISNDLENLRDLLHVL |
| 263 | RVG29 | YTIWMPENPRPGTPCDIFTNSRGKRASNG-OH |
| 264 | CDX | GreirtGraerwsekf-OH |
| 265 | Apamin | C(&1)NC(&2)KAPETALC(&₁)-ARRC(&₂)QQH-NH₂ |
| 266 | MiniAp-4 | [Dap](&)KAPETALD(&) |
| 267 | GSH | γ-_{L}-glutamyl-CG-OH |
| 268 | G23 | HLNILSTLWKYRC |
| 269 | g7 | GFtGFLS(O-b-Glc)-NH₂ |
| 270 | TGN | TGNYKALHPHNG |
| 271 | TAT (47-57) | YGRKKRRQRRR-NH₂ |
| 272 | SynB1 | RGGRLSYSRRRFSTSTGR |
| 273 | Diketopiperazines | &(N-MePhe)-(N-MePhe)Diketopiperazines |
| 274 | PhPro | (Phenylproline)4-NH₂ |

Nomenclature for cyclic peptides (&) is adapted to the 3-letter amino acid code from the one described in Spengler et al., J Pept Res 65: 550-555 (2005); [Dap] stands for diaminopropionic acid.

In some aspects, a LRRC4 family mimic molecule useful for the present disclosure comprises a fusion protein. For instance, in some aspects, a LRRC4 family mimic molecule described herein can comprise: (i) any of the polypeptides of the present disclosure, and (ii) a half-life extending moiety. Any suitable half-life extending moieties known in the art can be used to generate the fusion proteins of the present disclosure. Non-limiting examples of such half-life extending moieties include: a Fc, albumin, an albumin-binding polypeptide, Pro/Ala/Ser (PAS), a C-terminal peptide (CTP) of the β subunit of human chorionic gonadotropin, polyethylene glycol (PEG), long unstructured hydrophilic sequences of amino acids (XTEN), hydroxyethyl starch (HES), an albumin-binding small molecule, or a combination thereof.

In some aspects, a LRRC4 family mimic molecule comprises a protein-drug conjugate. For instance, in some aspects, the polypeptide of the LRRC4 family mimic can be conjugated to a therapeutic agent, such as those that are useful for treating a disease or disorder.

The protein-drug conjugates described herein can be prepared by methods known in the art. In some aspects, conjugation methods result in linkages which are substantially (or nearly) non-immunogenic, e.g., peptide—(i.e., amide-), sulfide-, (sterically hindered), disulfide-, hydrazone-, and ether linkages. These linkages are nearly non-immunogenic and show reasonable stability within serum (see, e.g., Senter, P. D., Curr. Opin. Chem. Biol. 13 (2009) 235-244; WO 2009/059278; WO 95/17886, each of which is incorporated herein by reference in its entirety).

Depending on the biochemical nature of the moiety and the polypeptides, different conjugation strategies can be employed (see, e.g., Hackenberger, C. P. R., and Schwarzer, D., Angew. Chem. Int. Ed. Engl. 47 (2008) 10030-10074). In some aspects, site specific reaction and covalent coupling is based on transforming a natural amino acid into an amino acid with a reactivity which is orthogonal to the reactivity of the other functional groups present. For example, a specific cysteine within a rare sequence context can be enzymatically converted in an aldehyde (see Frese, M. A., and Dierks, T., ChemBioChem. 10 (2009) 425-427). It is also possible to obtain a desired amino acid modification by utilizing the specific enzymatic reactivity of certain enzymes with a natural amino acid in a given sequence context (see, e.g., Taki, M. et al., Prot. Eng. Des. Sel. 17 (2004) 119-126; Gautier, A. et al., Chem. Biol. 15 (2008) 128-136; and Protease-catalyzed formation of C—N bonds is used by Bordusa, F., Highlights in Bioorganic Chemistry (2004) 389-403).

Site specific reaction and covalent coupling can also be achieved by the selective reaction of terminal amino acids with appropriate modifying reagents. The reactivity of an N-terminal cysteine with benzonitrils (see Ren, H. et al., Angew. Chem. Int. Ed. Engl. 48 (2009) 9658-9662) can be used to achieve a site-specific covalent coupling. Native chemical ligation can also rely on C-terminal cysteine residues (Taylor, E. Vogel; Imperiali, B, Nucleic Acids and Molecular Biology (2009), 22 (Protein Engineering), 65-96).

The moiety can also be a synthetic peptide or peptide mimic. In such cases, a polypeptide can be chemically synthesized, amino acids with orthogonal chemical reactivity can be incorporated during such synthesis (see, e.g., de Graaf, A. J. et al., Bioconjug. Chem. 20 (2009) 1281-1295). To obtain a mono-labeled polypeptide, the conjugate with 1:1 stoichiometry can be separated by chromatography from other conjugation side-products. This procedure can be facilitated using a dye labeled binding pair member and a charged linker. With this kind of labeled and highly negatively charged binding pair member, mono conjugated polypeptides are easily separated from non-labeled polypeptides and polypeptides which carry more than one linker, since the difference in charge and molecular weight can be used for separation. The fluorescent dye can be useful for purifying the complex from un-bound components, like a labeled monovalent binder.

### IV. Nucleic Acids, Vectors, Host Cells

Further aspect described herein pertains to one or more nucleic acid molecules (also referred to herein as “nucleic acids” or derivatives thereof) that encode a therapeutic agent (e.g., a LRRC4 family mimic molecule and/or FAM19A5 antagonist described herein). The nucleic acids can be present in whole cells, in a cell lysate, or in a partially purified or substantially pure form. In some aspects, the nucleic acid is a DNA sequence and/or an RNA sequence (e.g., mRNA). In some aspects, the nucleic acids comprise a modified nucleotide analog. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See, F. Ausubel, et al., ed. (1987) Current Protocols in Molecular Biology, Greene Publishing and Wiley Interscience, New York. In some aspects, a nucleic acid molecule can or cannot contain intronic sequences. In some aspects, the nucleic acid is a cDNA molecule. Nucleic acids described herein can be obtained using standard molecular biology techniques known in the art.

In some aspects, the present disclosure provides a vector comprising an isolated nucleic acid molecule encoding a therapeutic agent disclosed herein (e.g., a LRRC4 family mimic molecule and/or FAM19A5 antagonist described herein). Suitable vectors for the disclosure include, but are not limited to, expression vectors, viral vectors, and plasmid vectors. In some aspects, the vector is a viral vector.

As used herein, an “expression vector” refers to any nucleic acid construct which contains the necessary elements for the transcription and translation of an inserted coding sequence, or in the case of a RNA viral vector, the necessary elements for replication and translation, when introduced into an appropriate host cell. Expression vectors can include plasmids, phagemids, viruses, and derivatives thereof.

As used herein, “viral vectors” include, but are not limited to, nucleic acid sequences from the following viruses: retrovirus, such as Moloney murine leukemia virus, Harvey murine sarcoma virus, murine mammary tumor virus, and Rous sarcoma virus; lentivirus; adenovirus; adeno-associated virus; SV40-type viruses; polyomaviruses; Epstein-Barr viruses; papilloma viruses; herpes virus; vaccinia virus; polio virus; and RNA virus such as a retrovirus. Certain viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA.

In some aspects, a vector is derived from an adeno-associated virus. In some aspects, a vector is derived from a lentivirus. Examples of the lentiviral vectors are disclosed in WO9931251, WO9712622, WO9817815, WO9817816, and WO9818934, each which is incorporated herein by reference in its entirety.

Other vectors include plasmid vectors. See, e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operably encoded within the plasmid. Some commonly used plasmids available from commercial suppliers include pBR322, pUC18, pUC19, various pcDNA plasmids, pRC/CMV, various pCMV plasmids, pSV40, and pBlueScript. Additional examples of specific plasmids include pcDNA3.1, catalog number V79020; pcDNA3.1/hygro, catalog number V87020; pcDNA4/myc-His, catalog number V86320; and pBudCE4.1, catalog number V53220, all from Invitrogen (Carlsbad, CA). Additionally, plasmids can be custom designed using standard molecular biology techniques to remove and/or add specific fragments of DNA.

### V. Pharmaceutical Compositions

Provided herein are compositions comprising a therapeutic agent (e.g., LRRC4 family mimic molecule, FAM19A5 antagonist, or both; including nucleic acids, vectors, cells, or protein conjugates encoding and/or comprising the LRRC4 family mimic molecule and/or FAM19A5 antagonist) having the desired degree of purity in a physiologically acceptable carrier, excipient or stabilizer (Remington's Pharmaceutical Sciences (1990) Mack Publishing Co., Easton, PA). Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

In some aspects, a pharmaceutical composition useful for the present disclosure comprises any of the therapeutic agents described herein (e.g., LRRC4 family mimic molecule, FAM19A5 antagonist, or both; including nucleic acids, vectors, cells, or protein conjugates encoding and/or comprising the LRRC4 family mimic molecule and/or FAM19A5 antagonist), and optionally one or more additional prophylactic or therapeutic agents, in a pharmaceutically acceptable carrier. In some aspects, pharmaceutical compositions comprise any of the therapeutic agents described herein (e.g., LRRC4 family mimic molecule, FAM19A5 antagonist, or both; including nucleic acids, vectors, cells, or protein conjugates encoding and/or comprising the LRRC4 family mimic molecule and/or FAM19A5 antagonist), and optionally one or more additional prophylactic of therapeutic agents, in a pharmaceutically acceptable carrier. In some aspects, the therapeutic agents described herein are the only active ingredient included in the pharmaceutical composition

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

A pharmaceutical composition can be formulated for any route of administration to a subject. Specific examples of routes of administration include intranasal, oral, parenterally, intrathecally, intra-cerebroventricularly, pulmonarily, subcutaneously, or intraventricularly. Parenteral administration, characterized by either subcutaneous, intramuscular or intravenous injection, is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Preparations for parenteral administration of a therapeutic agent described herein (e.g., LRRC4 family mimic molecule, FAM19A5 antagonist, or both; including nucleic acids, vectors, cells, or protein conjugates encoding and/or comprising the LRRC4 family mimic molecule and/or FAM19A5 antagonist) include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Topical mixtures comprising a therapeutic agent are prepared as described for the local and systemic administration. The resulting mixture can be a solution, suspension, emulsions or the like and can be formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

A pharmaceutical composition can be formulated as an aerosol for topical application, such as by inhalation (see, e.g., U.S. Pat. Nos. 4,044,126, 4,414,209 and 4,364,923). These formulations for administration to the respiratory tract can be in the form of an aerosol or solution for a nebulizer, or as a microfine powder for insufflations, alone or in combination with an inert carrier such as lactose. In such a case, the particles of the formulation can, in some aspects, have diameters of less than about 50 microns, e.g., less than about 10 microns.

A pharmaceutical composition can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the antibody alone or in combination with other pharmaceutically acceptable excipients can also be administered.

Transdermal patches, including iontophoretic and electrophoretic devices, are well known to those of skill in the art, and can be used to administer any of the therapeutic agents described herein. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957.

In some aspects, a pharmaceutical composition described herein is a lyophilized powder, which can be reconstituted for administration as solutions, emulsions and other mixtures. It can also be reconstituted and formulated as solids or gels. The lyophilized powder is prepared by dissolving any of the therapeutic agents described herein, or a pharmaceutically acceptable derivative thereof, in a suitable solvent. In some aspects, the lyophilized powder is sterile. The solvent can contain an excipient which improves the stability or other pharmacological component of the powder or reconstituted solution, prepared from the powder. Excipients that can be used include, but are not limited to, dextrose, sorbitol, fructose, corn syrup, xylitol, glycerin, glucose, sucrose or other suitable agent. The solvent can also contain a buffer, such as citrate, sodium, or potassium phosphate or other such buffer known to those of skill in the art. In some aspects, the buffer is at about neutral pH. Subsequent sterile filtration of the solution followed by lyophilization under standard conditions known to those of skill in the art provides the desired formulation. In some aspects, the resulting solution can be apportioned into vials for lyophilization. Each vial can contain a single dosage or multiple dosages of any of the therapeutic agents described herein (e.g., LRRC4 family mimic molecule, FAM19A5 antagonist, or both; including nucleic acids, vectors, cells, or protein conjugates encoding and/or comprising the LRRC4 family mimic molecule and/or FAM19A5 antagonist). The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

In some aspects, a pharmaceutical composition comprising any of the therapeutic agents described herein can also be formulated to be targeted to a particular tissue, receptor, or other area of the body of the subject to be treated. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542, and 5,709,874.

The compositions to be used for in vivo administration can be sterile. In some aspects, this can be accomplished by filtration through, e.g., sterile filtration membranes.

### VI. Kits

Provided herein are kits comprising one or more FAM19A5 antagonists, wherein the kits are for treatment of a hearing disorder (e.g., described herein). In some aspects, provided herein is a pack or kit comprising one or more containers filled with one or more of the ingredients of the compositions described herein, such as one or more FAM19A5 antagonists, optional an instructing for use.

### EXAMPLES

### Example 1: Analysis of FAM19A5 and LRRC4 Family Member Expression within the Inner Ear

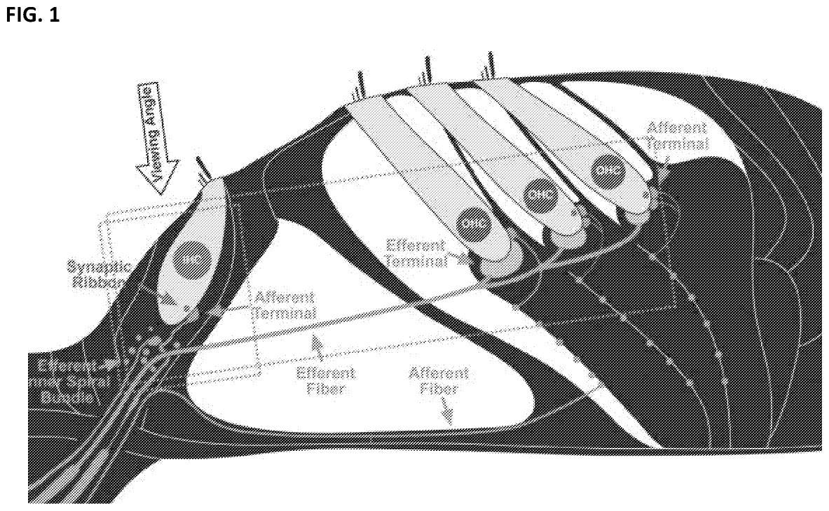

To begin assessing the therapeutic effects of the FAM19A5 antagonists provided herein on hearing loss, the expression of FAM19A5 and LRRC4 family members were assessed within the auditory system in mice. In particular, expression was assessed within the spiral ganglion neurons, which are known to interact with inner hair cells at the synaptic ribbon within the cochlea (see FIG. 1).

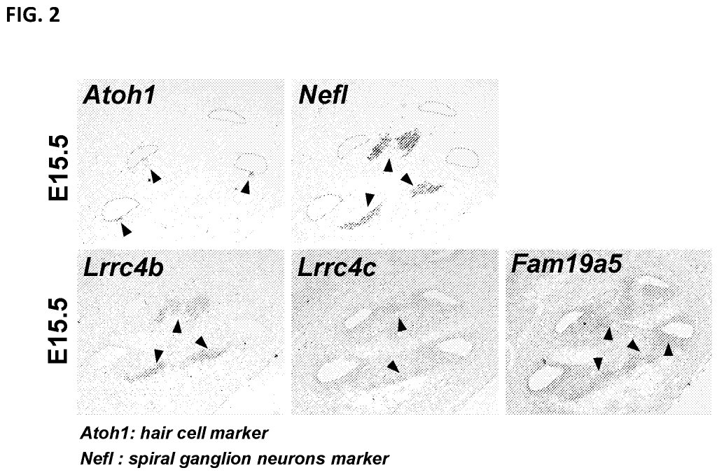

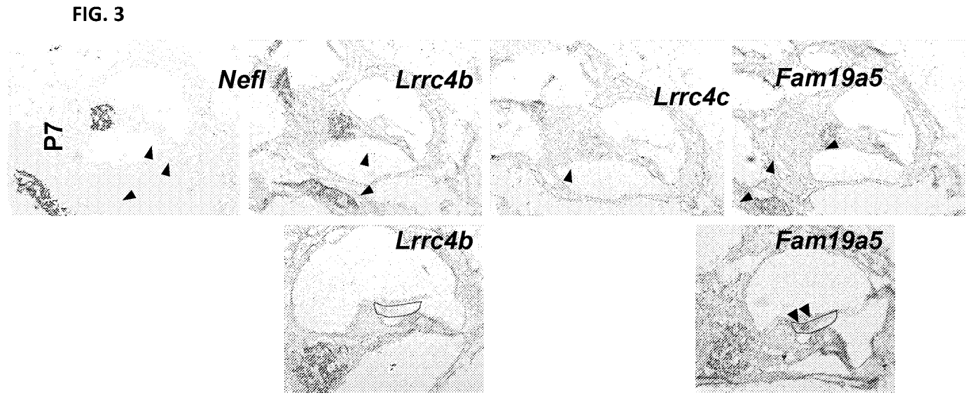

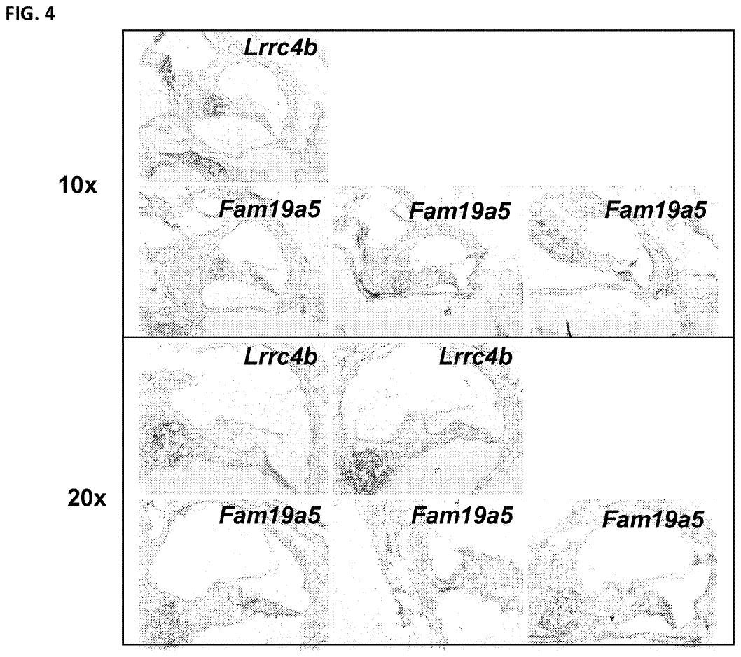

As shown in FIG. 2, there was noticeable expression of FAM19A5, LRRC4B, and LRRC4C observed within the cochlear tissue obtained from embryonic day 15.5 mice embryo. There was overlap in expression with Nefl (marker for spiral ganglion neurons) and Atoh1 (marker for inner hair cells), suggesting that FAM19A5, LRRC4B, and LRRC4C were expressed within the synaptic ribbon, and particularly among the spiral ganglion neurons. Similar results were observed in day 7 postnatal mice (see FIGS. 3 and 4).

The above results demonstrate the potential role that FAM19A5 and LRRC4 family members have on synapse activity (e.g., formation, loss, and/or signal transmission) within the auditory system.

### Example 2: Effect of FAM19A5 Antagonist Administration on Hearing Ability

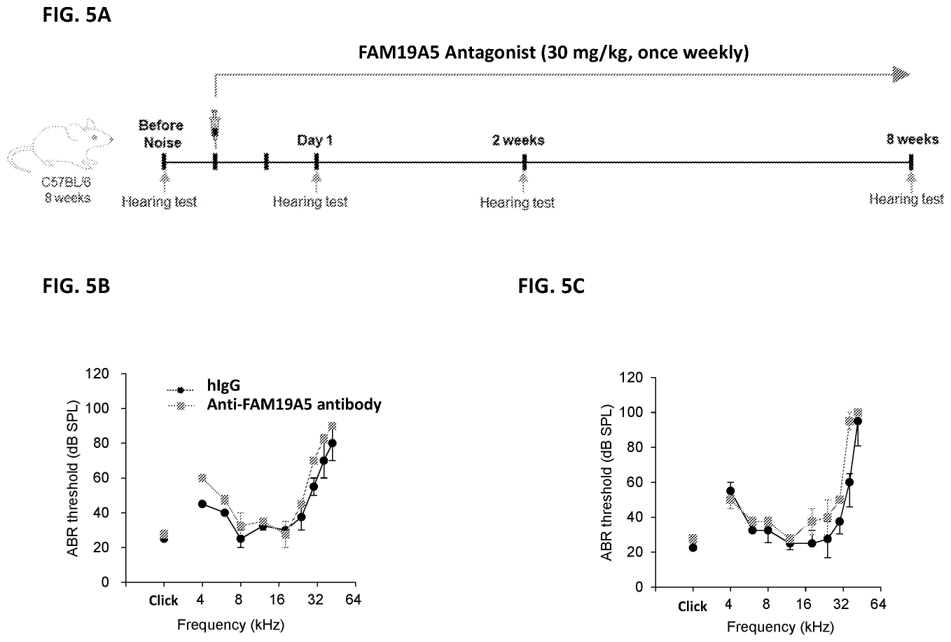

To assess whether the administration of a FAM19A5 antagonist itself has any effect on hearing, C₅₇BL/6 mice (8 weeks old) were intravenously administered with either an anti-FAM19A5 antibody (1-30 antibody) or a control human IgG antibody. Both the anti-FAM19A5 antibody and control antibody were administered once weekly for 8 total weeks and at a dose of 30 mg/kg per dose (see FIG. 5A). To assess hearing, auditory brainstem response (ABR) threshold was measured prior to the initial antibody administration and then at day 1, 2 weeks, and 8 weeks post antibody administration. It was expected that a high ABR threshold at a particular frequency suggests impaired hearing at the given frequency, whereas a low ABR threshold at a particular frequency suggests normal hearing at the given frequency.

As shown in FIG. 5B, prior to the antibody administration, animals from either of the treatment groups (i.e., anti-FAM19A5 antibody or the control IgG antibody) had comparable ABR threshold values at all frequencies tested. And, as shown in FIG. 5C, the ABR threshold values remained comparable at 2 weeks post antibody administration.

The above results demonstrate that the FAM19A5 antagonists described herein do not themselves have any negative effects on hearing.

### Example 3: Analysis of the Therapeutic Effects of FAM19A5 Antagonists on Hearing Loss

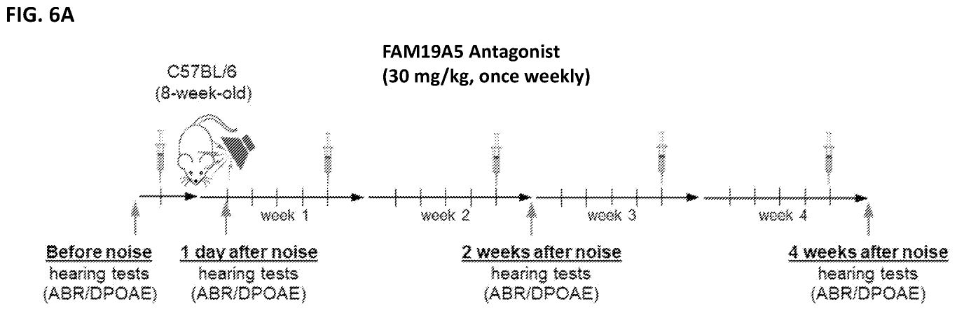

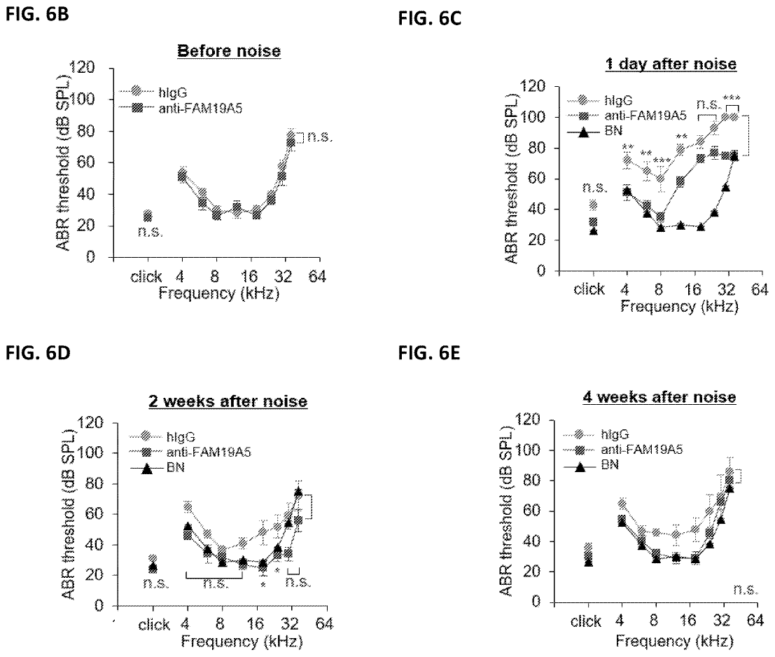

To assess whether the FAM19A5 antagonists described herein can treat hearing loss, a noise-induced hearing loss mouse model was used. Briefly, for hearing loss induction, the animals were exposed to a temporary threshold shift (TTS) of 105 decibels for 30 minutes. An anti-FAM19A5 antibody (1-30 antibody) or a control human IgG antibody was intravenously administered to C57BL/6 mice (8 weeks old) once weekly for 5 total administrations (30 mg/kg per dose). As shown in FIG. 6A, the first dose of the antibodies was administered to the mice one day prior to the noise exposure. To assess the effect on hearing loss, ABR threshold, DPOAE threshold, and/or DPOAE amplitude were measured at various time points (see “hearing test” in FIG. 6A).

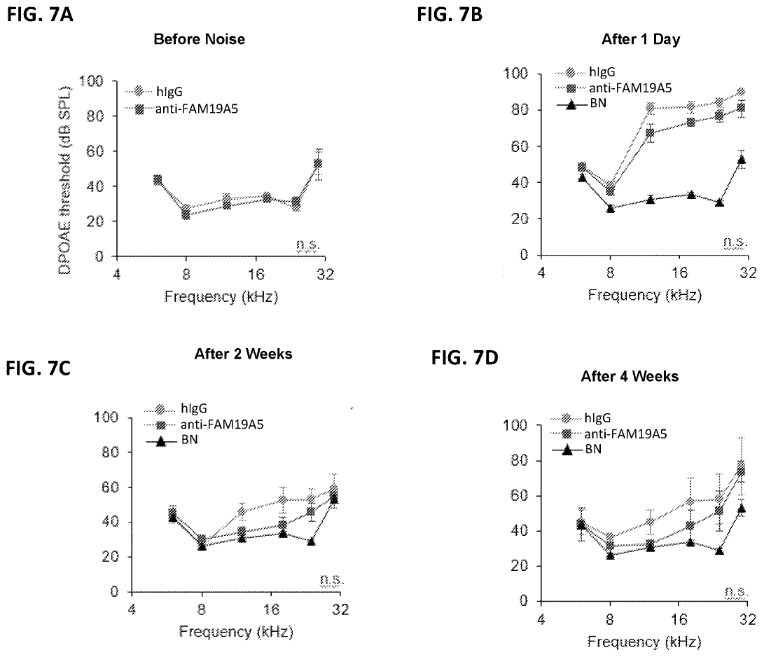

As expected, prior to noise exposure, there was no noticeable difference in the various hearing parameters assessed among the animals from the different treatment groups (see FIGS. 6B and 7A). At day 1 post exposure to TTS, noticeable increase in ABR threshold was observed in all the animals (see, e.g., FIG. 6C), confirming that the TTS exposure caused hearing impairment. However, the increase in ABR threshold was much more dramatic in animals treated with the control antibody as compared to animals treated with the anti-FAM19A5 antibody. Generally, at all time points assessed, the ABR threshold values in animals treated with the anti-FAM19A5 antibody were comparable to corresponding values prior to the TTS exposure (see, e.g., FIGS. 6C-6E). In animals from the control group, the greatest increase in ABR threshold was observed immediately after TTS exposure (e.g., at day 1—see FIG. 6C) and eventually started to return closer to baseline levels (i.e., values prior to TTS exposure) at about 2 weeks post TTS exposure.

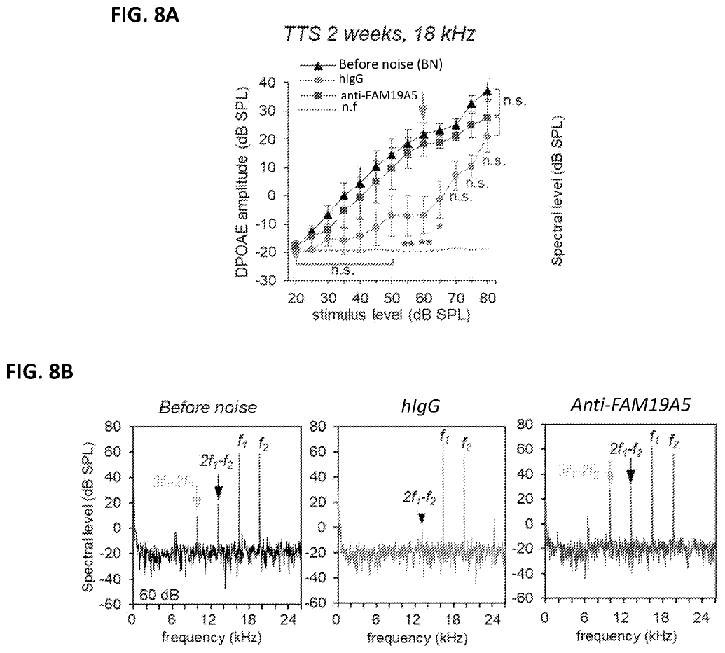

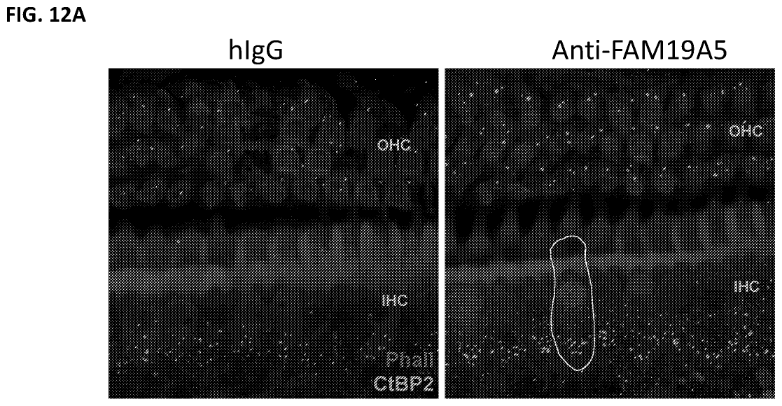

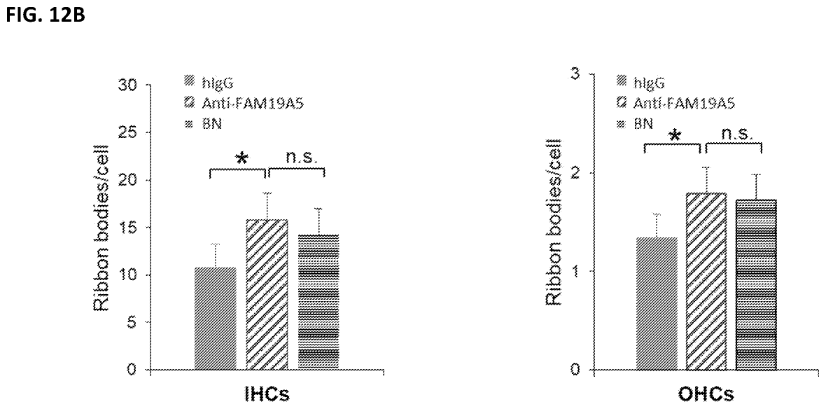

And, as shown in FIGS. 7A-7D, no noticeable differences were observed in DPOAE threshold values between the anti-FAM19A5 antibody treated and control IgG antibody treated groups. However, at least at 2 weeks after TTS exposure, noticeable differences in DPOAE amplitude were observed between the anti-FAM19A5 antibody treated group and the control IgG antibody treated group (see FIGS. 8A and 8B). More specifically, at about 18 kHz frequency, animals treated with the anti-FAM19A5 antibody had higher DPOAE amplitude as compared to animals treated with the control antibody. Additionally, as shown in FIG. 12A, there was a noticeable increase in the expression of CtBP2, a marker of cochlear ribbon synapses, in the IHC following anti-FAM19A5 antibody administration in comparison to the control antibody. Furthermore, FIG. 12B provides an increased number of ribbon bodies per cell, in the IHC (1^{st} chart) and OHC (2^{nd} chart) following anti-FAM19A5 antibody administration and in comparison to hIgG and before noise group. The greater number of cochlear ribbon synapses correlates to hearing function.

Next, to assess whether the timing of the anti-FAM19A5 antibody administration has an effect, the above-described noise-induced hearing loss mouse model was used again. Again, to induce hearing loss, the animals were exposed to a temporary threshold shift (TTS) of 105 decibels for 30 minutes. An anti-FAM19A5 antibody (1-30 antibody) or a control human IgG antibody was intravenously administered to the animals once weekly for 5 total administration (30 mg/kg per dose). In this experiment, the first dose of the antibodies was administered to the animals at 2 hours post noise exposure (as opposed to one day prior to the noise exposure as in the earlier described experiment). Hearing loss was assessed (e.g., by measuring ABR threshold, DPOAE threshold, and/or DPOAE amplitude) in the animals prior to the noise exposure and then again at one day, two weeks, and four weeks post noise exposure.

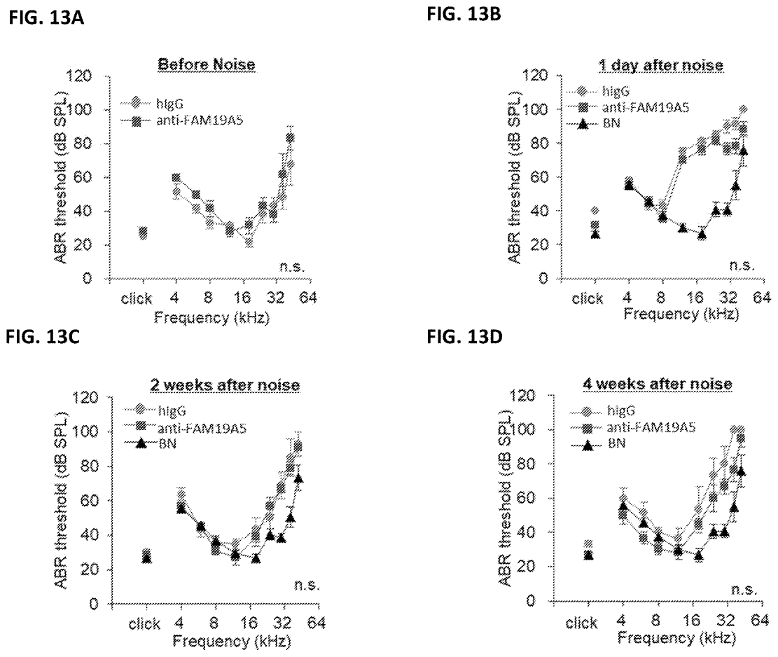

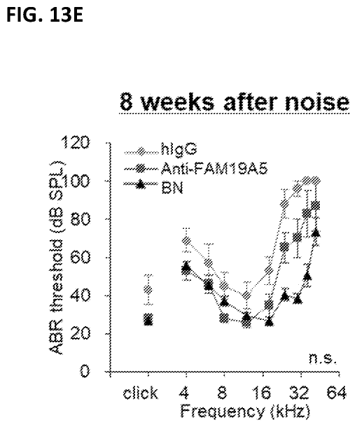

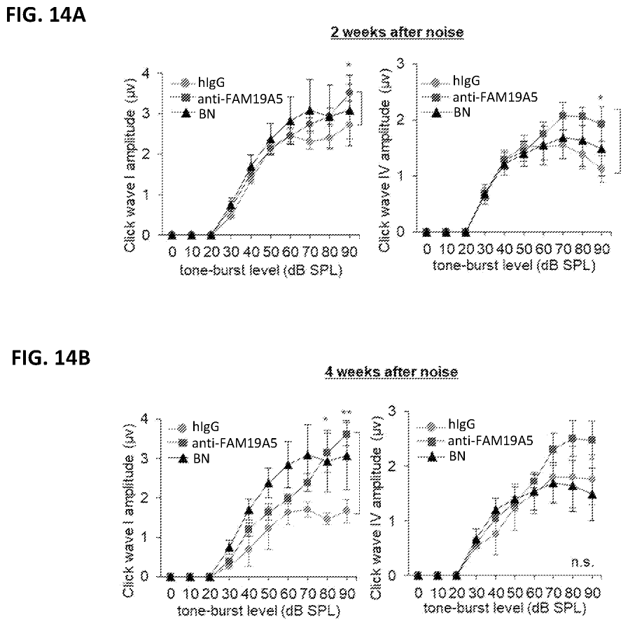

As shown in FIGS. 13A-13E, as to the ABR threshold, there did not appear to be any significant differences among animals treated with the anti-FAM19A5 antibody from those animals treated with the control antibody. However, as further described in Example 4, noticeable differences were observed between the treatment groups as to Wave I and Wave IV amplitudes, particularly at the higher tone-burst levels (see FIGS. 14A and 14B).

### Example 4: ABR Waveform Analysis after FAM19A5 Antagonist Administration

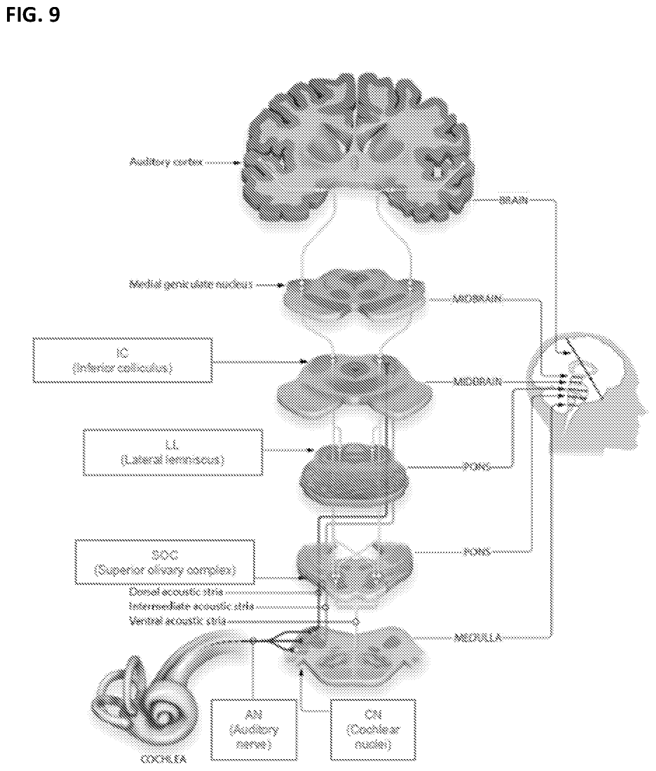

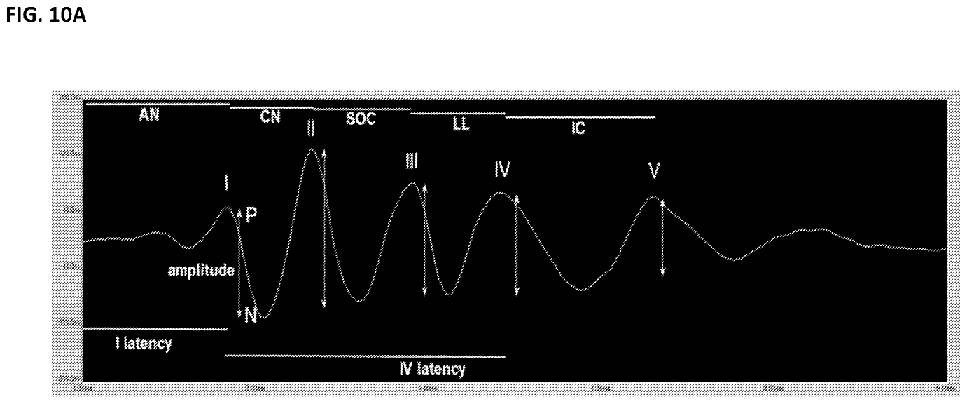

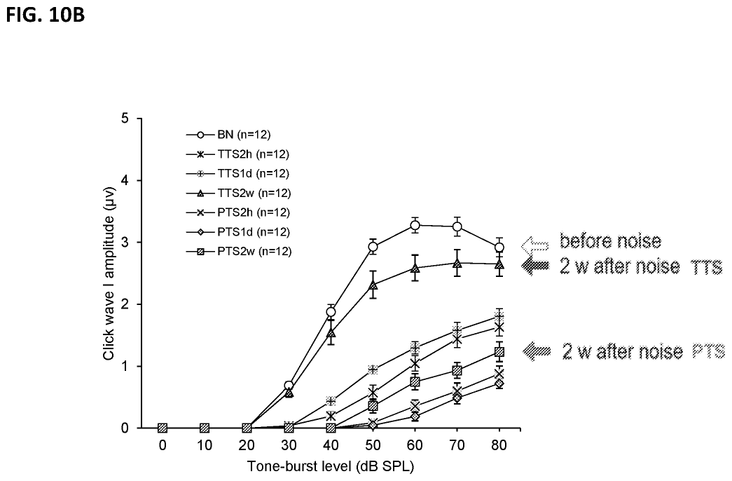

To further understand the therapeutic effects of the FAM19A5 antagonists described herein on hearing loss, an ABR waveform analysis was conducted. As illustrated in FIG. 9, upon entering the cochlea, sound is transmitted to the auditory nerve (AN), and then subsequently to the auditory cortex through the brain stem, via the cochlear nuclei (CN), superior olivary complex (SOC), lateral lemniscus (LL), and inferior colliculus (IC), respectively. Each time a wavelength passes through the AN, CN, SOC, LL, or IC respectively, an ABR waveform analysis can be conducted by first measuring the amplitude of the signal from the auditory nerve, and then measuring the sequential wavelengths via initial amplitude and latency of the first wavelength (see FIG. 10A), resulting in the exemplary click waive I amplitude profile shown in FIG. 10B.

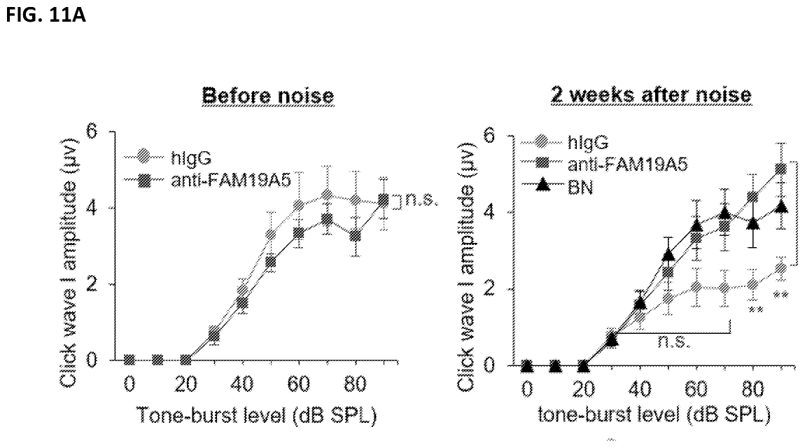

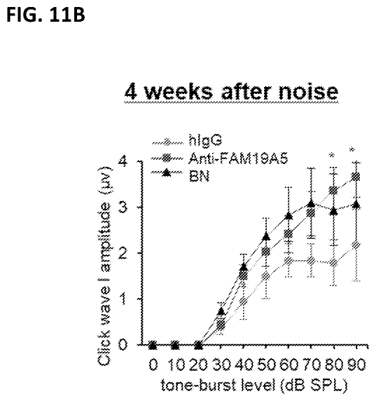

As shown in FIG. 11A, prior to TTS exposure (left graph), there were no significant differences observed for wave I amplitude. However, at both two weeks (FIG. 11A, right graph) and four weeks post noise exposure (FIG. 11B), noticeable differences were observed. Wave I is exemplary of the signal summation from the synapse. Wave I's amplitude correlates to its intensity of signals and its latency correlates to conductivity

The above results collectively demonstrate that the FAM19A5 antagonists described herein are useful in treating and/or preventing hearing loss.

### Example 5: Dosage Effect on Therapeutic Efficacy of FAM19A5 Antagonists on Hearing Loss

To assess whether the above-described therapeutic effects of FAM19A5 antagonists on hearing loss is dose dependent, the noise-induced hearing loss mouse model described in Example 3 was used. Briefly, after exposing the mice to noise, anti-FAM19A5 antibody (1-30 antibody) or a control human IgG antibody was intravenously administered three times: (i) one day before noise exposure, (ii) one week after noise exposure, and (iii) two weeks after noise exposure. The anti-FAM19A5 antibody was administered at one of the following doses: 1 mg/kg, 3 mg/kg, 7 mg/kg, 15 mg/kg, and 30 mg/kg. ABR threshold was measured prior to noise exposure, one day after noise exposure, and two weeks after noise exposure.

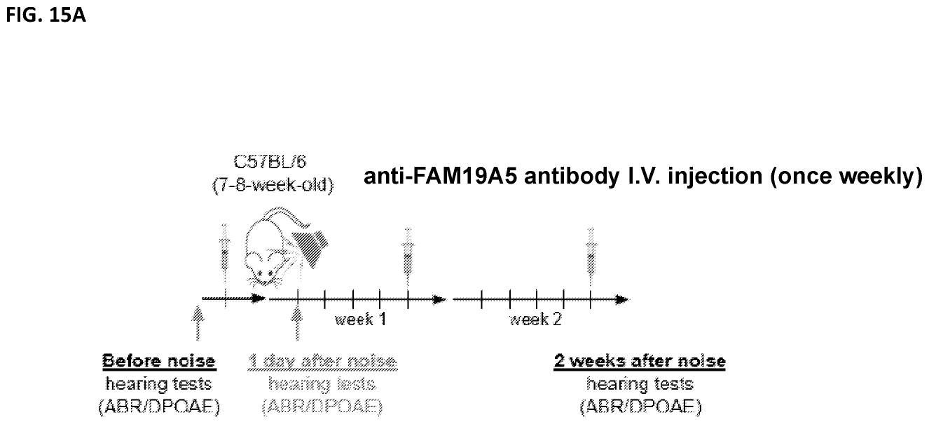

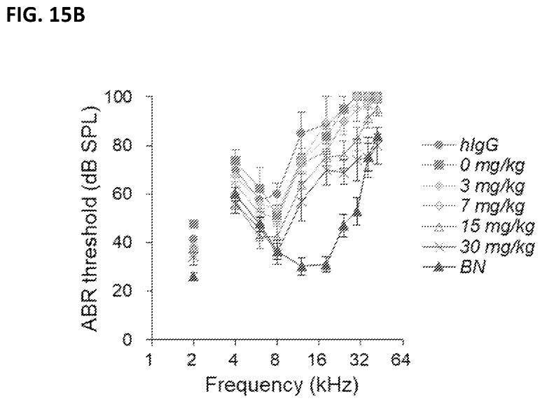

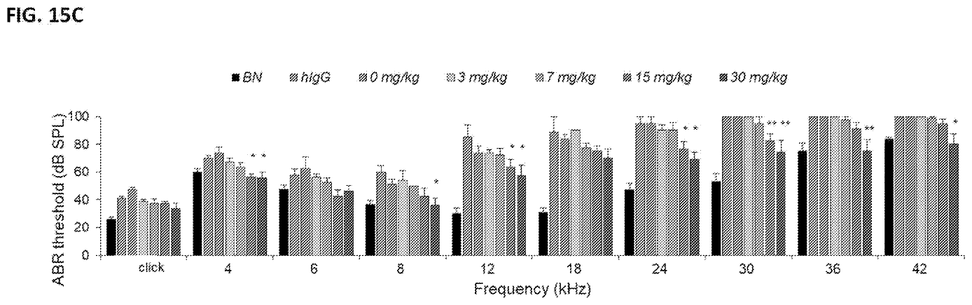

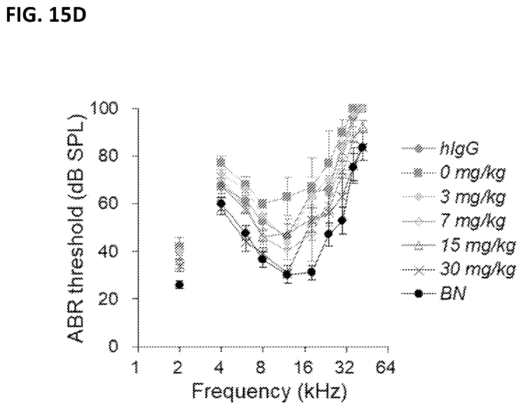

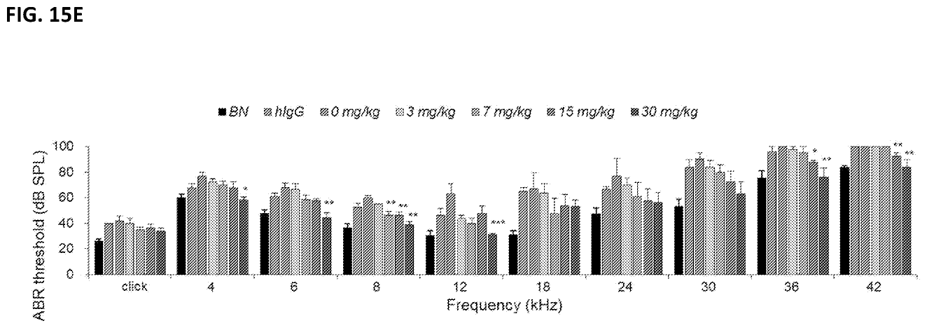

As shown in FIGS. 15B and 15C, in mice treated with the control antibody, an increase in ABR threshold was observed at all frequencies at one day post noise exposure. In animals treated with the anti-FAM19A5 antibody, there was a dose-dependent reduction in ABR threshold across the different frequencies tested. The greatest effects were observed in animals treated with 15 mg/kg and 30 mg/kg of the anti-FAM19A5 antibody. At two weeks post noise exposure, there was a slight decrease in ABR threshold in animals treated with the control antibody, as compared to the levels observed at one day post noise exposure. However, the levels were still higher than that measured prior to noise exposure. In animals treated with the anti-FAM19A5 antibody (e.g., at doses of 7 mg/kg, 15 mg/kg, or 30 mg/kg), significant reduction in ABR threshold was still observed at many of the frequencies (see FIGS. 15D and 15E).

These results demonstrate that noise exposure can result in damage of the neural connection between hair cells and spiral ganglia neurons. The results further demonstrate that the FAM19A5 antagonists provided herein (e.g., anti-FAM19A5 antibody) can restore such damages.

### Example 6: Therapeutic Effect on FAM19A5 Antagonist Administration on Age-Related Hearing Loss

To begin assessing the potential therapeutic effects of FAM19A5 antagonists on age-related hearing loss, various hearing parameters (e.g., ABR threshold, DPOAE amplitude, and ABR waveform analysis) were compared between aged mice (6-month old and 12-month old) and normal aged mice (10-week old).

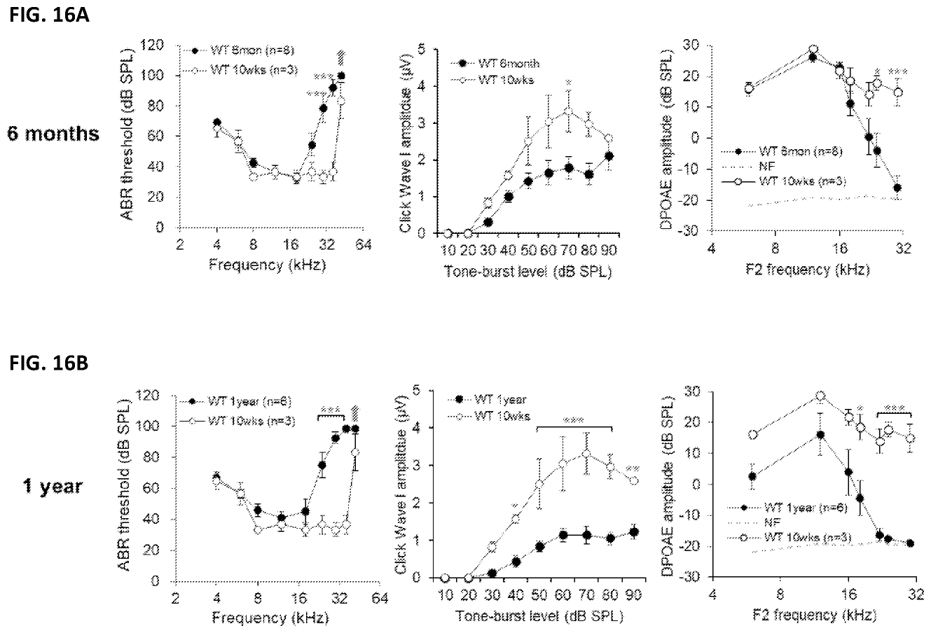

As shown in FIG. 16A, compared to the normal aged mice, the 6-month old aged mice exhibited increased ABR threshold at higher frequencies (e.g., 32 kHz and above). This different was much more pronounced when the normal aged mice were compared to 1-year old aged mice (see FIG. 16B). Similar results were observed when wave I amplitude and DPOAE amplitude were compared.

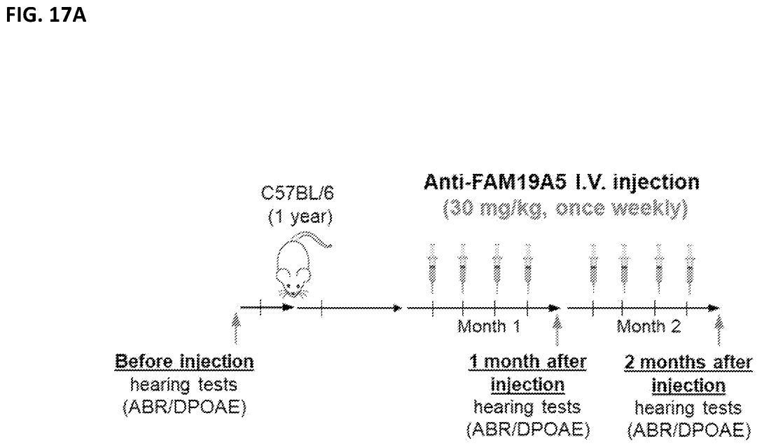

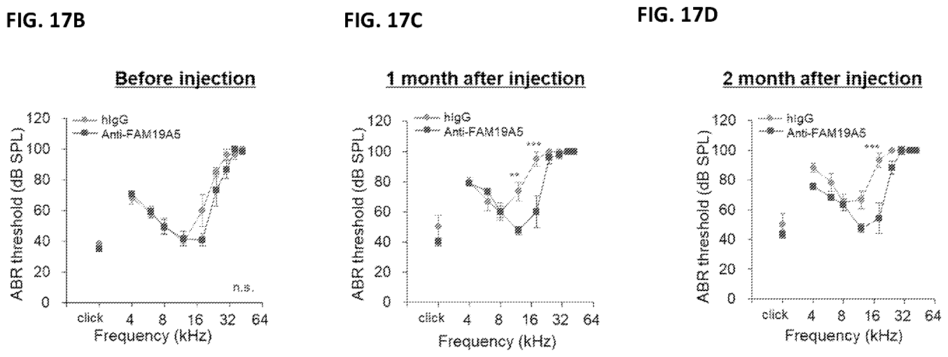

Next, to assess whether the FAM19A5 antagonists provided herein can also treat age-related hearing loss, aged mice (e.g., about 6 months to 1 year in age) were used. As shown in FIG. 17A, an anti-FAM19A5 antibody (1-30 antibody) or a control human IgG antibody was intravenously administered to the animals weekly for eight consecutive weeks (30 mg/kg per dose). Then, various hearing parameters (e.g., ABR threshold, DPOAE amplitude, and ABR waveform analysis) were assessed in the animals at one and two months after initial administration.

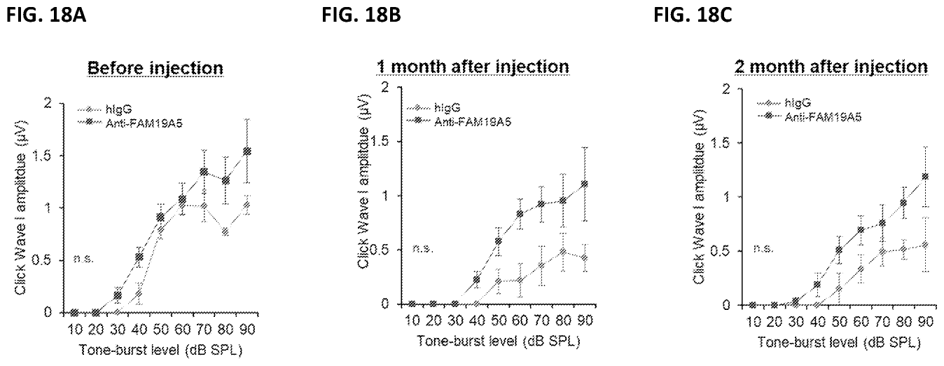

As shown in FIG. 17B, prior to anti-FAM19A5 antibody administration, there did not appear to be any significant differences in ABR threshold among the different animals. However, at both one-month and two-month post administration, the ABR threshold was significantly decreased in animals treated with the anti-FAM19A5 antibody as compared to the control animals (see FIGS. 17C and 17D, respectively). As to wave I amplitude, while the results were not statistically significant, the general trend was similar to that observed for the ABR threshold (i.e., decreased wave I amplitude in animals treated with the anti-FAM19A5 antibody) (see FIGS. 18A-18C).

The above results demonstrate that the FAM19A5 antagonists described herein (e.g., anti-FAM19A5 antibody) can also have therapeutic effects in age-related hearing loss.

### Example 7: Epitope Mapping Analysis Using FAM19A5 Epitope Fragments F1-F6

Overlapping peptide fragments (F1-F6 epitope fragments; SEQ ID NOs: 178-183) of the human FAM19A5 protein were synthesized and conjugated to BSA (SEQ ID NOs: 322-327). Binding of the different anti-FAM19A5 antibodies to the BSA-conjugated peptide fragments F1-F6 was determined by Western blot analysis or ELISA assay. For the Western blot analysis, BSA-conjugated FAM19A5 fragments F1-F6 were separated by SDS polyacrylamide gel electrophoresis and transferred to a nitrocellulose membrane by the standard procedure. The membrane was incubated with the anti-FAM19A5 antibody (e.g., 1-65, 2 g/ml, 1-65-scFv-rabbit Fc-SSS), and the antigen-antibody complexes were detected with the appropriate secondary antibody conjugated with horse-radish peroxidase (anti-rabbit IgG (Fc specific)-HRP, 1:4000 dilution). For the ELISA assay, the following protocol was used. FAM19A5 fragment F1-F6 (diluted to 1 μg/mL in 50 mM carbonate buffer (Biosesang) or to 20 μg/mL for high concentration analysis) were used to coat the wells of 96-well immuno plates (Thermo Scientific) (100 μL/well) overnight at 4° C. and then subsequently washed twice in 1×PBS. The plates were then blocked with the blocking buffer (100 μL/well) for 1 hour at room temperature. During the 1-hour incubation, the relavant anti-FAM19A5 antibodies were diluted to 1 μg/mL (or 20 μg/mL for high concentration analysis) in the diluent buffer. Once the plates were washed (2× using 1×PBS), the diluted anti-FAM19A5 antibodies were added to the appropriate wells, and the plates were incubated at room temperature for 1 hour. The plates were subsequently washed for a total of five times using the washing buffer. Next, the ODP substrate (prepared by dissolving one ODP tablet (O-phenylenediamine Dihydrochloride, Thermo) into 9 mL of sterilized deionized water and 1 mL of 10× stable Peroxide Stable buffer (Theromo)) was added to each of the wells, and the color change reaction was allowed to occur for 10 minutes. This reaction was stopped by adding 100 μL of 2N H2SO4 (Daejung) to the wells. The absorption value of each of the wells was detected at 492 nm using a 96-well microplate reader (Molecular Device).

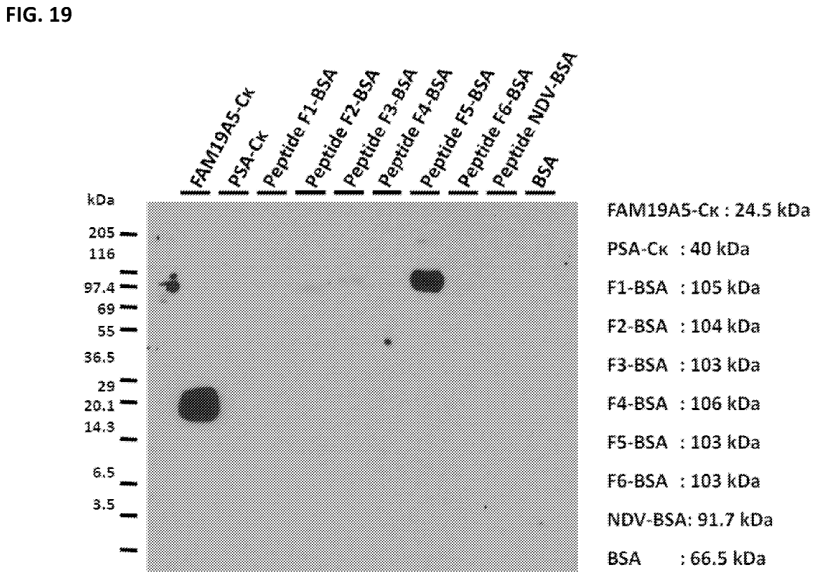

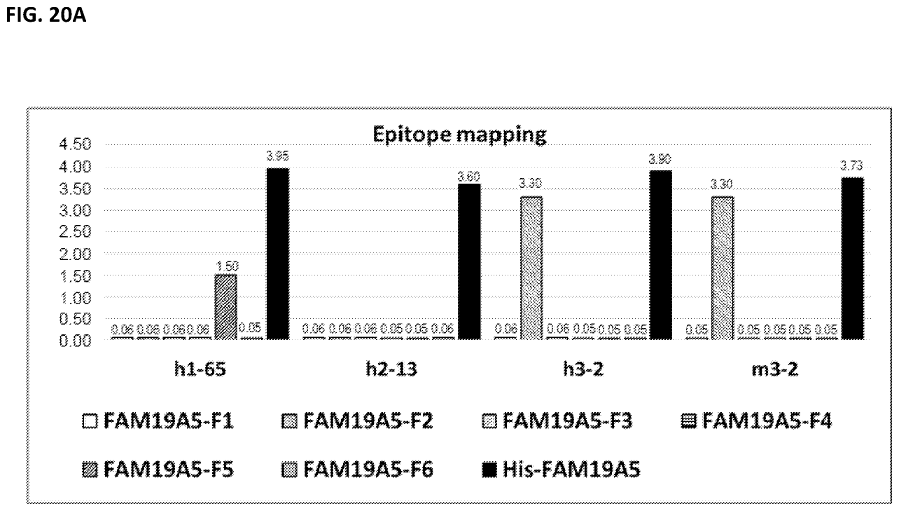

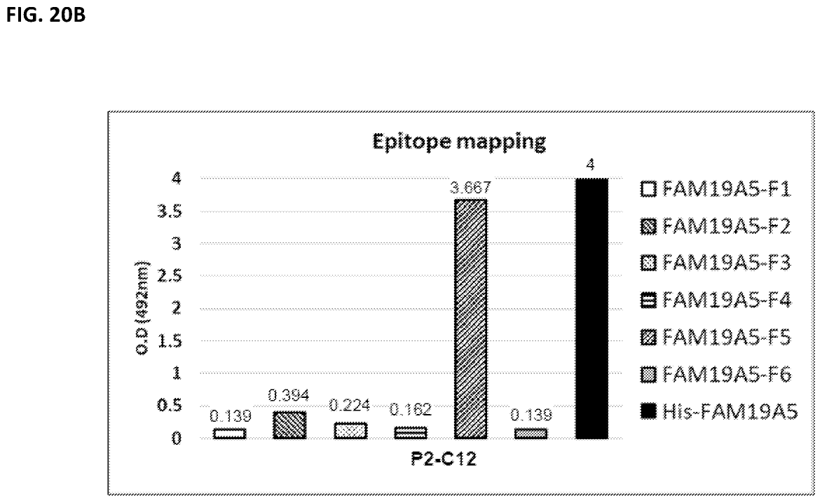

As shown in FIGS. 19 and 20A, the anti-FAM19A5 antibody 1-65 bound strongly to fragment F5 as measured by both Western Blot and ELISA assay, respectively. The anti-FAM19A5 antibody P2-C₁₂ also bound the epitope fragment F5 strongly but not significantly to the other fragments (F1-F4 and F6) (see FIG. 20B). The anti-FAM19A5 antibody 3-2, on the other hand, did not bind to fragment F5. Instead, the 3-2 antibody bound strongly to epitope fragment F2 with minimal binding to the other fragments (see FIG. 20A). This was also true for the anti-FAM19A5 1-28 antibody (data not shown). However, in contrast to the other antibodies, the anti-FAM19A5 antibody 2-13 did not appear to bind to any of the epitope fragments (see FIG. 20A).

Next, to identify the specific amino acid residues within the eptitope fragment F5 that the 1-65 and P2-C₁₂ antibodies bind to, different amino acid residues of the F5 fragment were replaced with alanine as shown in Table 11 (below). The mutated residues are bolded and underlined. The binding affinity of the indicated anti-FAM19A5 antibodies were measured using an ELISA assay as described above.

TABLE 11
Mutant F5 Peptide Fragments

| Mutant | |
|---|---|
| peptide (#) | Sequences |
| F5 | SDMLPSLEGEGSDLLINRSG (SEQ ID NO: 275) |
| F5-1 (#1) | SDMLPSLEGEASDLLINRSG (SEQ ID NO: 276) |
| F5-2 (#2) | SDMLPSLEGEGSALLINRSG (SEQ ID NO: 277) |
| F5-3 (#3) | SDMLPSLEGEGSDALINRSG (SEQ ID NO: 278) |
| F5-4 (#4) | SDMLPSLEGEGSDLLANRSG (SEQ ID NO: 279) |
| F5-5 (#5) | SDMLPSLAGEGSDLLINRSG (SEQ ID NO: 280) |
| F5-6 (#6) | SDMLPSLEGAGSDLLINRSG (SEQ ID NO: 281) |
| F5-7 (#7) | SDMLPSLEGEGSDLLIARSG (SEQ ID NO: 282) |
| F5-8 (#8) | SDMLPSLEGEGSDLLINASG (SEQ ID NO: 283) |

Double underline: Cysteine is reactive in peptide synthesis process so it was replaced with serine to reduce reactivity. Serine was substituted as its structure is the most closest to cysteine. Such replacements are indicated as double underlines.

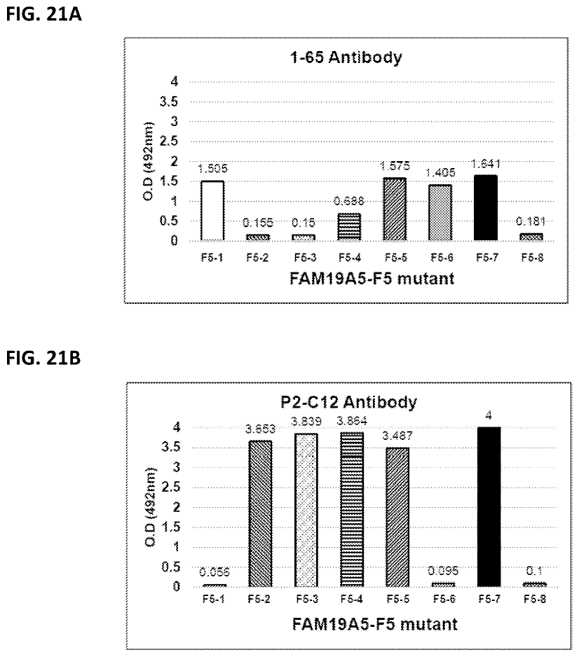

As shown in FIG. 21A, the 1-65 antibody was able to bind mutant peptides #1, 5, 6, and 7 with similar affinity. However, when amino acid residues D13, L14, 116, and R₁₈ of fragment F5 were mutated to alanine (numbering based on SEQ ID NO: 275 in Table 11, above), the 1-65 antibody was no longer able to bind the peptide fragment, suggesting that these amino acid residues were important binding sites for the 1-65 antibody. In contrast, the P2-C₁₂ antibody showed high binding to mutant peptides #2, 3, 4, 5, and 7 but not to mutant peptides #1, 6, and 8. See FIG. 21B. As shown in Table 11 (above), the mutant peptides #1, 6, and 8 comprise an alanine substitution at amino acid residues G11, E10, and R18 (numbering based on SEQ ID NO: 275), suggesting that these amino acid residues are important binding sites for the P2-C₁₂ antibody.

Next, to identify the specific amino acid residues within the epitope fragment F2 that the 3-2 and 1-28 antibodies bind to, different amino acid residues of the F2 fragment were replaced with alanine or valine as shown in Table 12 (below). The mutated residues are bolded and underlined. The binding affinity of the indicated anti-FAM19A5 antibodies were measured using an ELISA assay as described above.

TABLE 12
Mutant F2 Peptide Fragments

| Mutant peptide (#) | Sequences |
|---|---|
| Wild Type | TLDRDSSQPRRTIARQTARC |
| | (SEQ ID NO: 179) |
| F2-01-BSA (#1) | TADRDSSQPRRTIARQTARC |
| | (SEQ ID NO: 284) |
| F2-02-BSA (#2) | TLARDSSQPRRTIARQTARC |
| | (SEQ ID NO: 285) |
| F2-03-BSA (#3) | TLDRASSQPRRTIARQTARC |
| | (SEQ ID NO: 286) |
| F2-04-BSA (#4) | TLDRDASQPRRTIARQTARC |
| | (SEQ ID NO: 287) |
| F2-05-BSA (#5) | TLDRDSAQPRRTIARQTARC |
| | (SEQ ID NO: 288) |
| F2-06-BSA (#6) | TLDRDSSAPRRTIARQTARC |
| | (SEQ ID NO: 289) |
| F2-07-BSA (#7) | TLDRDSSQARRTIARQTARC |
| | (SEQ ID NO: 290) |
| F2-08-BSA (#8) | TLDRDSSQPRATIARQTARC |
| | (SEQ ID NO: 291) |
| F2-09-BSA (#9) | TLDRDSSQPRRAIARQTARC |
| | (SEQ ID NO: 292) |
| F2-10-BSA (#10) | TLDRDSSQPRRTAARQTARC |
| | (SEQ ID NO: 293) |
| F2-11-BSA (#11) | TLDRDSSQPRRTIRRQTARC |
| | (SEQ ID NO: 294) |
| F2-12-BSA (#12) | TLDRDSSQPRRTIAAQTARC |
| | (SEQ ID NO: 295) |
| F2-13-BSA (#13) | TLDRDSSQPRRTIARQTVRC |
| | (SEQ ID NO: 296) |

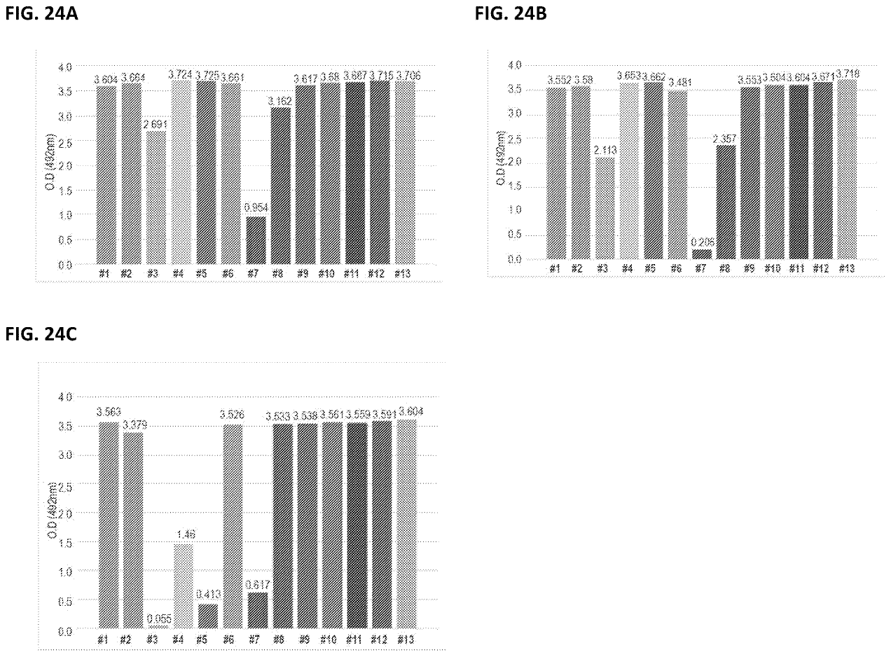

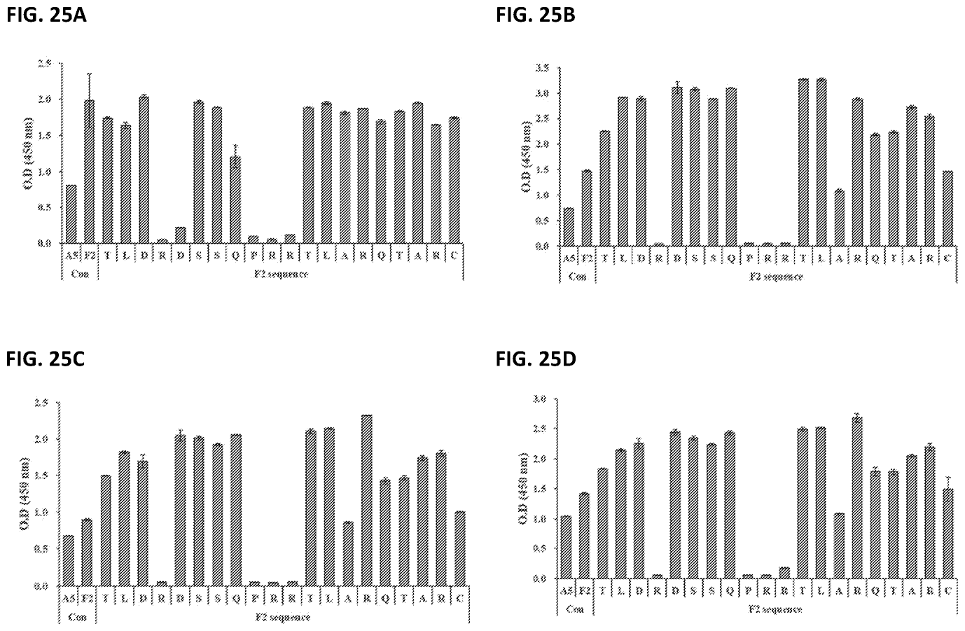

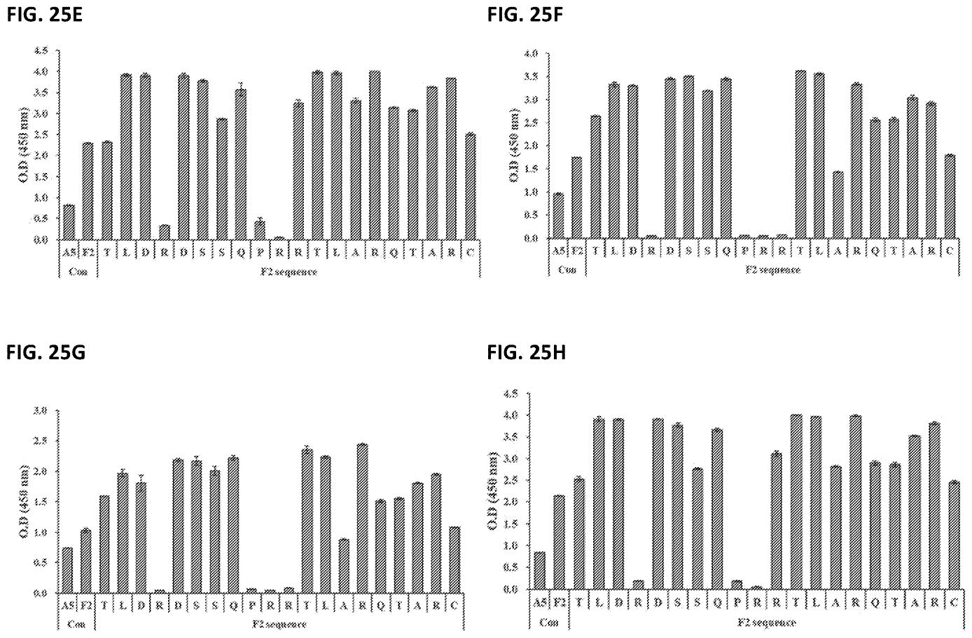

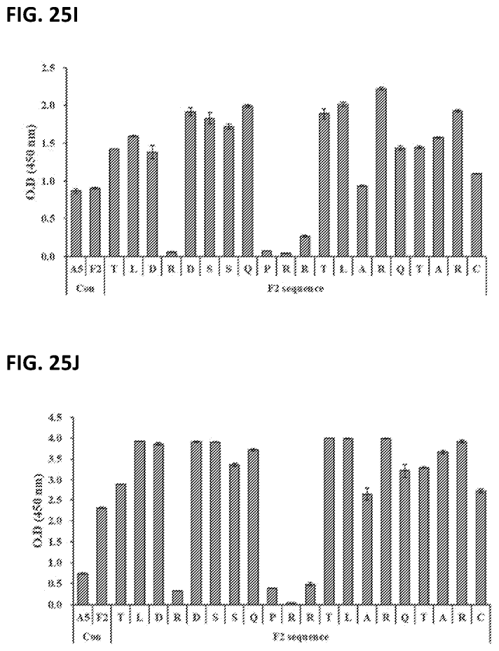

As shown in FIG. 25A, when amino acid residue R4, D5, P9, R10, or R11 was mutated to alanine, the ability of the 3-2 antibody to bind to epitope fragment F2 was greatly diminished. See also FIGS. 24A and 24B. Similar analysis was conducted for several de-immunized variants of the 3-2 antibody (see Example 8 for details regarding the de-immunization process). As shown in FIGS. 25B, 25C, 25D, 25F, 25G, 25I, and 25J, amino acid residues R4, P9, R10, and R11 were important for binding of antibodies 1-30, 1-32, and 6-10 to FAM19A5. For antibodies, 1-17 and 4-11, amino acid residues R4, P9, and R10 were important (see FIGS. 25E and 25H).

As for the 1-28 antibody, it bound strongly to mutant F2 peptide fragments #1, 2, 6, and 8-13, whereas to peptide fragments #3, 4, 5, and 7, the 1-28 antibody exhibited much reduced binding. See FIG. 24C. This data suggests that the anti-FAM19A5 antibody 1-28 binds to FAM19A5 primarily at amino acid residues D5, S6, S7, and P9 (numbering according to SEQ ID NO: 179 in Table 12, above) within the epitope F2 fragment.

### Example 8: Epitope Mapping Analysis Using FAM19A5 Mutants M1-M8

To further characterize the binding epitopes of the anti-FAM19A5 antibodies disclosed in the present disclosure, the amino acid sequences for the different FAM19 family members (i.e., FAM19A1-5) were aligned. Based on this alignment, eight regions where the amino acid sequences of the FAM19A5 protein differed the most significantly from the other members of the FAM19A family (i.e., FAM19A1-4) were identified (M1-M8). The amino acid sequences of these regions were replaced with the consensus sequence of the corresponding regions for the FAM19A1-4 proteins. See Table 13 (the mutated amino acid residues are bolded and underlined).

The mutant FAM19A5-expressing phages were prepared as follows. To prepare the media for phage culture, 55.6 ml of 2M Glucose (D-(+)-Glucose, Sigma), 5 mL of 1M MgCl2 (Magnesium chloride, Junsei) 1 mL of 34 mg/mL chloramphenicol (Sigma) were added to 2×YT media. Colonies obtained through mono-phage ELISA were selected and placed in 5 mL of prepared media (2×YT-GMC) to be cultured for 16 hours at 37° C. with shaking incubator (VS-8480, Vision). 100 μL of each culture was transferred to 10 mL 2×YT-GMC individually which were cultured at 37° C. in the incubator until detection value at O.D. 600 nm reached 0.5. Once detection value has reached 0.5 at O.D. 600 nm, 5 mL of each culture were obtained as samples to be infected. Following sample preparation, 50 μL of M1 helper phage was added to individual samples then incubated at 37° C. without shaking for 30 minutes and with shaking for additional 30 minutes. Individual cultures were centrifuged for 15 minutes at 3,850 rpm using Micro centrifuge (Micro12, Hanil). The supernatants of centrifuged cultures were removed to be kept separate and replaced with 5 mL of 2×YT media containing 1 mL of IM IPTG (AG Scientific), 5 mL of 1M MgCl2.1 ml of 70 μg/μL kanamycin (Biopure) and 1 mL of Chloramphenicol. The resulting pellets were dispersed thoroughly in newly added media followed by incubation at 30° C. for 16 hours with stirring.

TABLE 13
Mutant M1-M8 Peptide Fragments

| FAM19A5 | Sequences |
|---|---|
| Wild Type | QFLKEGQLAAGTCEIVTLDRDSSQPRRTIARQTARCACRKGQIAGTTRARPAC |
| | VDARIIKTKQWCDMLPCLEGEGCDLLINRSGWTCTQPGGRIKTTTVS |
| | (SEQ ID NO: 297) |
| M1 | QFLKEGQLAAGTCEVIAAHRDSSQPRRTIARQTARCACRKGQIAGTTRARPAC |
| | VDARIIKTKQWCDMLPCLEGEGCDLLINRSGWTCTQPGGRIKTTTVS |
| | (SEQ ID NO: 298) |
| M2 | QFLKEGQLAAGTCEIVTLDRCCNKNRRTIARQTARCACRKGQIAGTTRARPAC |
| | VDARIIKTKQWCDMLPCLEGEGCDLLINRSGWTCTOPGGRIKTTTVS |
| | (SEQ ID NO: 299) |
| M3 | QFLKEGQLAAGTCEIVTLDRDSSQPRIEERSQTARCACRKGQIAGTTRARPAC |
| | VDARIIKTKQWCDMLPCLEGEGCDLLINRSGWTCTQPGGRIKTTTVS |
| | (SEQ ID NO: 300) |
| M4 | QFLKEGQLAAGTCEIVTLDRDSSQPRRTIARQTVKCSCFPGQIAGTTRARPAC |
| | VDARIIKTKQWCDMLPCLEGEGCDLLINRSGWTCTQPGGRIKTTTVS |
| | (SEQ ID NO: 301) |
| M5 | QFLKEGQLAAGTCEIVTLDRDSSQPRRTIARQTARCACRKGQIAGTTRNKPSC |
| | VDARIIKTKQWCDMLPCLEGEGCDLLINRSGWTCTQPGGRIKTTTVS |
| | (SEQ ID NO: 302) |
| M6 | QFLKEGQLAAGTCEIVTLDRDSSQPRRTIARQTARCACRKGQIAGTTRARPAC |
| | VDARIILQRWWCQMELCLEGEGCDLLINRSGWTCTQPGGRIKTTTVS |
| | (SEQ ID NO: 303) |
| M7 | QFLKEGQLAAGTCEIVTLDRDSSQPRRTIARQTARCACRKGQIAGTTRARPAC |
| | VDARIIKTKQWCDMLPCLEGEECKTLPDNSGWTCTQPGGRIKTTTVS |
| | (SEQ ID NO: 304) |
| M8 | QFLKEGQLAAGTCEIVTLDRDSSQPRRTIARQTARCACRKGQIAGTTRARPAC |
| | VDARIIKTKQWCDMLPCLEGEGCDLLINRSGWTCSCSSGNKIKTTTVS |
| | (SEQ ID NO: 305) |

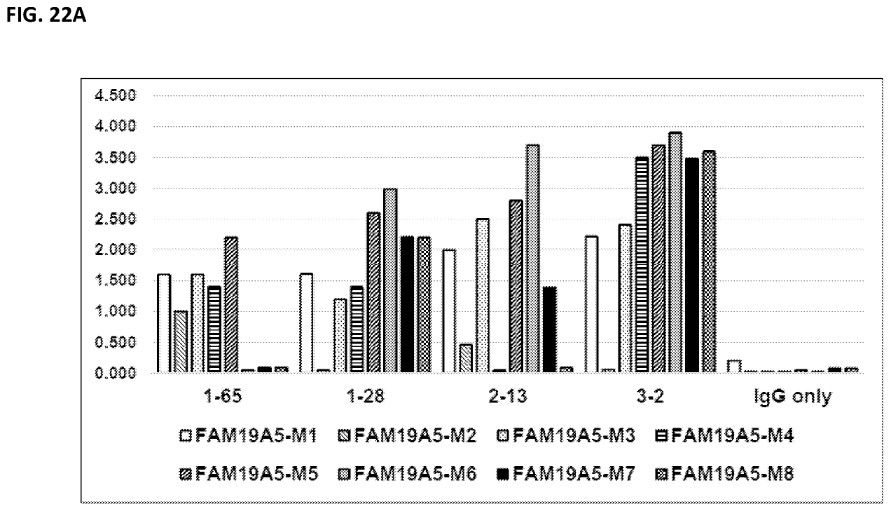

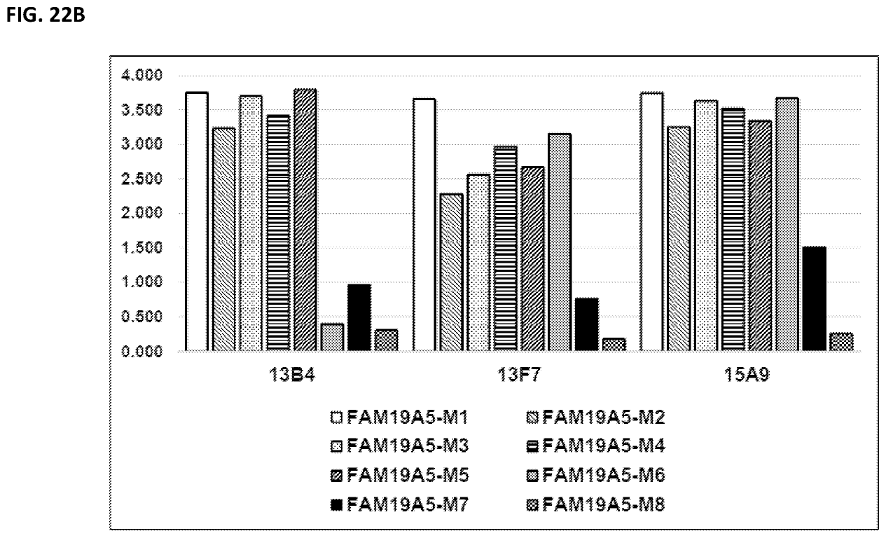

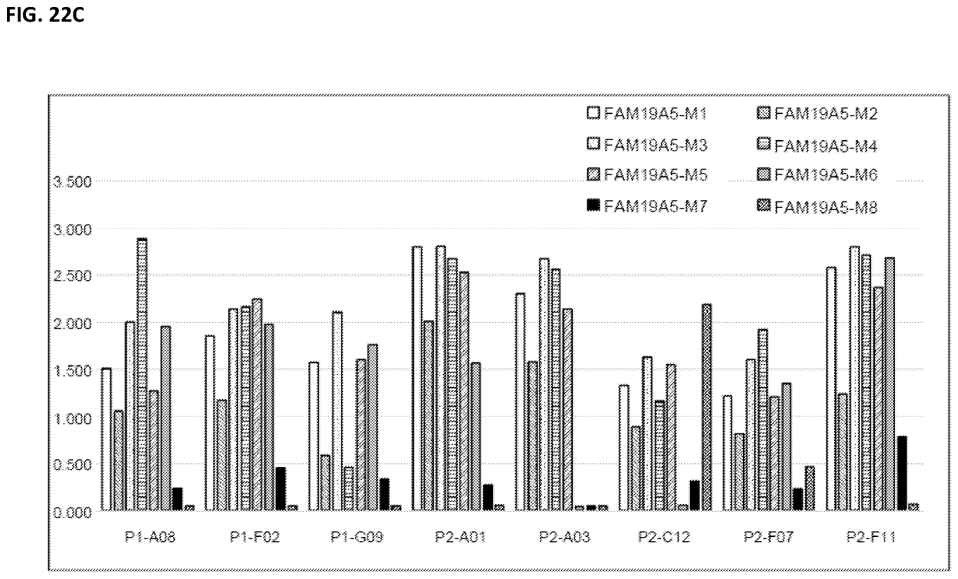

As shown in FIG. 22A and in agreement with the earlier data, the anti-FAM19A5 antibody 1-65 failed to bind to FAM19A5 mutants M6, M7, and M8. The M6 and M7 mutants comprise amino acid mutations at sites that correspond to regions within epitope fragment F5 of FAM19A5. See Table 13 (above).

Epitope analysis for additional anti-FAM19A5 antibodies are provided in FIGS. 22B and 22C. Anti-FAM19A5 antibodies 13B4, 13F₇, and 15A9 all failed to bind to FAM19A5 mutant M8 and had reduced binding to mutant M7. The 13B4 antibody additionally failed to bind to FAM19A5 mutant M6. See FIG. 22B. Similarly, antibodies P1-A08, P1-F02, P2-A01, P2-A03, P2-F07, and P2-F11 all failed to bind to FAM19A5 mutant M8 with many of the antibodies also having reduced to no binding to mutant M7. The P2-C12 antibody differed from these antibodies by strongly binding to FAM19A5 mutant M8. Instead, the P2-C12 antibody failed to bind to FAM19A5 mutant M6, which is in agreement with the earlier data demonstrating that epitope fragment F5 comprises important FAM19A5 binding sites for the P2-C12 antibody.

As shown in FIG. 22A, the anti-FAM19A5 antibodies 1-28 and 3-2 failed to bind to FAM19A5 mutant M2. As shown in Table 13 (above), the M2 mutant has substitutions at amino acid residues 21-25 (i.e., epitope EP2), which correspond to a region within epitope fragment F2 of FAM19A5. This is in agreement with the earlier data and confirms the importance of the epitope fragment F2 for the binding of the 1-28 and 3-2 antibodies to FAM19A5.

As for the 2-13 antibody, it appeared to bind to FAM19A5 at epitopes that are completely different from the 1-28, 3-2, and 1-65 antibodies. As shown in FIG. 22A, anti-FAM19A5 antibody 2-13 had moderate to high binding to FAM19A5 mutants M1-M3 and M5-M7 but failed to bind mutants M4 and M8, suggesting the importance of epitopes EP4 and EP8 in the 2-13 antibody binding to FAM19A5. Considering that the 2-13 antibody did not appear to bind to any of the FAM19A5 epitope fragments as discussed earlier (see FIG. 20A), this data also seems to suggest that the 2-13 antibody has a conformational epitope as opposed to a linear epitope.

### Example 9: Cross-Competition Analysis

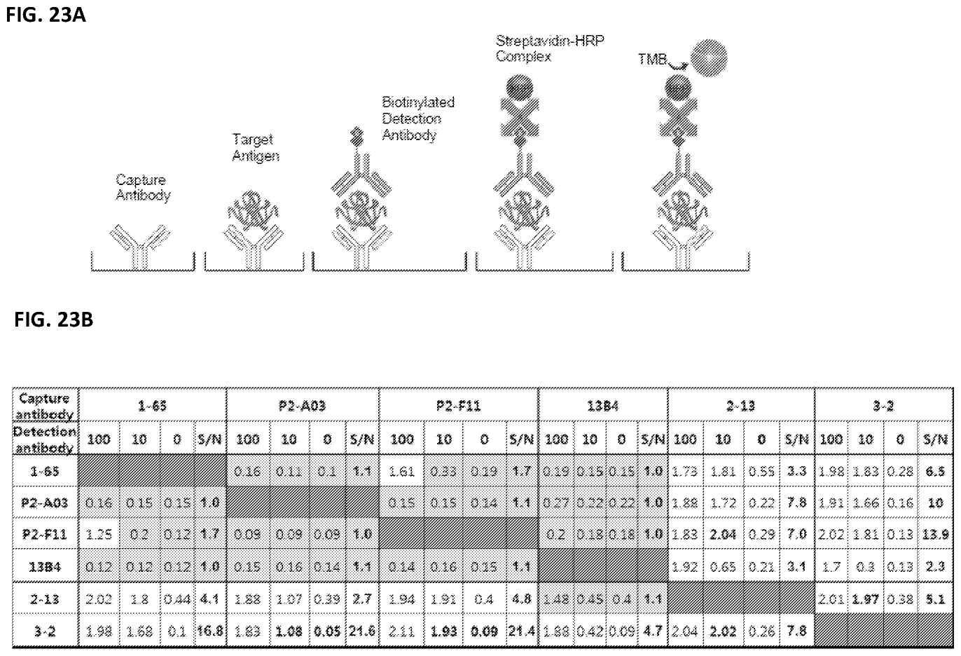

Next, a two-site sandwich ELISA was used as described below to assess whether the different anti-FAM19A5 antibodies with similar binding epitopes cross-compete with each other. See FIG. 23A.

First, the indicated anti-FAM19A5 antibodies were diluted in 1×PBS to a concentration of 10 μg/mL. The diluted anti-FAM19A5 antibodies (10 μg/mL in 1×PBS) (“capture antibody”) were used to coat the 96-well plates (100 μL/well) at 37° C. for approximately 1 hour. After the incubation, the plates were washed with the washing buffer (0.01% Tween-20/PBS; also called 0.01% PBST) for a total of five washes, blocked with the blocking solution (5% BSA/PBS; also called 5% PBSA) (250 μL/well) for one hour at 37° C., and then washed again. Next, 100 μg/mL of the FAM19A5 antigen (diluted in PBS containing 5% BSA, 0.01% Tween-20; also called 5% PBSAT; “diluent buffer) was added to each of the wells, and the 96-well plates were incubated for 2 hours at 37° C. After the incubations, the plates were washed using 0.01% PBST for a total of five washes. After the final wash, the indicated biotinylated anti-FAM19A5 antibodies (“detection antibody”) (diluted to lug/mL in 5% PBSAT) were added to the relevant wells (100 μL volume) and the plates were incubated for an additional 1 hour at 37° C. Afterwards, the plates were washed again with 0.01% PBST (five total washes). Next, 100 μL of the diluted (1/2000 in 5% PBSAT) Streptavidin-HRP (1 mg/mL, Sigma, USA) was added to the wells, and the plates were incubated for 30 minutes at room temperature. The plates were then washed and treated with 100 μL of the TMB substrate (3,3plates were then washed and treated with 100 μLution, Thermo Fisher Scientific). After an additional 30-minute incubation at room temperature, the color change reaction was induced by the addition of the TMB substrate. This reaction was stopped using 50 μL of sulfuric acid (2N H2SO4), and the extent of color change was detected via absorption at 450 nm with reference wavelength at 620 nm using a 96 well microplate reader (Molecular Device).

As shown in FIG. 23B, anti-FAM19A5 antibodies 1-65, P2-A03, P2-F11, and 13B4 all cross-competed with one another. Like the 1-65 antibody, the P2-A03, P2-F11, and 13B4 antibodies all bind to FAM19A5 at epitopes M6, M7, and/or M8 (i.e., within the F5 and/or F6 epitope fragments) (data not shown). In contrast, antibodies 2-13 and 3-2 did not cross-compete with the other anti-FAM19A5 antibodies, confirming the earlier epitope mapping analysis, which showed these antibodies bind primarily at the F2 epitope fragment.

### Example 10: HDX-MX Epitope Mapping of 2-13 Antibody

Hydrogen/deuterium exchange mass spectrometry (HDX-MS) was utilized as described below to probe the binding epitope of human FAM19A5 for the clone 2-13 anti-FAM19A5 antibody.

### 1. Optimization of Conditions for Maximal FAM19A5 Coverage

Prior to epitope mapping experiments, non-deuteriated experiments were carried out to generate a list of common peptides for FAM19A5 protein. Off-line and on-line pepsin digestion were used to digest the FAM19A5 protein. Houde, D., et al., Methods Mol Biol 988:269-89 (2013). With on-line digestion, pepsin immobilized column was used (~20° C.) and the efficiency of the reaction was regulated by adjusting the flow rate. With the off-line method, FAM19A5 protein was digested manually with pepsin at 4° C. for 5 minutes, and then, the mixture was loaded to liquid chromatography column for analysis. To identify the condition for maximal protein coverage, one or more of the following parameters were adjusted: potassium phosphate, sodium phosphate, quench holding time, reducing reagent (TCEP), urea concentration, and pepsin concentration. The Waters PLGS software was used to analyze the raw mass spectrometry (MS) data generated to determine whether maximal protein coverage was obtained. To confirm the reproducibility of the experiments, each of the experimental conditions were repeated at least twice, and peptides that were generated were confirmed. Maximum coverage was observed using the following conditions: 1M TCEP, 2M Urea (pH 2.66), and pepsin at 1:2 concentration.

### 2. FAM19A5 Epitope Mapping

To obtain maximum (100%) binding (K_{D}=1 nM) even when the antibody-protein complex is diluted 15-fold in the labeling buffer, the antigen-antibody complex samples were incubated for at least 3 hours prior to hydrogen/deuterium labeling. After labeling, the samples were brought to the same volume using equilibrium buffer.

To start the labeling reaction, 2.5 μL of the prepared samples (antigen alone, antibody alone, or antigen-antibody complex) diluted in 1:15 labeling buffer were mixed with the D₂O labeling solution (1.8 μM). The reactions were carried out for different time periods of time: 0 min (i.e., undeuterium), 20 seconds, 10 minutes, 60 minutes, and 240 minutes. For the undeuterium reaction, the prepared sample was mixed instead with the equilibration buffer. At the end of each labeling period, the reaction was stopped with quenching buffer. The samples were then vortexed, immediately frozen in liquid nitrogen, and stored at −80° C. until analysis.

Prior to analysis with the mass spectrometer, the stored frozen samples were allowed to melt and then, digested with pepsin for approximately 5 minutes on ice (i.e., off-line method described above). The resulting relative deuterium levels were plotted versus the exchange time with use of the software program DynamX 3.0™ (Waters).

Comparison of the deuterium uptake data (single antigen/antibody v. antigen-antibody complex) for each of the identified peptides were measured. As shown, significant deuterium uptake was observed for the following peptide sequences: (i) ACRKGQIAGTTRARPAC (residues 37-53 of SEQ ID NO: 304; i.e., SEQ ID NO: 306), (ii) ACRKGQIAGTTRARPACVD (residues 37-55 of SEQ ID NO: 304; i.e., SEQ ID NO: 307), (iii) ACRKGQIAGTTRARPACVDA (residues 37-56 of SEQ ID NO: 304; i.e., SEQ ID NO: 308), (iv) ARIIKTKQWC (residues 56-65 of SEQ ID NO: 304; i.e., SEQ ID NO: 309), (v) ARIIKTKQWCDM (residues 56-67 of SEQ ID NO: 304; i.e., SEQ ID NO: 310), (vi) ARIIKTKQWCDML (residues 56-68 of SEQ ID NO: 304; i.e., SEQ ID NO: 311), (vii) ARIIKTKQWCDMLPCL (residues 56-71 of SEQ ID NO: 304; i.e., SEQ ID NO: 312), (viii) RIIKTKQWCDM (residues 57-67 of SEQ ID NO: 304; i.e., SEQ ID NO: 313), and (ix) RIIKTKQWCDML (residues 57-68 of SEQ ID NO: 304; i.e., SEQ ID NO: 314).

Using the above results, a heat map was constructed to identify the regions within FAM19A5 protein with the most significant difference in deuterium uptake between single antigen/antibody sample and antigen-antibody complex. And, amino acid residues 38-50 (CRKGQIAGTTRAR; i.e., SEQ ID NO: 199) and 51-64 (PACVDARIIKTKQW; i.e., SEQ ID NO: 200) of SEQ ID NO: 304 were identified as important binding residues for the 2-13 antibody. FIG. 26 shows the location of these residues within the 3-dimensional structure of the FAM19A5 protein.

### Example 11: Additional Exemplary Materials and Methods

The following materials and methods were used in assessing the therapeutic effects of FAM19A5 antagonists on hearing loss described in Examples 12 and 13.

### Spiral Ganglion Neuron Culture

Sprial ganglion neuron were cultured in culture media consisting of high-glucose (4.5 mg/mL) DMEM, 0.1 mg/ml penicillin, 0.1 mg/mL streptomycin, N2 supplement, and 10 g/mL insulin and was designated here as “5K” because in this media, [K+]=5.4 mM. A depolarizing media, designated as “30K,” in which Na was replaced with equimolar K to raise [K] to 30 mM while maintaining osmolarity was also used.

Dissociated spiral ganglion cell cultures were prepared from postnatal day 4-5 (P4-5) cochleae and maintained using a procedure modified from the methods described by Bok et al., J Neurosci 23 (3): 777-87 (2003), which is incorporated herein by reference in its entirety. The cochleae were aseptically dissected from the temporal bone and submerged in ice-cold HBSS (Gibco). Subsequent steps involved the removal of the bony cochlear capsule and the spiral ligament. The organ of Corti, surrounding connective tissue and modiolar bone was removed while preserving the spiral ganglion neurons within the modiolus. Ganglia were collected in Ca²⁺—Mg²⁺-free HBSS with 0.1% trypsin and 0.1% collagenase and enzymic dissociation was then performed at 37° C. CO₂ incubator for 20 minutes. Cells were washed with 5K media for 3 times and ganglia were gently triturated approximately 15 times with pipette in 30K media. Cells were then seeded in the poly-D-lysine and laminin (Thermo Fisher Scientific) coated 4-well slide glass and cells were maintained at 37° C. in a 5% CO₂-humified incubator.

### Cochlear Explant Culture

Cochlear explant were cultured in medium consisting of plating media, which consist of Neurobasal media (Invitrogen), 1% N2 supplement, and 10 μg/mL Ampicillin with culture media with additional 1% FBS.

Postnatal day 4-5 (P4-5) C57BL/6 mice were sacrificed for inner ear dissection. Mouse head was bisected, and brain tissue was removed. Cochlea was carefully separated the from the extraneous bones in ice-cold HBSS (5 mM HEPES/1.3 mM CaCl₂), 0.9 mM MgCl₂, pH 7.4) or External solution (1.2 mM CaCl₂), 1 mM MgCl₂, 5 mM HEPES in HBSS). Then the stria vascularis and spiral bone plate was removed, and cochlea was cut into two to three sections. Reissner's membrane, and tectorial membrane was removed from the dissected tissues. Tissues were then moved to poly-D-lysine and laminin (Thermo Fisher Scientific) coated 4-well slide glasses. Cochlear explant tissues were positioned as the area of hair cells from which the tectorial membrane was removed faces upwards. Explants were stabilized overnight in plating media and full-changed to culture media and maintained at 37° C. in a 5% CO₂-humified incubator.

### Immunostaining

Primary spiral ganglion neurons and cochlear explants were fixed with 4% paraformaldehyde (PFA) for 20 minutes at appropriate DIV (days in vitro). The cells/tissues were blocked with 3% bovine serum albumin (BSA) and 0.1% Triton X-100 in phosphate-buffered saline (PBS) for 1 h at room temperature. Primary antibodies were then applied to the cells/tissues overnight at 4° C. Primary antibodies used in this study were mouse anti-NF200 (Sigma) and mouse anti-CtBP2 (BD bioscience). After several washes with PBS, appropriate fluorescent conjugated secondary antibodies were applied for 1 hour at room temperature. Subsequently, cells/tissues were imaged using a confocal microscope (Leica).

### Quantitative Analysis of Neurite Outgrowth

Neurite outgrowth was quantified by measuring the total length of neurites per neuron. Neurons were stained with NF200 at either 2 or 3 DIV, and neurite lengths were measured using the Fiji (Image J). Individual neurons were manually selected, and neurite lengths were determined using the Simple Neurite Tracer plugin.

### Quantitative Analysis of Synaptogenesis

Mouse cochlear explant cultures were treated with FAM19A5, FAM19A5 combined with a FAM19A5 antagonist (e.g., described herein, e.g., 1-30 antibody), or a FAM19A5 antagonist (e.g., described herein, e.g., 1-30 antibody) alone at 1 DIV to determine the level of synaptogenesis at 2 DIV by immunostaining CtBP2, a ribbon synapse marker. To quantify the number of CtBP2 puncta of individual inner hair cells, z-stack confocal images were exported in “multi-page” and the number of CtBP2 puncta from each individual inner hair cells were measured using ROI manger and multi-point tool from Fiji (Image J). Number of CtBP2 puncta from five individual inner hair cells were counted per image and 3 z-stack images were measured from each treatment group.

### Statistical Analysis

All statistical analysis was performed using GraphPad Prism 8 (GraphPad Software Inc.), and the data are shown as the mean±standard error of the mean (SEM). Statistical significance was evaluated using Student's t-tests and/or one-way analysis of variance (ANOVA) with Tukey's multiple comparisons tests. A p-value of less than 0.05 was considered statistically significant.

### Example 12: Effect on Fam19A5-Induced Inhibition of Neurite Outgrowth

Spiral ganglion neurons (SGN) are a key component of the auditory system. These specialized neurons serve as the primary conduits for transmitting electrical impulses generated by the mechanosensory hair cells within the cochlea to the central auditory pathways in the brain. Their role encompasses the encoding and transmission of critical auditory parameters, including sound frequency, intensity, and temporal features. However, these neurons are susceptible to impairment in cases of sensorineural hearing loss, the most prevalent form of hearing impairment. To evaluate the regenerative capacity of the SGNs under auditory dysfunction, the ability of FAM19A5 antagonists described herein in reversing FAM19A5-induced inhibition of neurite outgrowth in mouse primary spiral ganglion neurons was assessed.

Briefly, mouse primary spiral ganglion neurons (cultured and processed as described in Example 11) were treated with a FAM19A5 protein (10 μM) alone or in combination with an anti-FAM19A5 antibody (1-30) (100 nM) at 0 DIV (day in vitro). Non-treated cells (i.e., no FAM19A5 protein nor anti-FAM19A5 antibody) were used as control. Then, at 3 DIV, the neurons were stained for neurofilament protein (NF200) expression and the average neurite length quantified.

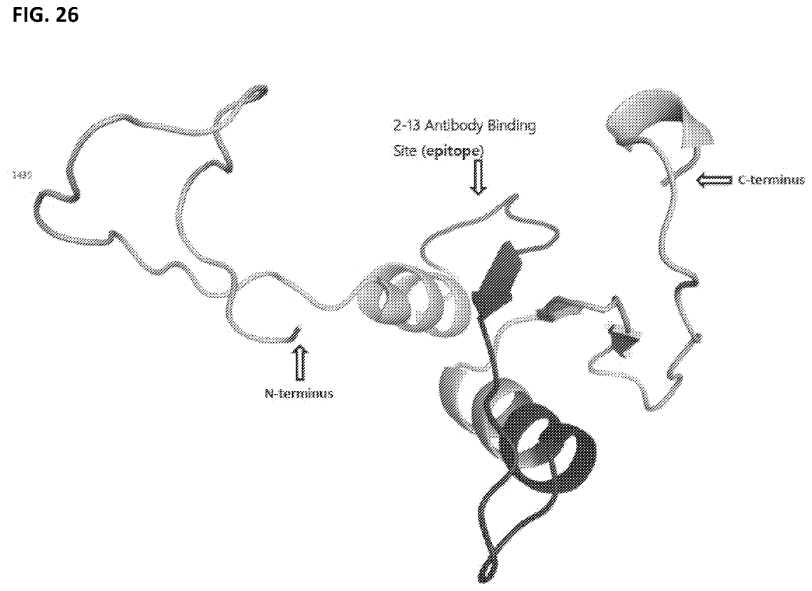

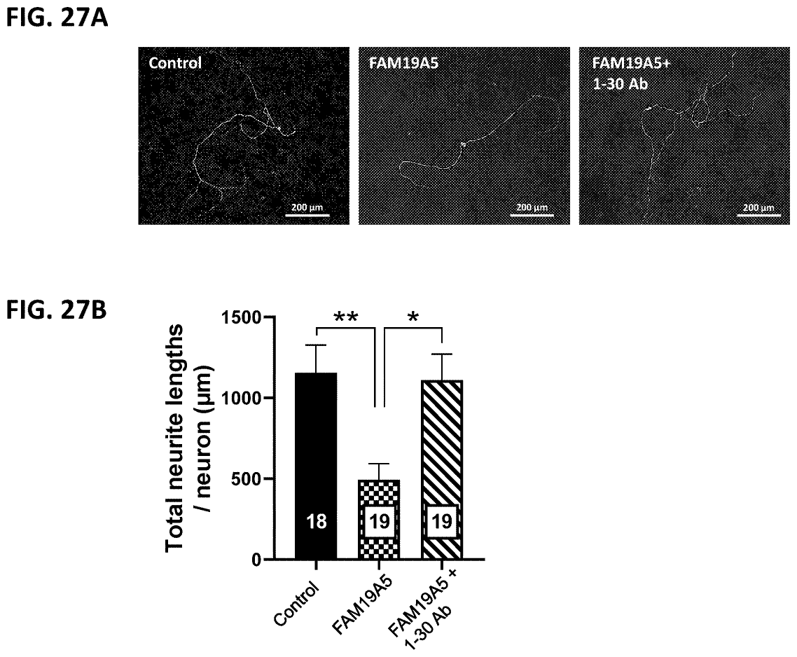

As shown in FIGS. 27A and 27B, neurons treated with the FAM19A5 protein exhibited significantly reduced total neurite length as compared to the non-treated cells (i.e., compare “control” and “FAM19A5” groups). However, in FAM19A5-treated neurons that were co-treated with the anti-FAM19A5 antibody, there was less reduction in neurite growth and comparable to the neurons from the control group. These results suggest that the FAM19A5 antagonists described herein (e.g., anti-FAM19A5 antibody, e.g., 1-30 antibody) co-treatment effectively counteracted the inhibitory effect of FAM19A5 protein on neurite outgrowth in spiral ganglion neurons.

### Example 13: Effect on the Promotion of Neurite Outgrowth

To further assess the effect on neurite outgrowth, primary mouse spiral ganglion neurons (isolated at postnatal day 4) were treated with one of the following FAM19A5 antagonists: (1) 1-30 anti-FAM19A5 antibody, (2) 2-13 anti-FAM19A5 antibody, (3) 1-65 SS01 anti-FAM19A5 antibody, or (4) a FAM19A5 targeting peptide (dFB-DY-JM31). Neurons treated with a control human IgG antibody were used as control. Following treatment, neurons were stained for neurofilament protein (NS200) expression and the average neurite length quantified.

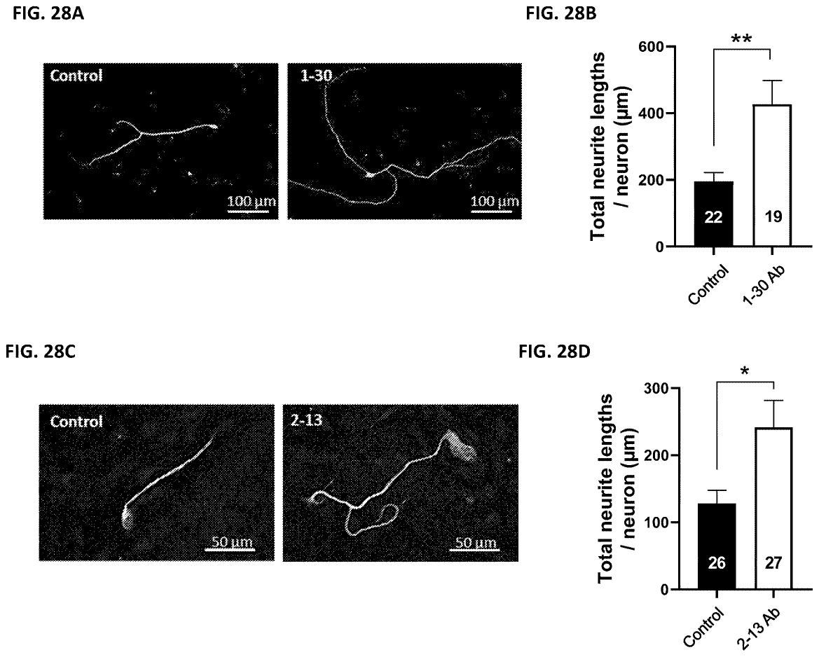

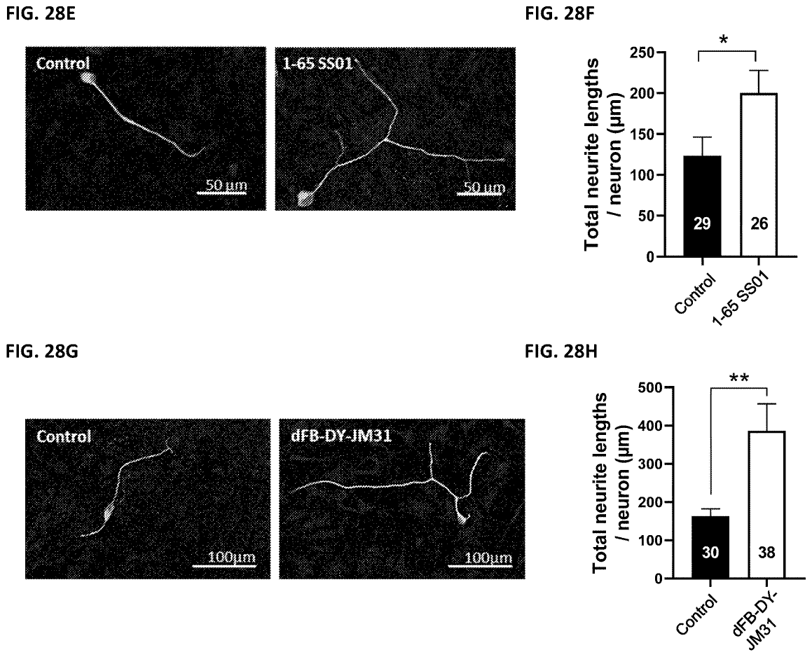

As shown in FIGS. 28A-28F, for all anti-FAM19A5 antibodies tested, there was much increased neurite length as compared to that observed in neurons treated with the control antibody. Similar results were observed in neurons treated with the FAM19A5 targeting peptide (see FIGS. 28G and 28H). These results further highlight the therapeutic effects of the FAM19A5 antagonists described herein (e.g., both anti-FAM19A5 antibodies and FAM19A5-targeting peptides) on neurite outgrowth.

### Example 14: Effect on FAM19A5-Induced Inhibition of Synaptogenesis

In the auditory system, hair cells and spiral ganglion neurons collaborate intricately to enable hearing. Hair cells, found in the cochlea of the inner ear, serve as mechanoreceptors responsible for converting incoming sound waves into electrical signals. These hair cells are categorized into inner and outer hair cells, with the former primarily responsible for transmitting auditory information. The crucial link in this process lies in the synapse between hair cells and spiral ganglion neurons. At this synapse, neurotransmitters are released by the hair cells in response to mechanical stimulation, facilitating the transmission of electrical signals to spiral ganglion neurons. This synapse is of paramount importance, as any disruption or damage to it, whether through aging, noise exposure, ototoxic drugs, or genetic mutations, can lead to sensorineural hearing loss. In such cases, impaired transmission at the hair cell-spiral ganglion neuron synapse, also known as ribbon synapse, results in diminished auditory signal transduction, ultimately compromising hearing sensitivity and auditory perception. Understanding the intricacies of this synaptic connection is vital for addressing the mechanisms and potential treatments for hearing loss.

In order to further examine the effect of FAM19A5 antagonists described herein on hearing loss, primary cochlear explant cultures were treated with a FAM19A5 protein (1 μM) alone or in combination with an anti-FAM19A5 antibody (1-30) at 1 DIV. Primary cochlear explant treated with a control anti-human IgG antibody alone were used as control. Then, at 2 DIV, the cochlear explants were immunostained for ribbon synapse marker (CtBP2) expression within the inner hair cell.

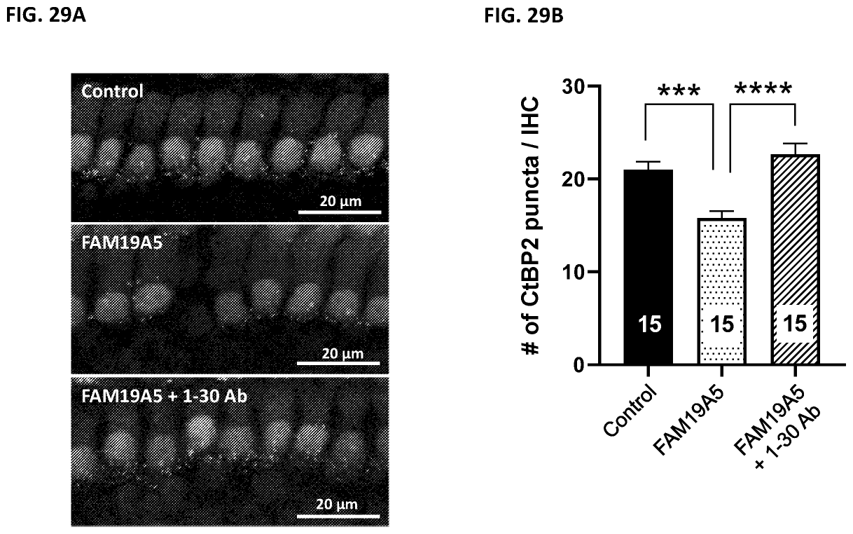

As shown in FIGS. 29A and 29B, the number of CtBP2 puncta was significantly decreased in the inner hair cells of FAM19A5 protein-treated cochlear explants as compared to the control group. However, in cochlear explants treated with both the FAM19A5 protein and the anti-FAM19A5 antibody, the number of CtBP2 puncta was increased (as compared to the FAM19A5 protein alone group) and comparable to that of the control group. These results highlight the neuroprotective properties of the FAM19A5 antagonists described herein (e.g., anti-FAM19A5 antibody, e.g., 1-30 antibody) on FAM19A5-induced inhibition of synaptogenesis in mouse cochlear explant.

### Example 15: Effect on the Promotion of Synaptogenesis

To further assess the synaptogenic evidence of the FAM19A5 antagonists described herein, mouse cochlear explants were treated with either the control human IgG antibody or an anti-FAM19A5 antibody (1-30; 1 μM). Then, the cochlear explants were immunostained for CtBP2 expression within the inner hair cell.

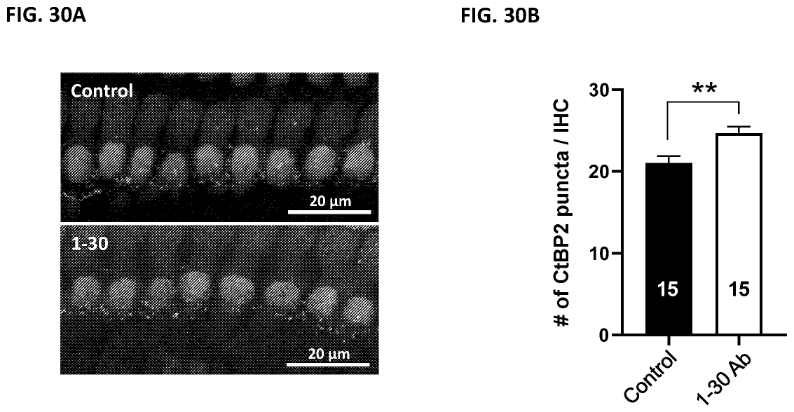

Consistent with the neurite outgrowth data provided earlier (see Example 13), there was significant increase in the number of CtBP2 puncta within the innher hair cells following anti-FAM19A5 treatment (see FIGS. 30A and 30B). These results highlight the ability of the FAM19A5 antagonists described herein (e.g., anti-FAM19A5 antibody, e.g., 1-30 antibody) to enhance synaptogenesis between hair cells and spiral ganglion neurons.

It is to be appreciated that the Detailed Description including the Summary and Abstract sections is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, internet sites, and accession numbers/database sequences (including both polynucleotide and polypeptide sequences) cited herein are hereby incorporated by reference in their entirety for all purposes to the same extent as if each individual publication, patent, patent application, internet site, or accession number/database sequence were specifically and individually indicated to be so incorporated by reference.

1-83. (canceled)

84. A method of treating or preventing a hearing disorder in a subject in need thereof, comprising administering to the subject a pharmaceutical composition comprising (a) an antagonist against a family with sequence similarity 19, member A5 (FAM19A5) protein (FAM19A5 antagonist) and (b) a pharmaceutically acceptable carrier,
wherein the FAM19A5 antagonist comprises (i) an antibody that specifically binds to FAM19A5 protein or an antigen binding fragment thereof (“anti FAM19A5 antibody”); (ii) a polynucleotide encoding the anti FAM19A5 antibody; or (iii) a vector comprising the polynucleotide, wherein the anti FAM19A5 antibody comprises heavy chain CDR1, CDR2, and CDR3, and light chain CDR1, CDR2, and CDR3, and wherein
(1) the heavy chain CDR1 consists of the amino acid sequence set forth in SEQ ID NO: 14; the heavy chain CDR2 consists of the amino acid sequence set forth in SEQ ID NO: 15; the heavy chain CDR3 consists of the amino acid sequence set forth in SEQ ID NO: 16; the light chain CDR1 consists of the amino acid sequence set forth in SEQ ID NO: 26; the light chain CDR2 consists of the amino acid sequence set forth in SEQ ID NO: 27; and the light chain CDR3 consists of the amino acid sequence set forth in SEQ ID NO: 28,
(2) the heavy chain CDR1 consists of the amino acid sequence set forth in SEQ ID NO: 220; the heavy chain CDR2 consists of the amino acid sequence set forth in SEQ ID NO: 221; the heavy chain CDR3 consists of the amino acid sequence set forth in SEQ ID NO: 16; the light chain CDR1 consists of the amino acid sequence set forth in SEQ ID NO: 225; the light chain CDR2 consists of the amino acid sequence set forth in SEQ ID NO: 226; and the light chain CDR3 consists of the amino acid sequence set forth in SEQ ID NO: 227,
(3) the heavy chain CDR1 consists of the amino acid sequence set forth in SEQ ID NO: 220; the heavy chain CDR2 consists of the amino acid sequence set forth in SEQ ID NO: 221; the heavy chain CDR3 consists of the amino acid sequence set forth in SEQ ID NO: 16; the light chain CDR1 consists of the amino acid sequence set forth in SEQ ID NO: 225; the light chain CDR2 consists of the amino acid sequence set forth in SEQ ID NO: 228; and the light chain CDR3 consists of the amino acid sequence set forth in SEQ ID NO: 227,
(4) the heavy chain CDR1 consists of the amino acid sequence set forth in SEQ ID NO: 220; the heavy chain CDR2 consists of the amino acid sequence set forth in SEQ ID NO: 221; the heavy chain CDR3 consists of the amino acid sequence set forth in SEQ ID NO: 16; the light chain CDR1 consists of the amino acid sequence set forth in SEQ ID NO: 26; the light chain CDR2 consists of the amino acid sequence set forth in SEQ ID NO: 229; and the light chain CDR3 consists of the amino acid sequence set forth in SEQ ID NO: 227,
(5) the heavy chain CDR1 consists of the amino acid sequence set forth in SEQ ID NO: 220; the heavy chain CDR2 consists of the amino acid sequence set forth in SEQ ID NO: 221; the heavy chain CDR3 consists of the amino acid sequence set forth in SEQ ID NO: 16; the light chain CDR1 consists of the amino acid sequence set forth in SEQ ID NO: 26; the light chain CDR2 consists of the amino acid sequence set forth in SEQ ID NO: 228; and the light chain CDR3 consists of the amino acid sequence set forth in SEQ ID NO: 227,
(6) the heavy chain CDR1 consists of the amino acid sequence set forth in SEQ ID NO: 220; the heavy chain CDR2 consists of the amino acid sequence set forth in SEQ ID NO: 221; the heavy chain CDR3 consists of the amino acid sequence set forth in SEQ ID NO: 16; the light chain CDR1 consists of the amino acid sequence set forth in SEQ ID NO: 26; the light chain CDR2 consists of the amino acid sequence set forth in SEQ ID NO: 230; and the light chain CDR3 consists of the amino acid sequence set forth in SEQ ID NO: 231, or
(7) the heavy chain CDR1 consists of the amino acid sequence set forth in SEQ ID NO: 220; the heavy chain CDR2 consists of the amino acid sequence set forth in SEQ ID NO: 221; the heavy chain CDR3 consists of the amino acid sequence set forth in SEQ ID NO: 16; the light chain CDR1 consists of the amino acid sequence set forth in SEQ ID NO: 26; the light chain CDR2 consists of the amino acid sequence set forth in SEQ ID NO: 232; and the light chain CDR3 consists of the amino acid sequence set forth in SEQ ID NO: 233.

85. The method of claim 84, wherein the hearing disorder comprises a sensorineural hearing loss.

86. The method of claim 85, wherein: (a) the sensorineural hearing loss is with tinnitus, (b) the sensorineural hearing loss is without tinnitus, or (c) the sensorineural hearing loss comprises an ototoxic drug-induced hearing loss, noise-induced hearing loss, age-related hearing loss, sudden hearing loss, or combinations thereof.

87. The method of claim 84, wherein the pharmaceutical composition (a) reduces or prevents an impairment of ribbon synapses within an inner ear of a subject in need thereof, (b) induces a formation of ribbon synapses within an inner ear of a subject in need thereof; or (c) improves a function of ribbon synapses within an inner ear of a subject in need thereof.

88. The method of claim 87, wherein the impairment of ribbon synapses comprises (a) a decrease in the number of ribbon synapses as compared to that of a corresponding subject who does not have an impairment of ribbon synapses within an inner ear, (b) an abnormal function of the ribbon synapses, or (c) both (a) and (b).

89. The method of claim 87, wherein the function comprises the ability to release one or more neurotransmitters in response to a signal from an inner hair cell.

90. The method of claim 84, wherein the pharmaceutical composition reduces an expression or activity of the FAM19A5 protein within a spiral ganglion neuron of the subject.

91. The method of claim 84, wherein the anti-FAM19A5 antibody comprises a heavy chain variable region (VH) and a light chain variable region (VL).

92. The method of claim 91, wherein the VH comprises the sequence set forth in SEQ ID NO: 235 and the VL comprises the sequence set forth in SEQ ID NO: 240.

93. The method of claim 84, wherein the pharmaceutical composition is administered to the subject via intracochlear injection, intravestibular injection, intravenous administration, or intratympanic administration.


SEQUENCE LISTING (320 sequences)

SEQ ID NO: 1 - protein, 132 aa, synthetic construct
  source 1..132: note = FAM19A5 Isoform 1
MAPSPRTGSRQDATALPSMSSTFWAFMILASLLIAYCSQLAAGTCEIVTLDRDSSQPRRTIARQTARCACRKGQIAGTTRARPACVDARIIKTKQWCDMLPCLEGEGCDLLINRSGWTCTQPGGRIKTTTVS

SEQ ID NO: 2 - protein, 125 aa, synthetic construct
  source 1..125: note = FAM19A5 Isoform 2
MQLLKALWALAGAALCCFLVLVIHAQFLKEGQLAAGTCEIVTLDRDSSQPRRTIARQTARCACRKGQIAGTTRARPACVDARIIKTKQWCDMLPCLEGEGCDLLINRSGWTCTQPGGRIKTTTVS

SEQ ID NO: 3 - protein, 53 aa, synthetic construct
  source 1..53: note = FAM19A5 Isoform 3
MYHHREWPARIIKTKQWCDMLPCLEGEGCDLLINRSGWTCTQPGGRIKTTTVS

SEQ ID NO: 4 - protein, 653 aa, synthetic construct
  source 1..653: note = Human LRRC4 protein
MKLLWQVTVHHHTWNAILLPFVYLTAQVWILCAAIAAAASAGPQNCPSVCSCSNQFSKVVCTRRGLSEVPQGIPSNTRYLNLMENNIQMIQADTFRHLHHLEVLQLGRNSIRQIEVGAFNGLASLNTLELFDNWLTVIPSGAFEYLSKLRELWLRNNPIESIPSYAFNRVPSLMRLDLGELKKLEYISEGAFEGLFNLKYLNLGMCNIKDMPNLTPLVGLEELEMSGNHFPEIRPGSFHGLSSLKKLWVMNSQVSLIERNAFDGLASLVELNLAHNNLSSLPHDLFTPLRYLVELHLHHNPWNCDCDILWLAWWLREYIPTNSTCCGRCHAPMHMRGRYLVEVDQASFQCSAPFIMDAPRDLNISEGRMAELKCRTPPMSSVKWLLPNGTVLSHASRHPRISVLNDGTLNFSHVLLSDTGVYTCMVTNVAGNSNASAYLNVSTAELNTSNYSFFTTVTVETTEISPEDTTRKYKPVPTTSTGYQPAYTTSTTVLIQTTRVPKQVAVPATDTTDKMQTSLDEVMKTTKIIIGCFVAVTLLAAAMLIVFYKLRKRHQQRSTVTAARTVEIIQVDEDIPAATSAAATAAPSGVSGEGAVVLPTIHDHINYNTYKPAHGAHWTENSLGNSLHPTVTTISEPYIIQTHTKDKVQETQI

SEQ ID NO: 5 - protein, 38 aa, synthetic construct
  source 1..38: note = Human LRRC4 protein signal peptide
MKLLWQVTVHHHTWNAILLPFVYLTAQVWILCAAIAAA

SEQ ID NO: 6 - protein, 489 aa, synthetic construct
  source 1..489: note = Human LRRC4 ectodomain protein
ASAGPQNCPSVCSCSNQFSKVVCTRRGLSEVPQGIPSNTRYLNLMENNIQMIQADTFRHLHHLEVLQLGRNSIRQIEVGAFNGLASLNTLELFDNWLTVIPSGAFEYLSKLRELWLRNNPIESIPSYAFNRVPSLMRLDLGELKKLEYISEGAFEGLFNLKYLNLGMCNIKDMPNLTPLVGLEELEMSGNHFPEIRPGSFHGLSSLKKLWVMNSQVSLIERNAFDGLASLVELNLAHNNLSSLPHDLFTPLRYLVELHLHHNPWNCDCDILWLAWWLREYIPTNSTCCGRCHAPMHMRGRYLVEVDQASFQCSAPFIMDAPRDLNISEGRMAELKCRTPPMSSVKWLLPNGTVLSHASRHPRISVLNDGTLNFSHVLLSDTGVYTCMVTNVAGNSNASAYLNVSTAELNTSNYSFFTTVTVETTEISPEDTTRKYKPVPTTSTGYQPAYTTSTTVLIQTTRVPKQVAVPATDTTDKMQTSLDEVMKTTK

SEQ ID NO: 7 - protein, 713 aa, synthetic construct
  source 1..713: note = Human LRRC4B protein
MARARGSPCPPLPPGRMSWPHGALLFLWLFSPPLGAGGGGVAVTSAAGGGSPPATSCPVACSCSNQASRVICTRRDLAEVPASIPVNTRYLNLQENGIQVIRTDTFKHLRHLEILQLSKNLVRKIEVGAFNGLPSLNTLELFDNRLTTVPTQAFEYLSKLRELWLRNNPIESIPSYAFNRVPSLRRLDLGELKRLEYISEAAFEGLVNLRYLNLGMCNLKDIPNLTALVRLEELELSGNRLDLIRPGSFQGLTSLRKLWLMHAQVATIERNAFDDLKSLEELNLSHNNLMSLPHDLFTPLHRLERVHLNHNPWHCNCDVLWLSWWLKETVPSNTTCCARCHAPAGLKGRYIGELDQSHFTCYAPVIVEPPTDLNVTEGMAAELKCRTGTSMTSVNWLTPNGTLMTHGSYRVRISVLHDGTLNFTNVTVQDTGQYTCMVTNSAGNTTASATLNVSAVDPVAAGGTGSGGGGPGGSGGVGGGSGGYTYFTTVTVETLETQPGEEALQPRGTEKEPPGPTTDGVWGGGRPGDAAGPASSSTTAPAPRSSRPTEKAFTVPITDVTENALKDLDDVMKTTKIIIGCFVAITFMAAVMLVAFYKLRKQHQLHKHHGPTRTVEIINVEDELPAASAVSVAAAAAVASGGGVGGDSHLALPALERDHLNHHHYVAAAFKAHYSSNPSGGGCGGKGPPGLNSIHEPLLFKSGSKENVQETQI

SEQ ID NO: 8 - protein, 35 aa, synthetic construct
  source 1..35: note = Human LRRC4B protein signal peptide
MARARGSPCPPLPPGRMSWPHGALLFLWLFSPPLG

SEQ ID NO: 9 - protein, 541 aa, synthetic construct
  source 1..541: note = Human LRRC4B ectodomain protein
AGGGGVAVTSAAGGGSPPATSCPVACSCSNQASRVICTRRDLAEVPASIPVNTRYLNLQENGIQVIRTDTFKHLRHLEILQLSKNLVRKIEVGAFNGLPSLNTLELFDNRLTTVPTQAFEYLSKLRELWLRNNPIESIPSYAFNRVPSLRRLDLGELKRLEYISEAAFEGLVNLRYLNLGMCNLKDIPNLTALVRLEELELSGNRLDLIRPGSFQGLTSLRKLWLMHAQVATIERNAFDDLKSLEELNLSHNNLMSLPHDLFTPLHRLERVHLNHNPWHCNCDVLWLSWWLKETVPSNTTCCARCHAPAGLKGRYIGELDQSHFTCYAPVIVEPPTDLNVTEGMAAELKCRTGTSMTSVNWLTPNGTLMTHGSYRVRISVLHDGTLNFTNVTVQDTGQYTCMVTNSAGNTTASATLNVSAVDPVAAGGTGSGGGGPGGSGGVGGGSGGYTYFTTVTVETLETQPGEEALQPRGTEKEPPGPTTDGVWGGGRPGDAAGPASSSTTAPAPRSSRPTEKAFTVPITDVTENALKDLDDVMKTTK

SEQ ID NO: 10 - skipped

SEQ ID NO: 11 - protein, 5 aa, synthetic construct
  source 1..5: note = 2-13 VH-CDR1
SHGMF

SEQ ID NO: 12 - protein, 17 aa, synthetic construct
  source 1..17: note = 2-13 VH-CDR2, 2-13D VH-CDR2, 2-13D-37 VH-CDR2, 2-13D-37-1.5W-41 VH-CDR2, 2-13D-37-3W-16 VH-CDR2
EITNDGSGTNYGSAVKG

SEQ ID NO: 13 - protein, 20 aa, synthetic construct
  source 1..20: note = 2-13 VH-CDR3, 2-13D VH-CDR3, 2-13D-37 VH-CDR3
STYECPGGFSCWGDTGQIDA

SEQ ID NO: 14 - protein, 5 aa, synthetic construct
  source 1..5: note = 3-2 VH-CDR1
SFNMF

SEQ ID NO: 15 - protein, 17 aa, synthetic construct
  source 1..17: note = 3-2 VH-CDR2
QISSSGSSTNYAPAVRG

SEQ ID NO: 16 - protein, 22 aa, synthetic construct
  source 1..22: note = 3-2 VH-CDR3, 1-7A-IT VH-CDR3, Low-PI VH-CDR3, 1-30 VH-CDR3, 1-17 VH-CDR3, 1-32 VH-CDR3, 4-11 VH-CDR3, 6-10 VH-CDR3
SSYDCPYGHCSSGVDSAGEIDA

SEQ ID NO: 17 - protein, 5 aa, synthetic construct
  source 1..5: note = 1-65 VH-CDR1, SS01-13 VH-CDR1, SS01-13-s5 VH-CDR1, S5-2.GKNG VH-CDR1
SYQMG

SEQ ID NO: 18 - protein, 10 aa, synthetic construct
  source 1..10: note = 1-65 VH-CDR2, SS01-13 VH-CDR2
VINKSGSDTS

SEQ ID NO: 19 - protein, 13 aa, synthetic construct
  source 1..13: note = 1-65 VH-CDR3, SS01-13 VH-CDR3, SS01-13-s5 VH-CDR3, S5-2.GKNG VH-CDR3
GSASYITAATIDA

SEQ ID NO: 20 - protein, 8 aa, synthetic construct
  source 1..8: note = 1-28 VH-CDR1
GFDFSDYG

SEQ ID NO: 21 - protein, 8 aa, synthetic construct
  source 1..8: note = 1-28 VH-CDR2
IRSDGSNP

SEQ ID NO: 22 - protein, 29 aa, synthetic construct
  source 1..29: note = 1-28 VH-CDR3
AKDGNGYCALDAYRSGGYSCGVYPGSIDA

SEQ ID NO: 23 - protein, 8 aa, synthetic construct
  source 1..8: note = 2-13 VL-CDR1
SGGSYSYG

SEQ ID NO: 24 - protein, 7 aa, synthetic construct
  source 1..7: note = 2-13 VL-CDR2, 2-13D VL-CDR2, 2-13D-37 VL-CDR2, 2-13D-37-1.5W-41 VL-CDR2, 2-13D-37-3W-16 VL-CDR2
WDDERPS

SEQ ID NO: 25 - protein, 11 aa, synthetic construct
  source 1..11: note = 2-13 VL-CDR3, 2-13D VL-CDR3, 2-13D-37 VL-CDR3, 2-13D-37-1.5W-41 VL-CDR3, 2-13D-37-3W-16 VL-CDR3
GTEDISGTAGV

SEQ ID NO: 26 - protein, 13 aa, synthetic construct
  source 1..13: note = 3-2 VL-CDR1, 1-7A-IT VL-CDR1, 1-17 VL-CDR1, 1-32 VL-CDR1, 4-11 VL-CDR1, 6-10 VL-CDR1
SGGGSYAGSYYYG

SEQ ID NO: 27 - protein, 7 aa, synthetic construct
  source 1..7: note = 3-2 VL-CDR2
ESNKRPS

SEQ ID NO: 28 - protein, 10 aa, synthetic construct
  source 1..10: note = 3-2 VL-CDR3, 1-7A-IT VL-CDR3
GSWDSSNGGI

SEQ ID NO: 29 - protein, 11 aa, synthetic construct
  source 1..11: note = 1-65 VL-CDR1
SGGGSSGYGYG

SEQ ID NO: 30 - protein, 7 aa, synthetic construct
  source 1..7: note = 1-65 VL-CDR2
WNDKRPS

SEQ ID NO: 31 - protein, 14 aa, synthetic construct
  source 1..14: note = 1-65 VL-CDR3, SS01-13 VL-CDR3, SS01-13-S5 VL-CDR3, S5-2.GKNG VL-CDR3
GNDDYSSDSGYVGV

SEQ ID NO: 32 - protein, 5 aa, synthetic construct
  source 1..5: note = 1-28 VL-CDR1
GYGYG

SEQ ID NO: 33 - skipped

SEQ ID NO: 34 - protein, 11 aa, synthetic construct
  source 1..11: note = 1-28 VL-CDR3
GSEDSSTLAGI

SEQ ID NO: 35 - protein, 129 aa, synthetic construct
  source 1..129: note = 2-13 VH
AVTLDESGGGLQTPGGALSLVCKASGFTFSSHGMFWVRQTPGKGLEYVAEITNDGSGTNYGSAVKGRATISRDNGQSTVRLQLNNLRAEDTGTYFCARSTYECPGGFSCWGDTGQIDAWGHGTEVIVSS

SEQ ID NO: 36 - protein, 131 aa, synthetic construct
  source 1..131: note = 3-2 VH
AVTLDESGGGLQTPGGALSLVCKASGFTFSSFNMFWVRQAPGKGLEYVAQISSSGSSTNYAPAVRGRATISRDNGQSTVRLQLNNPGAEDTGTYYCAKSSYDCPYGHCSSGVDSAGEIDAWGHGTEVIVSS

SEQ ID NO: 37 - protein, 122 aa, synthetic construct
  source 1..122: note = 1-65 VH
AVTLDESGGGLQTPGGALSLVCKASGFTFSSYQMGWVRQAPGKGLEWVGVINKSGSDTSYGSAVKGRATISRDNGQSTVRLQLNNLRAEDTGTYFCAKGSASYITAATIDAWGHGTEVIVSS

SEQ ID NO: 38 - protein, 136 aa, synthetic construct
  source 1..136: note = 1-28 VH
AVTLDESGGGLQTPGGALSLVCKASGFDFSDYGMGWVRQAPGKGLEWVAAIRSDGSNPSYGSAVKGRATISKDNGRSTVRLQLNNLRAEDTATYYCAKDGNGYCALDAYRSGGYSCGVYPGSIDAWGHGTEVIVSS

SEQ ID NO: 39 - protein, 104 aa, synthetic construct
  source 1..104: note = 2-13 VL
ALTQPSSVSANPGETVKITCSGGSYSYGWFQQKSPGSALVTVIYWDDERPSDIPSRFSGALSGSTNTLTITGVQADDEAVYFCGTEDISGTAGVFGAGTTLTVL

SEQ ID NO: 40 - protein, 108 aa, synthetic construct
  source 1..108: note = 3-2 VL
ALTQPSSVSANPGETVKITCSGGGSYAGSYYYGWYQQKAPGSAPVTLIYESNKRPSDIPSRFSGSTSGSTATLTITGVQADDEAIYYCGSWDSSNGGIFGAGTTLTVL

SEQ ID NO: 41 - protein, 110 aa, synthetic construct
  source 1..110: note = 1-65 VL
ALTQPSSVSANPGETVKITCSGGGSSGYGYGWYQQKSPSSAPLTVIYWNDKRPSDIPSRFSGSKSGSTHTLTITGVQAEDEAVYFCGNDDYSSDSGYVGVFGAGTTLTVL

SEQ ID NO: 42 - protein, 104 aa, synthetic construct
  source 1..104: note = 1-28 VL
ALTQPSSVSANLEGTVEITCSGSGYGYGWYQQKSPGSAPVTVIYQNDKRPSDIPSRFSGSKSGSTGTLTITGVQVEDEAVYYCGSEDSSTLAGIFGAGTTLTVL

SEQ ID NO: 43 - skipped

SEQ ID NO: 44 - protein, 10 aa, synthetic construct
  source 1..10: note = LRRC4 mimic #1
YTYFTTVTVE

SEQ ID NO: 45 - protein, 10 aa, synthetic construct
  source 1..10: note = LRRC4 mimic #2
YSFFTTVTVE

SEQ ID NO: 46 - protein, 10 aa, synthetic construct
  source 1..10: note = LRRC4 mimic #3
FSYFSTVTVE

SEQ ID NO: 47 - protein, 20 aa, synthetic construct
  source 1..20: note = LRRC4 mimic #4
GYTYFTTVTVETLETQPGEE

SEQ ID NO: 48 - protein, 16 aa, synthetic construct
  source 1..16: note = LRRC4 mimic #5
GYTYFTTVTVETLETQ

SEQ ID NO: 49 - protein, 28 aa, synthetic construct
  source 1..28: note = LRRC4 mimic #6
GYTYFTTVTVETLETQPGEKEPPGPTTD

SEQ ID NO: 50 - protein, 21 aa, synthetic construct
  source 1..21: note = LRRC4 mimic #7
GYTYFTTVTVETLETQPGEEA

SEQ ID NO: 51 - protein, 20 aa, synthetic construct
  source 1..20: note = LRRC4 mimic #8
GYTYFTTVTVEPYETQPGEE

SEQ ID NO: 52 - protein, 20 aa, synthetic construct
  source 1..20: note = LRRC4 mimic #9
GYTYFTTVTVEMRETQPGEE

SEQ ID NO: 53 - protein, 20 aa, synthetic construct
  source 1..20: note = LRRC4 mimic #10
GYTYFTTVTVEIFETQPGEE

SEQ ID NO: 54 - protein, 20 aa, synthetic construct
  source 1..20: note = LRRC4 mimic #11
GYTYFTTVTVEHFETQPGEE

SEQ ID NO: 55 - protein, 20 aa, synthetic construct
  source 1..20: note = LRRC4 mimic #12
GYTYFTTVTVEWYETQPGEE

SEQ ID NO: 56 - protein, 20 aa, synthetic construct
  source 1..20: note = LRRC4 mimic #13
GYTYFTTVTVEQRETQPGEE

SEQ ID NO: 57 - protein, 20 aa, synthetic construct
  source 1..20: note = LRRC4 mimic #14
GYTYFTTVTVEWFETQPGEE

SEQ ID NO: 58 - protein, 20 aa, synthetic construct
  source 1..20: note = LRRC4 mimic #15
GYTYFTTVTVEERETQPGEE

SEQ ID NO: 59 - protein, 20 aa, synthetic construct
  source 1..20: note = LRRC4 mimic #16
GYTYFTTVTVEDYETQPGEE

SEQ ID NO: 60 - protein, 20 aa, synthetic construct
  source 1..20: note = LRRC4 mimic #17
GYTYFTTVTVEFFETQPGEE

SEQ ID NO: 61 - protein, 20 aa, synthetic construct
  source 1..20: note = LRRC4 mimic #18
GYTYFTTVTVEHYETQPGEE

SEQ ID NO: 62 - protein, 20 aa, synthetic construct
  source 1..20: note = LRRC4 mimic #19
GYTYFTTVTVEMMETQPGEE

SEQ ID NO: 63 - protein, 20 aa, synthetic construct
  source 1..20: note = LRRC4 mimic #20
GYTYFTTVTVEDFETQPGEE

SEQ ID NO: 64 - protein, 20 aa, synthetic construct
  source 1..20: note = LRRC4 mimic #21
GYTYFTTVTVEDIETQPGEE

SEQ ID NO: 65 - protein, 20 aa, synthetic construct
  source 1..20: note = LRRC4 mimic #22
GYTYFTTVTVELIETQPGEE

SEQ ID NO: 66 - protein, 20 aa, synthetic construct
  source 1..20: note = LRRC4 mimic #23
GYTYFTTVTVEEIETQPGEE

SEQ ID NO: 67 - protein, 20 aa, synthetic construct
  source 1..20: note = LRRC4 mimic #24
GYTYFTTVTVEAFETQPGEE

SEQ ID NO: 68 - protein, 20 aa, synthetic construct
  source 1..20: note = LRRC4 mimic #25
GYTYFTTVTVEHHETQPGEE

SEQ ID NO: 69 - protein, 20 aa, synthetic construct
  source 1..20: note = LRRC4 mimic #26
GYTYFTTVTVEPFETQPGEE

SEQ ID NO: 70 - protein, 20 aa, synthetic construct
  source 1..20: note = LRRC4 mimic #27
GYTYFTTVTVEDWETQPGEE

SEQ ID NO: 71 - protein, 21 aa, synthetic construct
  source 1..21: note = LRRC4 mimic #28
GYTYFTTVTVEPYETQPGEEA

SEQ ID NO: 72 - protein, 21 aa, synthetic construct
  source 1..21: note = LRRC4 mimic #29
GYTYFTTVTVEHFETQPGEEA

SEQ ID NO: 73 - protein, 21 aa, synthetic construct
  source 1..21: note = LRRC4 mimic #30
GYTYFTTVTVEQRETQPGEEA

SEQ ID NO: 74 - protein, 21 aa, synthetic construct
  source 1..21: note = LRRC4 mimic #31
GYTYFTTVTVEWYETQPGEEA

SEQ ID NO: 75 - protein, 21 aa, synthetic construct
  source 1..21: note = LRRC4 mimic #32
GYTYFTTVTVEMRETQPGEEA

SEQ ID NO: 76 - protein, 21 aa, synthetic construct
  source 1..21: note = LRRC4 mimic #33
GYTYFTTVTVEIFETQPGEEA

SEQ ID NO: 77 - protein, 23 aa, synthetic construct
  source 1..23: note = LRRC4 mimic #34
NYSFFTTVTVETTEISPEDTTRK

SEQ ID NO: 78 - protein, 23 aa, synthetic construct
  source 1..23: note = LRRC4 mimic #35
NFSYFSTVTVETMEPSQDERTTR

SEQ ID NO: 79 - protein, 9 aa, synthetic construct
  source 1..9: note = LRRC4 mimic #36
LDEVMKTTK

SEQ ID NO: 80 - protein, 9 aa, synthetic construct
  source 1..9: note = LRRC4 mimic #37
IDEVMKTTK

SEQ ID NO: 81 - skipped

SEQ ID NO: 82 - protein, 640 aa, synthetic construct
  source 1..640: note = Human LRRC4C protein
MLNKMTLHPQQIMIGPRFNRALFDPLLVVLLALQLLVVAGLVRAQTCPSVCSCSNQFSKVICVRKNLREVPDGISTNTRLLNLHENQIQIIKVNSFKHLRHLEILQLSRNHIRTIEIGAFNGLANLNTLELFDNRLTTIPNGAFVYLSKLKELWLRNNPIESIPSYAFNRIPSLRRLDLGELKRLSYISEGAFEGLSNLRYLNLAMCNLREIPNLTPLIKLDELDLSGNHLSAIRPGSFQGLMHLQKLWMIQSQIQVIERNAFDNLQSLVEINLAHNNLTLLPHDLFTPLHHLERIHLHHNPWNCNCDILWLSWWIKDMAPSNTACCARCNTPPNLKGRYIGELDQNYFTCYAPVIVEPPADLNVTEGMAAELKCRASTSLTSVSWITPNGTVMTHGAYKVRIAVLSDGTLNFTNVTVQDTGMYTCMVSNSVGNTTASATLNVTAATTTPFSYFSTVTVETMEPSQDEARTTDNNVGPTPVVDWETTNVTTSLTPQSTRSTEKTFTIPVTDINSGIPGIDEVMKTTKIIIGCFVAITLMAAVMLVIFYKMRKQHHRQNHHAPTRTVEIINVDDEITGDTPMESHLPMPAIEHEHLNHYNSYKSPFNHTTTVNTINSIHSSVHEPLLIRMNSKDNVQETQI

SEQ ID NO: 83 - protein, 44 aa, synthetic construct
  source 1..44: note = Human LRRC4C protein signal peptide
MLNKMTLHPQQIMIGPRFNRALFDPLLVVLLALQLLVVAGLVRA

SEQ ID NO: 84 - protein, 483 aa, synthetic construct
  source 1..483: note = Human LRRC4C ectodomain protein
QTCPSVCSCSNQFSKVICVRKNLREVPDGISTNTRLLNLHENQIQIIKVNSFKHLRHLEILQLSRNHIRTIEIGAFNGLANLNTLELFDNRLTTIPNGAFVYLSKLKELWLRNNPIESIPSYAFNRIPSLRRLDLGELKRLSYISEGAFEGLSNLRYLNLAMCNLREIPNLTPLIKLDELDLSGNHLSAIRPGSFQGLMHLQKLWMIQSQIQVIERNAFDNLQSLVEINLAHNNLTLLPHDLFTPLHHLERIHLHHNPWNCNCDILWLSWWIKDMAPSNTACCARCNTPPNLKGRYIGELDQNYFTCYAPVIVEPPADLNVTEGMAAELKCRASTSLTSVSWITPNGTVMTHGAYKVRIAVLSDGTLNFTNVTVQDTGMYTCMVSNSVGNTTASATLNVTAATTTPFSYFSTVTVETMEPSQDEARTTDNNVGPTPVVDWETTNVTTSLTPQSTRSTEKTFTIPVTDINSGIPGIDEVMKTTK

SEQ ID NO: 85-88 - skipped (4 entries)

SEQ ID NO: 89 - protein, 5 aa, synthetic construct
  source 1..5: note = P2-C12 VH-CDR1
TYAVT

SEQ ID NO: 90 - protein, 16 aa, synthetic construct
  source 1..16: note = P2-C12 VH-CDR2
YINWRGGTSYANWAKG

SEQ ID NO: 91 - protein, 15 aa, synthetic construct
  source 1..15: note = P2-C12 VH-CDR3
DASSGAAFGSYGMDP

SEQ ID NO: 92 - protein, 11 aa, synthetic construct
  source 1..11: note = P2-C12 VL-CDR1
QASQSISSYLS

SEQ ID NO: 93 - protein, 7 aa, synthetic construct
  source 1..7: note = P2-C12 VL-CDR2
EASKLAS

SEQ ID NO: 94 - protein, 12 aa, synthetic construct
  source 1..12: note = P2-C12 VL-CDR3
QQGYSSTNVWNA

SEQ ID NO: 95 - protein, 6 aa, synthetic construct
  source 1..6: note = 13B4 VH-CDR1
SSNWWS

SEQ ID NO: 96 - protein, 16 aa, synthetic construct
  source 1..16: note = 13B4 VH-CDR2
EIYHGGTTNYNPSLKG

SEQ ID NO: 97 - protein, 9 aa, synthetic construct
  source 1..9: note = 13B4 VH-CDR3
WQLVGGLDV

SEQ ID NO: 98 - protein, 11 aa, synthetic construct
  source 1..11: note = 13B4 VL-CDR1
SGDKLGNVYAS

SEQ ID NO: 99 - protein, 7 aa, synthetic construct
  source 1..7: note = 13B4 VL-CDR2
QDNKRPS

SEQ ID NO: 100 - protein, 9 aa, synthetic construct
  source 1..9: note = 13B4 VL-CDR3
QAWDSSTAV

SEQ ID NO: 101 - protein, 5 aa, synthetic construct
  source 1..5: note = 13F7 VH-CDR1
GYSWT

SEQ ID NO: 102 - protein, 16 aa, synthetic construct
  source 1..16: note = 13F7 VH-CDR2
EISHFGSANYNPSLKS

SEQ ID NO: 103 - protein, 15 aa, synthetic construct
  source 1..15: note = 13F7 VH-CDR3
ALRGTYSRFYYGMDV

SEQ ID NO: 104 - protein, 16 aa, synthetic construct
  source 1..16: note = 13F7 VL-CDR1
RSSQSLLHSNGYNYLD

SEQ ID NO: 105 - protein, 7 aa, synthetic construct
  source 1..7: note = 13F7 VL-CDR2
LGSNRAS

SEQ ID NO: 106 - protein, 9 aa, synthetic construct
  source 1..9: note = 13F7 VL-CDR3
MQARQTPLT

SEQ ID NO: 107 - protein, 5 aa, synthetic construct
  source 1..5: note = 15A9 VH-CDR1
SYYWS

SEQ ID NO: 108 - protein, 16 aa, synthetic construct
  source 1..16: note = 15A9 VH-CDR2
YIYPSGSTNYNPSLKS

SEQ ID NO: 109 - protein, 12 aa, synthetic construct
  source 1..12: note = 15A9 VH-CDR3
VNPFGYYYAMDV

SEQ ID NO: 110 - protein, 11 aa, synthetic construct
  source 1..11: note = 15A9 VL-CDR1
RASQSISTSLN

SEQ ID NO: 111 - protein, 7 aa, synthetic construct
  source 1..7: note = 15A9 VL-CDR2
GASTLQS

SEQ ID NO: 112 - protein, 9 aa, synthetic construct
  source 1..9: note = 15A9 VL-CDR3
QESASIPRT

SEQ ID NO: 113 - protein, 5 aa, synthetic construct
  source 1..5: note = P1-A03 VH-CDR1
SDYMS

SEQ ID NO: 114 - protein, 16 aa, synthetic construct
  source 1..16: note = P1-A03 VH-CDR2
IIYPSTTTYYASWAKG

SEQ ID NO: 115 - protein, 10 aa, synthetic construct
  source 1..10: note = P1-A03 VH-CDR3
GSNWSSGMNL

SEQ ID NO: 116 - protein, 11 aa, synthetic construct
  source 1..11: note = P1-A03 VL-CDR1
LASEDIYSGIS

SEQ ID NO: 117 - protein, 7 aa, synthetic construct
  source 1..7: note = P1-A03 VL-CDR2
GASNLES

SEQ ID NO: 118 - protein, 12 aa, synthetic construct
  source 1..12: note = P1-A03 VL-CDR3
LGGYSYSSTGLT

SEQ ID NO: 119 - protein, 5 aa, synthetic construct
  source 1..5: note = P1-A08 VH-CDR1
TYYMS

SEQ ID NO: 120 - protein, 16 aa, synthetic construct
  source 1..16: note = P1-A08 VH-CDR2
IVYPSGTTYYANWAKG

SEQ ID NO: 121 - protein, 8 aa, synthetic construct
  source 1..8: note = P1-A08 VH-CDR3
GDSFGYGL

SEQ ID NO: 122 - protein, 11 aa, synthetic construct
  source 1..11: note = P1-A08 VL-CDR1
TADTLSRSYAS

SEQ ID NO: 123 - protein, 7 aa, synthetic construct
  source 1..7: note = P1-A08 VL-CDR2
RDTSRPS

SEQ ID NO: 124 - protein, 13 aa, synthetic construct
  source 1..13: note = P1-A08 VL-CDR3
ATSDGSGSNYQYV

SEQ ID NO: 125 - protein, 5 aa, synthetic construct
  source 1..5: note = P1-F02 VH-CDR1
NYYMG

SEQ ID NO: 126 - protein, 16 aa, synthetic construct
  source 1..16: note = P1-F02 VH-CDR2
IIYASGSTYYASWAKG

SEQ ID NO: 127 - protein, 17 aa, synthetic construct
  source 1..17: note = P1-F02 VH-CDR3
IDIGVGDYGWAYDRLDL

SEQ ID NO: 128 - protein, 11 aa, synthetic construct
  source 1..11: note = P1-F02 VL-CDR1
LASEDIYSGIS

SEQ ID NO: 129 - protein, 7 aa, synthetic construct
  source 1..7: note = P1-F02 VL-CDR2
GASNLES

SEQ ID NO: 130 - protein, 10 aa, synthetic construct
  source 1..10: note = P1-F02 VL-CDR3
LGGYSYSSIT

SEQ ID NO: 131 - protein, 5 aa, synthetic construct
  source 1..5: note = P2-A01 VH-CDR1
GYYMS

SEQ ID NO: 132 - protein, 16 aa, synthetic construct
  source 1..16: note = P2-A01 VH-CDR2
IIYPSGSTDYASWAKG

SEQ ID NO: 133 - protein, 15 aa, synthetic construct
  source 1..15: note = P2-A01 VH-CDR3
VAGYVGYGYETFFDI

SEQ ID NO: 134 - protein, 11 aa, synthetic construct
  source 1..11: note = P2-A01 VL-CDR1
LASEDIYSGIS

SEQ ID NO: 135 - protein, 7 aa, synthetic construct
  source 1..7: note = P2-A01 VL-CDR2
GASNLES

SEQ ID NO: 136 - protein, 14 aa, synthetic construct
  source 1..14: note = P2-A01 VL-CDR3
LGGVTYSSTGTHLT

SEQ ID NO: 137 - protein, 5 aa, synthetic construct
  source 1..5: note = P2-A03 VH-CDR1
NYDMS

SEQ ID NO: 138 - protein, 16 aa, synthetic construct
  source 1..16: note = P2-A03 VH-CDR2
FMDTDGSAYYATWAKG

SEQ ID NO: 139 - protein, 11 aa, synthetic construct
  source 1..11: note = P2-A03 VH-CDR3
RGSSYYGGIDI

SEQ ID NO: 140 - protein, 11 aa, synthetic construct
  source 1..11: note = P2-A03 VL-CDR1
QASQSIGGNLA

SEQ ID NO: 141 - protein, 7 aa, synthetic construct
  source 1..7: note = P2-A03 VL-CDR2
RASTLAS

SEQ ID NO: 142 - protein, 14 aa, synthetic construct
  source 1..14: note = P2-A03 VL-CDR3
QSPAYDPAAYVGNA

SEQ ID NO: 143 - protein, 5 aa, synthetic construct
  source 1..5: note = P2-F07 VH-CDR1
SYYMN

SEQ ID NO: 144 - protein, 16 aa, synthetic construct
  source 1..16: note = P2-F07 VH-CDR2
IIYPSGTTYYAGWAKG

SEQ ID NO: 145 - protein, 8 aa, synthetic construct
  source 1..8: note = P2-F07 VH-CDR3
TVSGYFDI

SEQ ID NO: 146 - protein, 11 aa, synthetic construct
  source 1..11: note = P2-F07 VL-CDR1
LASEDIYSALA

SEQ ID NO: 147 - protein, 7 aa, synthetic construct
  source 1..7: note = P2-F07 VL-CDR2
GTSNLES

SEQ ID NO: 148 - protein, 9 aa, synthetic construct
  source 1..9: note = P2-F07 VL-CDR3
QGYSSYPLT

SEQ ID NO: 149 - protein, 5 aa, synthetic construct
  source 1..5: note = P2-F11 VH-CDR1
SYGVS

SEQ ID NO: 150 - protein, 16 aa, synthetic construct
  source 1..16: note = P2-F11 VH-CDR2
YIANNYNPHYASWAKG

SEQ ID NO: 151 - protein, 7 aa, synthetic construct
  source 1..7: note = P2-F11 VH-CDR3
DNYGMDP

SEQ ID NO: 152 - protein, 13 aa, synthetic construct
  source 1..13: note = P2-F11 VL-CDR1
QASQSVYNNKNLA

SEQ ID NO: 153 - protein, 7 aa, synthetic construct
  source 1..7: note = P2-F11 VL-CDR2
AASTLAS

SEQ ID NO: 154 - protein, 13 aa, synthetic construct
  source 1..13: note = P2-F11 VL-CDR3
QGEFSCSSADCNA

SEQ ID NO: 155 - protein, 121 aa, synthetic construct
  source 1..121: note = P2-C12 VH
QSLEESGGRLVTPGTPLTLTCTVSGFSLSTYAVTWVRQAPGKGLEWIGYINWRGGTSYANWAKGRFTISKTSSTTVDLKMTSPTTEDTATYFCARDASSGAAFGSYGMDPWGPGTLVTVSS

SEQ ID NO: 156 - protein, 118 aa, synthetic construct
  source 1..118: note = 13B4 VH
QVQLQESGPGLVKPSGTLSLNCAVSGGSISSSNWWSWVRQPPGKGLEWIGEIYHGGTTNYNPSLKGRVTMSVDKTKNQFSLRLSSVTAVDTAVYYCARWQLVGGLDVWGQGTTVTVSS

SEQ ID NO: 157 - protein, 123 aa, synthetic construct
  source 1..123: note = 13F7 VH
QVQLQEWGAGLLKPSETLSLTCAINAESFNGYSWTWIRQTPGKGLEWIGEISHFGSANYNPSLKSRATISADKSKNQFSLKLTSVTAVDTAVYYCARALRGTYSRFYYGMDVWGQGTTVTVSS

SEQ ID NO: 158 - protein, 120 aa, synthetic construct
  source 1..120: note = 15A9 VH
QVQLQESGPGLVKPSETLSLTCTVSGGSISSYYWSWIRQPPGKGLEWIGYIYPSGSTNYNPSLKSRVTISVDTSKNQFSLNLKSVTAVDTAVYYCARVNPFGYYYAMDVWGQGTTVTVSS

SEQ ID NO: 159 - protein, 116 aa, synthetic construct
  source 1..116: note = P1-A03 VH
QSVEESGGRLVTPGTPLTLTCTVSGFSLSSDYMSWVRQAPGEGLEWIGIIYPSTTTYYASWAKGRFTISKTSSTTVELKMTSLTTEDTATYFCARGSNWSSGMNLWGPGTLVTVSS

SEQ ID NO: 160 - protein, 113 aa, synthetic construct
  source 1..113: note = P1-A08 VH
QSLEESGGRLVTPGTPLTLTCTASGFSLSTYYMSWVRQAPGKGLEWIGIVYPSGTTYYANWAKGRFTISTASTTVDLMITSPTTEDTATYFCARGDSFGYGLWGPGTLVTVSS

SEQ ID NO: 161 - protein, 122 aa, synthetic construct
  source 1..122: note = P1-F02 VH
QSLEESGGRLVTPGTPLTLTCTASGFSLSNYYMGWVRQAPGEGLEWIGIIYASGSTYYASWAKGRFTISKTSTTVDLKMTSLTTEDTATYFCARIDIGVGDYGWAYDRLDLWGQGTLVTVSS

SEQ ID NO: 162 - protein, 121 aa, synthetic construct
  source 1..121: note = P2-A01 VH
QEQLVESGGRLVTPGTPLTLSCTASGFFLSGYYMSWVRQAPGKGLEWIGIIYPSGSTDYASWAKGRFTISKTSTTVDLKITTPTTEDTATYFCARVAGYVGYGYETFFDIWGPGTLVTVSL

SEQ ID NO: 163 - protein, 116 aa, synthetic construct
  source 1..116: note = P2-A03 VH
QSVEESGGRLVTPGTPLTLTCTVSGFSLNNYDMSWVRQAPGKGLEYIGFMDTDGSAYYATWAKGRFTISRTSTTVDLKMTSPTTEDTATYFCARRGSSYYGGIDIWGPGTPVTVSL

SEQ ID NO: 164 - protein, 113 aa, synthetic construct
  source 1..113: note = P2-F07 VH
QSLEESGGRLVTPGTPLTLTCTASGFSLSSYYMNWVRQAPGKGLEWIGIIYPSGTTYYAGWAKGRFTISKTSTTVDLKITSPTSEDTATYFCARTVSGYFDIWGPGTLVTVSL

SEQ ID NO: 165 - protein, 114 aa, synthetic construct
  source 1..114: note = P2-F11 VH
QEQLVESGGRLVTPGTTLTLTCTVSGFSLSSYGVSWVRQAPGKGLEWIGYIANNYNPHYASWAKGRFTISKTSSTTVDLKMTSLTTEDTATYFCARDNYGMDPWGPGTLVTVSS

SEQ ID NO: 166 - protein, 110 aa, synthetic construct
  source 1..110: note = P2-C12 VL
ELDMTQTPSSVSAAVGGTVTIKCQASQSISSYLSWYQQKPGQPPKLLIYEASKLASGVPSRFSGSGYGTEFTLTISDLECADAATYYCQQGYSSTNVWNAFGGGTNVEIK

SEQ ID NO: 167 - protein, 106 aa, synthetic construct
  source 1..106: note = 13B4 VL
SYELTQPLSVSVSPGQTASITCSGDKLGNVYASWYQQKPGQSPTLVIYQDNKRPSGIPERFSGSNSGKTATLTISGTQALDEADYYCQAWDSSTAVFGGGTKLTVL

SEQ ID NO: 168 - protein, 112 aa, synthetic construct
  source 1..112: note = 13F7 VL
DIVMTQTPLSLPVAPGEPASISCRSSQSLLHSNGYNYLDWYVQKPGQPPQLLIYLGSNRASGVPDRFSGSGSGTDFTLKISRVEAEDVGVYYCMQARQTPLTFGGGTKVEIK

SEQ ID NO: 169 - protein, 107 aa, synthetic construct
  source 1..107: note = 15A9 VL
DIQMTQSPSSLSASVGDRITISCRASQSISTSLNWYQQTPGKAPRLLIYGASTLQSGVPSRFSGGGSGTDFSLTITSLQPEDFATYYCQESASIPRTFGQGTKLDIK

SEQ ID NO: 170 - protein, 110 aa, synthetic construct
  source 1..110: note = P1-A03 VL
ELVMTQTPPSLSASVGETVRIRCLASEDIYSGISWYQQKPEKPPTLLISGASNLESGVPPRFSGSGSGTDYTLTIGGVQAEDAATYYCLGGYSYSSTGLTFGAGTNVEIK

SEQ ID NO: 171 - protein, 110 aa, synthetic construct
  source 1..110: note = P1-A08 VL
ELVLTQSPSVQVNLGQTVSLTCTADTLSRSYASWYQQKPGQAPVLLIYRDTSRPSGVPDRFSGSSSGNTATLTISGAQAGDEADYYCATSDGSGSNYQYVFGGGTQLTVT

SEQ ID NO: 172 - protein, 108 aa, synthetic construct
  source 1..108: note = P1-F02 VL
ELDMTQTPPSLSASVGETVRIRCLASEDIYSGISWYQQKPGKPPTLLIYGASNLESGVPPRFSGSGSGTDYTLTIGGVQAEDAATYYCLGGYSYSSITFGAGTNVEIK

SEQ ID NO: 173 - protein, 112 aa, synthetic construct
  source 1..112: note = P2-A01 VL
ELVMTQTPPSLSASVGETVRIRCLASEDIYSGISWYQQKPGKPPTLLIYGASNLESGVPPRFSGSGSGSDYTLTIGGVQAEDAATYYCLGGVTYSSTGTHLTFGAGTNVEIK

SEQ ID NO: 174 - protein, 112 aa, synthetic construct
  source 1..112: note = P2-A03 VL
ELDLTQTPASVSEPVGGTVTIKCQASQSIGGNLAWYQQKPGQPPKLLIYRASTLASGVPSRFKGSGSGTDFTLTISDLECADAATYYCQSPAYDPAAYVGNAFGGGTELEIL

SEQ ID NO: 175 - protein, 107 aa, synthetic construct
  source 1..107: note = P2-F07 VL
ELDLTQTPPSLSASVGGTVTINCLASEDIYSALAWYQQKPGKPPTLLISGTSNLESGVPPRFSGSGSGTDYTLTIGGVQAEDAATYFCQGYSSYPLTFGAGTNVEIK

SEQ ID NO: 176 - protein, 113 aa, synthetic construct
  source 1..113: note = P2-F11 VL
ELDLTQTPSSVSAAVGGTVTINCQASQSVYNNKNLAWYQQKPGQPPKLLIYAASTLASGVSSRFKGSGSGTQFTLTISDVQCDDAATYYCQGEFSCSSADCNAFGGGTELEIL

SEQ ID NO: 177 - skipped

SEQ ID NO: 178 - protein, 14 aa, synthetic construct
  source 1..14: note = F1 Epitope
QLAAGTCEIVTLDR

SEQ ID NO: 179 - protein, 20 aa, synthetic construct
  source 1..20: note = F2 Epitope
TLDRDSSQPRRTIARQTARC

SEQ ID NO: 180 - protein, 20 aa, synthetic construct
  source 1..20: note = F3 Epitope
TARCACRKGQIAGTTRARPA

SEQ ID NO: 181 - protein, 20 aa, synthetic construct
  source 1..20: note = F4 Epitope
ARPACVDARIIKTKQWCDML

SEQ ID NO: 182 - protein, 20 aa, synthetic construct
  source 1..20: note = F5 Epitope
CDMLPCLEGEGCDLLINRSG

SEQ ID NO: 183 - protein, 20 aa, synthetic construct
  source 1..20: note = F6 Epitope
NRSGWTCTQPGGRIKTTTVS

SEQ ID NO: 184 - protein, 5 aa, synthetic construct
  source 1..5: note = EP1 Epitope
IVTLD

SEQ ID NO: 185 - protein, 5 aa, synthetic construct
  source 1..5: note = EP2 Epitope
DSSQP

SEQ ID NO: 186 - protein, 5 aa, synthetic construct
  source 1..5: note = EP3 Epitope
RTIAR

SEQ ID NO: 187 - protein, 7 aa, synthetic construct
  source 1..7: note = EP4 Epitope
ARCACRK

SEQ ID NO: 188 - protein, 4 aa, synthetic construct
  source 1..4: note = EP5 Epitope
ARPA

SEQ ID NO: 189 - protein, 9 aa, synthetic construct
  source 1..9: note = EP6 Epitope
KTKQWCDML

SEQ ID NO: 190 - protein, 8 aa, synthetic construct
  source 1..8: note = EP7 Epitope
GCDLLINR

SEQ ID NO: 191 - protein, 8 aa, synthetic construct
  source 1..8: note = EP8 Epitope
TCTQPGGR

SEQ ID NO: 192 - protein, 9 aa, synthetic construct
  source 1..9: note = Epitope Fragment #1
DRDSSQPRR

SEQ ID NO: 193 - protein, 5 aa, synthetic construct
  source 1..5: note = Epitope Fragment #2
RDPRR

SEQ ID NO: 194 - protein, 4 aa, synthetic construct
  source 1..4: note = Epitope Fragment #3
RPRR

SEQ ID NO: 195 - skipped

SEQ ID NO: 196 - protein, 6 aa, synthetic construct
  source 1..6: note = Epitope Fragment #5
DSSQPR

SEQ ID NO: 197 - skipped

SEQ ID NO: 198 - protein, 4 aa, synthetic construct
  source 1..4: note = Epitope Fragment #7
DSSP

SEQ ID NO: 199 - protein, 13 aa, synthetic construct
  source 1..13: note = Epitope Fragment #8
CRKGQIAGTTRAR

SEQ ID NO: 200 - protein, 14 aa, synthetic construct
  source 1..14: note = Epitope Fragment #9
PACVDARIIKTKQW

SEQ ID NO: 201-207 - skipped (7 entries)

SEQ ID NO: 208 - protein, 11 aa, synthetic construct
  source 1..11: note = SS01-13 VL-CDR1, SS01-13-S5 VL-CDR1, S5-2.GKNG VL-CDR1
SGGASSGYGYG

SEQ ID NO: 209 - protein, 7 aa, synthetic construct
  source 1..7: note = SS01-13 VL-CDR2
KDDERPS

SEQ ID NO: 210 - skipped

SEQ ID NO: 211 - protein, 122 aa, synthetic construct
  source 1..122: note = SS01-13 VH
AVTLDESGGGLQTPGGALSLSCKASGFTFSSYQMGWVRQAPGKGLEWVGVINKSGSDTSYGSAVKGRATISRDNGQSTLYLQMNNLRAEDTAVYFCAKGSASYITAATIDAWGHGTEVIVSS

SEQ ID NO: 212 - protein, 109 aa, synthetic construct
  source 1..109: note = SS01-13 VL
ALTQPSSVSANPGETVRITCSGGASSGYGYGWYQQKPSSAPLTVIYKDDERPSDIPSRFSGSSSGSTHTLTITGVQAEDEAVYFCGNDDYSSDSGYVGVFGAGTTLTVL

SEQ ID NO: 213 - protein, 7 aa, synthetic construct
  source 1..7: note = SS01-13-S5 VL-CDR2, S5-2.GKNG VL-CDR2
KDSERPS

SEQ ID NO: 214 - protein, 122 aa, synthetic construct
  source 1..122: note = SS01-13-s5 VH
AVTLDESGGGLQTPGGALRLSCKASGFTFSSYQMGWVRQAPGKGLEWVSAINKSGSDTSYGSAVKGRATISRDNGQSTLYLQMNSLRAEDTAVYFCAKGSASYITAATIDAWGHGTEVIVSS

SEQ ID NO: 215 - protein, 109 aa, synthetic construct
  source 1..109: note = SS01-13-s5 VL, S5-2.GKNG VL
ALTQPSSVSANPGETARITCSGGASSGYGYGWYQQKPSSAPLTVIYKDSERPSDIPSRFSGSSSGSTHTLTISGVQAEDEAVYFCGNDDYSSDSGYVGVFGAGTTLTVL

SEQ ID NO: 216-217 - skipped (2 entries)

SEQ ID NO: 218 - protein, 10 aa, synthetic construct
  source 1..10: note = 1-7A-IT VH-CDR1
GFTFSSFNMF

SEQ ID NO: 219 - protein, 17 aa, synthetic construct
  source 1..17: note = 1-7A-IT VH-CDR2
QISSSGSSTNYAPAVKG

SEQ ID NO: 220 - protein, 10 aa, synthetic construct
  source 1..10: note = Low-PI VH-CDR1, 1-30 VH-CDR1, 1-17 VH-CDR1, 1-32 VH-CDR1, 4-11 VH-CDR1, 6-10 VH-CDR1
GFDFESFNMF

SEQ ID NO: 221 - protein, 17 aa, synthetic construct
  source 1..17: note = Low-PI VH-CDR1, 1-30 VH-CDR1, 1-17 VH-CDR1, 1-32 VH-CDR1, 4-11 VH-CDR1, 6-10 VH-CDR1
QISSSEEDENYAPAVKG

SEQ ID NO: 222 - protein, 10 aa, synthetic construct
  source 1..10: note = 2-13D VH-CDR1
GFTFSSHGMF

SEQ ID NO: 223 - protein, 10 aa, synthetic construct
  source 1..10: note = 2-13D-37 VH-CDR1, 2-13D-37-1.5W-41 VH-CDR1, 2-13D-37-3W-16 VH-CDR1
GFDFSSHGMF

SEQ ID NO: 224 - protein, 7 aa, synthetic construct
  source 1..7: note = 1-7A-IT VL-CDR2
ENNKRPS

SEQ ID NO: 225 - protein, 13 aa, synthetic construct
  source 1..13: note = Low-PI VL-CDR1, 1-30 VL-CDR1
SGGGSEEEQYYYG

SEQ ID NO: 226 - protein, 7 aa, synthetic construct
  source 1..7: note = Low-PI VL-CDR2
EDEERPS

SEQ ID NO: 227 - protein, 10 aa, synthetic construct
  source 1..10: note = Low-PI VL-CDR3 1-30 VL-CDR3 1-17 VL-CDR3, 1-32 VL-CDR3
GSWDSEDEDH

SEQ ID NO: 228 - protein, 7 aa, synthetic construct
  source 1..7: note = 1-30 VL-CDR2, 1-32 VL-CDR2
QDEERPS

SEQ ID NO: 229 - protein, 7 aa, synthetic construct
  source 1..7: note = 1-17 VL-CDR2
EDEQRPS

SEQ ID NO: 230 - protein, 7 aa, synthetic construct
  source 1..7: note = 4-11 VL-CDR2
EDHERPS

SEQ ID NO: 231 - protein, 10 aa, synthetic construct
  source 1..10: note = 4-11 VL-CDR3, 6-10 VL-CDR3
GSWDSSDEDH

SEQ ID NO: 232 - protein, 7 aa, synthetic construct
  source 1..7: note = 6-10 VL-CDR2
QDLLRPS

SEQ ID NO: 233 - skipped

SEQ ID NO: 234 - protein, 131 aa, synthetic construct
  source 1..131: note = 1-7A-IT VH
AVTLDESGGGLQTPGGALRLSCKASGFTFSSFNMFWVRQAPGKGLEYVSQISSSGSSTNYAPAVKGRATISRDNGQSTLYLQMNSLRAEDTGTYYCAKSSYDCPYGHCSSGVDSAGEIDAWGHGTEVIVSS

SEQ ID NO: 235 - protein, 131 aa, synthetic construct
  source 1..131: note = Low-PI VH, 1-30 VH, 1-17 VH, 1-32 VH, 4-11 VH, 6-10 VH
AVTLDESGGGLQTPGGALRLSCKASGFDFESFNMFWVRQAPGKGLEYVSQISSSEEDENYAPAVKGRATISRDNGQSTLYLQMNSLRAEDTGTYYCAKSSYDCPYGHCSSGVDSAGEIDAWGHGTEVIVSS

SEQ ID NO: 236 - protein, 129 aa, synthetic construct
  source 1..129: note = 2-13D VH
AVTLDESGGGLQTPGGALRLSCSASGFTFSSHGMFWVRQAPGKGLEYVSEITNDGSGTNYGSAVKGRATISRDNGQSTLYLQMNSLRAEDTGTYFCARSTYECPGGFSCWGDTGQIDAWGHGTEVIVSS

SEQ ID NO: 237 - protein, 129 aa, synthetic construct
  source 1..129: note = 2-13D-37-1.5W-41 VH
AVTLDESGGGLQTPGGALRLSCSASGFDFSSHGMFWVRQAPGKGLEYVSEITNDGSGTNYGSAVKGRATISRDNGQSTLYLQMNSLRAEDTGTYFCARSSYVCPGGFSCWGDTGQIDAWGHGTEVIVSS

SEQ ID NO: 238 - protein, 107 aa, synthetic construct
  source 1..107: note = 1-7A-IT VL
ALTQPSSVSANPGETVKITCSGGGSYAGSYYYGWYQQKPGSAPVTLIYENNKRPSDIPSRFSGSTSGSTATLTITGVQAGDEADYYCGSWDSSNGGIFGAGTTLTVL

SEQ ID NO: 239 - protein, 107 aa, synthetic construct
  source 1..107: note = Low-PI VL
ALTQPSSVSANPGETVKITCSGGGSEEEQYYYGWYQQKPGSAPVTLIYEDEERPSDIPSRFSGSTSGSTATLTITGVQAGDEADYYCGSWDSEDEDHFGAGTTLTVL

SEQ ID NO: 240 - protein, 107 aa, synthetic construct
  source 1..107: note = 1-30 VL
ALTQPSSVSANPGETVKITCSGGGSEEEQYYYGWYQQKPGSAPVTLIYQDEERPSDIPSRFSGSTSGSTATLTITGVQAGDEADYYCGSWDSEDEDHFGAGTTLTVL

SEQ ID NO: 241 - protein, 107 aa, synthetic construct
  source 1..107: note = 1-17 VL
ALTQPSSVSANPGETVKITCSGGGSYAGSYYYGWYQQKPGSAPVTLIYEDEQRPSDIPSRFSGSTSGSTATLTITGVQAGDEADYYCGSWDSEDEDHFGAGTTLTVL

SEQ ID NO: 242 - protein, 107 aa, synthetic construct
  source 1..107: note = 1-32 VL
ALTQPSSVSANPGETVKITCSGGGSYAGSYYYGWYQQKPGSAPVTLIYQDEERPSDIPSRFSGSTSGSTATLTITGVQAGDEADYYCGSWDSEDEDHFGAGTTLTVL

SEQ ID NO: 243 - protein, 107 aa, synthetic construct
  source 1..107: note = 4-11 VL
ALTQPSSVSANPGETVKITCSGGGSYAGSYYYGWYQQKPGSAPVTLIYEDHERPSDIPSRFSGSTSGSTATLTITGVQAGDEADYYCGSWDSSDEDHFGAGTTLTVL

SEQ ID NO: 244 - protein, 107 aa, synthetic construct
  source 1..107: note = 6-10 VL
ALTQPSSVSANPGETVKITCSGGGSYAGSYYYGWYQQKPGSAPVTLIYQDLLRPSDIPSRFSGSTSGSTATLTITGVQAGDEADYYCGSWDSLSSSHFGAGTTLTVL

SEQ ID NO: 245 - protein, 103 aa, synthetic construct
  source 1..103: note = 2-13D VL, 2-13D-37 VL, 2-13D-37-1.5W-41 VL, 2-13D-37-3W-16 VL
ALTQPSSVSANPGETAKITCSGGVYSYGWFQQKPGSALVTVIYWDDERPSDIPSRFSGALSGSTNTLTITGVQAEDEADYYCGTEDISGTAGVFGAGTTLTVL

SEQ ID NO: 246 - protein, 20 aa, synthetic construct
  source 1..20: note = 2-13D-37-1.5W-41 VH-CDR3
SSYVCPGGFSCWGDTGQIDA

SEQ ID NO: 247 - protein, 20 aa, synthetic construct
  source 1..20: note = 2-13D-37-3W-16 VH-CDR3
SNYACPGGFSCWGDTGQIDA

SEQ ID NO: 248 - protein, 8 aa, synthetic construct
  source 1..8: note = 2-13D VL-CDR1, 2-13D-37 VL-CDR1, 2-13D-37-1.5W-41 VL-CDR1, 2-13D-37-3W-16 VL-CDR1
SGGVYSYG

SEQ ID NO: 249 - protein, 122 aa, synthetic construct
  source 1..122: note = S5-2.GKNG VH
AVTLDESGGGLQTPGGALRLSCKASGFTFSSYQMGWVRQAPGKGLEWVSAINKGGSDTSYGSAVKGRATISRDNGQSTLYLQMNSLRAEDTAVYFCAKGSASYITAATIDAWGHGTEVIVSS

SEQ ID NO: 250 - protein, 129 aa, synthetic construct
  source 1..129: note = 2-13D-37 VH
AVTLDESGGGLQTPGGALRLSCSASGFDFSSHGMFWVRQAPGKGLEYVSEITNDGSGTNYGSAVKGRATISRDNGQSTLYLQMNSLRAEDTGTYFCARSTYECPGGFSCWGDTGQIDAWGHGTEVIVSS

SEQ ID NO: 251 - protein, 129 aa, synthetic construct
  source 1..129: note = 2-13D-37-3W-16 VH
AVTLDESGGGLQTPGGALRLSCSASGFDFSSHGMFWVRQAPGKGLEYVSEITNDGSGTNYGSAVKGRATISRDNGQSTLYLQMNSLRAEDTGTYFCARSNYACPGGFSCWGDTGQIDAWGHGTEVIVSS

SEQ ID NO: 252 - protein, 10 aa, synthetic construct
  source 1..10: note = SS01-13-s5 VH-CDR2
AINKSGSDTS

SEQ ID NO: 253 - protein, 10 aa, synthetic construct
  source 1..10: note = S5-2.GKNG VH-CDR2
AINKGGSDTS

SEQ ID NO: 254 - skipped

SEQ ID NO: 255 - protein, 19 aa, synthetic construct
  source 1..19: note = Angiopep-2
  MOD_RES 19: note = hydroxylation
TFFYGGSRGKRNNFKTEEY

SEQ ID NO: 256 - protein, 39 aa, synthetic construct
  source 1..39: note = ApoB (3371-3409)
SSVIDALQYKLEGTTRLTRKRGLKLATALSLSNKFVEGS

SEQ ID NO: 257 - protein, 9 aa, synthetic construct
  source 1..9: note = ApoE (159-167)2
  REPEAT 1..9: note = sequence repeats twice
LRKLRKRLL

SEQ ID NO: 258 - protein, 8 aa, synthetic construct
  source 1..8: note = Peptide-22
  MOD_RES 1: note = acetylation
  MOD_RES 8: note = amidation
  SITE 1..8: note = cysteine in position 1 is linked to cysteine in position 8 as cyclic peptides
CMPRLRGC

SEQ ID NO: 259 - protein, 12 aa, synthetic construct
  source 1..12: note = THR
  MOD_RES 12: note = amidation
THRPPMWSPVWP

SEQ ID NO: 260 - protein, 12 aa, synthetic construct
  source 1..12: note = THR retro-enantio
  MOD_RES 12: note = amidation
PWVPSWMPPRHT

SEQ ID NO: 261 - protein, 9 aa, synthetic construct
  source 1..9: note = CRT
  SITE 1..9: note = cysteine in position 1 is linked to cysteine in position 9 as cyclic peptides
CRTIGPSVC

SEQ ID NO: 262 - protein, 30 aa, synthetic construct
  source 1..30: note = Leptin30
YQQILTSMPSRNVIQISNDLENLRDLLHVL

SEQ ID NO: 263 - protein, 29 aa, synthetic construct
  source 1..29: note = RVG29
  MOD_RES 29: note = hydroxylation
YTIWMPENPRPGTPCDIFTNSRGKRASNG

SEQ ID NO: 264 - protein, 16 aa, synthetic construct
  source 1..16: note = CDX
  MOD_RES 16: note = hydroxylation
GREIRTGRAERWSEKF

SEQ ID NO: 265 - protein, 18 aa, synthetic construct
  source 1..18: note = Apamin
  MOD_RES 18: note = amidation
  SITE 1..11: note = cysteine in position 1 is linked to cysteine in position 11 as cyclic peptides
  SITE 3..15: note = cysteine in position 3 is linked to cysteine in position 15 as cyclic peptides
CNCKAPETALCARRCQQH

SEQ ID NO: 266 - protein, 8 aa, synthetic construct
  source 1..8: note = MiniAp-4
  MOD_RES 1: note = Dpr
  SITE 1..8: note = Diaminoproprionic acid in position 1 is linked to Aspartic acid in position 8 as cyclic peptides
KAPETALD

SEQ ID NO: 267 - skipped

SEQ ID NO: 268 - protein, 13 aa, synthetic construct
  source 1..13: note = G23
HLNILSTLWKYRC

SEQ ID NO: 269 - protein, 6 aa, synthetic construct
  source 1..6: note = g7
  CARBOHYD 6: note = O-linked (Glc...)
  MOD_RES 6: note = amidation
GFGFLS

SEQ ID NO: 270 - protein, 12 aa, synthetic construct
  source 1..12: note = TGN
TGNYKALHPHNG

SEQ ID NO: 271 - protein, 11 aa, synthetic construct
  source 1..11: note = TAT (47-57)
  MOD_RES 11: note = amidation
YGRKKRRQRRR

SEQ ID NO: 272 - protein, 18 aa, synthetic construct
  source 1..18: note = SynB1
RGGRLSYSRRRFSTSTGR

SEQ ID NO: 273 - skipped

SEQ ID NO: 274 - protein, 4 aa, synthetic construct
  source 1..4: note = PhPro
  MOD_RES 1..4: note = Phenylproline
  MOD_RES 4: note = amidation
PPPP

SEQ ID NO: 275 - protein, 20 aa, synthetic construct
  source 1..20: note = F5 Peptide
SDMLPSLEGEGSDLLINRSG

SEQ ID NO: 276 - protein, 20 aa, synthetic construct
  source 1..20: note = F5-1 Peptide
SDMLPSLEGEASDLLINRSG

SEQ ID NO: 277 - protein, 20 aa, synthetic construct
  source 1..20: note = F5-2 Peptide
SDMLPSLEGEGSALLINRSG

SEQ ID NO: 278 - protein, 20 aa, synthetic construct
  source 1..20: note = F5-3 Peptide
SDMLPSLEGEGSDALINRSG

SEQ ID NO: 279 - protein, 20 aa, synthetic construct
  source 1..20: note = F5-4 Peptide
SDMLPSLEGEGSDLLANRSG

SEQ ID NO: 280 - protein, 20 aa, synthetic construct
  source 1..20: note = F5-5 Peptide
SDMLPSLAGEGSDLLINRSG

SEQ ID NO: 281 - protein, 20 aa, synthetic construct
  source 1..20: note = F5-6 Peptide
SDMLPSLEGAGSDLLINRSG

SEQ ID NO: 282 - protein, 20 aa, synthetic construct
  source 1..20: note = F5-7 Peptide
SDMLPSLEGEGSDLLIARSG

SEQ ID NO: 283 - protein, 20 aa, synthetic construct
  source 1..20: note = F5-8 Peptide
SDMLPSLEGEGSDLLINASG

SEQ ID NO: 284 - protein, 20 aa, synthetic construct
  source 1..20: note = F2-01 Peptide
TADRDSSQPRRTIARQTARC

SEQ ID NO: 285 - protein, 20 aa, synthetic construct
  source 1..20: note = F2-02 Peptide
TLARDSSQPRRTIARQTARC

SEQ ID NO: 286 - protein, 20 aa, synthetic construct
  source 1..20: note = F2-03 Peptide
TLDRASSQPRRTIARQTARC

SEQ ID NO: 287 - protein, 20 aa, synthetic construct
  source 1..20: note = F2-04 Peptide
TLDRDASQPRRTIARQTARC

SEQ ID NO: 288 - protein, 20 aa, synthetic construct
  source 1..20: note = F2-05 Peptide
TLDRDSAQPRRTIARQTARC

SEQ ID NO: 289 - protein, 20 aa, synthetic construct
  source 1..20: note = F2-06 Peptide
TLDRDSSAPRRTIARQTARC

SEQ ID NO: 290 - protein, 20 aa, synthetic construct
  source 1..20: note = F2-07 Peptide
TLDRDSSQARRTIARQTARC

SEQ ID NO: 291 - protein, 20 aa, synthetic construct
  source 1..20: note = F2-08 Peptide
TLDRDSSQPRATIARQTARC

SEQ ID NO: 292 - protein, 20 aa, synthetic construct
  source 1..20: note = F2-09 Peptide
TLDRDSSQPRRAIARQTARC

SEQ ID NO: 293 - protein, 20 aa, synthetic construct
  source 1..20: note = F2-10 Peptide
TLDRDSSQPRRTAARQTARC

SEQ ID NO: 294 - protein, 20 aa, synthetic construct
  source 1..20: note = F2-11 Peptide
TLDRDSSQPRRTIRRQTARC

SEQ ID NO: 295 - protein, 20 aa, synthetic construct
  source 1..20: note = F2-12 Peptide
TLDRDSSQPRRTIAAQTARC

SEQ ID NO: 296 - protein, 20 aa, synthetic construct
  source 1..20: note = F2-13 Peptide
TLDRDSSQPRRTIARQTVRC

SEQ ID NO: 297 - protein, 100 aa, synthetic construct
  source 1..100: note = Mature Wild-Type FAM19A5 Isoform 2
QFLKEGQLAAGTCEIVTLDRDSSQPRRTIARQTARCACRKGQIAGTTRARPACVDARIIKTKQWCDMLPCLEGEGCDLLINRSGWTCTQPGGRIKTTTVS

SEQ ID NO: 298 - protein, 100 aa, synthetic construct
  source 1..100: note = M1 Peptide
QFLKEGQLAAGTCEVIAAHRDSSQPRRTIARQTARCACRKGQIAGTTRARPACVDARIIKTKQWCDMLPCLEGEGCDLLINRSGWTCTQPGGRIKTTTVS

SEQ ID NO: 299 - protein, 100 aa, synthetic construct
  source 1..100: note = M2 Peptide
QFLKEGQLAAGTCEIVTLDRCCNKNRRTIARQTARCACRKGQIAGTTRARPACVDARIIKTKQWCDMLPCLEGEGCDLLINRSGWTCTQPGGRIKTTTVS

SEQ ID NO: 300 - protein, 100 aa, synthetic construct
  source 1..100: note = M3 Peptide
QFLKEGQLAAGTCEIVTLDRDSSQPRIEERSQTARCACRKGQIAGTTRARPACVDARIIKTKQWCDMLPCLEGEGCDLLINRSGWTCTQPGGRIKTTTVS

SEQ ID NO: 301 - protein, 100 aa, synthetic construct
  source 1..100: note = M4 Peptide
QFLKEGQLAAGTCEIVTLDRDSSQPRRTIARQTVKCSCFPGQIAGTTRARPACVDARIIKTKQWCDMLPCLEGEGCDLLINRSGWTCTQPGGRIKTTTVS

SEQ ID NO: 302 - protein, 100 aa, synthetic construct
  source 1..100: note = M5 Peptide
QFLKEGQLAAGTCEIVTLDRDSSQPRRTIARQTARCACRKGQIAGTTRNKPSCVDARIIKTKQWCDMLPCLEGEGCDLLINRSGWTCTQPGGRIKTTTVS

SEQ ID NO: 303 - protein, 100 aa, synthetic construct
  source 1..100: note = M6 Peptide
QFLKEGQLAAGTCEIVTLDRDSSQPRRTIARQTARCACRKGQIAGTTRARPACVDARIILQRWWCQMELCLEGEGCDLLINRSGWTCTQPGGRIKTTTVS

SEQ ID NO: 304 - protein, 100 aa, synthetic construct
  source 1..100: note = M7 Peptide
QFLKEGQLAAGTCEIVTLDRDSSQPRRTIARQTARCACRKGQIAGTTRARPACVDARIIKTKQWCDMLPCLEGEECKTLPDNSGWTCTQPGGRIKTTTVS

SEQ ID NO: 305 - protein, 101 aa, synthetic construct
  source 1..101: note = M8 Peptide
QFLKEGQLAAGTCEIVTLDRDSSQPRRTIARQTARCACRKGQIAGTTRARPACVDARIIKTKQWCDMLPCLEGEGCDLLINRSGWTCSCSSGNKIKTTTVS

SEQ ID NO: 306 - protein, 17 aa, synthetic construct
  source 1..17: note = Peptide #1
ACRKGQIAGTTRARPAC

SEQ ID NO: 307 - protein, 19 aa, synthetic construct
  source 1..19: note = Peptide #2
ACRKGQIAGTTRARPACVD

SEQ ID NO: 308 - protein, 20 aa, synthetic construct
  source 1..20: note = Peptide #3
ACRKGQIAGTTRARPACVDA

SEQ ID NO: 309 - protein, 10 aa, synthetic construct
  source 1..10: note = Peptide #4
ARIIKTKQWC

SEQ ID NO: 310 - protein, 12 aa, synthetic construct
  source 1..12: note = Peptide #5
ARIIKTKQWCDM

SEQ ID NO: 311 - protein, 13 aa, synthetic construct
  source 1..13: note = Peptide #6
ARIIKTKQWCDML

SEQ ID NO: 312 - protein, 16 aa, synthetic construct
  source 1..16: note = Peptide #7
ARIIKTKQWCDMLPCL

SEQ ID NO: 313 - protein, 11 aa, synthetic construct
  source 1..11: note = Peptide #8
RIIKTKQWCDM

SEQ ID NO: 314 - protein, 12 aa, synthetic construct
  source 1..12: note = Peptide #9
RIIKTKQWCDML

SEQ ID NO: 315-316 - skipped (2 entries)

SEQ ID NO: 317 - protein, 10 aa, synthetic construct
  source 1..10: note = LRRC4 family mimic molecule
  VARIANT 1: note = X is tyrosine (Y), phenylalanine (F), valine (V), leucine (L), or isoleucine (I)
  VARIANT 2: note = T is threonine (T) or serine (S)
  VARIANT 3: note = Y is tyrosine (Y) or Phenylalanine (F)
  VARIANT 5: note = X is any amino acid
  VARIANT 6: note = T is threonine (T) or serine (S)
  VARIANT 10: note = E is glutamic acid (E) or valine (V)
  VARIANT 7: note = V is valine (V) or isoleucine (I)
XTYFXTVTVE

SEQ ID NO: 318 - protein, 10 aa, synthetic construct
  source 1..10: note = LRRC4 family mimic molecule
  VARIANT 1: note = Y is tyrosine (Y), tryptophan (W), or methionine (M)
  VARIANT 2: note = T is threonine (T) or tyrosine (Y)
  VARIANT 3: note = Y is tyrosine (Y) or tryptophan (W)
  VARIANT 4: note = F is phenylalanine (F), tyrosine (Y), or tryptophan (W)
  VARIANT 5: note = T is threonine (T) or tyrosine (Y)
  VARIANT 6: note = T is threonine (T) or serine (S)
  VARIANT 7: note = X valine (V), tyrosine (Y), phenylalanine (F), leucine (L), tryptophan (W), or methionine (M)
  VARIANT 8: note = T is threonine (T), serine (S), or tyrosine (Y)
  VARIANT 9: note = X is valine (V), isoleucine (I), tyrosine (Y), phenylalanine (F), leucine (L), tryptophan (W), or methionine (M)
  VARIANT 10: note = X is glutamic acid (E), aspartic acid (D), isoleucine (I), tyrosine (Y), phenylalanine (F), methionine (M), or tryptophan (W)
YTYFTTXTXX

SEQ ID NO: 319 - protein, 10 aa, synthetic construct
  source 1..10: note = LRRC4 family mimic molecule
  VARIANT 1: note = X is valine (V), isoleucine (I), tyrosine (Y), phenylalanine (F), or leucine (L)
  VARIANT 2: note = X is threonine (T) or serine (S)
  VARIANT 3: note = X is phenylalanine (F) or tyrosine (Y)
  VARIANT 5: note = X is any amino acid
  VARIANT 7: note = X is valine (V) or isoleucine (I)
  VARIANT 10: note = X is glutamic acid (E) or valine (V)
XXXFXTXTVX

SEQ ID NO: 320 - skipped